(19) United States
(12) Patent Application Publication
(10) Pub. No.: US 2007/0128174 A1
(43) Pub. Date: Jun. 7, 2007

(54) Methods and compositions for organ and tissue functionality

(76) Inventors: Donald A. Kleinsek, Elkart Lake, WI (US); Adriana Soto, Elkart Lake, WI (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/526,202

(22) Filed: Sep. 21, 2006

Related U.S. Application Data
(60) Provisional application No. 60/719,743, filed on Sep. 21, 2005.

(30) Foreign Application Priority Data
Sep. 14, 2006 (US) PCT/US06/35676

(51) Int. Cl. C12N 5/08; A61K 35/36

(52) U.S. Cl. 424/93.7; 435/371; 435/325

(57) ABSTRACT

Materials and methods for treating tissue defects in human or animal tissues using implantable cells are described. Further, culture techniques and factors for enhancing these procedures, and cell survival and adaptation are described. Many of the tissue defects may be treated with autologous cells, while applications involving non-autologous cells or stem cells are also described.

### RELATED APPLICATIONS

This Application claims priority to U.S. Patent Application Ser. No. 60/719,743, which is hereby incorporated by reference herein, and is a continuation-in-part of PCT Application ______ filed Sep. 14, 2006 entitled “Compositions and Methods for the Augmentation and Repair of Defects in Tissue”, which claims priority to U.S. patent application Ser. No. 11/229,237, filed Sep. 16, 2005, each of which are hereby incorporated by reference herein.

Other applications with common inventorship directed to related subject matter include: U.S. patent application Ser. Nos. 09/632,581 (filed Aug. 3, 2000) that claims priority to 60/037,961; and 10/129,180 (filed May 3, 2002) that claims priority to 60/163,734; each of which are hereby incorporated by reference herein to the extent they are consistent with the disclosure herein.

### FIELD OF INVENTION

The field of the invention relates to methods and compositions for the repair or augmentation of defects in human or animal tissues that are primarily due to aging, disease, tissue degeneration, medical disorders, cosmetic conditions, surgery or trauma.

### SUMMARY OF THE INVENTION

Some defects in the body can be treated by the implantation of cells, or particular cell types. Disclosed herein are methods and cell types for treating certain defects. Cells implanted into a patient must survive and adapt to the implant site; techniques for enhancing survival and adaptation are also disclosed.

Some aspects of the invention relate to correcting defect(s) with cells and/or extracellular matrix to improve or restore the functionality of a tissue. Defective tissue becomes structurally altered or dysfunctional as a result of age, disease, degeneration, medical disorders, cosmetic conditions, surgery or trauma, amongst other causes. Dysfunctional or structurally altered tissue can also cause an abnormal or unwanted condition or effect. These alterations are defined as defects. Materials and methods are described herein for augmentation and repair of various tissue defects. In many embodiments cells are taken from a patient, grown in vitro to expand their number, and reintroduced into the patient to treat a defect.

In general, a defect in a patient may be treated with autologous cells when applicable, although in some applications non-autologous cells (e.g,. stem cells) can be used. Usually the implantation is proximal to or in the defect, although some applications of the invention necessitate implantation at a site that affects at another tissue site or sites throughout the body. Alternatively, infusion of the cells into the bloodstream or other fluid cavities can affect a single tissue or a multitude of specific tissues depending on the intended application and homing site of the cells.

As discussed in detail, below, defects and conditions that may be treated include urological sphincter defects resulting in urinary incontinence, fecal incontinence, vesicoureteral reflux, bile duct and gastroesophageal sphincter defects such as gastroesophageal reflux. Skin defects include wrinkles or rhytids, depressed scar or other cutaneous depression, stretch marks, hypoplasia of the lip, prominent nasolabial fold, prominent melolabial fold, acne vulgaris scar, post-rhinoplasty irregularity, hypotrophic scar, and hypertrophic scar, wounds, cellulite, skin laxness, aging skin, need for skin augmentation, and skin thinning. Defects include a breast tissue deficiency, wounds and burns, hernias, periodontal disease and disorders, tendon and ligament tears, baldness, tissue mass adjustment, various tissue and organ fibrosis and sclerosis, tissue scarring, tissue wound, anal fissures, fistulas, hearing loss and disorders, bone defects including osteoporosis, osteomalacia, osteopenia, bone fractures, osteodystophy, bone metabolism defects, alveolar bone defects, cancer, cardiovascular and heart disease, arterial and venous disease, joint and cartilage defects, intervertebral disc defects, Alzheimer's disease, Parkinson's disease, neurological disease and disorders, spinal cord injury, spinal disc defects, hair graying, skin tanning and pigmentation, psoriasis, eczema, eye disease and disorders including cataracts, myopia, presbyopia, hyperopia, macular degeneration of the retina, eye muscle dysfimction, night vision and colorblindness, lacrimal gland dysfunction, interstitial and other lung diseases, kidney dysfunction and failure, renal osteodystrophy, liver dysfunction and failure, dysfunctional pancreas, acute and chronic pancreatitis and diabetes mellitus, endocrine organ dysfunction and disease including the glands of the thyroid, parathyroids, hypothalamus, pituitary, adrenals, pineal, suprachiasmatic nucleus, and endocrine pancreas, immune system disorders, chronic inflammation, adhesions, fibroids, infections, taste and smell defects, gut defects, blood disorders, blood pressure, tooth growth, nail growth, foot enhancement, body thermal regulation, skin and tissue cushioning, mechanical strength of skin and tissues, tissue hydration and elasticity, a deficiency due to aging, organ and tissue replacement, organ or tissue synthesis and whole body rejuvenation.

### DETAILED DESCRIPTION OF THE INVENTION

Tissues are subject to the effects of aging, and become deficient over time. Fortunately, however, it has been discovered that many tissue defects may be treated by adding living cells to the tissue. One effect of aging is the loss of elasticity in tissue. This affects the appearance of the tissue and its function. Described herein are methods of treating a tissue in a patient by expanding a culture of autologous cells in vitro and implanting the cells (preferably autologous) at the tissue to treat the tissue for a deficiency caused by aging. Aging and diseased tissue become dysfunctional in large part due to the loss of appropriate numbers of cell types. This in turn results in lower cell populations and changing gene expression that alter ECM matrix, protein and enzymatic activities (proteases), cell adhesion, cell migration, cell proliferation, cell differentiation, hormone and growth factor production, signaling pathways, feedback mechanisms, tissue homeostasis and dystrophic tissue morphology, amongst other actions, as described in greater detail below.

Many of the defects described herein are a consequence of the aging process. Other defects are due to various disease states and disorders. These tissue defects benefit by replenishment of appropriate cell types and numbers.

An abundance of living cells may be obtained from a relatively small tissue sample when modern cell culture techniques are used. It is thus possible to take a tissue sample from a patient or another source, obtain cells from the tissue, expand the number of cells, and reintroduce the cells into the patient to treat a defect in the patient's tissue. In general, cell types, descriptions of cell types in tissues, tissue architecture and suitable cell and tissue culture techniques are available for the isolation and expansion of the cells, including primary cells, stem cells, and pluripotent cells e.g., in Atlas of Functional Histology, Kerr, J. B., Mosby, 1999; Gray's Anatomy: The Anatomical Basis of Clinical Practice, 39^{th} Edition, Standring, S., Ed., Elsevier, 2005; and Culture of Animal Cells: A Manual of Basic Techniques, Freshney, R. I., Wiley-Liss, Inc., New York, 2000. Certain techniques for isolating and culturing some cell types, including fibroblasts, papillary and reticular fibroblasts are set forth in U.S. patent application Ser. Nos. 09/632,581 (filed Aug. 3, 2000) and 10/129,180 (filed May 3, 2002), which are hereby incorporated by reference herein. Isolation refers to obtaining a purified group of cells from a tissue sample. Expansion refers to increasing the number of cells. In general, expansion and differentiation are inversely related to each other, so that culture conditions that tend to differentiate the cells tend to suppress expansion.

Additionally, the implantation of cultured cells into a patient's tissue has the challenges of helping the implanted cells adapt or “take” to their new site. Even when autologous cells from the patient's own body are used, the cells must still be integrated into the new site and use, or develop, means for receiving oxygen, sources of nutrition, and means for maintaining metabolic activity, amongst other adaptable functions.

Cell culture techniques, treatable defects, factors that improve the successful adaptation of living cells to an implant site, and other information are described in U.S. patent application Ser. Nos. 09/632,581 (filed Aug. 3, 2000) that claims priority to 60/037,961; 10/129,180 (filed May 3, 2002) that claims priority to 60/163,734; PCT Application ______ bfiled Sep. 14, 2006 entitled “Compositions And Methods for the Augmentation and Repair of Defects in Tissue”; and priority document U.S. 60/719,743 filed Sep. 21, 2005; each of which are hereby incorporated by reference herein to the extent they are consistent with what is disclosed herein. Accordingly, the techniques and factors disclosed in these other applications may be combined with the disclosure herein. Thus some embodiments include treating a defect in a patient with in vitro expanded cells and implanting into the tissue defect the cells with a helpful protein or other factor (e.g., proteins, macromolecules, molecules). Examples of such factors include immunogenic proteins, cell adhesion mediating proteins, apoptosis inhibitors, anoikis inhibitors, protease inhibitors, gene of interest, signal transduction proteins, mitogens, differentiation factors, vasodilators, angiogenesis proteins, pro-inflammatory proteins, pro-coagulation proteins, promoters of ECM production, transport proteins, survival factors, a serum protein, cell culture serum-derived proteins and factors, chemoattractants, an ECM protein, growth factor, cytokines, chemokines, hormones, space filling proteins and factors, soluble proteins, insoluble proteins, recombinant proteins, domains and fragments of proteins, peptides, gellable factors, amongst others that are apparent throughout the text and in the art. Depending on the application, other proteins and factors can be used that promote survival of the cells and optimize cell functionality.

Further, these and other proteins and factors can also be useful for the in vitro expansion of the cells. Specific cells and/or proteins can also be useful for the three-dimensional synthesis in vitro of tissue to be implanted in vivo. Preferably, the tissue components simulate the in vivo environment closely. Alternately, the tissue components are functional, yet distinct from the natural in vivo environment.

Thus, various embodiments of the invention include the introduction of cells into a patient to treat a defect using techniques described herein for obtaining, culturing, and introducing cells into a patient. The cells may be introduced with or without the proteins, factors, and supplementing materials described herein. Autologous cells, allogenic cells, or xenogenic cells may be used. Cells include stem cells, various differentiated cells, and their precursors. The site of introduction may be at or near the defect or at a site distant from the defect, as described herein. The various techniques for cell culture and introduction of cells may be applied to any defect described herein, as appropriate for the particular defect.

Treatments for defects described herein generally describe placement of the proper cell type to restore the natural tissue anatomy to elicit the desired tissue functionality. As explained in detail, below, the defects described herein can, in general, be repaired by placing native cells into the defective tissue as guided by the description of the tissue anatomy, suitable cells, and suitable cell equivalents. In general, native cell types are suitable for use in treatment of defects, but native cells or equivalent cells, alone of in combination with various cell types disclosed herein may be used to accomplish the treatment, using the anatomical descriptions and cell functionality guidelines that are set forth. Thus equivalent functional cell types can be used. Defects can be corrected separately or in conjunction with other modalities of treatment, e.g., before, at the same time, or later and/or with proteins and other substances such as polymers. Moreover, stem cells or suitable precursor cells or cells may be used to create the desired cell types by differentiation or transdifferentation, as appropriate. Some of the defects described herein are attributed to particular disease conditions but can also result from aging processes or other diseases; in these cases, the protocols for treating that defect are generally suitable regardless of the exact cause of the defect.

Augmentation and Repair of Bone Defects-the Skeleton, its Function, and Bone Cells

The human skeleton is a complex organ system with two unique and major functions: mechanical and metabolic. The mechanical functions provide the structural framework for the organism that permits support, locomotion and protection of organs. The metabolic function of the skeleton consists of storage of calcium that can be mobilized when needed for vital bodily functions as blood clotting, tissue growth and regeneration and mucous membranes maintenance among others. Bone is also a site for hematopoiesis.

The mechanical properties of the bone result from the combined properties of the components of its extracellular matrix which is composed of organic osteoid (primarily collagen Type I) and an organic mineral phase, in the form of a crystalline hydroxyapatite.

Calcium homeostasis is basically regulated by three hormones: the parathyroid hormone (PTH), 1,25-dihydroxyvitamin D and calcitonin (CT). These hormones regulate calcium levels in serum by actions in mainly three targeting tissues, bone, intestine and kidney, and by controlling the levels of three ions, calcium, phosphate and magnesium. Calcium and phosphate enter the blood from the intestine, are removed through the kidney and stored in the bone. PHT increases bone resorption and calcium re-absorption in the kidney. PHT also regulates the intestinal absorption of calcium by controlling 1-25-dihydroxyvitamin D hydroxylation in the kidney. CT is the antihypercalcemic hormone and inhibits resorption of the bone and renal reabsorption of calcium.

The structure of bones can be defined by macroscopic and microscopic types. Macroscopically there are two general types of bone: dense or compact (cortical) and spongy or cancellous (trabecular). Cortical bone is found primarily in the shafts of the long bones and as the outer layer of virtually all bones. It is 90% calcified. The bony substance is densely packed with cells and intercellular matrix. The marrow cavities are limited. Cancellous bone is found primarily in the vertebrae and at the ends of the long bones. Cancellous bone is a network of interconnected columns and plates which enclose the bone marrow. It is filled with a honeycomb-like network of bony substance consisting of calcified, large, slender spicules called trabeculae with spaces in between. The marrow cavities are large and irregularly arranged. Cancellous bone is only 15-25% calcified tissue, the remainder being marrow, connective or fatty tissue. At the microscopic level, two main histological types of bone (osseous tissue) can be distinguished, woven and lamellar bone. In the woven bone there is a higher volume ratio of cells to matrix, and the matrix is either homogeneous or composed of coarse fibers in angular woven patterns. It is considered to be immature or provisional, to be replaced by more organized bone and is typical of active growth periods such as in fracture callus. Lamellar bone is the mature or adult-type configuration of bone and is transformed from immature bone after puberty. Microscopically it appears as a multilayered matrix synthesized by the orderly accretion of parallel sheets or as osteons (branching interconnected structures that are architectural units of cortical bones), with concentric plates surrounding a blood vessel. Cell density is lower than in woven bone but these cells are interconnected in radiating canaliculi. Not all osteons are equally mineralized at a given time, less mature osteons can contain only 70% of the mineral of mature ones. Osteons are continuously replaced by bone remodeling. Lamellar bone is basic to normal bone remodeling and evolved to repair microdamage inflicted on the tissue by normal wear and tear.

Except at its articular surfaces, bone is surrounded by the periosteum, a specialized connective tissue consisting of two layers, the outer fibrous layer and the inner cellular layer which has cell-forming properties. These bone lining cells reside as a layer of flat and elongated cells on top of the 1-2 μm thick layer of unmineralized collagen matrix covering normal bone. These bone lining cells may be the homing signal for targeting of osteoclasts. The osteocytes instruct the bone lining cells for the need to remodel at a specific time and place. The endosteum, located in the internal periosteum of the marrow cavity of the bones of the limbs, consists of reticular tissue containing osteogenic cells. These bone lining cells located in the periosteum and endosteum can be quiescent osteoblasts or precursors to osteoblasts. These bone lining cells surround the circulatory system of the osteon. These locations provide the progenitor cells to more mature bone cell types and are sources of progenitor cells for expansion and implantation to treat bone tissue defects. Both cancellous and compact bone contain the same cells and intercellular matrix, but differ in the arrangement of these components.

The osteon (Haversian system) is comprised of the Haversian canals, the osteocytes and the intercellular matrix. The Haversian canals are channels that run parallel to the long axis of the bone and carry blood vessels and nerves. These canals are surrounded by concentric layers or lamellae of mineralized intercellular matrix and osteocytes. Volkmann's canals run at right angles to the Haversian canals. These canals connect the osteon with adjacent osteons as well as to the periosteum and endosteum. Osteocytes are located within spaces called lacunae, that are part of the bony lamellae. Osteocytes with cytoplasmic extensions project into channels within the osteon and can contact other osteocytes. These channels, called canaliculi also traverse adjacent osteons and can be a communication means throughout cortical bone. Canaliculi are continuous with the Haversian canal and provide nutrients to the osteocytes. It is a site of exchange of minerals, especially calcium, from the bone to the blood vascular system.

A functional syncytium extends from the osteocytes to osteoblasts which in turn communicates to the adjacent bone marrow cells which extend cellular projections onto endothelial cells inside the sinusoids containing the vessel wall and thus an open circulation between the cells and bone structures.

Cancellous bone shows remnants of osteons remodeled into trabeculae lacking Haversian canals and the lamellae are thin, incomplete and irregularly arranged. Osteocytes inhabit each trabeculum. The surfaces of trabeculae contain osteoblasts, while hematopoietic tissue occupies the marrow cavities.

Blood vessels are numerous in the bone. The network of vessels or the location of the vessels running through the bone are areas that can be used to inject or implant cells into the bone.

Bone development comprises mainly two mechanisms which contribute to embryonic and postnatal skeletal growth. Endochondral ossification is the mechanism by which the bone develops from a cartilaginous template as occurs in the bone of the limbs and the axial skeleton. In contrast, membranous ossification consists of the embryonic condensation of fibrocellular mesenchyme that precedes the appearance of bony spicules that forms the vault of the skull, clavicle, maxilla, mandible and the facial bones. Thus, bones of the skull and face do not require cartilage for their formation whereas most other bones depend on the initial development of the cartilaginous model of the bone which is gradually replaced by the deposition of a mineralized matrix (bone).

Cellular components of bone tissue consist largely of osteoblasts, osteocytes and osteoclasts, bone-lining cells, osteogenic progenitor bone cells, stromal cells (e.g. fibroblasts) and minor quantities of monocytes/macrophages and mast cells. Osteogenic progenitor bone cells refer to stem cells (e.g., pluripotent, multipotent) or precursor cells that give rise to bone-forming and bone-destroying cells. A precursor refers to a stem cell or other cell that is not completely differentiated. A bone cell refers to an osteogenic progenitor bone cell, a differentiated osteogenic or bone-lining cell, and precursors thereof, including: osteocytes, osteoblasts, osteoclasts, and precursors of the same. A specialized bone cell refers to an osteoblast, osteoclast, or osteocyte.

Bone-lining and precursor bone cells can be osteogenic, in the lineage leading to the formation of osteoblasts. Precursor bone cells can be in the lineage leading to the formation of osteoclast cells, resulting in resorption of the bone. The precursors of osteoblasts are multipotent mesenchymal stem cells which can also give rise to further differentiated cell types such as chondrocytes, adipocytes and muscle cells. These osteoblast progenitors may originate from marrow stroma or pericytes, the mesenchymal cells adherent to the endothelial layer of vessels, such as is present in the inner layer of the outer periosteum. Precursors of osteoclasts are hematopoietic cells of the monocyte/macrophage lineage. Whereas osteoblast precursors most likely reach bone by migration of progenitors from neighboring connective tissue, osteoclast precursors reach bone from the circulation.

Autocrine, paracrine and endocrine signals influence the development of osteoblasts and osteoclasts, as well as cell-cell and cell-matrix interactions. Beside cell development and apoptosis, adhesion molecules are involved in the migration of progenitor cells from bone marrow to the sites of bone remodeling, as well as the cell polarization of osteoclasts and the beginning and end of osteoclastic bone resorption. Some of the adhesion molecules are the integrins (αᵥβ₃ and α₂β₁), selectins, and cadherins, and a family of transmembrane proteins containing a disintegrin and metalloprotease domain (ADAMS). These proteins interact and recognize other ligands, such as some integrins that recognize the RGD amino sequence present in collagen, fibronectin, osteopontin, thrombospondin, bone sialoprotein and vitronectin. Thus cell adhesion proteins in tandem with the bone cells described can assist in cell survival after implantation.

The osteoblasts are mono-nucleated cells derived from mesenchymal progenitor cells present in the bone marrow and other connective tissue. Their main major functions are to synthesize and secrete collagen (type 1) and proteoglycan complexes that constitute osteoid and to play a role in matrix mineralization. Other functions of osteoblasts are to regulate the movement of calcium, magnesium and phosphate in and out of bone fluids and mediate the stimulation of bone resorption by responding to the systemic hormones parathyroid hormone (PTH), growth hormone, thyroid hormone, androgens, and insulin. Glucocorticoids are potent inhibitors of osteoblastic activity. Growth factors such as the bone morphogenetic proteins (BMPs, e.g., BMP 2,7) are involved in skeletal development during embryonic life and fracture healing. BMPs stimulate an osteoblastic-specific transcription factor, core binding factor al (Cbfal). Other growth factors such as transfonning growth factor Beta (TGFBeta), platelet-derived growth factor (PDGF), insulin-like growth factors (IGFs) and members of the fibroblast growth factor (FGF) family influence the replication and differentiation of committed, not uncommitted, osteoblast progenitors toward the osteoblastic lineage. Cells of the stromal/osteoblastic lineage produce interleukin 6 (IL-6) in response to the above growth factors including interleukin 1. IL-6 influences the differentiation of osteoblasts. Osteoblasts synthesize intercellular matrix comprising type I collagen, osteocalcin, osteonectin, biglycan, decorin (implicated in collagen fibrillogenesis), osteopontin, bone sialoprotein, fibronectin, vitronectin and thrombospondin, hydroxyapetite (Ca, phosphate).

Osteocytes are osteoblasts completely surrounded and reside in a lacunae with mineralized matrix, but maintain cytoplasmic connections with other osteocytes and with surfaced osteoblasts. There are 10 times the amount of osteocytes than osteoblasts and are the most abundant cell type in bone. This network of cells provides continuity with the vascular circulation. The functions of the osteocytes are to maintain minute-to-minute exchange of mineral in the bone matrix and to serve as transducers of mechanical loading of bone. The piezoelectric property of bone matrix allows for transmission of load throughout the skeleton sensed by the osteocytes and osteoblasts that respond to external forces, compression and tension and effect changes in the internal architecture of the bone. The osteocytes are candidates for mechanosensory cells to detect the need for bone augmentation or reduction in functional adaptation of the skeleton, the need for repair of microdamage. They are the only cells in bone that senses the need for remodeling at a specific time and place.

Osteoclasts are large, multinucleated cells (50 to 100 μm diameter) found mainly on the surface of the bone. They are the major cells responsible for bone resorption and remodeling. This is accomplished by a cytoplasm concentrated with lysosomes containing lytic enzymes. Osteoclasts have abundant calcitonin receptors. Osteoclastic bone resorption is stimulated by PTH and 1-25-dihydroxyvitamin D₃ and inhibited by calcitonin. PTH and 1-25-dihydroxyvitaminD₃, by stimulation of osteoclast development and regulation of calcium absorption and excretion from the intestine and kidney, respectively, are key elements in extracellular calcium homeostasis. Osteoclast development is stimulated by the interleukins 1, 3, 6, 11, leukemia inhibitory factor (LIF), oncostatin M, ciliary neurotropic factor, tumor necrosis factor, granulocyte macrophage-colony stimulating factor (GM-CSF, M-CSF) and c-kit ligand. Interleukins 4, 10, 18 and γ inhibit osteoclast development. Osteoclasts are formed from a branching off of the early osteoblastic lineage committed by mesenchymal progenitors and prior to further differentiation into the osteoblast or adipocyte pathways by a commitment of the mesenchymal progenitor cells.

Two models of osteoblast recruitment are the serial and parallel. In the serial model, resorbed bone releases factors and local increases in mechanical strain stimulate osteoblast precursor cell proliferation and differentiation. In the parallel model, both osteoblast and osteoclast precursor proliferation and differentiation occur concurrently in response to a signal for the initiation of new BMUs. Both models require the osteoblasts to be in the right location.

Osteoclast and osteoblast development is stimulated by IL-6 made by osteoblasts. The two cell types work in temporal and spatial tandem to remodel bone.

The bone marrow stroma contains stem cells that can convert between the osteoblast and adipocyte phenotype. Stromal fibroblasts, pre-adipocytes and adipocytes, epithelial and endothelial cells reside in the stroma. Stromal cells can be also used in other tissue defects than bone and can be converted into specific cell types of other tissues (e.g. mesenchymal stem cells). Cells of the bone marrow support hematopoiesis, osteoclastogenesis, fat and bone formation. The conversion of stromal cells among phenotypes and commitment to a specific lineage with suppression of alternative phenotypes is dictated by transcription factors and signal transduction pathways through external stimuli such as growth factors and hormones.

Remodeling is defined as the removal and replacement of bone tissue without altering its overall shape. Remodeling of bone is accomplished by the processes of bone removal (resorption), done by osteoclasts and bone formation, done by osteoblasts. In the uninjured skeleton, osteoclasts and osteoblasts belong to a temporary structure called the basic multicellular unit (BMU). The BMU is about 1-2 mm long and 0.2-0.4 mm wide, is comprised of a team of osteoclasts in the front, a team of osteoblasts in the rear, a central vascular capillary, a nerve supply, and associated connective tissue. The cellular components maintain a well orchestrated spatial and temporal relationship to each other. Osteoclasts adhere to bone and remove it by acidification and proteolytic digestion. As the BMU advances, osteoclasts leave the resorption site and osteoblasts move in to fill new bone formation in the excavated area by secreting osteoid, which is later mineralized into new bone. In cortical bone, the BMU moves through the bone, excavating and replacing a tunnel. In cancellous bone, the BMU moves across the trabecular surface, excavating and replacing a trench. The first phase of origination, begins at a specific location and time followed by the second phase, progression, an advancement toward a region of bone in need of replacement and for a variable distance beyond until coming to rest, known as the third phase, termination. The lifespan of a BMU is 6-9 months, longer than the 2 weeks of an osteoclast or 3 months of an osteoblast. Thus a supply of new osteoclasts and osteoblasts from their progenitors is needed from the bone marrow for the origination of BMUs and their progression on the bone surface. To maintain bone homeostasis, there is a balance between the supply of new cells and their lifespan to determine the number of cells and the work performed by each type of cell. Bone resorption and formation are happening simultaneously in which osteoblasts assemble at sites only where osteoclasts have recently completed resorption. This activity is known as coupling. Thus, while resorption advances bone formation begins to occur. In healthy adults, 3-4 million BMUs are initiated annually and at any one time about one million BMUs are active. Remodeling can be enhanced by the introduction of MMP (matrix metalloproteases) in tandem with bone forming cells.

Modeling is defined as alterations in bone tissue shape by the resorption and appositional bone growth in the periosteum and endosteum. During the process of modeling, an anatomical BMU is not distinguishable, but the growing skeleton still requires spatial and temporal orchestration of the destination of osteoclasts and osteoblasts that are different from remodeling of bone.

The bone extracellular matrix can be considered as the interstitial or intercellular matrix. The osteoblast secretes individual collagen (type 1) molecules that aggregate in fibers constituting the osteoid or organic phase of the bone. The mature collagen fibrils are rendered less soluble. Proteoglycans and hyaluronan comprise the ground substance in the organic matrix of the bone, also produced by the osteoblast. The rigidity of the bone is provided by the mineralized fraction. Bone hydroxyapatite is an imperfect crystal of calcium phosphate salt having substitutions of magnesium, sodium, strontium, carbonate, citrate and fluoride. The hydroxyapatite crystal structure within the bone has a high surface area capable of exchange with the extracellular fluid. Mineralization of the organic matrix of the bone occurs by precipitating mineral. Alkaline phosphatase contributes to mineralization by increasing the local concentration of inorganic phosphate to cause spontaneous precipitation of hydroxyapatite. In lamellar bone the early mineral crystals appear within collagen fibrils. In woven bone mineralization begins with membrane-bound matrix vesicles in the extracellular tissue space.

Treatment of Bone Defects

Bone defects may be treated by introducing bone cells into the patient at appropriate sites, as explained herein. Specialized bone cells or their precursors (e.g. bone marrow mesenchymal stem cells or other stem cell types such as muscle derive stem cells) may be introduced at or near a bone defect site. The introduced cells adapt to the bone architecture at or near the site to effect a repair. Osteogenic precursor cells may be introduced at a point distant from the site, e.g., vascularly. Such precursors can home-in on bone defect sites, where they adapt to the site to effect a repair. Cells may also be introduced at a site calculated to bring the cells into close proximity to a bony defect. For instance, bone cells may be introduced into a blood vessel that flows into or near the defect. In particular, bone cells may be introduced into an artery, arteriole, vein, or venule that flows through the bony defect. Or, for instance, bone cells may be introduced into a biological space that communicates with the defect. In particular, bone cells may be introduced into a marrow cavity that serves a bone having a defect. Or bone cells may be introduced into the network of vessels and/or canals that serve the defect. In particular, bone cells may be introduced into cancellous bone that is associated with a defect, for instance, at a distance of about 1 cm to about 50 cm of the defect site; persons of ordinary skill will appreciate that all ranges within these bounds are contemplated, e.g., within about 1 cm to within about 30 cm, as well as other distances not set forth within the explicitly stated range.

As explained in greater detail, below, bone cells may be introduced with or without additional materials such as matrices, extracellular matrix, fillers or carriers such as hydroxyapatite. In general, such materials may be readily used when the cells are introduced at or near the defect. When cells are introduced at relatively more remote positions, the effect of such materials on delivery of the cells must be considered; for example, large amounts of filler are not suited for delivery into the vascular system. In some embodiments, the cells are introduced with helpful proteins (such as TGFB3, bone morphonogenic proteins 2, 3 and 7) or other factors (such as gene therapy to deliver the specific inducing growth factors) calculated to help the cells adapt to the patient, e.g., as in PCT Application ______ filed Sep. 14, 2006 entitled “Compositions And Methods for the Augmentation and Repair of Defects in Tissue” which are hereby incorporated herein by reference. Bone cells may be administered in a single treatment, or repeatedly administered over time. Further, treatments may be combined, e.g., with different sites of delivery or in combination with drug therapies.

Metabolic Diseases of the Bone

Bone defects caused by a bone resorption diseases, by decreased bone formation, and by other causes of bone loss or bone disease may be treated by introducing bone cells into the patient. Bone-resorption diseases are characterized by abnormal increased bone resorption, and include osteoporosis, osteopenia and several others.

Osteoporosis is the consequence of the loss of bone strength and is the most common metabolic bone disease. It is estimated that osteoporosis causes 1.5 million fractures annually in the U.S. These fractures occur mainly in the spine with great morbidity, resulting indirectly in higher mortality rates. Although a gradual decline in bone density occurs with aging in both sexes, osteoporosis results from an exaggerated imbalance between resorption and formation. Type I (high turn-over) osteoporosis is related to estrogen deficiency, which affects post-menopausal women between the ages of 50-65. Accelerated trabecular bone loss occurs, mainly affecting the vertebrae, and therefore increasing the risk for fractures. Type II (low turn-over) osteoporosis afflicts most women and men over 75 years of age and involves loss in both types of bone, trabecular and cortical. This result in an increased risk for hip and vertebral fractures. Type II is due to an age-related declined in osteoblast function and number that can not surpass the osteoclast activity.

The three most common causes of bone loss are sex steroid deficiency, glucocorticoid excess and aging. In sex steroid deficiency or glucocorticoid excess, notable cellular changes in osteoblastogenesis and osteoclastogenesis in which there is an oversupply of osteoclasts relative to the need for remodeling. The lifespan of osteoclasts are increased, but decreased for osteoblasts or osteocytes. In sex steroid deficiency osteoclasts erode deeper than normal cavities due to an increased lifespan of the osteoclasts (delay of apoptosis) resulting in trabecular perforation. Increased adipogenesis is seen with glucocorticoid excess.

Osteopenia is a decrease in wall thickness, especially in trabecular bone, and is a hallmark of aging bone. The change in thickness or loss of bone density is determined by the number or activity of osteoblasts at the remodeling site. In aging this effect is local and relative to the demand created by resorption. In aging there is a decrease in osteoblastogenesis and osteoclastogenesis and a decrease in the lifespan of osteocytes, in which there is an undersupply of osteoblasts relative to the need for repair. There is an increase in adipogenesis as well.

Other bone diseases displaying abnormal increased bone resorption occurs in Paget's Disease due to excessive remodeling of the bone caused by the presence of an abnormally larger number of active osteoclasts, in Osteitis Fibrosa Cystica due to parathyroid hormone excess and in Humeral Hypercalcemia of Malignancy due to active metabolic bone metastasis located in the humerus that may occur as a result of cancers such as breast, lung, esophagus, cervix, vulva, ovarian, amongst others.

In some embodiments, bone resorption diseases can be treated by the introduction into the patient of osteoblasts or osteoblast progenitor cells to offset osteoclast activity and/or factors that inhibit osteoclast activity. As already described, cells may be introduced at or near the defect or at a relatively more distant point.

Diseases characterized by decreased bone formation include osteopenia, osteomalacia, and renal osteodystrophy. Osteomalacia is a deficient mineralization of the skeleton that is called rickets in children and osteomalacia in adults. Both forms are the result from a deficiency in the factors important in bone formation, calcium, phosphorus, vitamin D, and alkaline phosphatase. Although dietary deficiency of vitamin D is rare in developed countries, malabsorption disorders or impairment of renal activation of vitamin D (congenital or acquired) results in osteomalacia with the characteristic weakness of the skeleton, flattening of the skull and pelvis, bowing of the legs in children and bone pain and radiological lesions in adults. Renal Osteodystrophy occurs in chronic and advance renal failure and it is due to impaired kidney metabolism of vitamin D and secondary hyperparathyroidism. Osteogenesis Imperfecta (OI) is a congenital genetic disorder caused by a defective type I collagen. A treatment for OI should be aimed toward improving bone strength by enhancing the structural integrity of collagen to prevent the numerous fractures characteristic of the disorder.

Bone Fractures

Bone defects caused by a fracture may be treated by introducing bone cells into the patient, for instance, as already described with respect to bone diseases, above. For instance, bone cells may be introduced directly into the fracture, or nearby. Bone fractures caused by osteoporosis comprise the hip, wrist and vertebrae. Bones of the hip, wrist and vertebrae, consist primarily of the more delicate spongy bone. This is why these areas are more prone to fracture. Spongy bone is also more metabolically active than compact bone. This means that bone turnover is higher in spongy bone. Increased bone turnover hastens bone loss, making spongy bone more susceptible to fracture. Vertebral compression fractures and hip fractures are particularly devastating consequences of osteoporosis.

Vertebral compression fractures happen most often in the thoracic region, or middle section, of the spine. A simple movement, such as bending or lifting, may cause the fracture. Over a period of time, multiple fractures of the fronts of the vertebrae may collapse and wedge together. This will cause the spine to bend forward, and develop a rounded back, commonly called a dowager's hump, or kyphotic deformity. Complications of vertebral fractures include loss of height, back pain and stooped posture. With multiple vertebral fractures, bending, lifting, reaching, climbing and walking become difficult.

The most serious consequence of osteoporosis is the hip fracture. Women are two to three times more likely than men to break a hip. Nearly one-third of patients who fracture a hip will enter a nursing home within a year. A hip fracture is also associated with a 10% to 20% death rate within the first year.

According to the American Academy of Orthopaedic Surgeons (AAOS) fractures are among the most common orthopedic complaints, with approximately 7 million broken bones each year in the U.S., comprising five basic types of bone fractures. These fractures include a simple fracture in which the bone is broken in one place but the skin is not broken; a compound fracture in which the skin is broken; a transverse fracture in which the break is at a right angle to the length of the bone; a greenstick fracture in which the break is only on one side of the bone and the bone bends; and a comminuted fracture in which there are at least three bone fragments.

Bone Healing After a Fracture

There are several stages in bone healing after a fracture. Stage 1 is inflammation. In this stage bleeding from the fractured bone and surrounding tissue causes the fractured area to swell. This begins on the day of the fracture and can last for 2 to 3 weeks. The bleeding brings cells such as immune cells into the fracture site that are needed to perform several ftmctions, such as cleansing of the site from debris. The influx of new cells, such as osteoblasts, may start the bone healing by forming granular tissue. After the pain and swelling decreases, the soft callus stage begins in which the site of the fracture stiffens and new bone begins to form, but is not visible on x-rays. This stage can last for 4 to 8 week post-injury.

The new bone begins to bridge the fracture and can be seen on x-rays. In stage 3, 8 to 12 weeks post-injury, the hard callus stage occurs in which new bone has filled the fracture and the fracture site remodels itself. This bone remodeling stage 4 corrects any deformities that may remain as a result of injury. This final stage of fracture healing can last up to several years.

Current treatments for fractures include mechanical and grafting procedures. Since the bone is constantly in a state of turnover in a process known as remodeling, the process of healing bone often comes about naturally. In order for the fracture to heal as quickly as possible, without any deformity, the bones must sometimes be first put back in proper position. This is called “reduction” and involves putting the broken bone in a cast, after the doctor manipulates the bone into proper alignment. The use of casts is called external fixation. On the other hand, surgery may be required for more complicated breaks such as comminuted fractures. Surgery is known by the term internal fixation and uses several materials such as wires, plates, nails, rods and screws. When bone is lost in a fracture and a gap needs to be filled in order to promote bone healing, both vascularized and non-vascularized autologous bone is used. Frequently not enough autologous material is available and a bone allograft from a bone bank is required. Some of the drawbacks in the use of allografts are host rejection and viral contamination.

There is a need to increase the availability of the patient's own bone material. This can be accomplished by the in vitro cell expansion and use of autologous bone cells or bone precursor cells or a combination of these cells with different biomaterials (e.g., biologically active glass or polymers), minerals (e.g., calcium phosphates), combination of growth factors (e.g., vascular endothelial and fibroblast growth factors), extracellular matrix and its components to restore form and function to the deficient (osteoporotic) or healing (fracture) bone.

Placement of Bone Cells

Bone cells can, in general, be obtained by removal from the bone marrow. Bone marrow can be obtained from the donor's pelvic bone (ileum) or by needle aspiration into other bone areas. Bone cells from the peritoneum can be obtained, for example, by scraping the outside of the bone or from the endosteum.

One method to place bone cells into a bone defect is to inject the cells into a vein of the patient, particularly a vein that flows through the defect area. From the site of injection, the bone cells travel to the bone marrow space, where they produce new cells and/or travel to the BMU. Another method to treat local defects in the bone is to inject the cells at or near the site of the defect. The network of vessels or the location of the vessels running through the bone, such as canals or at the ends of articular bones, are areas that can be used for injection or implantation of cells back to the bone. For example, the Haversian canal or other canals or vessels that allow the delivery of bone forming cells can be used. In a preferred embodiment, placement of cells into or under the periosteum can be suitable for delivery of cells within the bone site of interest. Cells and/or extracellular matrix, polymers, other compounds, factors, compositions can be packed into bony voids or gaps of the bone. These defects can be surgically created as well as from traumatic injury to the bone or due to other defects described. The packing can be accomplished by injection directly in or near defect or by inclusion in a paste or matrix that adheres to the defect site. Placement sites for bone grafting can be the extremities, spine and pelvis for example. Another method to return the proper bone cells and/or extracellular matrix is by direct injection through a syringe into the bone body. Alternately, a balloon injection techniques can be employed or cutting and patching of the bone site can be used. Methods including injection, engraftment, engraftment by threading and direct placement, direct placement with or in conjunction with a suitable vehicle can be used. Repetitive treatments can be used such as repetitive direct injections into a bone site. Other placement procedures may be used.

For instance, osteogenic cells can be used in bone grafts. Such treatment can be indicated for acute long bone fractures, bone trauma defects, voids and gaps that are not dependent on the stability of the bone structure.

Another approach to prevention and reduction or elimination of osteoporosis, osteopenia, rickets or osteomalacia is to augment the patient's skin with cells from the dermal, subcutaneous and fascial layers. In particular, connective tissue cells such as fibroblasts (e.g., dermal, fascia), preadipocytes and keratinocytes that can increase the production of vitamin D in the skin can increase bone formation through the pathways discussed above. This can be accomplished by exposure of the implanted skin areas of the patient to sunlight or artificial UV, such as the back of the hands, forearms, face, legs, torso, etc. This distal placement of cells to the bone can also be used for the prevention, health and healing ability of bone fractures or of bone and its constituents and metabolic processes, bone density augmentation, bone defects, amongst others. This includes, for example, the treatment of osteoporosis (types I and II), osteopenia, ostoemalacia, amongst others. This approach can also be used for other defects in other tissues that vitamin D is known to treat. This includes an increase in immunity, muscle strength, cancer prevention and treatment (e.g., colon, breast, ovarian cancers), psoriasis, periodontal disease, autoimmune disease such as rheumatoid arthritis, inflammatory bowel disease, multiple sclerosis, high blood pressure and heart disease.

Various pharmacological approaches are used to prevent and treat bone loss, irrespective of the cause. This includes estrogen replacement therapy, bisphophonates, relixifene, calcitonin, sodium fluoride, calcium, and vitamin D. Glucocorticoid induced osteoporosis can be treated with parathyroid hormone. These treatments can be an adjunct treatment with the introduction of osteogenic cells.

The various growth factors in bone development and metabolism (e.g., Bmp-2, -7) can be used in tandem with cell introduction for the variety of bone forming, repair or remodeling processes.

Osteogenic cells can be obtained from several sites in the bone and can be used for the variety of metabolic and bone defects herein. The imbalance of osteoclast activity and osteoblast activity in the turnover of bone in several metabolic disease states can be corrected by the addition of osteoblasts or osteoprogenitor cells and/or in tandem with osteoclast reducing activity agents such as calcitonin. Osteogenic cells include osteoprogenitor cells (mesenchymal stem cells that lead to osteoblasts formation), osteoblasts, osteocytes and fibroblasts from bone marrow stroma or fibroblasts from other areas of the body (e.g. dermal fibroblasts).

Treatment can be effected by placing an effective volume of cultured bone cells and/or extracellular matrix into bone tissue or site of defect of bone tissue. Bone cells can be obtained from locations described in the text such as from bone marrow or bone biopsy. For example, osteoprogenitor cells can be obtained from the bone marrow or bone line cells in the periosteum or endosteum. Osteoblasts can be obtained from the bone marrow or intercellular matrix. Osteocytes can be obtained from the osteon. Osteoclasts can be obtained from bone marrow.

Osteoclasts and osteoclast progenitor cells can be used where the bone needs to be remodeled and/or repaired. Examples would be bone pseudo-arthrosis due to abnormal or incomplete consolidation of a fracture (e.g., non-union) and/or the formation of temporal or incomplete bone callus in otherwise normal individuals, in fractured bones compromised by osteomielitis as well as in patients with diseases characterized by decreased bone formation include osteopenia, osteomalacia and renal osteodystrophy.

The replacement of BMUs can be done for bone defects by the proper kinetic and sequential introduction of osteoclasts and osteoblasts. Implantation into the bone site with these cells to effect proper bone remodeling can occur by separate introduction spatially and temporally of these cells. Alternately, matrices that release these cells in the proper manner can be used. Thus, for example, a matrix can have spatially different cell components. Natural or synthetic polymers can be front-loaded and effect the release with osteoclasts first followed by a back-loaded osteoblast filled polymer layer that allows the preferential release of the osteoblasts.

Prior to implantation, bone cells can be placed in matrices, such as in a patient's clot, fibrin compound, pastes, bone cell ECM, other connective tissue ECM or its constituent proteins single or in combination, other biomaterials (biodegradable, acellular, biologically active glass, polymers), or minerals (calcium phosphates), or a combination of growth factors (vascular endothelial and fibroblast growth factors) and cells with polymers and minerals or matrices (collagen), amongst other matrices that are described in the text or known in the art. The goal, as with bone cells alone, is to restore form and function to the deficient (osteoporotic, osteopeniac, osteomalactic or osteodystrophic) or healing (fracture) bone. Additionally, these non-cell additives can be used without cells to treat bone defects in certain cases.

The bone cells can be used to correct a simple, compound or comminuted bone fracture. This can be performed with repetitive injections and/or open applications of the cells into the fracture site. The viable expanded bone cells can be used to correct a vertebral fracture, a collapsed vertebral body, a hip fracture, a wrist fracture or damage to these bone sites caused by osteoporosis or osteopenia by using repetitive injections or applications into the bone defect area.

The bone cells can be used to treat bone defects and conditions due to osteoporosis, osteopenia, aging, sex-steroid insufficiency, glucocorticoid excess, fractures, bone grafts, amongst others. Thus certain embodiments include methods and devices for the treatment of chronic (e.g., osteoporosis, osteomalacia, osteodystrophy or any other bone metabolic deficiency) and acute bone defects (fractures) by means explained above.

Augmentation and Repair of Hearing and Ear Defects

The ear is anatomically divided in three portions: the outer ear, the middle ear and the inner ear. The outer ear starts with the ear itself or pinna, a cartilaginous structure. The outer ear is continuous with the ear canal, the length of which is approximately 1 inch in the adult. This area is cartilaginous in its external half and bone layered and covered by skin in its internal half before ending at the eardrum. This skin is provided with specialized ceruminous and sebacious glands that produce the ear wax. The eardrum or tympanic membrane, which divides the outer and middle ear, has three layers. It is divided into portions, the upper portion is the pars flaccida and the lower portion is the pars tensa.

The middle ear is formed by three small bones. The first bone, the malleus (hammer) is attached to the tympanic membrane. The small bone in the middle is the incus (anvil) and the inner bone is the stapes (stirrup). The Eustachian tube connects the middle ear with the nasopharynx. The middle ear ends at the oval or round window, which divides the middle ear and the inner ear.

The inner ear contains the cochlea, a snail's shell like structure that is the sensory organ of hearing. The cochlea is filled with liquid and layered with specialized cells featuring cilia (hairs). These hair cells, originate from embryologic ectoderm. The auditory nerve originates within the cochlea, joining the vestibular portion coming from the vestibular labyrinth, (which senses the body's position and rotation to reach equilibrium) and going into the VIII cranial nerve or vestibulocochlear nerve. The labyrinth is a group of canals and two rounded structures (the utricle and the saccule) that contain fluid and fine cellular hair-like sensors.

The Eustachian tube (pharyngotympanic tube) connects the middle ear to the lateral wall of the nasopharynx just above the plane of the floor of the nose. Its total length is approximately 36 mm, and its direction downward, forward, and inward, forming an angle of about 45° with the sagittal plane and one of from 30° to 40° with the horizontal plane. It is lined with respiratory type columnar epithelium perpendicular to the basal laminae forming mucous membrane. The cartilaginous or medial portion of the Eustachian tube closest to the nasopharynx is about 24 mm long. The osseous portion extending from the middle ear is approximately 12 mm long. The diameter is greatest at the nasopharyngeal end, narrowing to an isthmus at the junction of the cartilaginous and bony portions. The function of the Eustachian tube is to provide a passage from the nasopharynx to the ear, equalizing the pressure on both sides of the eardrun. If the pressure of the external ear canal is greater than that in the middle ear, the eardrum is displaced inward. If the pressure in the middle ear is greater than that of the external canal, the eardrum bulges outward.

Hearing Loss

Hearing is an extremely dynamic and fast process. The pinna gathers and pushes sound into the ear canal, where the sound waves hit. The eardrum then vibrates rapidly, transferring the sound waves to the three bones. These bones then vibrate and transfer the mechanical impulse to the oval window. The oval window itself vibrates and moves the cilia of the hair cells inside the cochlea. This process causes depolarization, converting a mechanical impulse into an electrical one, that is then delivered to the auditory nerve which passes into the brain to integrate, relate and respond properly to the sound.

The intensity of sound is measured in decibels (dB). A whisper is about 20 dB, loud music (some concerts) is around 80 to 120 dB, and a jet engine is about 140 to 180 dB. Usually, sounds greater than 85 dB can cause hearing loss in a few hours, louder sounds can cause immediate pain, and hearing loss can develop in a very short time. The tone of sound is measured in cycles per second (cps). Low bass tones range around 50 to 60 cps, while shrill, high-pitched tones range around 10,000 cps or higher. The normal range of human hearing is about 16 cps to 16,000 cps. Some people can hear within a slightly higher range, and animals can hear up to about 50,000 cps.

Types of Hearing Loss

Minor decreases in hearing, especially of higher frequencies, are normal after age 20. Some nerve deafness (or loss of hearing) affects 1 out of 5 people by age 55. It usually comes on gradually and rarely ends in complete deafness. There are three different categories of hearing loss depending on the area of the ear affected.

Sensorineural hearing loss occurs when the “inner” ear and/or the actual hearing nerve itself becomes damaged. About 90% of all people with hearing impairments are in this category making it the most common type of hearing impairment. Sensorineural hearing loss is often referred to as “nerve deafness”. Nerve deafness is not a good description because the damage usually occurs within the inner ear (the hair cells of the cochlea) and not the hearing nerve. Common causes of sensorineural hearing loss are ageing and exposure to loud noises.

Conductive hearing loss occurs when the “outer” or “middle” ear fail to work properly. Sounds become “blocked” and are not carried all the way to the inner ear. Conductive hearing losses are often treatable with either medicine or surgery. Common causes of conductive hearing loss are fluid buildup in the middle ear or a blockage of wax in the ear canal. Children are more likely to have a conductive hearing loss than a sensorineural hearing loss.

Mixed hearing losses are simply combinations of the above two types of hearing loss. It can occur when a person has a permanent sensorineural hearing loss and then develops a temporary conductive hearing loss.

Age-related hearing loss (presbycusis) involves a progressive series of events. For example, it can begin with high-frequency sounds, such as speech. This can occur as a result of hereditary factors, various health conditions, and side effects of some medicines (aspirin and certain antibiotics). Presbycusis may be caused by changes in the blood supply to the ear because of heart disease, high blood pressure, vascular conditions such as that caused by diabetes, or other circulatory problems. It is unknown if there is a specific cause such as noise trauma, but there appears to be a genetic predisposition. Age-related hearing loss tends to occur in families. The disorder occurs in about 25% of people ages 65 to 75 years old and in 50% of those over age 75.

The loss associated with presbycusis is usually greater for high-pitched sounds. There are many causes of presbycusis. Most commonly it arises from changes in the inner ear of a person as he or she ages, with hair cells being lost in the basal end of the cochlea. Presbycusis can also result from changes in the middle ear or from complex changes along the nerve pathways leading to the brain. Presbycusis most often occurs in both ears, affecting them equally. Because the process of loss is gradual, people who have presbycusis may not realize that their hearing is diminishing.

Sensorineural hearing loss is usually not medically or surgically treatable using conventional treatments. Usually an otolaryngologist evaluates the individual with a hearing problem to make the diagnosis and exclude related systemic disorders that may contribute to the problem. An audiologist is a professional who measures the hearing and identifies the type of hearing loss. The audiologist conducts a complete hearing evaluation and determines if a hearing aid may be useful. The individual is counseled about how a hearing aid may improve listening situations. Then the audiologist conducts tests to find an appropriate aid, selecting one that maximizes a person's hearing and understanding of speech. Most older adults with hearing loss can benefit from using a hearing aid, although the degree of benefit may vary according to the type and amount of hearing loss.

Certain embodiments of the invention include the following methodologies to treat sensorineural hearing loss caused by the lost of hair cells. For example, it is possible to replace the lost hair cells with cultured in vitro hair cells or hair progenitor cells. Either autologous or non-autologous hair cells may be used. Hair cells are obtained from a donor or are retrieved from the patient and cultured in vitro, expanding the number of cells and introducing them into the patient. Introduction may be accomplished, for example, by accessing the mastoid process and the cochlea. Cell types from ear structures, such as the cochlea, or from other tissue containing the same cell type or progenitor cell, can be recovered, expanded, and re-implanted. Further precursor cells or stem cells may be implanted, alone or in combination with relatively more differentiated cells. The precursor or stem cells then differentiate to form specialized cells to address the hearing defect. The cells may be introduced with or without the proteins, factors, and supplementing materials described herein. The appropriate cell types can be used for other causes of hearing loss.

Ear Infections

The Eustachian tube is a tubular structure that connects the middle ear to the lateral wall of the nasopharynx and allows equalization of atmospheric air pressure between the middle ear and the external auditory meatus. The Eustachian tube normal patency allows for the middle ear's ventilation as well. This function is crucial to maintain an intact conductive hearing capability. The most common infectious disease in children is the middle ear infection. It occurs in two modalities: Acute Otitis Media (AOM) and the Otitis Media/Chronic Otitis Media with Effusion (OME). OME is the most common chronic disorder of childhood. It is more frequent in children under the age of 6 years, with an incidence that declines with age. Middle ear effusions develop when the mucociliary transport system is disturbed or when the avenue of evacuation is obstructed. The mucociliary transport system may be altered by changes in the quality of the secretion or by disturbances of ciliary function (e.g. in children with Cystic Fibrosis). The pathways of evacuation may be affected by obstruction or physiologic dysfunction of the Eustachian tube. Several factors predispose children to OME, the most prevalent being horizontal position and flaccid cartilaginous support of the Eustachian tube which impairs the tube patency. After some time the fluid that has accumulated in the eardrum becomes contaminated. Drainage, most commonly through perforation of the tympanic membrane, is required. Otherwise necrosis of the ear's small bones can occur causing conductive hearing loss that is frequently irreversible. Other undesirable complications of OME is the occurrence of acquired choleasteatoma or the invasion of the middle ear with squamous epithelium. Traditional treatments of OME include the use of wide spectrum antibiotics (systemic and topical) and careful identification and treatment of other causes that may be contributing to the obstructive problem (allergies, sinusitis, upper respiratory tract infections, congenital abnormalities of the face, adentonsillar hyperplasia, amongst others). Chronic OME or repetitive episodes call for more drastic treatments along with antibiotics. These treatments include the surgical placement of pressure equalization tubes (PET) or ventilation tubes, inserted through a hole made in the tympanic membrane (myringotomy) to drain the middle ear into the ear canal. The tubes are left in place for weeks to months and require permanent surveillance and frequent maintenance. Often the tubes obstruct, extrude or move, creating the need for a surgical re-intervention. When the treatment is complete the hole in the tympanic membrane (used to insert the tube into the middle ear) needs to be closed by surgical myringoplasty. Complications of the long-term use of ventilation tubes are not uncommon and include acquired choleasteatoma, structural changes in the middle ear, recurrent perforation of the tympanic membrane and further damage to the Eustachian tube and the regulation of the air pressure between it and the ear canal.

Abnormal patency of the Eustachian tube may mimic the symptoms of serous otitis media in adults. This occurs when there is loss of tissue about the Eustachian tube orifice. The most common cause is a recent and severe loss of weight. Nasopharyngeal surgery (tumors) and trauma (barotrauma) may be causes as well. The symptoms are otophony and fullness of the ear, which are relieved when the patient lies down. Patients can hear themselves breathe and are bothered by the free exchange of air along the tube. Infusion of solutions that cause hypertrophy of the secretory glands around the orifice of the tube are usually temporary remedies to the symptoms that eventually recur as are the injection of polytetrafluoroethylene paste into the anterior wall of the Eustachian tube orifice.

Certain embodiments herein relate to the treatment of abnormalities of the patency and functionality of the Eustachian tube, e.g., defects that may cause chronic middle ear infections (Otitis Media) and other disorders. The Eustachian tube may be repaired or remodeled by bulking or augmentation of tissue at or near the Eustachian tube using cells, for instance, autologous cells. For instance fibroblasts from skin, fibroblasts from other tissues, or cell types from the ear structure tissue may be used. The cells may be injected or otherwise introduced to the patient. Thus, various embodiments of the invention include the introduction of cells into a patient to treat the defect using techniques described herein for obtaining, culturing, and introducing cells into a patient. The cells may be introduced with or without the proteins, factors, and supplementing materials described herein. Autologous cells, allogenic cells, or xenogenic cells may be used. Cells include stem cells, various differentiated cells, and their precursors. The site of introduction may be at or near the defect or at a site distant from the defect, as described herein.

Some treatments may involve the injection of cells into the basal lamina along the cartilaginous portion of the Eustachian tube to reinforce the whole structure which may be a preferred application to treat children with OME. The injection of cells into the basal lamina around the orifice of the cartilaginous portion of the Eustachian tube to bulk the orifice of the tube, is a preferred way to apply the invention to treat adults with abnormal patency of the tube. Alternately chondrocytes can be injected into a cartilaginous portion. An alternate approach is the surgical engraftment of “strands” derived from cells which are cultured in such a manner as to form three-dimensional “tissue-like” structure similar to that which is found in vivo. Also, the injection of extracellular matrix produced from the cultured cells, alone or in conjunction with cells can be used.

Balance Conditions

Dizziness, vertigo and motion sickness are abnormalities of the sense of the balance. These disorders can have their cause in alterations of the labyrinth inside the inner ear. An embodiment for this invention is the augmentation, injection, replacement or transference of the cells containing the fine hair-like sensors.

Augmentation and Repair of Eye Defects and Vision Defects

Eye Anatomy and Function

The eye is shaped like a round ball, with a slight bulge at the front. The eye has three main layers. These layers lie flat against each other and form the eyeball. The eye's genesis is from the neuroectoderm, surface ectoderm, and mesoderm. The neuroectoderm develops into brain and forebrain outgrowth gives rise to the optic stalk, vesicle and double layered optic cup. The inner layer develops into the neural retina, the outer layer develops into the retinal, iris and ciliary body pigmented epithelium and the dilator and sphincter muscles for the pupil. The surface ectoderm forms the lens vesicle which is segregated and develops into the lens. The contiguous surface ectoderm develops into the corneal epithelium and eyelid lining. The mesoderm develops into the stroma of the sclera and cornea and the uvea containing stroma of choroids, iris and ciliary body.

The outer layer of the eyeball is a tough fibrous, white, opaque membrane called the sclera (the white of the eye). The sclera is a coat of fibroblasts producing extracellular matrix including predominantly collagen and elastic fibers in 3 layers. The outermost layer is loose connective tissue and in contact with the eye socket. The middle layer is the sclera proper (Tenon's capsule), a dense network of collagen fibers and tendons of extraocular muscles attached to Tenon's capsule. The inner layer is the lamina fuscia, adjacent to choroids, and made of collagen and elastic fibers and contains pigmented cells.

The slight anterior bulge in the sclera at the front of the eye is a clear, thin, dome-shaped tissue called the cornea. The outer surface of the cornea is a shallow nonkeratinized stratified squamous epithelium and cuboidal shaped epithelial cells throughout most of the thickness of the epithelium of 5 to 6 layers of cells that rest on a thick basement membrane, Bowman's membrane, a lamina of collagen. The epithelial layers are populated with sensory nerves, have a high regenerative capacity and a cell turnover of seven days. The stroma, also called the substantia propria, is about lmm thick and contains fibroblasts and myofibroblasts in collagen fibers embedded in ground substance extracellular matrix. The inner surface of the cornea is bounded by a thick basement membrane, Descemet's membrane (made up of collagen type VIII fibers), located between the substantia propria and the corneal endothelium, and containing a single layer of low cuboidal corneal endothelial cells. The transparency of the cornea is due to the regularity of its tissue components, which minimize the scattering of light. Unlike the irregular arrangement of collagen in the sclera or dermis in the skin, the collagen fibers of the stroma are arranged into uniform layers with parallel fibers within each layer. Thus, the cornea is comprised of beneath the tear level, a three level epithelium: a stratified surface epithelium, a wing cell layer containing the corneal nerves, and the mitotically active basement membrane. Below the epithelium is the Bowman's membrane (a structure to prevent penetrating injuries), ˜250 lamellar sheets of stroma, Descemet's membrane and then the endothelium. The anterior chamber components of the eye may have some immunoprivileges, in particular the cornea, since few if any blood vessels are present.

The middle layer of the eye ball is the choroid. The choroid contains fibroblasts, leucocytes and some melanocytes. The front of the choroid contains eye muscles (ciliary muscles) and the round, colored part of the eye is called the iris. The posterior surface of the iris consists of two layers of pigmented columnar epithelium. The anterior aspect contains vascular connective tissue consisting of melanocytes, the number of which determines eye color (fewer is blue to abundant is brown). In the center of the iris is a circular hole or opening called the pupil. The pupil is surrounded by fibers of involuntary smooth muscle that act as a sphincter. The dilator pupillae muscles are located in the remaining iris stroma with a well-vascularized loose connective tissue. The choroid underlies the retina and supplies the retina with essential nutrients. At the outer margin of the lens the choroid is modified as part of the core of the ciliary processes, a double epithelial layer derived from the ora serrata, the anterior extension of the retina. Aqueous humor is secreted by the ciliary epithelium and enters into the anterior and posterior chambers between the cornea and lens, and is the nutrient supply for the cornea and lens. It nourishes the area around the iris and behind the cornea, and the pressure it exerts helps determine eye shape. This fluid is continually drained by the canal of Schlemm and into the veins at the iridiocorneal angle. Inadequate drainage raises intraocular pressure (IOP) and may damage the retina and optic nerve. The smooth muscle of the ciliary body is lateral to the ciliary processes. The body and processes extend elastic-type zonular fibers to the lens for support. The body is an expansion of the stroma of choroids near the lens. The body's stroma contains two layers, a vascular loose connective tissue layer lined with two layers of columnar cells in which the basal layer is pigmented with melanocytes and the ciliary muscle (two bundles of smooth muscle) layer. Changes in refraction and, thus, focus on near and far objects are done by altering the shape of the lens, called accommodation. In distant vision, the circular muscles of the ciliary body relax, stretching the zonular fibers and causing the lens to flatten. In near vision, the circular muscles constrict, relaxing the zonular fibers and increasing the curvature of the lens.

The inner layer of the eye ball is composed of the retina, which lines the back two-thirds of the eyeball. The retina consists of two layers: the sensory (neural) retina, which contains several layers of nerve cells that process visual information and send it to the brain, and the retinal pigment epithelium (RPE), which lies between the sensory retina and the wall of the eye (choroid). This pigmented epithelium consists of a single layer of hexagonal epithelial cells loaded with pigmented-granules and serves as a part of a barrier between the bloodstream and retina. It is important to the survival of photoreceptors. The neural retina contains the photoreceptors (rods and cones).

Rods sense black, white, shades of gray and shapes. Cones sense color, enable more detail to be seen and require more light than rods to work well. Three types of cones exist: red, green and blue. An eye has about 120 million rods and 7 million cones. Bipolar cells and ganglion cells together form a path from the rods and cones to the brain. A complex array of interneurons form synapses with the bipolar and ganglion cells and modify their activity. The ganglion cells generate the action potentials and conduct them back to the brain along the optic nerve. Contrary to the senses of smell, taste or hearing there is not a direct link between the visual stimulus in the rods and cones and the action potential.

When examined microscopically by means of vertical sections all vertebrate retinas are composed of three layers of nerve cell bodies and two layers of synapses. The outer nuclear layer, which is much thinner than the inner layer, contains cell bodies of the rods and cones on top a dense network of fibrils. The inner nuclear layer is made up of a number of closely packed cells, of which there are mainly three different kinds. Bipolar nerve-cells are the most numerous, are large and oval in shape. The horizontal cells are located at the outermost part of this inner layer. The amacrine cells are located at the innermost part of the layer. The ganglion cell layer contains cell bodies of ganglion cells and a few displaced amacrine cells. Dividing these nerve cell layers are two neuropils where synaptic contacts occur. The optic nerve contains about 1.2 million nerve fibers comprised of ganglion cells.

Thus the retina contains a vascularized cellular layer and from out to in, four cell layers, the retinal pigmented epithelium (rests upon Bruch's membrane of choroids), the photosensitive layer (contains the rod and cone cells), the intermediate layer of bipolar cells and the internal layer of ganglion cells. The inner segment of the rod and cone cells synapse with the bipolar cells. The bipolar cells synapse with the ganglion cells. Additional cells of retina include horizontal cells that connect photoreceptor cells (integrative function), the amacrine cells (conducting cells) that contact ganglion cells and the Muller cells (support function) that occupy throughout the retina and form a basement membrane adjacent to the vitreous humor. The fovea is a thin depression in the retina comprised of bipolar and ganglion cells and devoid of cone cells. The optic papilla is devoid of photosensitive cells and is located at the exit of the optic nerve from the eye.

The inside of the eye is divided into three sections called chambers. The anterior chamber is the front part of the eye between the cornea and the iris. The iris controls the amount of light that enters the eye by opening and closing the pupil. The iris uses special muscles to change the size of the pupil. These muscles can control the amount of light entering the eye by making the pupil larger (dilated) or smaller (constricted). The posterior chamber is positioned between the iris and the lens. The lens is located behind the iris and is normally clear. Light passes through the pupil to the lens. The lens is held in place by small tissue strands or fibers (zonules) extending from the inner wall of the eye. The lens is very elastic. Small muscles attached to the lens can change its shape, allowing the eye to focus on objects at varying distances. Tightening (contraction) or relaxing these muscles causes the lens to change shape, allowing the eyes to focus on near or far objects (accommodation). The vitreous chamber is located between the lens and the back of the eye. The back two-thirds of the inner wall of the vitreous chamber is lined with a special layer of cells (the retina) that is covered with millions of highly sensitive nerve cells that convert light into nerve impulses. Nerve fibers in the retina merge to form the optic nerve, which leads to the brain. Nerve impulses are carried through the optic nerve to the brain. The macula, near the center of the retina at the back of the eyeball, provides the sharp, detailed, central vision for focusing on what is in front of the person. The rest of the retina provides side (peripheral) vision, which allows you to see shapes but not fine details. Blood vessels (retinal artery and vein) travel along with the optic nerve, and enter and exit through the back of the eye.

Fluid fills most of the inside of the eye. The chambers in front of the lens (both the anterior and posterior chambers) are filled with a clear, watery fluid called aqueous humor. The large space behind the lens (the vitreous chamber) contains a thick, gel-like fluid called vitreous humor or vitreous gel. These two fluids press against the inside of the eyeball and help the eyeball maintain its shape. The vitreous body keeps lens and retina in place. The vitreous chamber fluid is 99% water with the remaining 1% composed of mostly collagen, vitrosin and hyaluronic acid. The vitreous chamber is 80% of the globe or about 4 ml of fluid. The fluid appears to be made by the neural retinal in early embryonic stages, whereas in later development cells within the vitreous body, synthesize the fluid, e.g. hyalocytes. Vitreous fluid is clear and avascular. A layer of cells called the internal limiting membrane separates the inner surface of the retina from the vitreous, forming a potential space, the subhyaloid space.

The eye is like a camera. Light passes through the cornea and the pupil at the front of the eye and is focused by the lens onto the retina at the back of the eye. The cornea and lens bend light so it passes through the clear substance (vitreous gel) in the back chamber of the eye and is projected onto the retina. The retina converts light to electrical impulses. The optic nerve carries these electrical impulses to the brain, which converts them into the visual images that are then seen.

Vision Defects

Refraction Problems.

Myopia (nearsightedness) is a common cause of blurred vision. A nearsighted person's distance vision is blurry and out of focus, making it hard to see objects that are far away but easy to see them up close. Most nearsightedness is caused by a natural variation in the length of the eyeball that makes it too long, so that it is oval (egg-shaped) rather than round. The effect of this variation is a refractive error that makes light rays entering the eye focus in front of the retina. As a result, the person has trouble seeing objects that are far away. In eyes with normal vision, light focuses directly on the retina. Less frequently, nearsightedness may also be caused by a change in the ability of the cornea and lens to focus on what a person is looking at. Most cases of nearsightedness are considered a variation from normal, not a disease. The common form of nearsightedness is called physiological myopia. Uncommon forms of nearsightedness include pathological myopia (rare condition in which the eye globe continues growing after adulthood) and secondary myopia (myopia develops as a result of another medical condition). Nearsightedness is classified as mild to moderate (less than 6 diopters) or high (6 diopters or more). Eyeglasses or contact lenses can help correct nearsightedness. Some nearsighted people may also choose to have refractive surgery, which can reduce nearsightedness by changing the shape of the cornea. Myopia can be treated with, as described below, and can also be done following deep sclerectomy.

Hyperopia (far-sightedness) is a condition in which a person has difficulty seeing objects that are located close to the eye, although vision of distant objects (far vision) is good. In most cases, far-sightedness is an inherited condition that is caused by an abnormally short eye, as measured from front to back. This situation reduces the distance between the cornea and the retina. As a result, images tend to focus behind the retina, rather than on the retina itself. Sometimes, the eye is able to partially or totally compensate for this focusing problem through a process called accommodation.

Accommodation takes place by the action of the ciliary muscle. The ciliary muscle is composed of smooth muscle cells that are organized into fibers. These fibers form a circular band that embraces the outer surface of the forepart of the eye globe just behind the pupil. The ciliary muscle consists of two sets of muscular fibers that run in three directions: circular, radial and meridional. Contraction of the ciliary muscle will alter the shape of the lens bringing the viewed object into focus. Eyeglasses or contact lenses can help correct far-sightedness. Surgical techniques are available but not in widespread use.

Presbyopia is a refraction-related problem that is a universal aging phenomenon of the lens resulting in blurriness of close objects. As people age past 40 years, the lens becomes harder, less elastic and changes shape less easily to see nearby objects clearly. The normal lens changes shape in order to properly focus on objects. The ciliary muscles contract to thicken the lens to bring objects into focus. As a result, the accommodation process becomes more difficult, making it harder to see objects up close. “Reading” glasses are the prescribed treatment.

Astigmatism results in blurry vision. Astigmatism is usually congenital. The refraction error is due to uneven curvature of the cornea. A normal cornea is symmetrically curved whereas an astigmatic cornea has steeper or flatter areas that produce distorted vision. Glasses are the standard treatment.

A prolonged increase in blood sugar concentration often causes a metabolic change in the lens and alters its shape, so as to create a refraction error. Typically, this is due to diabetes mellitus.

Refractive strength is measured in diopters. The cornea contributes 43 diopters, and is the primary refractive component of the eye. The lens contributes 17-25 diopters depending on its accommodation. Thus the cornea focuses roughly ⅔ of the light entering the eye, while the lens focuses ⅓.

In a preferred embodiment, cultured cell types comprising the eye structures affecting the refraction of light can be used to restore or improve vision. The primary structures include the cornea, the lens, the ciliary muscle, the vitreous chamber, the sclera and the eyeball.

Various materials and methods are provided in this application for introducing suitable cells into a patient. In some embodiments, these techniques may be used for nearsightedness, far-sightedness, or presbyopia, by, for example, obtaining smooth muscle cells, e.g., from the ciliary muscle, and introducing them into the anatomy of the eye as needed to enhance ocular muscle tissues. For example, smooth muscle cells can be implanted into the ciliary muscle or fiber area. Smooth muscle cells from other tissue or muscle cells from other tissue can be used as well. In alternate methods for presbyopia, lens cells can be introduced to restore the refraction error. Lens cells may be obtained, for example, from the patient, family members or other donors and expanded and implanted as described herein. In astigmatism, the correction of the refraction error can be done with the implantation of corneal fibroblasts into the cornea, with the corneal cells being obtained and introduced as described herein. In a preferred embodiment, corrections in the accommodation structures of the eye, primarily cornea, lens and ciliary muscle can be performed to correct the various accommodation defects, myopia, presbyopia, hyperopia and astigmatism with the cell type of these structures. In a preferred embodiment the corneal contribution to accommodation is performed by the implantation of corneal fibroblasts. Similar cell types from other tissues may be used in lieu of the cell types from the various eye structures. Thus, various embodiments of the invention include the introduction of cells into a patient to treat the defect using techniques described herein for obtaining, culturing, and introducing cells into a patient. The cells may be introduced with or without the proteins, factors, and supplementing materials described herein. Autologous cells, allergenic cells, or xenogenic cells may be used. Cells include stem cells, various differentiated cells, and their precursors. The site of introduction may be at or near the defect or at a site distant from the defect, as described herein.

Corneal Defects

Injuries due to corneal abrasion or corneal lacerations or keratitis can also be treated using techniques described herein for obtaining, culturing, and introducing cells into a patient, including use of proteins, factors, and matrix materials, as appropriate. Scars and ulcers can occur in the eye structures due to injury or disease. Native cells taken from the same tissue or similar tissue as the structure that is to be treated can be used to repair the ulcer or scar. For instance, in the cornea, fibroblasts from the cornea can be used. In other eye structures (e.g., scleral fibroblasts), fibroblasts or other cell types similar to the treated tissue can be used, as well as fibroblasts from other tissue types to correct scars and ulcers of the other eye structures. Repair or replacement of the cornea with corneal cell types or sclera cell types can be performed. Thus, various embodiments of the invention include the introduction of cells into a patient to treat a defect using techniques described herein for obtaining, culturing, and introducing cells into a patient. The cells may be introduced with or without the proteins, factors, and supplementing materials described herein. Autologous cells, allogenic cells, or xenogenic cells may be used. Cells include stem cells, various differentiated cells, and their precursors. The site of introduction may be at or near the defect or at a site distant from the defect, as described herein.

When injured, the corneal fibroblast differentiate to myofibroblasts. Corneal fibroblasts produce a clear extracellular matrix whereas myofibroblasts do not. Accordingly, fibroblasts that can be effective at repairing corneal defects can be used (e.g. corneal fibroblasts).

Keratocytes, which are epithelial cells, are also involved in corneal wound healing. Keratocytes can be expanded in numbers by factors produced by corneal fibroblasts, e.g, by co-culture or use of medium enriched with corneal fibroblast-produced factors. Keratocytes can be used to accelerate wound healing of the cornea. Co-culture with corneal fibroblasts can enhance the proliferation of keratocytes in vitro.

Thus it is possible to use these techniques as an alternative to corneal transplants. Corneal cells, described above, and/or extracellular matrix can be used in the implant. Other tissue fibroblasts can be used, such as sclera fibroblasts.

Macular Degeneration of the Retina (MD)

Anatomy & Histology

The retina is a thin layer of neural tissue lining the inner eye. In a histologic section it is stratified and described as having 10 layers consisting of neurons or cell bodies, synapses, one principal type of glial cell, the photoreceptive cells called rods and cones, and an outermost pigmented epithelium.

The central zone of the retina is located in the center of the posterior part of the retina, corresponding to the axis of the eye. It is at a point where the most critical vision is enabled, a yellowish spot called the macula lutea. It is very rich in photoreceptive cells: the rods and the cones. The most concentrated collection of photosensitive cells is in the retina, including those that enable critical color and fme detail vision, are found in the Bulls-Eye center zone in the macula. Rods are receptive in dim light whereas cones function in bright light and are responsible for color vision. The light falling onto these cells in the retina is transformed into electrical signals which are transmitted to the brain centers that process and interpret them.

Macular degeneration (MD) is the imprecise historical name given to that group of diseases that causes sight-sensing cells in the macular zone of the retina to malfunction, lose function and eventually die. This results in a debilitating loss of vital central and detailed vision, while peripheral vision is retained. Because the brain cleverly learns to compensate and fill in the missing part of the picture in early cases with spotty macular cell damage or dysfunction, most people only present to their ophthalmologist when the disease is fairly advanced.

Adult macular degeneration (AMD) is traditionally described as that form of the disease that affects individuals over the age of 55 years. However, it has been recently discovered that a significant number of these individuals may have a major genetic component that contributes to the disease. Each year 1.2 million of the estimated 12 million people with macular degeneration will suffer severe central vision loss. Each year 200,000 individuals will lose all central vision in one or both eyes. While the causes of macular degeneration are unknown, the ABCR genes may increase the likelihood of an individual developing macular degeneration by approximately 30 percent. However, most macular diseases have a complex genetic makeup compared with single gene-causation diseases. In most individuals macular degeneration is likely due to both environmental and genetic factors that combine to cause damage and disease.

Juvenile Macular Degeneration (JMD) occurs more rarely than AMD. It occurs in younger people, infants and young children, occurring in clusters within families. JMD is inherited, caused by mutated genes. These types of macular degeneration are collectively called Juvenile Macular Degeneration (JMD). Following is a list of the major types of JMD that are inherited in either an autosomal dominant or recessive fashion: Stargardt's disease, Best's vitelliform macular dystrophy, Doyne's honeycomb retinal dystrophy, Sorsby's fundus dystrophy, Malattia levintinese, Fundus flavimaculatus and Autosomal dominant hemorrhagic macular dystrophy.

Clinical Manifestations

MD can cause different symptoms in different people. Sometimes only one eye loses vision while the other eye continues to see well for many years. The condition may be hardly noticeable in its early stages. But when both eyes are affected, reading and close up work can become difficult. In a good number of cases retinal angiography or an electroretinogram confirms the diagnosis.

There are two types of MD the dry and the wet type. Both types cause vision loss due to damage to the nerve cells in the macula. The dry type occurs with advancing age in certain people due to the blood vessels supplying the macula harden and break down. Transport of vital oxygen into, and waste materials/fluids out, becomes more difficult leading to accumulation of broken down material that contributes to drusen. As drusen continues to accumulate, the photoreceptive cells are lifted further and further away from their blood supply, progressively impairing the transport of vital substances to the macular area of the retina. This causes the central point of the retina (macula/fovea) to bow upwards causing loss and distortion of vision. Ten percent of people with dry MD will go on to develop the wet form of the disease, which is associated with blood vessel leakage and bleeding, causing the most severe vision loss. Wet MD is caused by growth of abnormal blood vessels under the macula (i.e., choroidal neovascularization).

Treatment

Once the disease has been diagnosed and classified the patient may modify some environmental risks known to worsen the disease, that further decreases the oxygen supply to the macula, such as smoking or a high cholesterol diet. Laser photocoagulation is a specific treatment for the forms of macular degeneration, including leakage from submacular neovascularizations.

Restoration of the central portion of the retina can be accomplished by implantation of the destroyed macular nerve cells (e.g., photoreceptive cells) that impart sight-sensing in the macular zone of the retina. Thus, various embodiments of the invention include the introduction of cells, e.g., retinal pigmented epithelial cells, into a patient to treat a defect e.g., at the site of the retina using techniques described herein for obtaining, culturing, and introducing cells into a patient. This includes the use of neural progenitor cells as an alternate cell type. The cells may be introduced with or without the proteins, factors, and supplementing materials described herein. Autologous cells, allogenic cells, or xenogenic cells may be used. Cells include stem cells, various differentiated cells, and their precursors. The site of introduction may be at or near the defect or at a site distant from the defect, as described herein.

Cataracts

A cataract is a cloudiness or opacity in the normally transparent crystalline lens of the eye. This cloudiness can cause a decrease in vision and may lead to eventual blindness.

The lens lies behind the pupil and iris in the anterior chamber of the eye. It is covered by a cellophane-like lens capsule. The lens is normally transparent (the second most transparent tissue in the body, second to the cornea), elliptical in shape and somewhat elastic. The anterior surface of the lens consists of an extracellular capsule with a simple cuboidal epithelium of transparent, polygonal, nucleated cells. Toward the equator of the lens, these epithelial cells proliferate and elongate, losing their nuclei but retaining a high concentration of proteins (crystallins). New fibers become arranged like layered shells on top of each other, and are produced throughout life, the older located at the center of the lens. Thus the lens contains embryonic, fetal and postnatal cells and retains every cell that it has formed. The basal surface of the lens cells is attached to a basement membrane, the lens capsule. The basement membrane of the epithelial cells is a translucent connective tissue. Zonule fibers attach to the capsule around the periphery of the lens. The lens is avascular and receives its nutrition from the surrounding aqueous and vitreous humor. The lens is made up of approximately 35% protein and 65% water. The water soluble crystalline (e.g. β/γ crystalline superfamily) proteins are important for lens clarity and its ability to refract light. As people age, degenerative changes in the lens' proteins occur. Changes in the proteins, water content, enzymes, and other chemicals are some of the reasons for the formation of a cataract.

The major areas of the lens are the nucleus, the cortex, and the capsule. The nucleus is in the center of the lens, the cortex surrounds the nucleus, and the capsule is the outer layer. Cataracts in the elderly are so common that they are thought to be a normal part of the aging process. Cataracts associated with aging (senile or age-related cataracts) most often occur in both eyes, with each cataract progressing at a different rate. If the cataract remains small or at the periphery of the lens, the visual changes may be minor.

Cataracts that occur in people other than the elderly are much less common. Congenital cataracts occur very rarely in newborns. Traumatic cataracts may develop after a foreign body or trauma injures the lens or eye. Systemic illnesses, such as diabetes, may result in cataracts. Cataracts can also occur secondary to other eye diseases such as an inflammation of the inner layer of the eye (uveitis) or glaucoma. Such cataracts are called complicated cataracts. Toxic cataracts result from chemical toxicity, such as steroid use. Cataracts can also result from exposure to the sun's ultraviolet (UV) rays.

Clinical Manifestations

Opacities of the lens can occur in any area of the lens. Cataracts, then, can be classified according to location (nuclear, cortical, or posterior subcapular cataracts). The density and location of the cataract determines the amount of vision affected. If the cataract forms in the area of the lens directly behind the pupil, vision may be significantly impaired. A cataract that occurs on the outer edges or side of the lens will create less of a visual problem. Between the ages of 52-64, there is a 50% chance of having a cataract, while at least 70% of those 70 and older are affected.

The elasticity of the lens allows it to focus on both near and far objects. Muscles, can then change the shape of the lens. This process is called accommodation-the lens focuses images to help make vision clear. The lens is thinner when focused on distant objects since ciliary muscles relax and the lens is thicker when focusing on near object since ciliary muscles contract, relaxing tension on zonule fibers.

The common symptoms of cataracts are the gradual, painless onset of blurry, filmy, or fuzzy vision, poor central vision, frequent changes in eyeglass prescription, changes in color vision, increased glare from lights (e.g. oncoming headlights when driving at night), “second sight” improvement in near vision (no longer needing reading glasses) and a decrease in distance vision, poor vision in sunlight, and the presence of a milky whiteness in the pupil as the cataract progresses.

Cataracts are easily diagnosed from the symptoms, a visual acuity exam using an eye chart, and by examination of the eye itself. Shining a penlight into the pupil may reveal opacities or a color change of the lens even before visual symptoms have developed. A microscope instrument called a slit lamp is used to examine the front of the eye, the lens and determine the location of the cataract. Other diagnostic tests may be used to determine if cataracts are present or how well the patient may potentially see after surgery. These include a glare test, potential vision test, and contrast sensitivity test. Prevention of cataract development includes the protection from UV radiation, steroid and other medication avoidance and the use of antioxidants in the diet.

Treatment

In the early stages of cataract development, no treatment or increased strength in eyeglass prescription is called for. Cataract surgery, the only option for patients whose cataracts interfere with vision to the extent of affecting their daily lives, is the most frequently performed surgery in the United States. It generally improves vision in over 90% of patients. A “ripe” or mature cataract is when the lens is completely opaque. Most cataracts are removed before they reach that stage. Sometimes cataracts need to be removed so that the doctor can examine the back of the eye more carefully. This is important in patients with diseases that may affect the eye. If cataracts are present in both eyes, only one eye at a time should be operated on. Healing occurs in the first eye before the second cataract is removed, sometimes as early as the following week. A final eyeglass prescription is usually given about 4-6 weeks after surgery. Patients will still need reading glasses. The overall health of the patient needs to be considered in making the decision to operate.

Removal of the cloudy lens can be done by several different procedures. Extracapsular cataract extraction is the most common. The lens and the front portion of the capsule are removed. The back part of the capsule remains, providing strength to the eye. A replacement lens is usually inserted at the time of the surgery. A plastic artificial lens called an intraocular lens (IOL) is placed in the remaining posterior lens capsule of the eye. In a rarely used method, the lens and the entire capsule are removed by intracapsular cataract extraction. This method carries an increased risk for detachment of the retina and swelling after surgery. When the intracapsular extraction method is used, an IOL may be clipped onto the iris. Phacoemulsification is a type of extracapsular extraction requiring a very small incision, resulting in faster healing. Ultrasonic vibration is applied to the lens to break it up into very small pieces which are then aspirated out of the eye with suction. A folding IOL is used when phacoemulsification is performed to accommodate the small incision. Contact lenses and cataract glasses (aphakic lenses) are prescribed if an IOL was not inserted.

Thus, various embodiments of the invention include the introduction of cells, e.g., ciliary muscle cells, lens cells, corneal cells, and fibroblasts into a patient to treat a defect using techniques described herein for obtaining, culturing, and introducing cells into a patient. The cells may be introduced with or without the proteins, factors, and supplementing materials described herein. Autologous cells, allogenic cells, or xenogenic cells may be used. Cells include stem cells, various differentiated cells, and their precursors. The site of introduction may be at or near the defect or at a site distant from the defect, as described herein.

The implantation of ciliary muscle cells to enhance accommodation can be used to offset cataract distortion to vision. Also further accommodation of the cornea by cell implantation can be used to offset cataract distortion. Implantation of lens cells into lens containing the cataract can be used to remove the cataract or supply additional lens area for vision. Fibroblasts can be used to remove cataract by injection of fibroblasts into the cataract region. Corneal fibroblasts are preferred. Crystalline proteins can be added with the cell implant. The lens epithelial cells can be implanted into the cataract area to reconstruct the lens in vivo. A synthetic lens made of lens cells in vitro can be implanted after removal of lens.

Eye Muscle Control

Each eye is held in place by three pairs of taut, elastic muscles which constantly balance the pull of the others. The superior rectus acts to roll the eyeball back and up, but it is opposed by the inferior rectus. In the same way, the lateral rectus pulls to the side, while the medial rectus pulls toward the nose, and the two oblique muscles roll the eye clockwise or counterclockwise. The muscles of each eye work together to move the eyes in unison. Because of the constant tension in the muscles, they can move the eye very quickly, much faster than any other body movement. The eye muscles work together to carry out no less than seven coordinated movements and allow the eye to track many different kinds of moving object. The first three movements (tremor, drift and flick) are the result of the constant, opposing muscle tension. Tremor causes an almost unseen trembling of a point image, and drift makes the image move slowly off-center. Before the movement becomes really noticeable, there is a quick flick to bring the image to the center. These movements make sure that the image constantly moves over unused parts of the retina and, as a result, the receptors at any spot do not get overloaded with images and effective vision is maintained. Smooth pursuit movements are used to follow objects at a high speed; for example, from word to word and line to line when reading. Binocular vision is created by the separation of the eyes, so that each eye has a slightly different view of the same scene, giving a three dimensional effect. To prevent this from causing double vision, the sixth eye movement, called “vergence,” helps out. The eyes turn inward to direct the images directly onto small, rodless areas of the retina. During these movements, the brain registers the amount of tension and uses it to estimate the distance of the object. The complex of the eye movements is the vestibulo-ocular system. It works to keep the image of an object on the rodless areas while the head and body are in motion. This is aided by the vestibular apparatus in the inner ear, which provides the brain with a flow of information about the way that the head is moving. Infants are not able to focus their eyes close up until they are three to six months old, and it may be a year before their eyes can work together all the time, rather than wandering around individually. The extraocular muscles of the eye are largely white fibers of skeletal muscle.

Strabismus is a visual disorder where the eyes are misaligned and point in different directions. This misalignment may be constantly present, or it may come and go. Muscle cells (e.g., smooth muscle cells) can be used to mitigate or eliminate visual disorders due to dysfunctional eye movement caused by eye muscle hypoplasia or dystrophy by implantation into the eye muscle structure that is defective and using techniques described herein for obtaining, culturing, and introducing cells into a patient. Muscle cells may be obtained from the patient, other donors, the eye, or other tissues having muscle cells. Muscle cell precursors or stem cells may be used alone or in combination with relatively more differentiated cells. Thus, various embodiments of the invention include the introduction of cells, e.g., smooth muscle cells into a patient to treat a Strabismus using techniques described herein for obtaining, culturing, and introducing cells into a patient. The cells may be introduced with or without the proteins, factors, and supplementing materials described herein. Autologous cells, allogenic cells, or xenogeneic cells may be used. Cells include stem cells, various differentiated cells, and their precursors. The site of introduction may be at or near the defect or at a site distant from the defect, as described herein.

Glaucoma

Glaucoma is not a single disease but a group of diseases of the eye. Glaucoma affects about 2 million of Americans or 3 percent. The common feature is increased pressure within the eyeball resulting in progressive damage to the optic nerve. The aqueous humor is produced constantly and needs to be drained constantly. The drain is at the site where the iris and cornea meet. The tissue for this exit is the trabecule. This channel, the trabecular meshwork, a sponge-like, porous network is responsible for 80-90% of the fluid outflow. The remainder of the fluid passes through the channel located behind it, the uveoscleral pathway. This drainage angle directs fluid into the canal of Schlemm, a channel that leads the fluid to a network of small veins outside the eye. Without proper drainage pressure builds up within the eye, the space between the cornea and the iris and in the vitreous humor behind the lens. The latter pressure presses on the retina and affects the fibers of the optic nerve. Normal intraocular pressure is maintained between 10 to 20 mm Hg.

Acute glaucoma occurs primarily in the elderly who are far-sighted. The lens becomes enlarged as the eye ages, pushing the iris and ciliary body forward. The drainage angle is then blocked by the iris resulting in a closed-angle glaucoma. Iridotomy is sometimes used to create a drainage hole in the iris to relieve the pressure.

Chronic glaucoma affects 85-95% of people with glaucoma. Fluid does not drain properly from the front chamber of the eye and this type of glaucoma is called open-angle glaucoma. Fluid passes from the posterior chamber behind the iris into the anterior chamber between the iris and the front of the eye. Drug treatments or eyedrops that decrease the pressure in the eye are helpful. Surgery by laser to open blocked drainage channels in the front chamber of the eye may be needed. Sclerectomy may be done to relieve the pressure.

Normal tension glaucoma in which the IOP remains in the normal range. Other factors are present that cause optic nerve damage. Congenital glaucoma is rare and occurs in patients in which the eye's drainage canals fail to develop correctly. Microsurgery can be used to correct the defect. Other glaucoma types occur in which drainage is blocked. Pseudoexfoliation syndrome occurs when protein flakes from the outer layer of the lens collects in the drainage angle. Pigment glaucoma occurs when pigment granules that color the iris flake off into the intraocular fluid. Irido corneal endothelial syndrome results in cells from the back surface of the cornea spreading to the drainage angle and at times forming scars that connect the iris to the cornea. Secondary glaucomas, such as neovascular glaucoma, due often to diabetes or other disorders, forms abnormal blood vessels on the iris and in the drainage system. Other secondary glaucomas can be due to the local or systemic use of corticosteroids.

Certain embodiments of the invention may be used to restore the tissue removed, the trabecular tissue, the canal of Schlemm and the sclera with the cell types that inhabit these eye structures. These techniques can be used to restore eye tissue or space maintenance due to sclerectomy, trabeculectomy, phacoemulsification, phacotrabeculectomy, phacotrabeculectomy combined operations and iridocetomy. These techniques can be used in combined cataract-glaucoma operations. In a preferred embodiment, cells, such as fibroblasts and extracellular matrix produced in vitro, are sutured in place of the dissected tissue, such as sclera. For example, following sclerectomy three-dimensional sclera can be made with autologous sclera fibroblasts and implanted. Thus, various embodiments of the invention include the introduction of cells, e.g., fibroblasts into a patient to treat a glaucoma defect using techniques described herein for obtaining, culturing, and introducing cells into a patient. The cells may be introduced with or without the proteins, factors, and supplementing materials described herein, e.g., the cells may be introduced with extracellular matrix. Autologous cells, allogenic cells, or xenogeneic cells may be used. Cells include stem cells, various differentiated cells, and their precursors. The site of introduction may be at or near the defect or at a site distant from the defect, as described herein.

Colorblindness and Nightblindness

As high as 8 percent of males in some populations are affected with colorblindness. Three kinds of cones absorb light to distinguish colors and are located in a region opposite the lens on the retina called the fovea. Red cones absorb long-wavelength light (peak of 565 nm), green cones absorb middle-wavelength light (peak of 535 nm) and blue cones absorb short-wavelength light (peak of 440 nm). Each type of cone, as well as the rods, has a transmembrane protein, opsin, coupled to the prosthetic group retinal. A different amino acid sequence for the four types of opsins accounts for the different absorption spectrum. The majority of colorblindness is due to red-green spectrum. Determination of colorblindness by examination can indicate what cone (red, green or in rarer cases blue) are needed for implantation into the retinal region.

Nightblindness or the inability to see in reduced light is due to the absorption of light by the rods of the retina. Rods are extremely sensitive to light and contain rhodopsin as the light-absorbing pigment. Several rods can share a single circuit to one ganglion cell and a single rod can send signals to several different ganglion cells. Techniques disclosed herein can be used to restore sensitivity of light to eyes by the implantation of rods to the retinal region. Thus, various embodiments of the invention include the introduction of cells, e.g., rod-cells into a patient to treat a colorblindness or nightblindness defect using techniques described herein for obtaining, culturing, and introducing cells into a patient. The cells may be introduced with or without the proteins, factors, and supplementing materials described herein. Autologous cells, allogenic cells, or xenogeneic cells may be used. Cells include stem cells, various differentiated cells, and their precursors. The site of introduction may be at or near the defect or at a site distant from the defect, as described herein.

Age-Related Vision Defects

Glaucoma, cataract, macular degeneration, retinal detachment, retinal vessel occlusion, retinitis pigmentosa, color perception and scarring from choroiditis are largely age-related eye problems.

The cause of choroiditis, however, is largely unknown although infections such as toxoplasmosis can be associated with the associated inflammation process. Choroiditis is the inflammation of the choroid layer and may scar the choroids and the retina, impairing vision. Symptoms are blurred vision and discomfort in one eye. Scars can be removed with fibroblasts such as with choroid fibroblasts.

Retinitis Pigmentosa is known as night blindness. There is difficulty in seeing at night or in reduced light, poor central vision and loss of peripheral vision. In this uncommon disorder the rods in the retinal are affected the most. Implantation of healthy rods or progenitor cells to the rods (e.g., lateral ventricle astrocytes) can be used to correct night blindness.

Retinal Detachment has the symptoms of blurred vision, floaters and the sensation of flashing lights. These symptoms often occur before complete detachment. Lasers or cryopexy can be used to cover the defect, but inflammation leads to scar formation. Cells, such as fibroblasts, can be used to remove these scars. Scleral sheaths formed in vitro can be used to pave the re-attachment of the retina. Holes and tears can be treated with wound healing fibroblasts or myofibroblasts, preferably from that retinal eye region or alternately from other eye areas (e.g., cornea).

Diabetic retinopathy is a deterioration of the blood vessels of the retina that can lead to blindness. Similarly, damage to the retina due to hypertension can lead to vision problems. Implantation of endothelial cells, or with growth factors such as VEGF, can improve the vessel maintenance and genesis for this type of damage. Pericytes can be used to increase blood flow and to induce angiogenesis in the eye retina. The invention can be used to repair the retina with the cells contained in the eye area, including the implantation of retinal pigmented epithelial cells.

Many of the vision defects are affected by accommodation. These defects can be corrected by augmenting or repairing the structures involved in accommodation with the appropriate cells native to the structures. Most notably this includes the structures of lens, the cornea, ciliary muscles, suspensory ligaments of the lens and their cells. An example is implantation into the cornea by corneal epithelial cells into the epithelial layer, corneal fibroblasts into the connective tissue layer or corneal endothelial cells into the inner layer. In a preferred embodiment the connective tissue layer is implanted. Muscle cells into the ciliary muscle region is another example.

Thus, various embodiments of the invention are directed to the treatment of accommodation, Diabetic retinopathy, Retinal Detachment, Retinitis Pigmentosa, and choroiditis. Techniques described herein may be used for obtaining, culturing, and introducing cells into a patient. Examples of cells are choroid fibroblasts, rods or progenitor cells to the rods, fibroblasts, wound healing fibroblasts, myofibroblasts, Pericytes, retinal pigmented epithelial cells, and corneal epithelial cells. The cells may be introduced with or without the proteins, factors, and supplementing materials described herein. Autologous cells, allogenic cells, or xenogeneic cells may be used. Cells include stem cells, various differentiated cells, and their precursors. The site of introduction may be at or near the defect or at a site distant from the defect, as described herein.

Eye trauma

Injury to the eye can cause a variety of problems such as retinal detachment, corneal abrasions, and others similar in nature to the defects listed above. In situ appropriate cells can be expanded and implanted into the appropriate eye structures to repair such injuries. For example, in corneal abrasions comeal stromal fibroblasts or epithelial cells can be implanted into the affected corneal layer for removal of the abrasion or in severe cases, the cornea can be made in vitro with the appropriate layer. Implantation into the outer layer of the retina can be achieved by in situ implantation of retinal pigmented epithelial cells to correct retinal injuries. Other eye trauma defects can be corrected by implanting cells that are native to the injured area. Cells native to the area is a term referring to the cell types that comprise the area. Cells native to an area can be obtained from the site of injury, from the same tissue type but one that is uninjured, or from a donor other than the patient.

Lacrimal Apparatus and Tear Production

The lacrimal apparatus is the system in the eye region that produces and drains tears. The apparatus is comprised of the main and accessory lacrimal glands. The main lacrimal gland, located at the upper region of the bony orbit, is the tear producing gland for extra tears during eye irritation and crying. The gland is a merocrine tuboloacinar gland with prominent mucous-type secretory granules, which, when released into the main excretory lacrimal duct, located at the outer region of the bony orbit, release tears from the lacrimal gland into the conjunctiva. The conjunctiva is the mucous membrane layer that covers and protects the internal surface of the eyelids, the surface of the eyeball (lateral margins of the cornea) and the front part (anterior aspect) of the sclera (white part of eye). The conjunctiva, predominantly in the upper and lower eyelids, contains the accessory lacrimal glands that maintain a normal amount of tears on the surface of the conjunctiva, helping to counteract the effect of tear evaporation. The lacrimal glands contain exocrine secretory epithelial cells to produce the tears. The conjunctiva contains nonkeratinizing, squamous epithelium, a thin, richly vascularized substantia propria (containing lymphatic vessels and cells, such as lymphocytes, plasma cells, mast cells and macrophages), lacrimal glands and goblet cells. The conjunctiva consists of stratified squamous near the cornea, columnar epithelia in other regions of the eyeball, and goblet cells in the ocular conjunctiva that are cover the orbit and in the palpebral conjunctiva that line the interior of the eyelid.

After bathing the front part of the eyeball, the lacrimal lake is a small open area of the conjunctiva where tears collect in a slit-like area called the conjunctival sac. The sac is located between the eyelids and the conjunctiva. Drainage of tears from the eyes occur through tiny openings towards the inner part of each eyelid, called the lacrimal puncta. These openings connect the tears to the superior and inferior lacrimal canals that travel into a hollow space of each eye, the lacrimal sac. Muscles covering the sac squeeze and release the sac during blinking which produces a suction effect to draw away extra tears. Lacrimal bones surround the lacrimal sac and are located on each side of the nose, within the inner part of the eye socket. Tears travel into tube shaped areas beneath the sac through nasolacrimal ducts that go through the bone and lead to an opening in the nose. Failure to drain properly can lead to “wet” eyes and cause serious infections. Also “wet eyes” can be due to the tear glands overproducing watery or reflex tears to compensate for a lack of a balanced tear film.

The tear film (40 μm deep) provides a moistening function and supplies the major refractive interface of the eye. Immunoglobulin A, lyzozymes, lactoferrin, and other substances in tears combat infection and participate in inflammatory reactions at the ocular surface. Tear functions are many and essential. In the cornea, tears lubricate and provide a smoother optical surface, so that vision remains clear. Tears also help keep the cornea properly moisturized and rich in oxygen. For the eye in general, tears also act as a “wiper fluid”, allowing the eyelids to wash the eye free of debris with every blink, protecting the eye's surface from the environment. Tears form a complex tri-layered (or tri-phased) film consisting of an inner mucin dominated layer, an aqueous layer, and outer lipid (oil) layer. The total thickness varies from the top to the bottom of the cornea, from before and after blinking, and is due to the output of the tear glands. The thickness is estimated to be an average of 3 mm. The secretions in each layer are tightly regulated. The mucous layer is made by specialized epithelial cells (goblet cells) located on the eye's surface and conjunctiva. The mucous layer is needed for tears to adhere to cells on the conjunctiva and cornea and to spread evenly over the eye's surface. The watery layer is produced by two different sets of lacrimal glands. Under normal conditions, the lacrimal cells in the accessory lacrimal gland produce the tears needed to keep the eye moist and is referred to as basal tear secretion. Under reflex tear production, the eye is irritated and the lacrimal cells (acinar cells) from the main lacrimal gland produce the watery layer. The aqueous layer contains growth factors, chemicals, substances and salts (isotonic) which nourish the eye surface, e.g. the conjunctiva and the cornea. The oily outer layer is produced by epithelial cells in the tarsal or meibomian glands (meibocytes) located under the conjunctiva and between the tarsi (fibroelastic tissue) of the eyelid. There are 20 to 30 tarsal glands per eyelid. Acinar epithelial cells and ductal elements containing progenitor cells that can give rise to differentiated epithelial oil producing cells are in the meibomian glands. The oily layer prevents excess evaporation of the watery layer and helps paste the inner two layers of the tear onto the eye surface. Tear production decreases primarily with age and is prevalent in post-menopausal women. Other causes of dry eye includes hormonal changes brought on by pregnancy, lactation, oral contraceptives, and menstruation. Additionally, excess tear drainage, environmental conditions due to smoke, fluorescent lights, air pollution, wind, heat, air conditioning, dry climates, and medications such as antihistamines, decongestants, antihistamines, tranquilizers, beta blockers and medications for breast cancer, depression, Parkinson's disease, incontinence, ulcers and blood pressure can cause Dry Eye.

A pathological condition known as ‘dry eye’ (lack of tears) is a very painful one in which the survival of the corneal surface epithelial cells is at risk because of the lack of normal lubrication. A dry eye condition can occur if any of the three layers of the tear film are deficient. There are two major types of dry eye. In the evaporative or tear-deficient type, the oily outer layer is defective and rapid evaporation of the tear film occurs depriving the eye of its moisture. The aqueous-deficient type is caused by a malfunction of the lacrimal gland, often due to inflammation processes, such as promoted by auto-immune disease (e.g., rheumatoid arthritis or Sjorgren's syndrome). In Sjogren's syndrome (3 million Americans affected) this autoimmune syndrome destroys the epithelial cells in the lacrimal gland. Other disease that result in side affects of Dry Eye Syndrome are rheumatoid arthritis, diabetes, thyroid abnormalities, allergies, asthma, cataracts, glaucoma and lupus. Dry Eye is the primary cause of contact lens, especially soft contacts, discomfort or intolerance. Soft contacts rapidly evaporate the tears from the eye resulting in irritation, protein deposits, infection and pain. Abnormal blinking processes such as present in computer users or patients that have undergone refractive surgery (e.g., RK, PRK, LASIK, LTK) can be at risk for dry eye. 75% of people over the age of 65 years and 59 million people in the U.S. suffer from dry eye, also referred to as keratitis secca, keratoconjunctivitis sicca or xerophthalmia.

Tear drops are the most prevalent treatment for dry eyes. Although these can provide temporary relief, artificial tears also disrupt the eye's natural production of tears and lead to further aggravation of the condition including the washing away of the natural infection fighting tear film on the eye. Omega 3 fatty acids supplementation in the diet may help improve the tear layers. Punctal occlusion, plugs, lasers or cauterization can be used to prevent excess drainage leading to dry eyes. Collagen plugs can be used for the temporary occlusion of tear drainage.

Embodiments of the invention can correct dry eye and prevent corneal scarring, death of the cornea and conjunctiva, and infections of the eye. In some embodiments, progenitor cells or mature cells are isolated from the appropriate tear gland, expanded in number and the appropriate tear-producing cells are implanted into the gland or tissue that makes the three layers or a specific layer of tear the subject is deficient in. Thus the respective cells that produce the watery (e.g. lacrimal acinar cells), mucous (e.g. goblet cells) and oil layers (e.g. meibocytes) can be isolated and implanted. Autologous-made ECM can be used to plug the drainage system such as the puncta so as to keep tears on the eye longer and prevent excess tear drainage. Cells, such as connective tissue cells (e.g. fibroblasts) can be used for the long-term augmentation or blockage of the drainage system. The appropriate cells (e.g., keratocytes or fibroblasts) implanted under the epithelial layers can increase production of the tear layers. Fibroblasts can be implanted into the tarsal plate to assist in the effectiveness of the mucous secreting epithelial cells of the conjunctiva.

Anal Defects-Anus

The anus is the last portion of the gastrointestinal tract. The anal canal begins at the anorectal junction and ends at the anal verge, measuring between 2.5 and 5 cm long in adults. The anus is basically a muscular tube with four main layers. Starting from the lumen and working outward are as follows these layers are: 1) The mucous membrane or mucosa consists of a stratified columnar squamous epithelium, connective tissue and thin, smooth muscle. The upper portion of the epithelium is similar to that of the rectum and contains secretory and absorptive cells with tubular glands or crypts. The middle portion of the anal mucosa shows a non-keratinized stratified squamous epithelium and the inferior portion (closer to the perianal skin) shows the transition into a hair-bearing, keratinizing stratified epithelium. Underneath the epithelium and through all the extension of the anal canal, the submucosa is a wide zone of connective tissue (containing fibroblasts), supporting tissue and fat tissue(containing preadipocytes/adipocytes) with profuse arterial and venous plexuses. 2) The muscularis externa consists of two thick layers of smooth muscle fibers forming the internal anal sphincter (IAS). The IAS is a well defmed ring of obliquely orientated smooth muscle fibers continuous with the circular muscle of the rectum and terminating at the junction of the superficial and subcutaneous components of the external anal sphincter (EAS). The IAS provides most of the resting anal pressure and is reinforced during voluntary squeeze by the EAS.

3) The EAS is an oval tube-shaped complex of striated muscle, composed mainly of type 1 (slow twitch) skeletal muscle fibers which are well suited to prolonged contractions. The EAS forms a single functional and anatomical entity. Its more uppermost fibers blend with the lowest fibers of the puborectalis muscle, some anterior fibers decussate into the superficial transverse perineal muscles while some posterior fibers are attached to the anococcygeal raphe. The majority of the middle fibers of the EAS surround the lower part of the IAS. Disruption or weakness of the EAS can cause urge-related or diarrhea-associated fecal incontinence. Damage to the endovascular cushions may produce a poor anal “seal” and an impaired anorectal sampling reflex. 4) The adventitia or serosa is a thin outlayer covering of connective and supporting tissue.

Fecal Incontinence

Fecal incontinence may be defined as the involuntary loss of solid or liquid stool sufficient enough to result in impaired quality of life for the individual. Frequent or involuntary passage of gas (flatus) without loss of fecal material, while not clinically defined as incontinence, may also impair a person's quality of life and warrant treatment. Fecal incontinence is a symptom attributable to a variety of disorders affecting one or more factors that maintain continence. Fecal continence is maintained primarily by anorectal functions. Fecal consistency, personal mobility, and the individual's mental status are also critical for maintaining continence. The most prominent association with fecal incontinence by far is nursing home residence. The prevalence of fecal incontinence is about 2% to 3% for community-dwelling persons and may increase with advancing age to greater than 10%. Among nursing home residents the prevalence approaches 50%. Urinary incontinence is the greatest risk factor for fecal incontinence (and fecal incontinence is the most prominent risk factor for urinary incontinence), followed in order by the loss of ability to perform daily living activities, tube feeding, physical restraints, diarrhea, dementia, impaired vision, constipation, and fecal impaction. Inverse associations were noted with body weight, heart disease, arthritis, and depression.

Pregnancy, although not the exclusive cause of fecal incontinence, is certainly a prominent association due to damage to the anal sphincter and/or the pudendal nerve after a traumatic delivery. Factors leading to incontinence during pregnancy, immediately after pregnancy, and long after pregnancy have been investigated. Irritable bowel syndrome has been shown to be an important correlate with postpartum fecal incontinence. Several specific diseases have been associated with fecal incontinence, and mechanisms to explain the associations have been investigated. These include diabetes, multiple sclerosis, Parkinson's disease, spinal cord injury, systemic sclerosis, myotonic dystrophy, and amyloidosis. Many of these conditions directly affect mobility and ability to perform daily living activities, or they cause diarrhea or fecal impaction. Children born with congenital abnormalities related or unrelated to the gastrointestinal system can show fecal incontinence. Children with congenital anal anomalies, such as imperforate anus, often have lifelong problems with incomplete evacuation and soiling despite anatomical correction. Other children are born without anomalies but-for various reasons-withhold stool at an age beyond which toilet training should be complete and develop fecal soiling or have megarectum. Failure to retrain the child at an early age often leads to chronic impaction and fecal incontinence. Ano-rectal surgery can frequently result in fecal incontinence.

Medical treatment of fecal incontinence is often aimed to treat underlying conditions such as chronic diarrhea, constipation and fecal impactation. Surgical sphincteroplasty, muscular transfer with or without adding nerve electrical stimulation, placement of an artificial anal sphincter device and sacral nerve stimulation are the current surgical approaches to treat fecal incontinence.

Treatments described herein can be used to augment, reform or repair the sphincter structure, the tissue surrounding the IAS and/or EAS, causing a reduction in the abnormally wide and loose lumen. This requires the implantation of compositions into the regions surrounding the EAS or IAS or directly into a pocket created in the region to be repaired or augmented by: 1) injection of autologous cells and/or cultured cell ECM, such as fibroblasts, myofibroblasts, smooth muscle cells, skeletal muscle cells, myoblasts, undifferentiated mesenchymal cells, adipocytes, preadipocytes, amongst others; or 2) engraftnent of surgical strands comprised of the aforementioned autologous cells and/or ECM or other matrices containing the autologous cells or matrices alone. These techniques can be done with the cell types native to the area that receives the cells or other cell types.

Anal Fissure

In other embodiments, augmentation or repair of the anal sphincter is used to prevent the onset of anal fissures. An anal fissure is a wound in the lining of the anal canal, often displaying a painful small linear ulcer. An anal fissure is a common occurrence that happens more frequently in young and middle aged adults and occur equally in males and females. The most common cause of a primary anal fissure is tension in the rectal muscle. This muscle, called the sphincter muscle, will spasm and decrease blood flow to the rectal area, causing pain. The tension on the sphincter most often is from trying to pass a hard bowel movement through the anus. Primary anal fissures are located in the posterior midline of the anal canal more than 90% of the time. This distribution is due to the elliptical arrangement of the anal sphincter offering less support to the anal canal posteriorly. Secondary anal fissures are due to underlying disease such as inflammatory bowel disease (Crohn's disease) proctitis, leukemia, carcinoma and rarely syphilis or tuberculosis.

The symptoms of an anal fissure are pain, usually severe, and bleeding related to defecation. Anal fissures are diagnosed by physical examination of the anus and anal canal and sometimes are difficult to distinguish from hemorrhoids. Anal fissure treatment will depend on whether the fissure is acute or chronic. 90% of fissures are acute and can be treated by eating a fiber-rich diet, fruits and vegetables, and increasing fluids. Other fissure treatments include sitting in a warm salt bath for 10-15 minutes several times a day, medicated creams and suppositories. Chronic fissures are fissures that do not heal and last longer than one month. These usually require surgery to cut a portion of the sphincter muscle so as to decrease pain and spasm. Cutting the sphincter muscle normally does not interfere with bowel control but fecal incontinence can be a long term complication of the surgery.

Some embodiments are thus directed to a method to achieve healing of a fissure by implanting autologous cells, e.g., fibroblasts, at or near the fissure area. For instance, the cells may be placed into and/or along the entire fissure area. Autologous fibroblasts may be native cells, e.g., cells of the same type as the cells in the tissue of which the fissure is comprised. And autologous fibroblasts may be derived from a tissue with the same characteristics as the tissue(s) of which the fissure is comprised. Alternatively, autologous fibroblasts may be derived from a tissue different to the tissue of which the fissure is comprised. The cells, e.g., autologous fibroblasts, may be administered more than once and in different amounts as repetitive treatments preferably but not exclusively in the form of injections, or topical application as to attempt complete closure of the defect

Examples of defects treated with these techniques are: an iatrogenic fissure, a spontaneous fissure, a fissure due to ischemia, and a fissure due to inflammation secondary to, but not exclusively to, infection.

Skin Pigmentation-Skin Defects

The skin consists of two layers: The most external one, called the epidermis, a superficial layer of stratified squamous keratinized epithelium rich in keratinocytes that controls the loss of water from the underlying tissue, thus preventing dehydration. The internal layer, the dermis, is the thickest one and the most dynamic. It is formed by cells called fibroblasts that produce extracellular matrix, contains blood vessels, nerves, a variety of glands and in most areas, hair follicles. Together the two layers form the structural support for the skin. Additional hypolayers of subcutaneous fat and fascia can also be considered part of the skin, especially since these layers can become mingled, imparting complementary properties to the upper layers of skin.

A wrinkle, scar or other skin defects often affect not only these layers of skin, but the subcutaneous layers of adipose and fascia and the muscle layer underlying these layers. Cells implanted into the various layers or combination of layers can correct many skin defects including a wrinkle or scar. These cells include fibroblasts, preadipocytes, adipocytes, myoblasts, myofibroblasts, muscle cells, amongst others. For example, after tattoo removal, any residual damage or scarring of the skin can be repaired by implantation of dermal fibroblasts into or proximal to the skin defect.

Skin Pigmentation

The color of the skin in healthy people is determined by the oxygen content of underlying blood vessels, the presence of carotene (yellowish pigment) from the diet and mainly from the pigmentation of the epidermis derived from the melanocytes. The melanocytes, originate in the neural crest ectoderm, but are capable of division as an adult cell, are dendritic in structure and reside dispersed among keratinocytes, at the level of the basal layer of the epidermis. Differentiated melanocytes synthesize melanin pigment from precursors such as tyrosine and dopa and transfer the pigment to surrounding keratinocytes within granules called melanosomes. Melanin absorbs and scatters the ultraviolet (UV) radiation that is present in sunlight and thereby protects cells from the possible mutagenic effects of UV light. Since the amount of melanocytes is similar in light and dark skin the skin color is more dependent in the amount of melanin produced by a certain pool of melanocytes. Melanin production increases with prolonged exposure to sunlight causing a suntan, whereas lack of melanin in albino conditions is associated with a higher risk of epidermal damage and skin cancer. Cultural tendencies favor a suntanned body, but the dangers of ultraviolet (UV) radiation from the sun, tanning beds, and sun lamps are well known to increase the risk of skin cancer from which melanoma. UVB has long been associated with sunburn while UVA has been recognized as a deeper penetrating radiation that causes more damage.

Progenitor cells, melanocyte stem cells or melanoblasts, are unpigmented precursor cells to differentiated melanocytes that are present in the skin, the dermis and epidermis. Stem cells can be Pax-3 stimulated to expand and to differentiate by a transcriptional factor, Mitf, whose expression is also Pax-3 stimulated. This stem cell transcription factor thus both maintains a partially undifferentiated melanocyte stem cell state and can determine the cell fate.

Augmentation or repair of skin color or tanning can be performed as described herein by implanting cells that supply the requisite functions. Thus embodiments include the implantation of melanocytes, melanoblasts, or other progenitor or stem cells that produce melanocytes or the phenotype of melanocytes. Melanocytes can be obtained for cell culture expansion without exposure or prior exposure to ultraviolet light. The cells can be exposed to ultraviolet light prior to implantation, while in culture or in suspension. Increasing the amount of melanocytes producing melanin is the preferred embodiment of the invention. Melanocytes or melanoblasts or progenitor cells to melanocytes can be obtained from the skin layers or from other tissues for implantation into the skin. These tissues include cells from the hair follicles. The respective cells can be implanted into their natural locations within the skin. A preferred embodiment is the implantation of the cells into the epidermal layer of the skin in which tanning or skin color is desired. The dermal layer is an option or can be done in tandem with implantation into the epidermal layer.

An additional benefit of the implantation is as a preventive to cancer of the skin, by protection of the implanted skin with melanin. Spotty skin pigmentation and other skin pigmentation defects, such as vitiligo, can be corrected with cell implantation with similar melanocyte cells. Vitiligo is the appearance of nonpigmented white patches of varied sizes and is usually bordered by hyperpigmented areas in which the hair in the affected area is often white. The epidermal melanocytes are missing in depigmented areas caused by an autoimmune process. Individuals with albinism can be treated with melanocytes capable of producing melanin.

Hair Graying

The hair follicle has a long tube like structure and is divided into the upper and lower sheath. The former retains its structure during all the hair growing phases while the cyclic remodeling changes of the hair follicle occur in the lower sheath. Therefore, all the follicular accessory structures (sebaceous gland, erector muscle, sensory nerve and the apocrine gland's duct) remain intact. The lower sheath, including the bulb of the hair follicle, contain cells that proliferate and migrate upward differentiating into three major groups: hair matrix, inner and outer sheath. The hair matrix further differentiates into the medulla, hair cortex and cuticle. The inner sheath forms the cells that constitute the inner wall of the pilary canal. The outer sheath cells differentiate into cuboidal cells that storage large amounts of glycogen as an energy source.

In the follicular bulb melanocytes can be observed and, although they do not migrate their products (melanin pigments) into the hair cortical cells, they are responsible for keeping the color of the hair. Outside the hair matrix, melanocytes can also be present along the outer root sheath, the infundibulum, the bulge and subbulge of follicles, near the sebaceous gland and in the epidermis. Melanoblasts or progenitor cells to melanocytes can be predominantly located outside the follicular bulb area, in the outer root sheath around the bulge area where the arrector pili muscle attaches below the sebaceous gland. These cells can also be present elsewhere in the follicular structure.

Melanocytes can be obtained from non-greying hair follicles. Additionally, melanoblasts, melanocyte stem cells or progenitor cells to melanocytes can be used. Melanocytes, melanoblasts or progenitor cells to melanocytes can be obtained from other tissues of the body including skin (e.g., epidermis). The respective cells can be implanted into their natural sites within the hair follicles, e.g. melanoblasts into the bulge area and melanocytes into the follicular bulb area. Alternately, the cells can be implanted in or around the follicles of interest. The implantation can control the color of the hair. In particular, a removal of grey color and the person's natural hair color or other color is preferred by the addition of melanocytic cells. Any hair region of the body can be implanted including facial hair (e.g. beards), eyebrows, scalp hair, pubic hair, arm and leg hair, amongst others.

Nails

Nails are plates of hard keratin equivalent to the stratum corneum of the epidermis. Beneath the nail is the nail bed, consisting of deeper layers of the epidermis (the basal epidermal layer or stratum germinativum, stratum spinosum, and the stratum granulosum). All layers are rich in keratinocytes that are at different stages of differentiation in their migration to the surface. Deep to the ridge of soft skin (cuticle) at the proximal end of the nail is the nail matrix (germinal layer), containing the proliferative cells that form the growing nail. The proliferative cells can be progenitor cells or the mature nail producing cells. The white crescent shaped lunula is the distal portion of the matrix; its color is determined partly by light scattering and partly by the thickness of the epithelial cells of the matrix.

The nail matrix is the source of the nail plate. There are three parts of the nail matrix: the undersurface of the proximal nail fold or the dorsal matrix, the intermediate matrix or germinal matrix which begins where the dorsal matrix folds on itself and extends to the distal portion of the lunule, and the ventral or sterile matrix, which makes up the rest of the nail bed that begins at the distal portion of the lunulea and ends at the hyponychium. The matrix epithelium contains typical basal and prickle cell layer keratinocytes, and a scattering of melanocytes and Langerhan's cells. The cornified cells of the dorsal and ventral portions of the matrix extrude distally to form the nail plate.

The epidermis of the nail bed is thin, lacks a stratum granulosum and consists of a couple of layers of nucleated cells lacking keratohyalin granules. A thin cornified layer moves distally with the growing nail plate. The dermis of the nail bed lies underneath and is anchored to the periosteum of the distal phalanx without a subcutaneous layer. Nail bed cells differentiate towards the nail plate in a ventral direction. Normally nails growth 2-4 mm per month. A fingernail grows complete in about 6 months, whereas toenails do the same in 12-18 months.

A preferred embodiment for nail growth is to expand nail matrix or progenitor cells obtained from in or around the nail matrix and then implant expanded matrix cells or progenitors of the nail matrix cells into the nail matrix or areas close to the matrix. Additionally, implantation of fibroblasts into the dermis layer can assist the growth of the nail plate. The cells may be introduced with or without the proteins, factors, and supplementing materials described herein. Autologous cells, allogenic cells, or xenogeneic cells may be used. Cells include stem cells, various differentiated cells, and their precursors.

### Additional Embodiments Related to Skin Defects

A number of defects in the skin have been enumerated in this text and in references incorporated herein. Additionally skin defects that are due to inflammation, dryness, loss of tone or tissue volume can be treated with specific cell types of the invention. Aging skin for example has less moisture, due to less ECM production (e.g. proteoglycans) in the dermal and subcutaneous layers. The papillary dermis has the highest concentration of hydrated ECM (e.g, proteoglycans, type III collagen) compared to other layers of the subepidermis. Implantation of fibroblasts and/or a proteoglycan or other hydrating factors (e.g., GAGs, hyaluronic acid) or proteins can increase the moisture content of skin, promote turgor and increase the volume of the skin. Additionally, this procedure can be used in all tissues and organs to improve the moisture or hydration content as well as confer additional elasticity. In a preferred embodiment, papillary fibroblasts are used to increase skin turgor and moisture, to improve skin mass, and to treat the various skin defects. In a preferred embodiment papillary fibroblasts are implanted into skin, in particular in the upper layer to increase cushioning or insulation of the skin. This can also be obtained by increasing skin volume with other cell types such as preadipocytes, fascia and reticular fibroblasts.

The papillary dermis contains vascular networks that support the avascular epidermis with vital nutrients and it provides a network for thermoregulation. The vasculature is organized so that heat can be either conserved or dissipated by increasing or decreasing blood flow. The vasculature interdigitates in the dermal papillae area. Thus implantation of papillary fibroblasts into, at or near the upper layer of dermis can control body thermoregulation. Other cell types in the skin, the reticular fibroblasts, fasia fibroblasts and preadipocytes/adipocytes may also assist.

Mechanical strength of the skin is determined in good part by the reticular dermis. Thus the introduction of reticular fibroblasts into or near the reticular layer of skin will strengthen the skin. With aging the skin is prone to bruising and tears and the mechanical strength embodiment can assist these skin conditions. Other cell types in the skin, the papillary fibroblasts, fascia fibroblasts and preadipocytes/adipocytes may also assist.

The elasticity of the skin is largely determined by the reticular dermis. Thus the implantation of reticular fibroblasts into or near the reticular layer of skin will make the skin more elastic and normal. Aged skin loses its elasticity. Other cell types in the skin, the papillary fibroblasts, fascia fibroblasts and preadipocytes/adipocytes may also assist. Aging affects the above described properties and promotes the defect. Similarly, these properties can be a function of aging, and are present in other organ and tissue systems.

Psoriasis

Psoriasis is one of the most common dermatologic diseases, affecting up to 2.5% of the world's population. It is a chronic autoimmune inflammatory skin disorder resulting from a complex and aberrant relationship between the skin and the immune system in an individual with genetic and environmental pre-disposition. There is a definitive link to some of the human leukocyte histocompatibility antigen system (HLA). Psoriasis can cause excess proliferation of the epidermis. It appears there is an interaction of T-cells with the keratinocytes of the epidermis.

Psoriasis is clinically characterized by erythematous sharply demarcated papules and rounded plaques, covered by silvery micaceous scales. The skin lesions are variably pruritic and occur most commonly on elbows, knees, gluteal cleft and the scalp. Microscoscopically the lesions show acanthosis, vascular proliferation and massive T cell infiltration. In up to 10% of individuals, especially in those with nail involvement, the joints are affected. This psoriatic arthritis characteristically shows asymmetric joint involvement, but are negative for rheumatoid arthritis factors in serum. Nail involvement consists of a punctate pitting and nail thickening.

The disease resides in the basal layer of the skin, the lower part of the epidermis adjacent to the dermis and in contact with the basal lamina. The basal layer is the layer where cell proliferation in the epidermis takes place. The majority of the basal layer cells are keratinocytes columnar to cuboidal in shape. Melanocytes, Langerhan's cells, occasional Merkel cells and intraepithelial lymphocytes are interspersed among the basal keratinocytes. From bottom up the basal layer is organized into three main layers. The deepest one is the prickle cell layer, followed upwards by the granular layer and topped by the cornified layer as the final product of epidermal differentiation. Cells are thought to form a series of columns. Several layers of prickle and granular cells overlie a cluster of six to eight basal cells forming a columnar proliferative unit. Each unit consists of a central multipotent stem cell that may self-renew or produce a daughter cell which is committed to differentiate, encircled by transit amplifying proliferative cells and postmitotic maturing cells.

In normal skin the total epidermal turnover time is between 52 and 75 days. In patients with psoriasis, the control of keratinocyte proliferation and differentiation is lost, it may be as little as 8 days. There has been controversy as to the causes of the disease, whether it is triggered and propagated within the skin or by the infiltrating T cells (CD4+ and CD8+ mainly) release of cytokines that activate growth factors and stimulate keratinocyte hyperproliferation. At the molecular level, the transcription factors can activate expression of a group of proteins, STAT 3, that mediate the interferon signaling in the basal stem-cell layer of the epidermis and have a role in the proliferation and migration of epidermal keratinocytes. Over-expression of STAT 3 proteins is widely observed in animal models of psoriasis

Patients with localized, plaque-type disease benefit from topical glucocorticoids, although long- term use can cause atrophy of the skin. A topical vitamin D analog (calciprotene) and retinol may benefit patients with localized and limited disease. Ultraviolet light (UV-B plus UV-A) treatment is beneficial for patients with widespread psoriasis. Methrotexate can be used especially for patients with psoriatic arthritis. The evidence linking psoriasis with a T cell-mediated disorder has directed therapeutic efforts to immunoregulation. Cyclosporine is commonly used for patients with severe and widespread psoriasis. Recent research has been focusing toward the development of biologic agents with selective immunosuppressive properties and less secondary effects. Inhibitors of tumor necrosis factors α are the subject of some recent clinical trials. Other agents in clinical trials target other proinflammatory cytokines, T cell activation, and lymphocyte trafficking in an attempt to suppress the inflammation. There is indication that estrogen may attenuate inflammation in psoriatic lesions by down-regulating the production of the neutrophil, T cell and macrophages attracting chemokines by keratinocytes.

In a preferred embodiment, papillary fibroblasts from skin tissue are taken from an unaffected skin site, expanded in vitro and implanted into the upper dermis. Alternately, these fibroblasts and others from skin (reticular, dermal, fascial fibroblasts) or other tissue fibroblasts (e.g., bone marrow stromal fibroblasts) are expanded and implanted into the dermis and subcutaneous layers. The fibroblasts can provide moisture to the dry epidermal layer to mitigate symptoms and can control the chronic inflammation accompanying the disease. Fibroblasts can secrete keratinocyte regulatory and growth factors to control cell proliferation and differentiation of the keratinocytes (e.g., KGF, βIFN). In another aspect of the invention, progenitor cells to fibroblasts can be used. In another aspect of the invention, immune cells or progenitor immune cells from the bone marrow can be implanted or infused so that these cells regulate in a normal fashion, such as immune surveillance and quench the autoimmune reaction in the epidermis.

Eczema

Eczema or dermatitis is a reaction pattern that presents with variable clinical and histologic findings and is the final common cutaneous expression for a number of disorders including atopic dermatitis, allergic contact and irritant contact dermatitis, dyshidrotic eczema, nummular eczema, lichen simplex chronicus, asteatotic eczema and seborreic dermatitis. The skin can become very dry.

Atopic Dermatitis (AD) is the skin expression of the atopic state, characterized by family history of asthma, hay fever or dermatitis in up to 70% of the patients. Clinically AD is a disease course lasting longer than 6 weeks and marked by pruritus and scratching, exacerbations, remissions, eczema lesions in flexural skin, hands or lichen type lesions, personal or family history of atopia (e.g., asthma, allergic rhinitis, food allergies or eczema). The etiology of AD is not completely understood but there is clear genetic predisposition. When both parents are affected 80% of their children will be, when one parent is affected up to 50% will be. Patients with AD display a number of immunoregulatory abnormalities including increase IgE synthesis, increased serum IgE, increased specific IgE to foods, aeroallergens, and bacteria, increased expression of CD23 (i.e., low-affinity IgE receptor) on monocytes and B cells, and impaired delayed type hypersensitivity reactions. Histologic examinations of the affected skin display features of acute or chronic dermatitis. Immunopathology shows activated, memory T helper cells, and Langerhan's cells with IgE bearing CD1a+ that mediate a hypersensitivity response to environmental antigens.

Contact Dermatitis (CD) is an inflammatory process in the skin caused by an exogenous agent or agents that directly or indirectly injure the skin. The most common type of CD is hand eczema, often related to occupational exposures. This injury may be caused by an inherent characteristic of a compound referred to as irritant contact dermatitis (ICD), or allergic contact dermatitis (ACD), that induces an antigen-specific immune response. The clinical lesions of CD may be acute (i.e., wet and edematous) or chronic (i.e., dry, thickened and scaly). ACD is a manifestation of delayed-type hypersensitivity mediated by memory T lymphocytes in the skin. The most common cause of ACD is skin exposure to plants such as poison ivy, oak and sumac that have a specific antigen urothiol that adheres to skin, clothing, tools, etc causing an often linear erythematous eruption with vesiculation and severe pruritus.

Therapy of AD may include avoidance of cutaneous irritants, the use of moisturizing and of topical anti-inflammatory agents. The wide use of topical glucocorticoids has been replaced by the use of non-glucocorticoid agents as tracolimus and primecrolimus (macrolide immunosuppressants) due to the undesirable secondary effects of glucocortiocoid induced skin atrophy. Anti-histamines are commonly added to the therapy to control the pruritus.

In a preferred embodiment, cells, e.g., fibroblasts, papillary fibroblasts from skin tissue, adipocytic cells, or precursors thereof are taken from an unaffected (skin) site, expanded in vitro and implanted preferably into the subepidermal dermis. Alternately, these fibroblasts and others from skin (reticular, dermal, fascial fibroblasts) or other tissue fibroblasts can be expanded and implanted into the dermis and subcutaneous layers. The fibroblasts can provide moisture to the dry epidermal layer to mitigate symptoms and can control the chronic inflammation accompanying the disease. Fibroblasts can secrete keratinocyte regulatory factors to control cell proliferation and differentiation of the keratinocytes (e.g., KGF, IFNβ). In another aspect of the invention, progenitor cells to fibroblasts can be used.

Tooth Growth and Defects

Teeth develop through a series of epithelial-mesenchymal interactions, forming a bud, followed by a cap around which ectomesenchymal cells aggregate into the inner and the outer enamel epithelium (IEE and OEE). Cells from the IEE differentiate into secretory ameloblasts which secrete the organic matrix of the enamel. Later these ameloblasts will form odontoblasts that produce dentin. Ameloblasts mineralize the developing enamel and degenerate after the enamel is fully mineralized and the crown of the tooth is completely formed. As opposed to bone formation, the dentin forming cells are outside this hard tissue. Up to 80% of the dentin mass is mineralized and form parallel tubules radiating from the pulp chamber. The pulp chamber is lined by a layer of non-mineralized matrix called predentin that is secreted by odontoblasts. The dentin tubules protrude from this odonotoblast layer. Each tubule is a cytoplasmic extension of an odontoblast cell surrounded by a collar of dentin that is calcified. The dental roots are covered by an avascular, bone like layer called the cementum. The cementum is derived from dermal follicle tissue. This layer contains on the inside cementocytes (similar to osteocytes in bone). On the outside, this layer contains cementoblasts (similar to osteoblasts in bone). Emanating from the cementum are collagen fibers that constitute the principal fiber component of the periodontal ligament that anchors into adjacent alveolar bone. New layers of cementum are deposited throughout life to compensate for tooth movements. Lack of cementum overlapping the enamel exposes the dentine in the mouth. Thus the teeth can be sensitive to cold or water stimuli. The root also may become exposed due to occlusal drift, gingival recession and loss of cementum by incorrect tooth brushing (additional dentine exposure). Cementoblasts can be implanted into areas (e.g., gingival sulcus) on the outside of the damaged or missing cementum layer to correct tooth sensitivity. The implantation can bye used for root canal cementum defects due to infection or abscesses, for example. Teeth can become loose due to gum disease. In another aspect of the invention implantation of cementoblasts can be used to firm the tooth setting in the sulcus area. In extensive gum disease, the periodontal area can be rebuilt in tandem by implantation of lamina propria fibroblasts or other tissue type fibroblasts.

Tooth development occurs fetally and postnatally. Two sets of teeth begin formation at 6 weeks in utero. There are 32 teeth in the adult preceded by 20 deciduous teeth that are shed from the sixth to about the twentieth year. The developing tooth bud lies in gums beneath the deciduous teeth. Osteoclasts resorb deciduous teeth roots as the adult teeth form. BMP- and FGF-family growth factors are expressed in dental epithelium during initiation of tooth development and their effects on the underlying mesenchyme copy those of the epithelium. They upregulate the expression of many genes, including the homeobox-containing Msx-1 and Msx-2, and stimulate cell proliferation acting as epithelial signals transmitting epithelial-mesenchymal interactions. During subsequent morphogenesis, the characteristic shapes of individual teeth develop as a result from folding of the dental epithelium and signal molecules such as sonic hedgehog, Bmps-2, 4, 7 and Fgf-4 expressed in transient epithelial cell clusters, called enamel knots. A local ectodermal thickening expressing several signaling molecules appears. It is believed that these in turn signal to the underlying mesenchyme triggering mesenchymal condensation and tooth development. Epithelial cells make the enamel and mesenchymal cells make the soft tissue of the tooth.

A tooth bud is a mass of tissue that can form the parts of a tooth. The tooth passes through three developmental stages: growth, calcification and eruption. Tooth buds are the patches of epithelial cells that eventually grow into underlying tissues. By the seventh week of fetal development, epithelium cells (skin cells of the mouth), thicken along the ridge of the developing jaws. The cells of the epithelium form the dental lamina, a horseshoe-shaped band in the mouth. The growth period then begins and is divided into three stages: bud, cap and bell. Permanent teeth tooth buds develop from the seventeenth week of fetal development until the age of five. The second stage of growth is the cap stage in which proliferation takes place. As the cells of the tooth grow, the tooth bud takes the shape of a cap. The area underneath the cap is called the dental papilla. In the final stage, the bell stage, the epithelium of the cap will form the enamel. The dental papilla will form the dentin, cementum, and the pulp. At this stage, the tooth takes on the shape and form of a tooth. The next stage of tooth development is calcification, in which the cells deposit calcium and mineral salts to harden the tissue, followed by layers of enamel to form the tooth from the top of the crown down. Once the tooth crown has formed, the root begins to develop, triggering eruption. During eruption the upward movement of the tooth positions into its assigned location in the mouth. For permanent teeth, three years elapse between the time of crown completion and the time of tooth emergence.

An adult tooth consists of a crown and a root, and is comprised mostly of dentin, an avascular and acellular but living connective tissue. It is formed slowly throughout life and attaches to the enamel by the intermingling of hydroxyapatite crystals. The crown projects from the gingiva and is covered with enamel, the hardest substance in the human body, mainly consisting of hydroxyapatite crystals. Most of the tooth is made up of the root which contains a central pulp cavity of loose connective tissue, suspended in and anchored by the periodontal ligament in an osseous socket of alveolar bone. Pulpal cells induce neurite outgrowth.

The root is covered by a thin layer of bone like tissue called cementum, containing cells and extracellular matrix. Enamel and cementum usually meet at the gingival sulcus. The tooth contains a central pulp cavity of loose connective tissue, narrowed in the deeper root(s) to form the pulp or root canal which, via the small foramen at the tip of each root, is continuous with the periodontal ligament, allowing the entry of vessels and nerves into the pulp cavity. The gingivae are specialized regions of oral mucosa consisting of parakeratinized stratified squamous epithelium which at the neck or cervical margin of the teeth, attach to adjacent bone. The gingival epithelium rests over a thick layer of stromal connective tissue called lamina propia, which is rich in fibroblasts and extracellular matrix. The ECM contains multiple collagen types, such as I, III, IV and V fibers in a very similar arrangement to the skin. Collectively, the gingiva, lamina propria, periodontal ligament, alveolar bone, and cementum are called the periodontium.

Certain embodiments of the invention can address tooth defects including reconstruction of tooth structures such as those damaged due to dental cavities, infections, abscesses, enamel hypoplasia, nerve root canal injuries, microdontia, hypodontia, pulp polyps, tooth reconstruction and the need for new tooth growth. The cell types described above for the various tooth structures can be isolated from and implanted into the in situ location of the defect, with the implanted cells preferably being native to the tissue that receives them. For example, implantation of ameloblasts and/or odontoblasts to produce new dentin and enamel can provide the subject with whitening of the teeth. Progenitor cells can be used, in particular for new tooth growth. New adult tooth growth can be achieved using dental bud stem epithelial cells and/or dermal papilla cells by implantation into the gum lamina propria or periodontal membrane area surrounding the current tooth's roots or the area in which the tooth location is desired.

Alveolar Bone Defects.

Alveolar bone forms the part of the maxilla and the mandible which supports and protects the teeth. As with other bones, alveolar bone functions as mineralized supporting tissue, gives attachment to muscles, provides a framework for bone marrow and acts as a reservoir for calcium. It is dependent on the presence of the teeth for its maintenance, thus in anodontia (congenital teeth absence) the alveolar bone is severely hypoplastic and it atrophies after tooth extraction. Alveolar bone reabsorption is particularly prominent in elder individuals who have wore dentures for a long period of time to the point that often it is a severe problem for these individuals to keep the dentures in place. The alveolar tooth-bearing portion of the jaws consists of an outer and inner alveolar plates. The individual tooth sockets are separated by plates of bone termed the interdental septa, and the compact layer of bone at the bottom of the socket is called the cribiform plate which is perforated to give passage to the blood vessels and nerves from and to the roots of the tooth. This passage is called the Volkmann's canal. The invention can use osteogenic cells for mandible and maxillar alveolar bone reconstruction and repair.

Augmentation of the Calcaneal and Plantar Overlying Fat Pads.

The foot can be submitted to severe stress caused by extensive walking or standing while wearing ill fitted shoes or very high heeled shoes. This stress can translate in feet pain especially in the heel area (ball of the foot) due to faulty biomechanics that create unbalanced weight support with one area of the foot withstanding the greater majority of the person's weight, as is the case with high heeled shoes. Feet pain caused by wearing high heeled shoes can be acute or can become chronic and potentially the cause of other more serious conditions as fascitis (chronic inflammation of the fascia of the foot) or even deformation of the arch of the foot over time causing even more pain.

For the foot to better withstand the stress of wearing high heeled shoes a potential solution is to augment the natural fat pad that overlays the calcaneal bone in the heel, an area known as the ball of the foot. The augmentation can be performed by injecting or surgically implanting or inserting fat cells, pre-adipocytes, fibroblasts, cells to make muscle, collagen, other ECM proteins or matrix or a combination in the area. Moreover, precursors to the same may be used. The cells may be implanted with or without helpful proteins or other factors set forth herein.

Muscle and Muscle Defects

The basic muscle types consist of cardiac, skeletal and smooth muscle cells. The central dogma has been that cardiac cells (cardioblasts) do not proliferate after birth, but do in the fetal stage. They grow by hypertrophy in the adult and function in involuntary regulation of pacemaker-generated heart beat by the autonomic nervous system. Muscle spindles are absent, synapses are en-passant and cell junctions at the intercalated disks are present as fascia adherens, desmosomes and gap junctions. The muscle type has intermediate sarcoplasmic reticulum and T tubules at Z disks forming diads with a terminal cirsterma. A, I, H bands and Z disks are present. Contraction occurs when extracellular calcium enters, inducing additional calcium release from the sarcoplasmic reticulum and terminal cisternae. Postganglionic sympathetic neurons release norepinephrine binding to the □₁ adrenergic receptor while postganglionic parasympathetic neurons release acetylcholine binding to the M₂ muscarinic acetylcholine. The cells have short branching cylinders and contain the central nucleus. In contrast to skeletal myocytes, these cells can contract and relax spontaneously.

Skeletal muscle cells do not proliferate in the adult but the satellite cells in skeletal muscle tissue gives rise to myoblasts. Its regeneration is thus limited. Skeletal muscle typically grows by hypertrophy and contracts by the voluntary regulation of “all-or-none” contraction of □—motor neurons which release acetylcholine and bind to the nicotinic acetylcholine receptor at the neuromuscular junctions. Muscle spindles are present and cell junctions are absent. The cells have extensive sarcoplasmic reticulum, contain A, I, H bands and Z disks with T tubules present at a A-1 junction and can form triads with terminal cisternae. Contraction occurs through the release of calcium stored in the sarcoplasmic reticulum and terminal cisterna, in which troponin C is the calcium binding protein. The cells are long parallel cylinders with multiple peripheral nuclei. Skeletal muscle types are red fibers (type 1), white fibers (type 2) and intermediate fibers. In the invention, fibroblasts can be obtained, expanded in vitro and converted into skeletal muscle cells by the transcription factors MyoD, myogenin, Myf-5 and Myf-6 or other transdifferentiation or differentiation factors. The resulting skeletal muscle cells can be used in many aspects as described. For example, age-related loss of muscle is known as sarcopenia and muscle cells can be implanted into muscle tissue and surrounding tissue to treat this disease. Myoblasts can be derived from satellite cells found on the surface of mature myofibers or from cells in bone marrow or interstitial connective tissue. Muscle can be added by cell implantation to increase physiological homeostasis, hormone balance, increase metabolic activity and blood flow, all of which is dysfunctional during aging. Muscle cells can be used to treat muscle-wasting diseases, muscular dystrophy, disuse atrophy (e.g., paralyzed patients, elderly), amongst others.

Smooth muscle cells can proliferate in the adult and pericytes can give rise to new cells. Growth in the adult is by hypertrophy and proliferation. The cells are under involuntary regulation of contraction by the autonomic nervous system and by hormonal control. Muscle spindles are absent and synapse en passant. The cells have a limited sarcoplasmic reticulum and its gap junctions are present in the single cells, but not the multiunit. Contraction occurs when extracellular calcium enters the cells and induces more calcium release from the sarcoplasmic reticulum under neural control. Calmodulin binds the calcium. The cells have actin and myosin filaments, dense bodies and plaques connected by intermediate filaments with caviolae present. Postgangionic sympathetic neurons release norepinephrine binding to the α₁ and β₂ adrenergic receptors while postganglionic parasympathetic neurons release acetylcholine binding to the M₃ muscarinic acetylcholine. The cells are spindle shaped with tapering ends containing a single central nucleus. The cells can be a single-unit, multiunit or combination unit. Single-unit smooth muscle is present in the uterus, ureter, urinary bladder and GI tract, whereas the multi-unit is present in the dilator and sphincter pupillae muscles of the iris, ciliary muscle of the lens and the ductus deferens. The combination unit is found in the tunica media of blood vessels. Smooth muscle cells is present in the uterine myometrium during pregnancy, in the gut and the skin also. Smooth muscle myoblast may occur as myoid, myoepithelial or myofibroblast cells.

Damage to any muscle type, (e.g. through injury, disease or aging), can be repaired by implanting expanded muscle cells. Preferably, the same muscle cell type is put back into the normal in situ location of that muscle cell type. Augmentation or repair of muscle, such as skeletal muscle can also be attained by the implantation of myoblasts derived from satellite cells in a preferred embodiment. This can build bigger muscle tissue, strength, increase distribution of physiological bloodflow, enhance physiological peripheral oxygen consumption and utilization and improve hormone balance. It can prevent bone loss such as that which occurs in osteoporosis or osteopenia. The enhanced muscle mass and functioning can restore normal glucose homeostasis in diabetes mellitus type II. To obtain cardiac muscle repair, other muscle cell types can be substituted, in a variety of combinations, such as smooth muscle or skeletal cell types. The different muscle cell types can be substituted for each other in an alternate method of repair of muscle tissue. Augmentation of muscle can be performed due to a patient's cosmetic reasons, such as skeletal muscle bulking or penile smooth muscle bulking.

The Cardiovascular System Defects—The Heart and Blood Vessels

The Heart

The heart can be considered as a complex modification of a tube which, during its development, becomes divided into two longitudinal compartments folded back on themselves such that the inflow and outflow vessels are located next to one another. The chambers of the heart share several features commonly seen in various blood vessels, including a three-layered wall, valves and nerve supply. As an organ responsible for propelling blood through the circulatory system, the heart resembles a demand pump, since its pumping mechanism is not fixed in terms of outflow, but responds to variations in circulatory flow periods of rest or exercise. The septa separate the atria and the ventricles from each other. The septum between the atria is mainly fibrous connective tissue while the septum between the ventricles is primarily myocardium with a layer of endocardium.

The walls of the heart contain three layers. The middle and thickest layer of the heart wall is the myocardium which is made up of bundles and layers of cardiac muscle consisting of cardiac myocytes described as myocardial fibers. These fibers are individual cells joined end to end by special intercellular junctions called intercalated discs. These discs also provide electrical coupling. Myocytes have a single central nucleus. The fibers branch, forming striations and sarcomeres (contraction units) that represent repeating regions of actin and myosin filaments, which slide along each other during contraction. The myocardium contains Purkinje myocytes and myocardial endocrine cells. The endocrine cells are found in the atria and secrete atrial natriuretic peptide (ANP) in response to increased blood volume and venous pressure within the atria. ANP increases glomerula filtration pressure and rate, decreases sodium reabsorption, inhibits secretion of antidiuretic hormone from the neurohypophysis, aldosterone form the adrenal cortex, renin from juxtaglomerular cells and causes vasodilation of peripheral and renal blood vessels. Muscle fibers attach to the fibrous skeleton, a system of rings of connective tissue and elastic fibers separating atria from ventricles. The fibrous skeleton also forms thick connective tissue bands around the heart valves for support. Each valve is a flap of fibroelastic connective tissue extending from the fibrous skeleton and covered by endocardium. The papillary muscle attaches to the valve leaflets and cusps by chordae tendineae and assists in the opening and shutting of the valves. Each cusp is a fold of endocardium with an intervening fibrous core. Principal elements of the fibrous skeleton of the heart are the valve annuli comprised of fila coronaria and sulcal connective tissue that is continuous with the valve cusps. Throughout the heart, from epicardium to endocardium, the intercellular spaces between contractile and conducting elements have varying amount of connective tissue. A thin layer of areolar tissue covers much of the mesothelium of the serosal visceral epicardium, accumulating fat during aging. Coronary vessels are embedded in this fat and it is located along the atriventricular and interventricular grooves and side channels. The fibrocellular components of the subepicardial and subendocardial layers blend with the endomysial and perimysial connective tissue on the myocardium. Thus each cardiac myocyte is composed of fine reticular fibers, collagen and elastin fibers embedded in ground substance. The fibrous skeleton serves as the attachment for cardiac muscle fibers and prevents the spread of electrical impulses from atria to ventricles except for that by the conducting system. The myocardium of these chambers is lined by supporting tissue of the inner endocardium (endothelial layer underlain with the subendocardial space containing Purkinje myocytes and continuous with veins and arteries that enter and leave the heart). The outer epicardium (a squamous-type mesothelium and basal lamina comprising connective tissue containing blood vessels and nerves that supply the heart). Purkinje myocytes are connected by gap junctions and specialize in conduction. The excitation waves for depolarization originate in the sinoatrial node, known as the heart pacemaker, which distributes electrical impulses to the atrioventricular node followed by the atrioventricular bundle of His. The nodes and bundle are made of small, slender transitional myocytes and the terminal branches of the Purkinje fibers, are made of cells larger than normal myocytes. Covering the epicardium is a connective tissue sac, the pericardium. Pericardial fluid is present (˜50 ML) in the pericardial cavity between the pericardium and epicardium. The pericardium consists of inner parietal and outer fibrous layers.

Cardiac efficiency depends on the timing of the function in interdependent structures. Thus there is passive filling of the atria and ventricles and stimulation by discharge from the sino-atrial node resulting in atrial systole that completes filling of the ventricles. Excitation and contraction of the atria occurs synchronously and is completed before ventricular contraction, that is caused by a delay in the conduction of excitation from the atrial to ventricles. Ventricular contraction proceeds in which a specialized ventricular conduction system ensures the closure of atriventriuclar valves followed by a rapid wave of excitation and contraction, which spreads from the apices of the ventricles towards the outflow tracts and orifices, accelerating the blood during ejection. The main pacemaker rhythm is generated in the sinuatrial node, influenced by nerves (sinus and its innervation) and is transmitted from atria to ventricles by the atriventriuclar node and bundle and within the ventricles to all the musculature.

Nodal cells or pacemaker cells (P cells) are grouped in an elliptical structure, 1-2 cm long, the sinoatrial node. Nodal tissue is located subepicardially within the terminal groove of the right atrial wall. The cells are embedded into a dense collagenous adventitia. Autonomic ganglions border the node, P cells are mostly in the center, are small (5-10 um maximal diameter) with a large central nucleus. Myofibrils are few and there is no proper sarcotublular system. P cells mix with slender fusiform transitional cells at the periphery, that are between a P cell and a normal cardiac cell in appearance. A similar arrangement of P cells is in the atrioventricular node. The atrioventricular bundle is a direct continuation of the AV node as it enters the central fibrous body and reaches the papillary muscles.

The cells of the myocardium, conducting tissue and the cardiac jelly (the specific ECM of the developing heart) derives from the midline splanchopleuric coelomic epithelium. The endocardium and cardiac mesenchymal cells producing valvular tissue derives from the angioblastic mesenchyme. The aorticopulmonary septum and the tunica media of the great vessels is derived from neural crest cells. Cardiac mesenchyme is produced by epithelial-mesenchymal transformation of a subset of endocardial cells that line the inflow tract at the atrioventricular canal and the outflow tract in the distal bulbus cordis and truncus arteriosus. The atriventricular cushions are formed from cardiac ECM containing fibronectin, hyaluronic acid and hyaluronidase in the cardiac jelly and from mesenchymally transformed endocardial cells. The cardiac jelly or myocardial basement membrane has inductive factors that can differentiate specific endocardial cells.

Postnatally, cardiac muscle cells do not proliferate but hypertrophy by synthesizing extra myofibrils. Degeneration or injury of cardiac muscle can lead to replacement of the area with scar or fibrous tissue. Embodiments of the invention can use fibroblasts or muscle cell types to replace or repair the scar or fibrous tissue, or to augment the function of the tissue near the scar by providing functioning cells.

The sarcomeres have two kinds of contractile filaments. Thick filaments are composed of myosin and the thin filaments are composed of actin. Both types are arranged in regularly repeating segments called the Z lines. A sarcomere is the region between and includes two successive Z lines. The area where overlapping of thick and thin bands occur is called the A band. Within the A band are H, M and I bands. The sarcoplasm contains abundant mitochondria, SR (sarcoplasmic reticulum) and TT (transverse tubular) systems. Calcium is required for the cardiac muscle to contract. It is supplied from outside the cell and enters through the sarcolemma in response to an action potential. It is also released into the sarcoplasm from stores within the SR.

The electrical conducting system of the heart are modified cardiac muscle cells referred to as Purkinje fibers. Intrinsic waves of excitation originate in the sinoatrial node (the heart pacemaker) which distributes electrical impulses to the atrioventricular node and then to the atrioventricular bundle of His.

Heart Failure and Abnormalities

Myocardial failure is a prominent heart failure condition. This is the condition in which an abnormality of cardiac function is responsible for the inability of the heart to pump blood into the vascular system at a rate commensurate with the requirements of the metabolizing tissues. Compensation can be at the expense of an abnormally elevated filling pressure. Heart failure is frequently, but not always, caused by a defect in myocardial contraction, in which the term myocardial failure is appropriate. The latter may result from a primary abnormality of the heart muscle, as occurs in the cardiomyopathies and are not the result of hypertension or congenital, valvular, coronary, arterial or pericardial abnormalities. Myocardial failure more frequently results from extramyocardial abnormalities, such as coronary atherosclerosis which leads to myocardial ischemia and infarction, as well as from abnormalities of the heart valves that cause an ultimate burden to the heart muscle.

Pure myocardial disease causing cardiomyopathy and heart failure can be 1) primary, with idiopathic and familiar causes being the most common or 2) secondary, due to infections, metabolic diseases, storage diseases, nutritional deficiencies, connective tissue disorders, infiltrative processes, neuromuscular diseases, toxic reactions, peripartum or fibroelastosis. Clinically the cardiomyopathies can be classified as dilated (congestive), restrictive or hypertrophic.

Myocardial failure causing heart failure may be described as a low output failure. To be low cardiac output must be depressed continuously, not only during exertion. Heart failure can be 1) acute, secondary to a massive cardiac muscle death due to infarction or 2) chronic, as secondary to a slow pathological process causing progressive and steady myocardial damage. Heart failure can also be classified as systolic when the principal abnormality is the inability to expel sufficient or diastolic when the problem is the failure of the chambers to relax and fill normally.

Tissue damage to the ventricular myocardium usually is due to ischemia, coronary artery disease, or infarction. Less frequently it is due to infection, infiltration and dysplasia. Tissue damage can result in tachyarrythmias which can be treated with implantable cardioverter defibrillators. 80% of patients with tachycardia have ventricular tachycardia. Tissue damage (e.g., necrosis, fibrosis) to the septa typically occurs from ischemia.

Atrial fibrillation has, as causative factors, structural abnormalities such as valvular heart disease, systolic or diastolic dysfunction, CHF, myocardial infarction, diabetes, and hypertension. The need for heart transplantation is usually due to dilated cardiomyopathies and end-stage coronary artery disease. Congestive heart failure (CHF) results from any structural or functional cardiac disorder that impairs the ventricle's ability to fill or eject blood. Diastolic dysfunction accounts to almost 50% of CHF cases and is more common in the elderly. 500,000 Americans each year are newly affected by CHF.

Cardiomyopathies can be classified as restrictive, hypertophic or dilated. Restrictive cardiomyopathy is the least common endomyocardial disease and is presented as diastolic dysfunction out of proportion to systolic dysfunction. This can be produced by myocardial fibrosis, myocardial infiltration by proteins (amyloids), endomyocardial scarring, and cardiac muscle hypertrophy (atrial enlargement). In endomyocardial fibrosis, the ventricular apices and subvalvular apparatus is involved. The ventricles may be obliterated by collagen tissue. Implantation of cardiac fibroblasts and other tissue types can be used and muscle cell types as well to remove the tissue fibrosis.

Hypertrophic cardiomyopathy (HCM) has myocytes myofibril disarray occupying more than 20% of at least one ventricular tissue block. It is not the myocardial hypertrophy that develops due to hypertension or other known causes. HCM is known as hypertrophic obstructive cardiomyopathy, idiopathichyprtrophic subaortic stenosis, asymmetric septal hypertrophy and muscular subaortic stenosis. Mitral regurgitation and atrial fibrillation are common manifestations of HCM.

Dilated cardiomyopathy (DC) is the most common cardiomyopathy. It is characterized by enlargement of one or both ventricles resulting in both systolic and diastolic contractile dysfunction. This disease may be primary, due to the cardiomyocytes or secondary, due to associated systemic diseases. The most common is ischemic, shown by left ventricular dilation due to myocardial infarction. DC and HCM can be treated with the implantation of muscle cells into the ventricular regions affected.

Myocardial hypertrophy can be due to fibrosis caused by excess collagen deposition, for example, from hormone stimulation in the case of hypertension due elevated aldosterone levels. Implantation of preferably autologous fibroblasts (e.g., cardiac, dermal) as for other heart tissue fibrosis can repair myocardial hypertrophy.

Connective tissue disorders affect the cardiovascular system. The three layers of cardiac muscle, the myocardium, endocardium and the pericardium can be damaged through different mechanisms by rheumatologic disease. The conducting system is affected by different mechanisms by a variety of connective tissue disorders. Fibrosis or inflammatory infiltration results in bundle branch blocks, AV blocks, among other electrophysiologic abnormalities. Valvular disease, coronary lesions and pulmonary hypertension can affect bundle branch blocks, atrial fibrillation and other arrhythmias. Subepicardial abscesses occur with pericarditis and enter the myocardium.

Sinus node dysfunction can occur due to infiltration (e.g. fibrosis), infection or infarction. It is a major cause of bradycardia (which increases with age), arrhythmias and other alterations of the heart rhythm that require and for pacemaker placement. Ventricular tachycardia is associated with structural heart disease, e.g. coronary artery disease.

Aging of the Heart

Loss of myocytes or increased peripheral vascular resistance can result in hypertrophy in the remaining myocytes. This can result in an increase in cardiac mass, which can be due to other factors such as an abundance of amyloid, collagen, fat fibrosis and advanced glycation products, ischemia or infarction. There can be increased interventricular septal thickness resulting in diastolic dysfunction. Valvular stiffening due to fibrosis and calcification of the aortic valve and mitral annulus occurs with aging. Aortic stenosis occurs in about 10% of aged greater than 62. Mitral regurgitation occurs from myxomatous (mesenchymal tissue) degeneration or annular dilatation. There is diminished intrinsic sinus and resting heart rates due to a 90% decrease in sinoatrial pacemaker cell number and separation from atrial musculature due to surrounding fatty tissue deposits. There is a slight PR interval prolongation and increased ventricular ectopy due to increased collagenous and elastic tissues in the conduction system. Decreased bundle fascicle density and distal conduction fibers can cause bundle branch blocks and abnormal conduction. Increased fibrosis and myocyte death results in a lower threshold for atrial and ventricular arrythmias and a reduced threshold for calcium overload, diastolic after depolarizations and ventricular fibrillation. Left ventricular ejection is only about a fifth of the contractile reserve as the young, with the peak rate of left ventricular diastolic filling reduced by 50%. The tissue is more susceptible to the symptomatic consequences of atrial fibrillation. The maximum heart rate during exertion decreases more than 30% with age, from a high of 180 to 200 beats per min in a 20 year old to 120 beats per min in a 80 year old. The incidence of CHF (congestive heart failure) increases linearly past age 45, half of which is due to diastolic dysfunction and to a lesser degree aortic stenosis. Atrial fibrillation (AF) is the most prominent superventricular arrhythmia in the elderly and occurs in greater than 14% of persons over the age of 85. Stroke and coronary artery disease can promote AF. Implantation of Purkinje fibers into the sinus node can be used to restore sinus node function preventing arrhythmia. Alternatively, cells that form Purkinje fibers, and/or precursors thereof, may be introduced into the patient, e.g., at the sinus node. In some embodiments, the Purkinje fibers are cultivated in vitro from autologous cells.

Dyspnea or respiratory distress is the most common symptom of heart failure. In general the basic treatment is divided into three components: 1) removal of the precipitating cause 2) correction of the underlying cause (when possible) and 3) control of the congestive heart failure state. Some improvement can be usually achieved by a) reducing the cardiac work load with less physical activity and helping the cardiac muscle to contract better by using cardiac glycosides such as digoxin, b) controlling excessive fluid retention by monitoring the diet, sodium consumption and using diuretics, and c) vasodilation therapy.

The prognosis in myocardial failure depends primarily on the nature of the underlying cause and the possibility of correction. If the cause can not be corrected most patients pursue an inexorable downhill course, and the majority, particularly those over 55 years of age, die within 2 years of the onset of the symptoms unless a heart transplant is performed. Spontaneous improvement or stabilization occurs in a minority. Orthotopic allograft cadaver cardiac transplantation is the only definitive cure at this time for a end-stage myocardial failure. The limited donor supply and the high cost of the procedure restricts it to patients most likely to survive and resume a functional life after transplantation. Pharmacological immunosuppression to avoid rejection is required for life.

Treatments

Defects associated with the cardiac muscle or system can be treated by introducing cells at or neat the defect to restore the function of the tissue at the defect. Options for cell types and delivery sites may be made in light of the descriptions herein of the defects and the cells types at the defect. Stem cells can be obtained from bone marrow and the peripheral blood supply in addition to the heart tissue itself and can develop upon arrival into heart tissue into cardiac muscle cells (myocytes). Embryonic, fetal, neonatal stem cells and adult cardiac myocytes and skeletal myoblasts also can be used to improve myocardial function. Blood flow progenitor cells, endothelial and pericytes can be used in tandem or singly to improve blood flow and the delivery of endogenous stem cells and adult cell types to the heart tissue.

Growth factors can be used singly or in tandem with cells. For example, GM-CSF can increase generation of bone-marrow derived cardiac myocytes. Selective homing of these cells to the heart area is needed and can be accomplished with tissue and cell type specific cell adhesion molecules.

Stem cells and differentiated cells (endothelial, fibroblasts, muscle cells (cardiac myoblasts, skeletal or smooth muscle myoblasts) can be obtained from any of the structures of the heart for in vitro expansion and implantation. These cells may be obtained from other tissues in the body for expansion. The locations in the heart that cells can be obtained from include the pericardium, both the outer fibrous and inner parietal layers, from the pericardial cavity, from the epicardium, myocardium, muscle fibers or endocardium. Cells from the papillary muscles, muscles that assist the opening and shutting of the heart valves, in particular the tricuspid and mitral valves, can be cultured and implanted into the papillary muscle that is dysfunctional leading to valve malfunction, stenosis and insufficiency. Connective tissue cells can be implanted into the chordae tendinae that is torn or dysfunctional. Valve leaflets can be repaired with connective tissue cells (e.g., fibroblasts). Prosthetic valves can be supported by reinforced chordae and papillary muscle with muscle cell implants. Other muscle cell types or myoblasts from other locations in the body (smooth muscle, skeletal muscle) can be used for implantation after expansion. The major pumping muscle of the heart, the myocardium present in the ventricles and atria, can be treated for disorders such as muscle ischemia or infarction by implanting expanded cultured cells containing muscle cells and/or fibroblasts. These cells comprise the myocardial tissue. Cells that are of the proper phenotype from other locations in the heart can be implanted into the myocardium. Leaflets comprising the heart valves can be repaired by implantation of fibroblasts into fibrous supporting (e.g., connective ) tissue attaching to the valve and implantation of muscle cells into the cardiac muscle fibers attaching to the valve in the proximal area of valve damage. Pacemaker cells can be cultured and implanted into the sinoatrial (S-A) node or atrioventricular node (A-V) node or proximal area to these nodes to control the rhythm and beating of the heart. Alternately the cells can be implanted into the atrioventricular bundle (bundle of His) or even other areas of the muscle of the heart to generate an electrochemical gradient. Pacemaker (P) cells can be used to restore normal heart rhythm. Pacemaker cells can be obtained from modified cardiac or atrial cardiomyocytes or nodal cells from the fetus located in the nodes or Purkinje fibers. The septa, in particular the separation between the ventricles, can be severely damaged from necrosis post-infarctum, amongst other causes. Implanted muscle cells can be used to repair the damaged septa. Fibroblasts and myofibroblasts are an alternate cell type that can be used and are preferred if the septa damage is between atria.

Some examples of adult stem cells are hematopoeitic stem cells, bone marrow stem cells, unfractionated bone marrow stem cells, mesenchymal stem cells, neural stem cells, vascular endothelial cells and multipotent adult progenitor cells. Neonatal and fetal cardiomyocytes can be used. Skeletal muscle and smooth muscle cells and pericytes can be used. Pericytes are slender mesenchymal like cells often found enveloping the outside wall of postcapillary venules, are almost totally undifferentiated and can become a fibroblast, macrophage or smooth muscle cell. An advantage of skeletal muscle cells is the ability of the cells to survive an ischemic tissue environment. Cardiomyocytes need a constant blood supply.

Stem cells (e.g, MSCs) can be used in addition to the heart tissue itself and can develop upon arrival into heart tissue into cardiac muscle cells (myocytes). Adult cardiac myocytes and skeletal myoblasts also can be used to improve myocardial function. Progenitor cells, endothelial and pericytes can be used in tandem or single to improve blood flow and thus the delivery of endogenous stem cells and adult cell types to the heart tissue.

Disorders resulting from heart tissue fibrosis or sclerosis, such as restrictive cardiomyopathy, myocardial hypertrophy, valvular stiffening, aortic stenosis, aging heart tissue, sinus node dysfunction, bundle branch blocks, AV blocks, and other electrophysiological abnormalities can be treated by removing the fibrosis with autologous fibroblasts or muscle cells, amongst other cell types.

Cells from various areas of the heart may be used for expansion and implantation. Cells, including cardioblasts (cardiac stem cells) from the different layers of the heart and from either the atria or ventricles can be used. Additionally cells from the nodal areas or from the Purkinje fibers can be used. A potential problem in implanting cells into an infarcted area is low survival and no blood supply. Thus co-injection with angiogenic factors or cells such as pericytes may be used or co-injection with vasodilators can be used. Cells can be 3 dimensionally implanted by in vitro growth on ECM, scaffolds or as cell aggregates. For cardiac stem cells, mesenchymal feeder layers can be deployed to maintain the ability of these cells to differentiate into the cardiac phenotype.

Valve replacement is currently performed with animal valves, pericardium, cadaver homografts or can be mechanical. In the mechanical cardiac assist devices, one aspect of the invention is to put in a biological intima, (e.g. endothelial cells) into the pump chamber to reduce the need for anticoagulation agents. Implantation of autologous cells such as muscle cells or fibroblasts into the valve tissue can strengthen the valvular structure and enhance its function. Alternately, valve layers can be made in vitro from autologous cells and engrafted for valve replacement.

For myocardial regeneration, cellular implants can be used to reduce the size and fibrosis of infarct scars, improve myocardial contractility, reduce ventricular dilation, control structural changes due to ECM changes, and change in ventricular wall thickness (increase). The benefits to diastolic function improves wall tension and elasticity, for systolic function through wall motion and pressure improvements.

Pericytes are found on the outer surface of capillaries and postcapillary venules. These cells are capable of contraction and can act as mesenchymal stem cells. These cells can repair through proliferation and form new blood vessel and connective tissue cells. Thus pericytes can be used in cardiac repair for the cardiac dysfunctions described, amongst others.

Delivery of cells to the damaged heart areas can be by direct injection through open heart surgery or preferably by a laparascopic means, such as by a percutaneous electromechanical guiding system. Cell infusion by intracoronary delivery, especially for the myocardium, or intravenous delivery, which requires a homing factor to guide the cell into the injured heart area are alternate means of cell implantation. Catheter based guidance by endoventricular or intravascular means may be used, amongst others.

Blood Vessel Defects

The Artery

The artery consists of three well-defined layers; the intima, the media and the adventitia. The intima is a single continuous layer of endothelial cells that line the lumen of all arteries. The intima is delimited on its outer aspect by a perforated tube of elastic tissue, the internal elastic lamina which is particularly prominent in the large and medium caliber elastic arteries and disappears in capillaries. The endothelial cells are attached to one another by a series of junctional complexes and also by a tenuous underlying mesh of loose connective tissue called the basal lamina.

The media is a layer that consists only of one cell type, the smooth muscle cell. It is arranged in a single layer as in small muscular arteries or multiple lamellae as in elastic arteries. These cells are surrounded by small amounts of collagen (type III) and elastic fibers. They are closely apposed to one another and may be attached by junctional complexes. The smooth muscle cell appears to be the major connective tissue-forming cell of the artery wall producing collagen, elastin and proteoglycans, amongst other ECM. On the luminal side the media is bounded by the internal elastic lamina and on the abluminal side by the external elastic lamina, which are very prominent in the elastic arteries (e.g., aorta) and the pulmonary arteries, which expand largely with the pulse during systole. Located about midway through the media of most arteries is a “nutritional watershed”. The outer portion is nourished from the small blood vessels (vasa vasorum) in the adventitia. The inner portion receives its nutrients from the lumen. Sympathetic innervation activity controls the tonus through the smooth muscle cells. Vasoconstrictors (e.g., thromboxane, endothelin-1, angiotensin II, serotonin) and vasodilators (e.g. prostaglandins, prostacyclin, bradykinins, histamine, nitric oxide, calcium channel blockers, hydralazine, minoxidil) act on smooth muscle cells. Vasodilators are used to treat hypertension and angina by decreasing peripheral vascular resistance.

The adventitia is the outermost layer of the artery which is delimited on the luminal aspect by the external elastic lamina. This external coat consists of a loose interwoven layer of collagen (type 1) bundles, elastic fibers, smooth-muscle cells and fibroblasts. This layer also contains the vasa vasorum and nerves.

ECM is present throughout the vessel structure. Elastin as part of individual elastic fibers (0.1 to 10 μum in diameter) form net like structures with each other and extend mainly in a circumferential direction. The internal elastic lamina, present between the intima and media of the arteries allows the vessel to recoil after distension. The outer elastic lamina is less well developed than the internal one and lies at the outer aspect of the media and the adventitia. In elastic arteries, these fibers are less evident, in which the fibers occupy much of the media. Collagen fibrils are in all three layers. Type III is in the intima and in the space between smooth muscle cells (produced by these cells) in the media. This space transmits force to the circumference of the vessel. Type I collagen is abundant in the adventitia and has a supportive role. Collagen is the main protein component of veins, accounting for more than half its mass. Other ECM proteins are present such as the proteoglycans and fibronectin, etc. Fibers of collagen and elastin run parallel to the axes of muscle cells and are thus circumferentially positioned. In the adventitia collagen fibers are longitudinal and contain changes in larger vessels under pressure. For example, the radial distension is much greater than longitudinal in the large arteries under a pulse.

Endothelium functions in many ways. The endothelial cells (ECs) secrete ECM (e.g., collagen III, IV, fibronectin, vitronectin, elastin, glycosaminoglycans, proteoglycans, proteases, protease inhibitors, amongst others) into the subendothelial layer preventing blood escape into the extravascular space. The cells act as an anti-coagulant surface by secretion of tissue plasminogen activator and urokinase (converts plasminogen to plasmin), secretes prostacyclin (PGI₂) and endothelium-derived relaxing factor (EDRF) causing vasodilation and inhibition of platelet adhesion and aggregation, and expresses anti-coagulant cell surface molecules (e.g., glycosaminoglycans, heparin sulfate-antithrombin III system, thrombin-thrombomodulin-protein C system and plasminogen-plasmin activator system). In response to injury ECs can vasconstrict the media (secrete endothelin-1) and secrete molecules that coagulate (e.g., tissue factor, von Willebrand factor, factor V, plasminogen activator inhibitors PAI-1 and 2, interleukin 1, tumor necrosis factor). ECs can vasodilate the media by secretion of nitric oxide (NO). NO increases levels of cGMP in smooth muscle cells that causes vasodilation. Viagra increases cGMP levels for vasodilation in penile erection. Angina drugs (nitroglycerin, amyl nitrite) are metabolized by smooth muscle cells to form nitric oxide, relaxing venous and arterial smooth muscle producing vasodilation. ECs, especially in lung capillaries, convert angiotensin I to II producing vasoconstriction and aldosterone and ADH secretion. ECs of the skeletal muscle and adipose tissue capillaries have lipoprotein lipase to catalyze removal of triacylglycerides of VLDL and chylomicrons. ECs are a diffusion barrier that allows passage of lipid-soluble molecules, O₂ and CO₂ by diffusion, water-soluble molecules (water, amino acids, glucose) by movement through intercellular spaces and larger water-soluble molecules such as proteins by pinocytosis.

Blood flow to an organ can be modified by an increase in tissue activity through release of vasodilator metabolites (e.g. can function to increase metabolism by implanting cells into an organ, such as skeletal muscle), by autoregulation in which an organ remains with constant blood flow over a wide range of pressures, and by increased blood flow to an organ after a period of occlusion.

The arterial-venous system is organized from the heart as large elastic arteries, muscular arteries, arterioles, capillaries, sinusoids, venules and veins.

The large elastic arteries (e.g., pulmonary artery, aorta) and its largest branches (e.g., brachiocephalic, common carotid, subclavian and common iliac arteries) conduct blood to the medium-sized distributing arteries. The media has a prominent elastic fiber that responds to high systolic pressure from the heart. It contains some 30 to 50 fenestrated layers of elastin, with ECM and smooth muscle cells in between each layer. The subendothelial layer is a connective tissue layer comprised of fibroblasts and smooth muscle like myointimal cells, that can accumulate lipid. The elastic lamina measures 0.1 um, is stretched under the effect of systolic pressure and recoils under diastole. The adventitia contains flattened fibroblasts, macrophages and mast cells, nerve bundles and lymphatic vessels.

Muscular (distributing) arteries (diameter greater than 0.5 mm) have a prominent internal elastic lamina and smooth muscle cells in the media, occupying some 75% of the mass. The external elastic lamina is made of sheets of elastic fibers that are not as compact as the internal elastic lamina. The adventitia is thick.

Arterioles (diameters 30 to 200 um) have only 1 to 2 layers of large smooth muscle cells, the external elastic lamina may be absent and the adventitia is thin. The ECs are smaller than in large arteries. The internal elastic lamina is absent or highly fenestrated in which the cytoplasm of muscle cells or endothelial cells pass through. Small arterioles act as sphincters to control blood flow. Along with the larger arterioles, they play a major role in blood pressure by contributing to vascular resistance as gauged by the relaxation or contraction of their smooth muscle cells. Sphincter closure is under myogenic and not neurogenic control and is responsive to local vasoactive and metabolic factors. Discontinuous smooth muscle cells surround the arterioles. The blood pressure is only 30% of that in the aorta.

The capillary (4 to 8 um diameter) wall is comprised of the endothelium, basal lamina and a few pericytes. These are vessels closest to the tissue they supply and the wall is a minimal barrier between blood and tissue. These are the sites of exchange between blood and cells of O₂, CO₂, water, glucose, proteins, amino acids, etc. The permeability of these vessels is determined by the type of tissue. Gases and small molecules diffuse across endothelium. Larger molecules and water soluble substances are selectively transported by segments of the tight junctions, through pores or vesicle transcytosis through the endothelium. Continuous capillaries are in the brain (blood brain barrier), lung, muscle and testis, which need efficient barriers to large molecule diffusion and thus the capillaries have tight junctions joining the continuous endothelial cells and extending into a perimeter around the cells. Fenestrated (50-100 nm in diameter) capillaries with diaphragms contain ECs with a tight junction that only partially extends around the perimeter of the cells resulting in a slit like intercellular spaces and fenestrae (pores) with diaphragms. These are found in the endocrine glands, intestina and kidney. The kidney glomerulus contains fenestrated capillaries without diaphragms.

Sinusoidal capillaries are expanded capillaries with a large diameter and with discontinuities in their walls (a single layer of endothelial cells with wide gaps between cells and having fenestrae) allowing contact between blood and the tissue parenchymal. Whole cells can pass between blood and tissue. These vessels are present in liver, spleen and bone marrow.

Venules (postcapillary venule) are formed from two or more converging capillaries (10 to 30 um). Venules contain endothelial cells surrounded by basal lamina and in larger venules also contain adventitia of sparse fibroblasts and collagen fibers. Pericytes surround the venule walls. Since there are few tight junctions venules are permeable vessels. The cross-sectional area of the vascular tree is maximum and a large fall in pressure (25 mm Hg in capillaries to 5 mmHg in venules). Since the pressure is lower than even present in tissue, venules collect fluid. When venules are larger than 50 um, smooth muscle cells are present. Venules enlarge to form veins.

The Veins

Veins show a considerable variation in structure depending upon the venous pressure. As a general rule, veins have a larger diameter than any accompanying artery, with a thinner wall that has more connective tissue and less elastic and muscle fibers. Small- and medium-sized veins have a well developed adventitia. The intima lacks a continuous internal elastic lamina, and the media is thin, consisting of two or three separated layers of smooth muscle. Large veins have diameters of more than 10 mm. These vessels have a thicker intima and a poorly developed media, but the adventitia is very thick and contains collagen, elastic fibers, ECM and a variable amount of smooth muscle. Assisting with venous function are the valves, found in most veins. Valves are inward extensions of the intima supported by elastic fibers and ECM (e.g., collagen fibers). They form semilunar pockets or cusps, are attached by their convex edges to the venous wall and by occurring in pairs they prevent backflow and regulate the pressure in more distal veins. Often two valves lie opposite each other and ECs are positioned transversely on the surface facing the vessel wall and longitudinally in the direction of blood flow on the luminal surface. The concave margins are with the flow of blood and lie against the wall, but when blood flow reverses, valves close and fill with blood an expanded region of the wall. Valves also inhibit backpressure in distal veins and works as a partition pump holding isolated segments of blood. They are found in small veins and where tributaries join each other, especially in the legs where venous return is against gravity. Muscle action moves the blood towards the heart by intermittent pressure. Valves are not in the veins of the abdomen or thorax. Pressure does not exceed 5 mm Hg in the venous system and it decreases as veins become larger and fewer in number and is close to zero as it approaches the heart.

The vasa vasorum are a system of microvessels (the blood vessels in the larger blood vessels) in which the capillaries from adjacent small arteries attach to the adventitia of larger blood vessels, while the veins in these vessels can go into the intima. Anastomoses are links between arteries and veins or arterioles and venules, bypassing the capillary network. These occur mainly in the skin of the digits, nose and lips to regulate heat loss by directing arterial blood into the venous plexus beneath the skin. Anastomoses also can be links between arteries to supply the territory of the other. An angiosome is a three-dimensional portion of tissue supplied by an artery source and its accompanying veins. It can be skin, fascia, muscle or bone. Each block of tissue is linked to other blocks of tissue angiosomes and if one block of tissue is compromised, the blood flow of another angiosome, through anastomoses, can take over the blood supply.

Pathology of the Arteries and Veins.

The maintenance of the endothelial cell lining is critical to the health of the vessels, the active transport through the endothelial cell cytoplasm of multiple circulating substances, the production of connective tissue components and the prevention of clotting. When the endothelial cell lining is damaged platelets adhere to it and form a clot and ultimately an atherosclerotic lesion begins to form with cholesterol deposits.

Aging changes the vasculature. In arteries, there is thickening of the medial and subendothelial layers with increased calcium, cholesterol and fatty acid deposition. There is decreased vessel compliance and increased hemodynamic shear stress. Arteries have increased tortuosity and the large elastic arteries such as aorta and carotid artery, become thicker and harder, resulting in increased peripheral vascular resistance, earlier reflected pulse waves and late augmentation of systolic pressure. The blood flow is less laminar due to tortuosity and the endothelial cells are greater in heterogeneity of size, shape and axial orientation. Smooth muscle cells overproliferate and produce excess ECM. Increased elastase results in less elastin. There may be less repair due to senescence of endothelial cells and fibroblasts. There can be increased cross-linking of the ECM and glycation of the vessel proteins. The result is increased stiffness and thickness of the arteries. The average thickness of the carotid artery doubles by age 80, from 30 um to 60 um. There is a 50% decrease of peak oxygen utilization by age 80, half of which is due to poor peripheral oxygen extraction and utilization from the inefficient redistribution of blood flow to skeletal muscles. The elastic to collagen ratio decreases in the layers of the vessels.

Implantation of cells (e.g., endothelial cells, endothelial precursor cells, pericytes) to increase angiogenesis can be used to enhance blood flow in aging tissues. These same cells can improve the integrity of the arteries and reduce the thickness of aging arteries. In some embodiments, the cells are introduced into a tissue with or without helpful proteins or factors, e.g., angiogenesis factors. In other embodiments, cells are introduced into an artery, in one of the layers already described, e.g., the media or adventitia. The cells contribute to pre-existing blood vessel structure or organize blood vessels, e.g., capillaries or capillary-like structures, that interconnect to existing blood vessels to enhance blood flow.

Stroke accounts for 20% of all cardiovascular deaths in the elderly. Strokes can be due to aneurysms or stenosis. Peripheral arterial occlusive and aneurysmal disease increases four-fold with age. The invention can be used with fibroblast or smooth muscle cells to strengthen vessel wall layers to prevent aneurysms or after removal of plaque. Endothelial cells can implanted in the intima layer to provide enhanced homeostasis and anticoagulation mechanisms to the vessels to prevent clots.

A major change that occurs with normal aging in the arterial wall in humans is a slow, apparently continuous, symmetric increase in the thickness of the intima due to a gradual accumulation of smooth-muscle cells surrounded by additional connective tissue. These changes result in gradually increasing rigidity of vessels. The larger vessels may become dilated, elongated, and tortuous with the potential formation of aneurysms.

Blood Flow

Angiogenesis is the creation of new blood vessels by sprouting off existing vessels. Hypoxia and inflammation are the two major stimuli and VEGF is an important vessel growth factor. Vasculogenesis is the creation of new blood vessels de novo by differentiation of new blood cells. Endothelial cell precursors in the blood or bone marrow can develop new vessels and help growth, such as during embryonic development. Arteriogenesis is the recruitment of existing vessels to increase their capacity and thus blood flow to ischemic tissue. Endothelial cells activated by increased shear stress attract circulating monocytes to the intima surface. Monocytes convert to macrophages which digest the ECM, and produce new fibronectin, proteoglycans and vascular growth factors which increase proliferation of smooth muscle cells and endothelial cells. Platelets adhere to the vascular wall and release IL-4 which stimulates adhesion molecules. As the walls become thinner and leaky the lymphocytes and macrophages destroy myocardium and ECM to open space for the growing collateral vessels. VEGF is not important, but macrophage growth factors are for arteriogenesis. Cells can be infused around the stenosis to recreate arteriogenesis to grow new blood flow for blocked coronary arteries, for example.

Peripheral vascular blood supply maintained by cell and enzyme activates regulate blood flow by controlling 1) vascular constriction and dilation, 2) coagulation and clot dissolution by fibrinolytic cascades, and 3) angiogenesis or the growth of new vessels. Much of this can be controlled locally by endothelial cells.

Vascular dysfunction, in particular due to aging, involves a combination of increased atherosclerosis, thrombosis, decreased vasodilation and angiogenesis, and impaired maintenance and repair of such tissue the vessels are in. This can lead to decrease delivery of restorative stem cells and other cells to organs. Also a decrease in nutrient delivery, hormone, growth factors, amongst others and toxin removal can injure tissue, deprive tissue of normal metabolism, retard in situ stem cell activation in the tissue and result in other deleterious events.

In cases of injury, degeneration or aging of tissues, there is a decrease blood flow in those tissues. Often this is due to decreased capillary formation or maintenance.

In a preferred embodiment, endothelial or endothelial precursor cells or pericytes are used to populate tissues and blood vessels to produce new vasculature or repair vasculature. Homing mechanisms of the cells can be deployed by infusion into the bloodstream or implantation in or around the desired area with or without cell adhesion proteins. Endothelial precursor cells (EPCs) needed to repair aging blood vessels can be added to the bloodstream. EPCs come from the bone marrow and peripheral blood supply as do cardiac myocyte precursors and neuron precursor cells. EPCs can be obtained by selection methods such as antibody affinity to EPC surface antigens. These cells can be expanded and implanted or infused into the subject. Alternately bone marrow or peripheral progenitor cells can be used without selection, expanded and returned to the subject. The inclusion of a statin treatment can increase the pool of peripheral blood EPCs or bone marrow from which to obtain the EPCs. Cell adhesion molecules (e.g., VCAM-1) can be added in tandem with the cells to assist in homing the cells to the vasculature. This includes implantation of adhesion molecules into the target organ in tandem with cells or in which cells are infused and targets the cells to a specific area of the vasculature. Growth factors (e.g., VEGF) in tandem with bone marrow cells or EPCs can restore blood vessel function, particularly in need in older subjects. This can counteract age-associated impairment of pro-angiogenic growth factor pathways or increase in pro-apoptotic pathways (e.g., TNF receptors and TNF□. Implantation of progenitor or endothelial cells into tissue can restore local vasculature and stem cell function of the tissue. Systemic infusion of progenitor cells can promote the long-term restoration of stem cell pathways throughout the aging vasculature. The outcome of such implantations can also increase through EC action vasodilation to the tissues of interest.

Endothelial stem cells called angioblasts form the vascular plexus during embryogenesis. Angioblasts or hemangioblasts and endothelial cell precursors can be used as the cells to promote blood vessel or plexus formation in tissues. Endothelial cells from arteries or veins can be used to induce angiogenesis and neovascularization.

Pericytes are found on the outer surface of capillaries and postcapillary venules. These cells are capable of contraction and can act as mesenchymal stem cells. These cells can repair through proliferation and form new blood vessel and connective tissue cells. Thus pericytes can be used in cardiac and blood vessel repair. Pericytes can be used to increase blood flow and to induce angiogenesis for all tissues.

Implantation of pro-inflammatory factors can be used with or without endothelial cells or EPCs to promote tissue angiogenesis or vasculogenesis. Macrophages and/or macrophage growth factors can be implanted into tissue to promote arteriogenesis or blood vessel growth. Smooth muscle cells and/or EC cells and/or macrophages can be added to the implantat. Spatial and temporal implantation may be used.

The degeneration of the valves in the distal deep venous system causes the development of varicose veins. Implantation of fibroblasts or smooth muscle cells and/or supporting ECM into the interior of the valve and/or endothelial cells onto the surface of damaged valves can be used to rebuild valves. 3 dimensional valves can be crafted in vitro and implanted into the veins using these cell types. Vein segments with or without valves can be made in vitro using these cell types and then engrafted into the appropriate location in vivo.

Three-dimensional vessels can be assembled together in layers by cell aggregation. Pericytes can be used to stabilize the vessels (e.g. small vessels). Arteries and veins of different sizes can be made. Biodegradable scaffolds can be employed in vitro to make even small capillary beds and venules. Scaffolds can be degraded in vitro before implantation or in vivo after implantation. Spatial and temporal synthesis of layers can be done to properly assemble the layers of the blood vessels before implantation into tissue.

In a preferred embodiment cells are isolated for expansion and implantation from the particular vessel type that is being repaired. For example, EC cells from muscular arteries can be used for implantation into muscular arteries whereas EC cells that have a different morphology and exhibit different properties from the capillaries are isolated from and expanded for use to populate the particular capillary blood vessels. In a similar vessel type fashion smooth muscle cells can be used. In an alternate method, cells from different types of blood vessels can be used in non-native blood vessel type locations. In addition cells from other tissues can be used so as the phenotype of the cells in the blood vessels performs its proper function in situ. The walls of the veins can be supported, strengthened and the lumen tightened by the implantation of connective tissue cells (e.g., smooth muscle cells, fibroblasts).

Cells and/or proteins or factors to increase blood flow to tissue can improve the functioning, synthesis and development of that tissue. This aspect of the invention can be used for any tissue or tissue defect to improve the functioning of that tissue and the “take” and functionality of other cells, implanted or present in situ.

The Atherosclerotic Plaque

Atherosclerosis is a chronic inflammatory disease. The plaque represents arterial wall thickening. Plaque development arises from monocyte and lymphocyte interaction with the endothelium and transmigration into the intima. Leukocyte integrins interact with the endothelium selectins and VCAM-1, which are stimulated to be expressed by inflammatory cytokines, such as oxLDL in the serum and MCP-1, IL-8 and acute phase protein CRP within the plaque. This spurs on the transmigration process of leukocytes into subendothelial tissue and the differentiation of monocytes into macrophages. Macrophages can express tissue factor and become foam cells stimulated by M-CSF and CRP. This is a reversible phase in plaque formation. As the inflammatory process continues, smooth muscle cells from the media proliferate and produce collagen, stimulated by PDGF-BB, TGF-α from stimulated endothelial cells and T-lymphocytes, produce a fibrous cap. The cap covers a mixture of collagen, leukocytes, lipids and cell debris, called the lipid core. The core is very thrombogenic due to cell-bound and extracellular tissue factor and production of pro-inflammatory cytokines from cell activation. The plaque stability is dependent on thickness and components of the fibrous cap. High collagen content stabilizes the plaque. If leukocytes and smooth muscle cells inside the plaque produce matrix-degrading proteases more than collagen synthesis, rupture of the cap at the edge of the lesion where the cap is thinnest occurs and a thrombus can be formed. Mechanical and hemodynamic forces like increased blood pressure or pulse rate can trigger the rupture. Arterial thrombosis occurs when tissue factor in the vascular wall or underneath the fibrous cap interacts with coagulation factors in the blood. Implanted fibroblasts can be used to remove the chronic inflammation causing atherosclerosis. Implanted fibroblasts and macrophages (e.g., preferably that are not activated to produce tissue factor or are genetically designed not to produce tissue factor), can be used to degrade ECM and remove the lipid core. Implantation into the media and intima or proximal to the plaque is a preferred location. Implanted smooth muscle cells can be used for this reason as well. In a preferred embodiment, select plaques can be implanted with cells by direct injection or placement. Alternately, infusion of the cell types into the bloodstream can be used in which a general removal of arterial plaques or thickening can be achieved. The walls of the arteries can be supported and strengthened by the implantation of connective tissue cells (e.g., smooth muscle cells, fibroblasts) in particular at a site previously treated by intervention with coronary stents, angioplasty, clot or plaque removal. Autologous cells and/or tissue can be used to cover the medical devices to anchor a stent for example, without immune rejection and also to assist in its function.

Embodiments of the invention can be used for blockages in the blood vessels for specific diseases such as renal artery, aortic, pulmonary, carotid stenosis, peripheral arterial disease, amongst other blood vessel disease. Embodiments of the invention can be used to control blood pressure changes, in particular in the elderly and blood vessel diseased, by repairing the integrity of the blood vessels. As already explained, cells can be introduced into the affected tissue or directly into an artery or other blood vessel.

Endothelial cells can be implanted to control the coagulation status of the vascular system. These cells can be put into a one location or spread throughout the vasculature. ECs can be used to induce vasoactive substances or as an adjunct to drug therapy (e.g., angina drugs). Autologous endothelial cells can be used to coat the inner surface of stents, reducing or removing the need for platelet inhibitor drugs such as clopidogel (II/IIIa platelet inhibitor) during and after perfusion treatments (1 month) for acute coronary syndromes. For instance, cells may be cultured with a stent and then the stent may be implanted. In some embodiments, endothelial cells or their precursors from the patient are associated with the stent, e.g., by culture, by mixing the cells with a protein or other substance to make a three-dimensional gel, paste, or other delivery vehicle that is applied to the stent. Or the endothelial cells are cultured in vitro as a layer on a synthetic sheet or other optionally degradable support that is then applied to the inside and/or outside of the stent. In other embodiments, ECM collected from in-vitro cultured cells is coated onto the stent, which may then be optionally associated with endothelial cells or precursors as described. The ECM provides an improved environment to promote the adhesion, spreading, and/or mitosis of the cells. In some embodiments, the cells are associated with factors that enhance endothelial cells mitosis so that endothelial cell proliferation is enhanced in vitro or in vivo.

Pulmomary Defect—The Lung

The conducting system comprises all of the pathways by which air travels to the lungs. They include the nasal cavity, pharynx, larynx, trachea, and bronchi. The system warms, filters, moistens and delivers the air to the gas exchange area of the lungs. The respiratory unit consists of the respiratory bronchiole, alveolar duct, alveolar sac, and millions of thin walled alveoli. Inside the air sacs oxygen inhaled diffuses into blood and carbon dioxide from the blood into the alveoli and exhaled. The pleural membrane covers the lobes of the lungs. The serosa made by the visceral pleural mesothelium covers the submesothelial (lamina propria) connective tissue. It contains a single layer of mesothelial cells that secretes a serous fluid to moisten the pleural surface. In the mesothelium is the pleural cavity, parietal pleura and the outside layer, endothoracic fascia. Each lung is free in its own pleural cavity except for the attachment to the heart and trachea at the hilum and pulmonary ligaments, respectively.

In breathing and during inspiration, the diaphragm and external intercostals muscles contract to expand the rib cage and thoracic cavity volume. Air rushes in to equalize the negative pressure. During expiration, air is pushed out of the lungs as the lungs passively recoil when the diaphragm and intercostals muscles relax. Breathing exposes the lungs to environmental agents such as gases, dust particles, microorganisms and viruses. The defense is the mucous barrier, mucociliary escalator, the anatomical branching of the airways and the cough reflex.

Most of the volumetric change during ventilation occurs in the alveoli. The diaphragm and the costomediastinal regions of the chest wall expand most of all surrounding lung area. The diaphragm accounts for 67% of the vital capacity during inspiration. The external intercostal muscles are active during inspiration, the internal intercostals muscles during expiration. The main role of the intercostal muscles is to stiffen the chest wall. During inspiration, a decrease in intrapleural pressure occurs from the increase in vertical, transverse and anteroposterior dimensions of the chest. The contraction of the diaphragm pulls down the central tendon. During expiration, the diaphragm relaxes and air is expelled from the lungs as the elastic recoil of the lung produces subatmospheric pressures, returning the lateral and anteroposterior dimension of the thorax to normal. The abdomen is the major muscle of expiration. There are bucket handle and pump handle movements of the ribs that work in tandem with a central tendon movement and muscles during inspiration. Pharyngeal muscles also play a role in ventilation.

Six types of epithelial cells are in the conducting airways. Lymphocytes and mast cells migrate into the epithelium from underlying connective tissue. Ciliated columnar cells are responsible for the mucociliary current in the bronchial tree. Goblet cells are present from the trachea (7,000 per mm²) to the smaller bronchi, but not the bronchioles. When the epithelium is irritated by chemicals these cells increase in numbers and contain vacuoles filled with mucinogen. Clara cells are cuboidal non-ciliated cells and bulge into the lumen. They produce surfactant lipoprotein, sharing function with alveolar cells and regulate ion transport. Basal cells are rounded, pseudostratified respiratory epithelium, and are stem cells for other epithelial cell types. Basal cells are in contact with the basal lamina in larger conducting passages. Brush cells are slender, non-ciliated, with apical microvilli and infrequently present in all parts of the conducting air passages with a sensory receptor function. Neuroendocrine cells in the neuroepithelial bodies are single or aggregated. These cells act on bronchiolar smooth muscle and are chemoreceptors that secrete peptides and amine into capillaries.

Lymphocytes, mainly T cells derived from mucosa associated lymphoid tissue, are present in all the conducting airway tissues and function with the immune surveillance of the epithelium. Mast cells present in basal regions of the epithelium are released in response to irritants, including allergens. They are present in the connective tissue of the respiratory tree and can affect the contraction of smooth muscle fibers surrounding the bronchial tree.

Submucosal glands contain mucous and serous cells that are the source of the mucous layer at the surface of ciliated respiratory epithelium. The secretions include mucins, protease inhibitors (□anti-trypsin) to neutralize elastase, a leukocyte derived protease. The glands are surrounded by myoepithelial cells innervated by autonomic fibers.

Connective tissue (e.g., contains fibroblasts, myofibroblasts, amongst other cell types) and muscle engulfs the conducting system. Smooth muscle is confined to the posterior non-cartilaginous part of the trachea and extrapulmonary bronchi. Smooth muscle forms two helical tracts along the intrapulmonary bronchial tree, which becoming thinner, until not present at the alveoli level. These muscle fibers are under nervous and hormonal tonal control. Longitudinal bands of elastin are present in the submucosa of the respiratory tree and joins the elastin network in the interalveolar septa. This is important for the elastic recoil during expiration and is an essential mechanical element of the lung.

The respiratory surfaces, downstream of the bronchiolar epithelial cell types, contain the alveolar cells (pneumocytes). These epithelial cells comprise two cell types. Type I alveolar cells are squamous and cover more than 90% of the alveolar wall. In the adult there is more than 300 million alveoli with a cell lifespan of 3 weeks. Type I cells do not divide and are derived from Type II cells. Type II alveolar cells are cuboidal in shape and account for less than 10% of the alveolar wall or surface area, but have the important function of producing surfactant. Surfactant reduces surface tension, allowing ventilation of the alveoli to be very efficient. Due to the very small alveoli size, surface tension is very high at the surface, opposes alveoli expansion during inspiration and collapses alveoli during expiration. The alveolar wall, the lamina propria, is in close apposition with the lamina propria and thin endothelium of capillaries that constitute the blood-air barrier. The lining of the epithelium can be as little as 0.05 um and the back to back lamina propria with alveoli and capillary epithelium can be as thin as 0.2 um for blood-air interchange. The alveolar cells form sacs known as alveoli that have a honeycomb pattern sustained by this fine connective tissue. Fibroblasts produce elastic fibers and collagen fibrils (type III) in the connective tissue (lamina propria), and resident and migratory cells are present, including smooth muscle cells. Small pores, lined by type II alveolar epithelium, cross interalveolar septa linking adjacent alveolar air spaces and help sustain the flow of air, especially when one of the alveolar ducts is blocked. The small pores are pathways for macrophage migration. Alveolar macrophages are derived from monocyte precursors in the bloodstream derived from hematopoietic tissue in the bone marrow. The macrophages, via the bloodstream and underlying connective tissue, are located on the epithelial surface of the alveoli. The macrophages have an average lifespan of 4 days and they remove inhaled particles that are small enough to reach the alveoli. After phagocytosing the particles the macrophages migrate to the bronchioles and are removed from the lung by mucociliary currents. A smaller number also drain into the lymphatics. Alveolar macrophages also turnover surfactant, secreting proteases during phagocytosis while normal alveoli counter with anti-proteases (αanti-trypsin).

Interstitial Lung Diseases (ILDs) and Idiopathic Pulmonary Fibrosis (IPF)

Interstitial Lung Diseases (ILDs) are a heterogeneous and large group of conditions that involve the parenchyma of the lung— the alveoli, the alveolar epithelium, the capillary endothelium and the spaces between these structures, as well as the perivascular and lymphatic tissues. ILDs are not malignant diseases nor are they caused by any defmed infectious agents. The individual may show acute symptoms, but often the onset is insidious and the disease is chronic in duration. The precise pathway(s) leading from injury to fibrosis is not known. Although there are multiple initiating insults the mechanisms of repair have common features. ILDs have been difficult to classify, because approximately 200 known individual diseases are characterized by diffuse parenchymal lung involvement, either as the primary condition or as a significant part of a multiorgan process, as may occur in the connective tissue diseases (CTDs). A useful approach for classification is to separate the ILD's into two groups based on the major underlying histopathology: (1) those associated with predominant inflammation and fibrosis, and (2) those with a predominant granulomatosis reaction in interstitial or vascular areas. Each of these groups can be further subdivided according to whether or not the cause is known or unknown The first group are ILD's of unknown etiology from which sarcoidosis, idiopathic pulmonary fibrosis (IPF), and ILDs associated with collagen vascular disorders (e.g systemic lupus erythematosus, rheumatoid arthritis, systemic sclerosis, poly and dermatomyositis, amongst others) are the most common. The second group is comprised of known causes. ILDs caused by occupational and environmental inhalant exposures are the largest subgroup.

Histopathology of ILDs

In inflammation and fibrosis the initial insult is an injury to the epithelial surface causing inflammation of alveolar walls, known as alveolitis. If the disease is chronic and smoldering, inflammation spreads into adjacent portions of the interstitium and vasculature, producing interstitial fibrosis with the resulting irreversible scarring and distortion of the lung tissue and impairment of the breathing function and gas exchange. Depending on the area of inflammation the types of ILD's include usual interstitial pneumonia, (UIP), non-specific interstitial pneumonia, respiratory bronchiolitis, organizing pneumonia (bronchiolitis obliterans with organizing pneumonia (BOOP) pattern), diffuse alveolar damage (acute or organizing), desquamative interstitial pneumonia, and lymphocytic interstitial pneumonia.

In granulomatous lung disease there is the presence or absence of granulomas (e.g., nodular inflammatory lesions that are small, granular, fimn containing compactly grouped T lymphocytes, macrophages and epitheloid cells) in the interstitial or vascular areas. The granulomatous lesions can progress into fibrosis. The main differential diagnosis is between sarcoidosis and hypersensitivity pneumonitis.

Idiopathic Pulmonary Fibrosis (IPF) is described as idiopathic, meaning that the etiology of the disease is unknown. However, IPF is a well-defined clinical entity with multiple causes. The average incidence is with patients that are middle aged, although incidence can range from infancy to old age. IPF affects several parts of the alveolar structure, the wall of the alveoli lined with type I and II pneumocytes and the interstitial supporting structure composed of mesenchymal cells such as fibroblasts and myofibroblasts, and extracellular matrix contain collagen, various adhesive proteoglycans and other proteins. The capillary endothelium is affected as well and may show sclerosis. The proportion of assorted immune cells normally present in the alveolar structure changes early in the disease process and is a good indicator of the type of alveolar injury (e.g, reversible or not). In early and reversible IPF, leakiness of the alveolar type I cells and the adjacent capillary endothelial cells occurs, causing alveolar and interstitial edema and the formation of intra-alveolar hyaline membranes. When the disease persists increased permeability of the capillary endothelium exists with more loss of alveolar cells due to desquamation, mural inflammation and interstitial fibrosis. The normal immune cell profile is completely disrupted, reflecting severe inflammatory response.

UIP is characterized by a heterogeneous appearance with alternating areas of normal lung, interstitial inflammation, foci of proliferating fibroblasts, dense collagen fibrosis and honeycomb changes affecting most severely the peripheral and subpleural parenchyma. The interstitial inflammation is usually patchy and consists of a lymphoplasmacytic infiltrate in the alveolar septa, associated with hyperplasia of type 2 pneumocytes. The fibrotic zones are composed mainly of dense collagen and scattered foci of proliferating fibroblasts. The extent of fibroblastic proliferation is predictive of disease progression. Areas of honeycomb change are composed of cystic fibrotic air spaces that are frequently lined by bronchiolar epithelium and filled with mucin. Smooth-muscle hyperplasia is commonly seen in areas of fibrosis.

Determination of the clinical manifestations starts with a physical examination of patients with ILDs that may help to determine the nature and severity of the pulmonary condition. Unfortunately, the pulmonary response is the development of a limited number of nonspecific physical signs and symptoms including chronic persistent cough (productive or dry), shortness of breath, weight loss, intermittent low grade fever and generalized chest pain. The patient history is of paramount importance in assessing any potential occupational or environmental exposure, as well as chronic disease that may involve the lungs in the form of an ILD. IPF is characterized by dyspnea, effort intolerance, and a dry and persistent cough without obvious cause and other systemic symptoms, such as fatigue, appetite loss, weight loss and generalized joint pain.

Pulmonary function tests and radiographic examinations of the chest are common tools used to gather information regarding the possible cause of the ILDs and are especially useful to diagnose occupational or environmental causes. Exposure to several mineral dusts and chemicals result in pulmonary function tests with distinctive restrictive patterns. They produce asthma-like obstructive patterns in the function tests. Chest X-rays are usually less helpful because several ILD's may share the same imaging patterns as well as with some unrelated lung diseases. General blood, serologic and antibody testing may be conducted to clarify the diagnosis. Direct visualization of the airways by fiberoptic bronchoscopy may be part of the evaluation as well. A lung biopsy to permit a full histologic evaluation may be necessary in many cases in which all other testing has failed to give an accurate diagnosis. In Idiopathic Pulmonary Fibrosis, the beginning of the disease commonly displays the absence of definitive fmdings upon physical examination or chest X-rays. As the disease progresses dry rales or coarse crackles are heard at auscultation, as well as a faster than normal breathing rate and cyanosis. In late stages cor pulmonale (failure of the right chamber of the heart due to lung chronic disease) appears.

General treatment for ILDs and IPF is aimed at reducing the local inflammatory response. This is usually achieved with the chronic use of prednisone. If the disease continues to progress immunosuppressive agents, such as cyclophosphamide, may be necessary. It is imperative that the patient discontinue any exposure to the agent suspected or proven to cause the disease, as well as discontinue cigarette smoking. Supplemental oxygen therapy is frequently indicated as well as bronchodilators to help with obstructive patterns of breathing. As the disease progresses other lung complications such as pulmonary hypertension may occur, as well as congestive heart failure, and they must be treated accordingly. If the disease is limited to the lungs and turns refractory to all these measurements, unilateral lung transplant may be considered.

Chronic obstructive pulmonary disease (COPD) is defined as a disease state characterized by airflow limitation that is not fully reversible. COPD is the fourth leading cause of death in the U.S., affecting more than 16 million people. COPD includes emphysema, characterized by the destruction and enlargement of the lung alveoli, chronic bronchitis, a condition with chronic cough and phlegm, and small airways disease, the narrowing of small bronchioles. Risks factors to develop COPD are cigarette smoke (main risk factor), respiratory infections (predominantly during childhood), occupational exposures (e.g., coal mining, gold mining, cotton textile dust and dust in general), airway responsiveness (e.g., asthma), ambient air pollution and passive or second hand smoke. Genetic risk factors include α1 anti-trypsin deficiency.

Large airway changes cause cough and sputum. Mucous gland enlargement, goblet cell hyperplasia, neutrophil influx, elastase production and smooth muscle hypertrophy can limit airflow or cause chronic bronchitis. Small airway changes cause physiologic alterations. In small airways of less than 2 mm, there is goblet cell metaplasia, loss of Clara cells, mucous secretions with infiltrating mononuclear inflammatory cells and smooth muscle hypertrophy. Thus excess mucus, edema and cell infiltrates result. Surfactant reduction or wall fibrosis may cause the collapse or reduction of airways.

Emphysema is characterized by the destruction of gas exchanging airspaces (respiratory bronchioles, alveolar ducts and alveoli). The alveolar walls become perforated and progressively coalesce into small, abnormal, distinct airspaces that lead to larger airspaces. Breathing is difficult as the lost fine architecture of the lung results in holes in the lungs, obstructed airways, trapped air and poor exchange of oxygen due to reduced elasticity of lungs. Emphysema is classified into distinct pathologic types in which the most prominent types are centriacinar and panacinar. Centriacinar emphysema (most frequently associated with smoking) displays enlarged airspaces in association with respiratory bronchioles. Centracinar emphysema is quiet often focal and most prominent in the upper lobes and superior segments of the lower lobes. Panacinar emphysema refers to abnormally large airspaces evenly distributed within and across acinar units. It is more often observed in patients with αl anti-trypsin deficiency. The pathogenesis of emphysema comprises three interrelated events. First, chronic exposure to environmental insults, mainly cigarette smoke leading to inflammation caused by activation of lung epithelial cells and alveolar macrophages. These cells release cytokines/chemokines followed by acute neutrophil recruitment within the terminal airspaces of the lungs. Second, there is damage to the extracellular matrix of the lungs. Inflammatory cells (e.g., neutrophils) release elastolytic proteases that degrade elastin which is critical to the integrity of both the small airways and the lung parenchyma. Finally, death of endothelial and epithelial cells is coupled with the ineffective repair of elastin and other ECM components. The end result is defective and reduced alveogenesis and re-septation of the lungs leading to pulmonary emphysema.

Lung functions display several marked changes with aging. The lungs are pink at birth, in adults they can be dark grey and mottled in patches and in the aged they can be black patches due to inhaled carbonaceous material in the loose connective tissue near the lung surface. There is a significant loss of functionality.

The number of alveoli dramatically decrease with aging. Numbers of these cells can be increased by the implantation of type II alveolar epithelial cells into the alveolar surface. Type I can be converted in vivo or alternately type I alveolar cells can be differentiated in vitro and implanted. Type I is the preferred type of alveolar epithelial cells to be used in the invention. The cell can be sprayed into the lung cavity with or without homing cell adhesion molecules or implanted by injections.

The ventilation dynamics decrease with aging due to chest wall stiffness and a loss of elasticity occurs that can compromise the lung functions. The maximal expiratory volume decreases by 45%. Increased compliance through elastin production can be effected by implantation of fibroblasts by injection or inhalation into the affected lung parenchymal connective tissue. The location includes the alveoli wall's connective tissue layer or septa. Increased muscle contraction can be obtained by muscle cell implantation into the intercostal and abdomen muscles. Additionally, tendocytes can be implanted into the main central tendon to increase its activity during ventilation. Chondrocytes can be implanted into the rib cartilage for additional rib movement.

During aging there is a decreased cough reflex that can result in microaspiration. Dyspnea, hypoxia and aspiration pneumonia are due to lung disease, not age.

Implanted fibroblasts can be used to digest fibrotic tissue present in IPF and the ILDs. Depending on the degree of progression of the lung diseases after fibrosis, other cells types (e.g. alveolar cells) can be added back to the lung tissue. Implanted fibroblasts can be used to remove the fibrosis and produce new connective tissue. Without being bound to a particular theory of action, the fibroblasts are believed to remodel scars or fibrotic tissue, as evidenced by experiments for other tissue scars previously described by the inventors in other patent applications. These fibroblasts can also stop inflammatory processes such as that present in the initial stages of lung diseases (e.g. alveolitis, festering inflammation). Advanced COPDs, such as emphysema, can be treated with removal of scar tissue followed by populating the connective tissue built by fibroblasts with alveolar cells in advanced stages of the disease. Alveolar cells can be used to increase surfactant production so as to increase ease of ventilation in aging, as well as in premature babies and a number of other lung diseases. Surfactant can also neutralize excess tissue degradation by proteases released from macrophages, prevalent in certain diseases or conditions. Implanted macrophages can be used to rid the lung areas (e.g. alveoli) of inhaled environmental particles.

LVRS (lung volume reduction surgery) is a surgery to remove the most damaged lung tissue (from emphysema, cancer) and improve the movement of ventilation improving lung function. Pericardial tissue can be used to cover the resection or staples used. Pericardium can be made in vitro from the patient's own connective tissue cells.

Kidney Function and Renal Failure

In a simple perspective, the function of the kidneys is to filter the blood that flows through them, and to remove the waste products. Waste products are only 5% of the total volume of the urine, the remaining 95% is water. In a more complicated perspective, the kidneys have to comply with several other functions of utmost importance in maintaining body homeostasis. These major functions include: the regulation of water, electrolyte and acid-base balance; the regulation of body fluid osmolarity and electrolyte concentrations; the regulation of arterial pressure; the secretion of, conversion of and response to, hormones and peptides such as renin (juxtaglomerular cells), angiotensin I, and the active form of Vitamin D, amongst others; the production of erythropoietin (EPO) the erythrocyte producing growth factor, by cells of the peritubular capillary endothelium; and the excretion of metabolic wastes. In the production of urine, the kidneys perform four processes: the filtration of plasma, tubular reabsorption, tubular secretion and concentration of the final product, urine. These functions can be lost due to aging and disease and can be improved by implantation of the appropriate cell types listed below into the respective tissue area.

Structure and Histology

The kidney is composed of three main regions: a pale outer region, the cortex and a darker inner region, the medulla, divided into the outer medulla and the inner medulla. The inner medulla generates a concentrated or diluted urine. The outer medulla is divided into 8-18 conical masses, the renal pyramids. The renal pyramids are flanked by extensions of the cortex. The renal pyramids provide anatomical support for the intricate circulatory system that traverses the most intimate parts of the nephron, facilitating the renal tissue/blood exchange. The kidney is composed of many tortuous, closely packed uriniferous tubules bounded by delicate connective tissue. Each tubule consists of two embryonic distinct parts. The nephron is the functional unit of the kidney and produces urine. The collecting duct completes the concentration of urine.

In essence the nephron is a blind-ending, epithelial-lined hollow tubule, which typically originates in the renal cortex and terminates by emptying into the collecting duct system in the inner medulla. Collecting ducts may receive distal tubules from several nephrons and the ducts join together to form openings or tiny orifices at the papillary tip of the pyramid. The nephron has a first portion that consists of a renal corpuscle (0.2 mm in diameter) that filters the plasma and a renal tubule that selectively resorbs from the filtrate to form urine.

There are one to two million renal corpuscles in each kidney and their number decreases with age. Each has a central glomerulus of vessels and a glomerular (Bowman's) capsule, from which the renal tube originates. The glomerulus proper is the dilated, blind-ending proximal part of a renal tubule. It consists of a tuft of convoluted branched capillaries supplied by an afferent arteriole. Blood emerges into the efferent arteriole which supplies the capillary beds and the vasa recta. The entry point of the glomerulus is known as the vascular pole of the renal corpuscle. The glomerulus is covered by a thin, specialized layer of epithelial cells in the inner or visceral layer and turns back at the vascular pole to form an outer or parietal epithelial layer in continuity with the cuboidal cells of the renal tubule. The lumen of the renal tubule is molded to accommodate the glomerulus. It forms a hollow space around the capillaries that constitutes the Bowman's space, which along with its parietal and visceral cell layers, are known as the Bowman's capsule. The parietal layer is a simple squamous epithelium, while the visceral layer is composed by a specialized epithelial cells called podocytes. Plasma circulating through the glomerulus is filtered into the Bowman's space to form an ultra-filtrate that can exclude larger protein molecules that are selectively resorbed. The podocytes are stellate cells in intimate association with capillaries. Podocytes are highly specialized epithelial cells with long cytoplasmic processes, foot processes or pedicles interdigitating with the primary foot processes of other podocytes and wrapping around the capillary loops. Foot processes make contact with the basal lamina of the capillary endothelial cells branching into secondary and tertiary processes known as pedicels. There is a space between the foot processes called the filtration slit, which is bridged by a membranous slit diaphragm adjacent to the basal lamina. On the opposite side of the basal lamina is the thin fenestrated endothelium of the capillaries. The association of foot processes and their slit diaphragm, basal lamina and the fenestrated endothelium comprise the structural tissue for glomerular filtration, which separates blood from the ultrafiltrate in Bowman's space. The central region of the glomerulus is occupied by the mesangium, a supporting framework of specialized connective tissue made up of mesangial cells and its extracellular matrix. These mesangial cells have contractile and phagocytic properties and the ability to respond to vasoactive agents. Phylogenetically, mesangial cells are related to vascular pericytes (undifferentiated mesenchymal like stem cells) and clear the glomerular filter of immune complexes and cellular debris. Their contractile properties help regulate local blood flow.

The second portion of the nephron, the renal tubule, is located in the cortex and called the proximal convoluted tube (PCT). The PCT's lumen is lined throughout by a simple (single-layered) low cuboidal epithelium with a brush border of tall microvilli. Microscopically these cells show a strongly eosinophilic cytoplasm and their bases show faint striations due to the presence of complex series of infoldings (thus multiplying the active surface area) of the basal plasma membrane, for the reabsorption of fluid and solutes against steep concentration gradients.

Upon entering the outer medulla, the PCT shows an abrupt transition into the thin descending limb of Henle's loop which is 30 μm in diameter lined with low and cuboidal epithelial cells with protruding nuclei. The function of this portion of the Henle's loop is to maintain a hypertonic medulla to promote the mechanisms that concentrate urine. Following this thin portion of the Henle's loop is the thick ascending limb of the Henle's loop in which its lumen shows low cuboidal epithelial cells, and deep basolateral folds and short apical microvilli. This portion of Henle's loop is the source of protein traces found in normal urine. This portion of the loop ascends towards the cortex again and very close to the glomerulus. Its cells turn into a narrow cluster of approximately 40 cells closely packed side by side to form the macula densa (MD), a sensory component, chemoreceptor type of structure monitoring the concentration of NaCl⁻ in the filtrate after its passage through the loop of Henle and adjusting the glomerular filtration rate (GFR). Beyond the macula densa is the distal convoluted tube (DCT) showing a wider lumen lined by cuboidal epithelium, but without microvilli. The main function of the DCT is to reabsorb NaCl. The DCT then makes the transition into the connecting tubes (CT) to finally turn into the long cortical collecting ducts (CCD) that extend into the papillary region. The function of the CCDs is to reabsorb water and Na+via aquaporins (water channels) formed by the lining epithelium of tall columnar cells. The reabsorption of water is regulated by vasopressin receptors present in MD cells.

The juxtaglomerular apparatus consists of three cell components, the macula densa (MD) described above, the juxtaglomerular cells located in the wall of the afferent arteriole, which is the vessel that supplies the glomerulus, and the extraglomerular mesangial cells located in the cleft formed between the afferent and efferent arterioles of the glomerulus, in which the function remains unknown. The MD regulates the release of renin from the juxtaglomerular cells. Renin is a participant in the renin-angiotensin system (RAS) to regulate the glomerular filtration rate (GFR) and ultimately control the body fluid homeostasis in response to falls in the blood pressure. Renin-angiotensin system (RAS) is an endocrine network that is the main regulator of blood pressure, intravascular volume and electrolyte balance. Juxtaglomerular apparatus (JGA) cells produce renin which cleaves circulating angiotensinogen to angiotensin I (Ang I). Ang I is activated by ACE (angiotensin converting enzyme) to Ang II, the main effector of RAS. Ang II is a vasoconstrictor and stimulator of aldosterone release. Thus, RAS responds to low blood pressure or diminished intravascular volume by Ang II synthesis.

Interstitial cells, mainly fibroblasts-like, and macrophages and lymphocytes along with extracellular matrix are components of approximately 10% of the cortex. This percentage increases within the medulla that shows a larger proportion of lipid-rich interstitial cells. Renal cells, such as cortical tubular cells (e.g., capillary endothelial cells), and/or interstitial fibroblasts (e.g., cortex, medulla) produce EPO. Renal cells, such as the proximal tubular cells, produce the active form of vitamin D in which 25-hydroxycholcalciferol is converted to the 1, 25-dihydoxy form. The active form of vitamin D is needed for calcium absorption in the intestine and osteoclast activity in the bone and can prevent glomerulosclerosis.

Renal Failure

Renal failure (RF) is broadly defined as a fall in the GFR (to 30 ml/min or less) with a resulting accumulation of nitrogenous wastes in the body. RF can be acute (ARF) occurring over days or weeks, subacute or rapidly progressive when it develops over weeks or a few months, and chronic (CRF) when it develops over months or even years. All types can be caused by numerous health problems. The major causes of ARF can be classified as prerenal, caused by hipovolemia and cardiovascular failure or postrenal, caused by extrarenal obstruction, intrarenal obstruction and bladder rupture. Specific renal diseases of ARF include vascular diseases, in which malignant hypertension is the most common. Vascular diseases leading into glomerular sclerosis are known as glomerulonephritis and interstitial nephritis. Also includes is acute tubular necrosis that is due to post-ischemia, pigment-induced, toxin and drug-induced, pregnancy-related or advance liver disease related. CRF results from a wide variety of renal diseases affecting nephrons or the vasculature, in which a gradual decline in renal function is associated with progressive and irreversible loss of functioning nephrons. CRF is the result of all chronic renal diseases. Examples of chronic diseases affecting adults and the elderly population are diabetes, hypertension, and glomerulonephritis of diverse causes, and are the most common culprits for terminal CRF.

Histopathology

The terms glomerulonephritis and glomerulopathy are used interchangeably to denote glomerular injury. Glomerular diseases and terms to describe them are as follows: Primary glomerular disease is when the pathology is confined to the kidney and secondary glomerular disease is when the kidney fails due to a systemic disease. Lesions can be segmental or global when they involve part of or almost all of the glomerular tuft, respectively. Lesions are classified as focal or diffuse when they involve the minority (<50%) or majority (>50%) of glomeruli, respectively. Proliferative disease is an increase in glomerular cell numbers. Proliferation of resident glomerular cells is defined as intracapillary or endocapillary when referring to endothelial or mesangial cells and extracapillary when referring to cells in the Bowman's space. Membranous disease is applied to glomerulonephritis dominated by expansion of the glomerular basement membrane (GBM) by immune deposits. Sclerosis refers to an increase in the amount of homogeneous nonfibrillar ECM of similar composition to GBM and mesangial ECM. Fibrosis involves deposition of ECM including collagen type I and II and is more commonly a consequence of healing inflammations.

Glomerular disease can be classified according to major morphological features. Examples are: 1) Proliferative glomerulonephropathies (GN) include focal proliferative glomerulonephritis (due to mesangial proliferative glomerulonephritis showing predominantly proliferation of mesangial cells). It also includes diffuse proliferative glomerulonephritis marked by increased cellularity due to infiltration of macrophages and monocytes or proliferation of endothelial or mesangial cells or a combination of all these cell types. A third category is crescentic glomerulonephritis which are glomeruli containing areas of fibrinoid necrosis and crescents in Bowman's space composed of proliferative parietal epithelial cells. 2) GN affecting the glomerular basal membrane (GMB) include membranous glomerulopathy characterized by diffuse thickening of the GMB and immune deposits, minimal change disease (MCD) marked by foot process effacement, and focal and segmental glomerulosclerosis (FSGS). FSGS is characterized by segmental capillary collapse with deposition of abnormal hialinous material affecting greater than 50% of glomeruli. 3) Membranoproliferative GN combines glomerular proliferative features with GMB involvement. 4) Glomerular deposition diseases display extravascular deposition of fibrillar material. 5) Thrombotic microangiopathies display microthrombi in glomerular capillaries and endothelial damage.

Diagnosis, Clinical Manifestations and Treatment

The diagnosis of both ARF and CRF calls for a complete battery of biochemical tests of blood and urine analyzing renal function. The collection of urine over a period of 24 hours, the detailed microscopic analysis of the urinary sediment, imaging of the kidneys by X-ray, ultrasound, CT scan or MRI, renal biopsy are examples that simultaneously assess the suspected underlying cause these tests. ARF is usually recognized by fmding a rising blood urea nitrogen and/or serum creatinine concentration during the biochemical monitoring of the seriously ill patient. Another important sign is the sudden significant reduction in the urinary volume in a well hydrated patient. CRF is recognized by the unequivocal appearance of signs of uremia, a constellation of signs and symptoms shown by the patient that is retaining urea and other end products of the metabolism that affect every single organ of the body. Uremia is the result of profound and progressive loss of renal function to below 20 to 25% of the normal GFR. Most cases of ARF are reversible if early detected and properly treated. A principle of the therapy is to exclude causes of deterioration in renal function that are potentially remedial. Conservative therapy is capable of controlling many of the manifestations of ARF. Conservative therapies include the correction of the intravascular volumes, the adjustment of the fluid intake versus fluid output, the corrections in the electrolyte balance and protein intake, and the normalization of the blood pressure among other general measurements. In the presence of acute and extensive tubular necrosis, kidney dialysis is indicated. The treatment for CRF is limited to dialysis, either as hemodialysis or peritoneal dialysis. Ultimately a kidney transplant may be needed.

Kidney function during aging. Aging accounts for 40 to 50% loss of function in glomerular filtration rate. Renal disease increases with age. 11% of people over the age of 65 develop primary renal disease with renal function (e.g., glomerular filtration rate) less than 60% of that seen in a normal individual. Although the underlying cause of age-related renal disease is unknown, it has been suggested that the development and progression of renal disease is associated with loss of functioning nephrons, specifically related to the decrease in the number of renal corpuscles and the development of sclerosis in the tuft of capillaries forming the glomerulus. This process is irreversible. Other histological changes have been found in age-related renal disease. It has been observed that when renal disease progresses rapidly the glomerular size continues to increase, whereas other aspects of the kidney architecture remain appropriate relative to both the overall body and kidney size. Increase glomerular size can result either from an increase in the number of cells (hyperplasia) or an increase in the cell size (hypertrophy).

During aging there is an overall (20%) decrease in the maximal urine-concentrating ability. This function is not related to glomerular changes and is assessed by three parameters: (i) maximum urine osmolality (the ability of the kidney to reabsorb or conserve water after overnight water deprivation); (ii) minimal urine flow over a 12-hour period; and (iii) the ability to conserve solute by reabsorbing NaCl and/or urea. The elderly exhibit a 20% reduction in maximum urine osmolality, a 100% increase in minimal urine flow rate, and a 50% decrease in the ability to conserve solute. All of the three renal fimctions described above take place in the two distinct limbs of the loop of Henle in the renal medulla.

Renal papilla is a source of adult kidney stem cells that can be used in the invention.

Placement of mesangial cells and/or macula densa cells and/or juxtaglomerular cells into the renal corpuscle can increase nephron functioning and increase in nephron number. Other cells such as the podocytes and epithelial cells of the parietal layer can be used for introduction to the Bowman's capsule. This method can repair or augment the glomerular filtration rate, that decreases during aging and other glomerular diseases. This method can regulate blood pressure, electrolyte balance abnormalities and deficiencies in urine concentration functions.

Fibroblasts (e.g., interstitial) or mesangial cells can be used to remove fibrosis or sclerosis of the glomerulus to improve glomerular functions such as glomerular filtration rate, urine concentration, electrolyte balance, and blood pressure regulation

The epithelial cells of the DCT can be placed into the DCT to improve resorption functions that decline in renal disease and aging.

Hormone functions can be enhanced with appropriate cell types. The appropriate renal cells can be introduced into the cortex or medulla to produce EPO to increase red blood cell production from the bone marrow and to treat anemias. Renal cells producing the active form of vitamin D can be used to control calcium metabolism and treat osteoporosis, amongst other diseases. Juxtaglomerular cells can be introduced to produce renin to regulate blood pressure and improve mineralcorticoid function and deficits in certain diseases. Introduction of macula densa cells can be used to increase concentration of urine. This method is beneficial to aged patients and those with disease such as diabetes insipidus.

Alzheimer's Disease (AD)

AD is the most common and devastating brain degenerative disease causing dementia in the absence of other prominent neurological signs. Alzheimer's disease is clearly age-related. The prevalence of AD doubles every 5 years beyond the age of 65, affects greater than 20 percent in people older than 80 years old and afflicting over 4.5 million people in the U.S. Alzheimer's disease is multifactorial with both genetic and environmental factors implicated in its pathogenesis. Genetic predisposition to AD emerges has a clear-cut pattern in some families, particularly in those with early-age of onset (usually before 60 years). Some AD even follows an autosomal dominant pattern of inheritance in which mutations in three genes. APP (amyloid precursor protein gene), PS-1 (pre-senilin 1 gene or PSEN1), PS-2 (pre-senilin 2 gene or PSEN2) and ApoE (encoding for apolipoprotein E) have been directly implicated with sporadic AD. In the case of ApoE, carrying one copy of the □4 isoform allele increases the risk of developing AD about 3-fold, whereas carrying two copies increases the risk up to 15-fold. Other reported gene risks factors involve polymorphisms in genes that encode the inflammatory cytokines interleukin 1α, interleukin β, and tumor necrosis factor α (TNFα).

The outstanding pathology feature in AD is death and disappearance of neurons in the cerebral cortex, the massive loss of neuronal synapsis, and the histologic presence of neurofibrillary tangles (NFTs, aggregates of Tau proteins), and senile plaques (complex extracellular lesions primarily composed of aggregated β-amyloid protein and reactive glial cells) and the widespread sclerosis or fibrosis (e.g., hyaline degeneration) of the medium and smaller blood vessels of the brain. Plaques can be associated with dystrophic neuritis. A major constituent of NFTs is a hyperphosphorylated form of the axonal protein tau, which is normally found in the cells microtubule system. A major constituent of senile plaques is beta-amyloid protein (Aβ), which is derived from the neuronally produced amyloid precursor protein (APP) via the action of β and γ secretase. Beta-amyloid protein(Aβ) shows up in many body tissues and is overproduced in the brain of patients with AD. The exact reason for the overproduction is unknown but the steady-state concentrations of Aβ are determined by the dynamic balance between anabolic and catabolic activities. Research has shown an elevation of Aβ anabolism with reduced catabolism in the brain of individuals with AD. The Aβ degrading enzyme neprilysin, a metallopeptidase, as well as an endothelin converting enzyme may represent up to 80% of the total Aβ degrading activity in the brain.

There are two main theories as of the cause of AD known as the Tau and Aβ theories. One theory is that the cause of AD is due to tau hyperphosphorylation that leads to neuronal loss as well as the accumulation of extracellular deposits of Aβ. The amyloid cascade hypothesis indicates the accumulation of Aβ is the true cause of AD, with NFTs and dystrophic neuritis developing as a consequence of Aβ accumulation. Both, tau and Aβ pathologies seem to operate fairly independently at early stages of the disease but later at some stage, the two pathologies become interactive and facilitate each other. An alternate theory is that neither plaques nor tangles initiate the sequence of neuropathological cell death. Instead, plaques and tangles might be “tombstones” of the earlier cell carnage caused by free-floating fibrils of β amyloid.

Aging is associated with decrease levels of estrogen in women and androgens in men. These hormonal reductions might be risk factors for cognitive impairments and the development of AD. Apolipoprotein E (apoE) plays an important role in the metabolism and redistribution of lipoproteins and cholesterol. There are three major human apoE isoforms, ε2, ε3 and ε4. In the brain apoE has been implicated in the neuronal development and regeneration, neurite outgrowth, and neuroprotection. In AD, glial cells, a major cellular source of apoE may recycle cholesterol from neuronal membranes that can then be used to promote the growth of new neuronal processes. In individuals with AD the presence of two alleles encoding for the apo E ε4isoform has been associated with the pathological hallmarks of AD and may be due to an innate impairment in the neuronal remodeling mechanism. There may be an important relationship between the location of the senile plaques and the neuritic pathology and the associated neuronal loss. In multiple animal models dense plaques were invariably located in the neocortex, hippocampus, thalamus and subiculum inside blood vessel walls in there is endothelial lining thinning and basement membrane thickening or splitting to accommodate the amyloid plaque. This finding is indicative of amyloid angiopathy.

The presence of NFTs and senile plaques are characterized by the presence of a broad spectrum of inflammatory mediators. These mediators, which include complement proteins, inflammatory cytokines, prostaglandins and acute phase reactants such as C reactive protein and amyloid P, are produced in resident brain cells, including neurons. Chronic inflammation is prominent in AD and may be spurred on by the plaques and tangles and a subsequent influx of astrocytes and microglia. Normally, these cells clean away the debris, but instead, the inflammation causes damage to host tissue. Thus inflammation exacerbates the neuronal loss in AD. In particular, NFTs and senile plaques show evidence of self-attack by the complement system in a specific way that is called cell autotoxicity, instead of the usual autoimmunity response.

All these processes usually start in the hippocampus and amygdala (the internally convoluted structures that form the medial margin of the cortical mantle of the cerebral hemisphere), but ultimately lead to extensive brain cortex atrophy, especially in the frontal, parietal and temporal regions, the brain regions that control memory, cognition and emotions. There is a corresponding enlargement of the ventricular system, but this is usually not extreme.

The brain consists of the cerebrum, cerebellum and brain stem and each part consists of gray and white matter. AD affects mainly three structures in the brain, the cerebral cortex, the hippocampus and amygdala. The cerebral hemispheres are the largest part of the brain. They each have an external highly convoluted cortex (organized into gyri, sulci and the frontal, parietal, temporal and occipital lobes) beneath which lies an extensive internal mass of white matter that contains the basal ganglia. The cerebral hemispheres contain primary motor and sensory areas. These represent the highest level at which motor activities are controlled and the highest level to which general and special sensory systems project, providing the neural substrate for conscious experience of stimuli. Association areas are modality-specific and also multi-modal, and they enable complex analysis of the internal and external environment and the relationship of the individual with the external world. Parts of the hemisphere, termed the limbic system are concerned with memory and the emotional aspects of behavior. Other areas, primary within the frontal region are concern with the highest aspects of cognitive function.

The cerebral cortex is comprised of grey matter, in which most of the grey matter in the brain is located. The cerebral cortex can be divided into a phylogenetially old allocortex, consisting of the archicortex, paleocortex and a newer neocortex. In general, grey matter is composed of neuron cell bodies of three basic functional types, afferent (sensory), efferent (motor) and interneurones. Each individual neuron may make synaptic contact with hundreds, or even thousands of other neurons with profuse axonal or dendritic branching (arborization).

The cortex exhibits mainly two neuronal cell types; the pyramidal cell type which is the most abundant (70% of the cortical neurons) and the non-pyramidal cells, also called stellate or granule cells (spiny and non-spiny neurons). Spiny stellate cells are the second most common cell type. Both neuron types have numerous dendrites (short, threadlike processes that extend from the cell body branching profusely) and an axon (a long tail-like extension measuring up to a meter that conduct nerve impulses away from the cell body to reach a target). Pyramidal cells are universally projection neurons (which axon leaves the cortex to project into the white matter) using excitatory amino acids, either glutamate or aspartate, exclusively as neurotransmitters. The smallest group of cells comprises the heterogeneous non-spiny or sparsely spinous stellate cells are interneurons. This is a heterogeneous group of cells with a multitude of forms including basket, chandelier, double bouquet, neurogliaform, bipolar/fusiform and horizontal.

The other important cell group and by far the most numerous group of cells populating the cortex are the neuroglial cells (specialized, non-neuronal supporting cells) of 7 types: astrocytes, oligodendrocytes, microglia cells, ependymal cells, choroid epithelial cells, tanycytes and Schwann cells. They are derived from three lineages, the neuroectoderm of the neural tube, the neural crest; and angyoblastic mesenchyme. The neuroglia is responsible for creating and maintaining an appropriate environment in which the neurons can operate efficiently. Astrocytes project foot processes to capillaries that contribute to the blood-brain barrier, play a role in the metabolism of neurotransmitters and buffer the potassium of the CNS extracellular space, form glial scars in damaged areas of the CNS, undergo hypertrophy or hyperplasia in reaction to CNS injury. These cells provide nutrients to and remove toxins from neurons. They contain glial fibrillary acidic protein (GFAP) and glutamate synthetase. Oligodendrocytes produce myelin in the CNS. One oligodendrocyte can myelinate up to 30 axons. Microglia are derived from monocytes and have phagocytic fimction such as damaged myelin from injured axons. Ependymal cells line the central canal and ventricles of the brain. These cells are not joined by tight junctions therefore allowing free exchange between the cerebrospinal fluid and the CNS extracellular fluid.

Choroid epithelial cells are the continuation of the ependymal layer that is reflected over the choroids plexus villi, and these cells secrete cerebro spinal fluid (CSF). These cells are joined by tight junctions, which are the basis for the blood-CSF barrier. Tanycytes are modified ependymal cells that project to both capillaries and neurons. These cells mediate transport between ventricles and the neurons. These cells project to the hypothalamic nuclei that regulate the release of gonadotrophic hormones from the adenohypophysis. Schwann cells produce myelin in the peripheral nervous system (PNS) and are derived from neural crest cells. One Schwann cell myelinates one axon, they invest all myelinated and unrmyelinated axons of the PNS and are separated from each other by the Ranvier nodes.

The grey matter also contains a rich supply of blood vessels. Microscopically the neocortex is cytoarchitectonically and horizontally laminated into 6 layers from the surface to the limit of the white matter. 1) The molecular or plexiform layer is cell sparse, containing only scattered horizontal cells and their processes enmeshed in their axons and dendrites. Inside this layer there is a specialized type of neuronal cell, the Cajal-Retzius (CR) cell, that can be vulnerable in the initial stages of AD. The CR cells secrete reelin, a protein important for cortical and hippocampal development and synaptogenesis. Their loss in AD may play a role in the synaptic and other pathologies associated with the disease. 2) The external granular lamina contains small neuronal bodies. These include small pyramidal and non-pyramidal cells. 3) The external pyramidal lamina contains pyramidal cells of varying sizes, together with scattered non-pyramidal neurons. This layer is often divided into IIIa, IIIb and IIIc from more superficial to the deepest, with IIIc containing the largest pyramidal neurons. 4) The internal granular lamina contains densely packed, small round cell bodies of non-pyramidal cells, notably spiny-stellate cells and some small pyramidal cells. 5) The internal pyramidal (ganglionic) lamina typically contains the largest pyramidal cells in any cortical area. Scattered non-pyramidal cells are also present. 6) The multiform (or fusiform/pleiomorphic) layer consists of neurons with a variety of shapes, including pyramidal, spindle, ovoid and many others. Typically, most cells are small to medium in size.

The white matter is composed mainly of myelinated axons from cortical neurons and neuroglial cells and provides routes (i.e., nerve tracts, fibers) that connect one part of the brain to the other. These routes are categorized on the basis of their course and connections. They are either association fibers, which link different cortical areas in the same hemisphere; commissural fibers, which link corresponding cortical areas in the two hemispheres or projection fibers, which connect the cerebral cortex with the corpus striatum, diencephalons, brain stem and the spinal cord.

The Hippocampus.

The hippocampal formation is part of the limbic lobe which includes large parts of the cortex on the medial wall of the cerebral hemisphere. The hippocampal formation consists of the hippocampus proper, the dentate gyrus, the subicular complex and the entorhinal cortex. Papez (1937) observed the emotional disturbances of patients with damage to the hippocampus, proposed that emotional expression is organized in the hippocampus, experienced in the cingulated gyrus and expressed via the mammillary bodies. The Papez neuronal circuit was described between the hippocampus and the hypothalamus inside which the peripheral expressions of emotional states are controlled. This circuit has been linked with spatial short-term memory. Later the term “limbic system” became popular to describe the limbic lobe. The hippocampus itself is a curved elevation, 5 cm long, along the floor of the inferior horn of the lateral ventricle and it is covered by ependyma (cellular membrane lining the cerebral ventricles and the central canal of the spine). The hippocampus is a trilaminar archicortex. It consists of a single pyramidal cell layer, with plexiform layers above and below. It may be divided into three distinct fields, CA1, CA2 and CA3. Field CA1 is the most complex of the hippocampal subdivisions. The thickness of the pyramidal cell layer in this field varies from 10 to 30 cells. The CA2 field has the most compact layer of pyramidal cells. Field CA3 has the largest pyramidal cells in the hippocampus and is 10 cells thick all along the field. The subicular complex is divided into subiculum, presubiculum and parasubiculum. The subiculum consists of a superficial molecular layer containing apical dendrites of subicular pyramidal cells, a pyramidal cell layer 30 cells thick, and a deep polymorphic layer. The presubiculum is distinguished by a densely packed superficial layer of pyramidal cells and a plexiform layer superficial to the dense one. The parasubiculum also has a superficial plexiform layer and a primary cell layer.

The entorhinal cortex (Broadmann's area) extends to the anterior limit of the amygdala and overlaps a portion of the hippocampus. This cortex is divisible into six layers. Layer I is acellular and plexiform. Layer II is a narrow cellular layer of islands of large pyramidal and stellate cells that is visible to the naked eye as bumps known as the “verrucae hippocampae”. Layer III consists of medium-sized pyramidal cells. Layer IV is acellular and displays dense fibers called the lamina dissecans. Layer V consists of large pyramidal cells 5 or 6 deep. Layer VI is thin, only readily distinguishable from layer V and consists of large pyramidal cells as well. The dentate gyrus is the point of entry into the hippocampal circuitry. It receives fibers from layers II and III of the entorhinal cortex, passing into the molecular layer of the dentate gyrus, located on the dendritic spines of granular cells. These cells project heavily onto the proximal dendrites of CA3 pyramidal cells of the hippocampus (also called Schaffers collaterals) and terminate in the CAI hippocampal field. Glutamate, and/or aspartate appears to be the major excitatory transmitter in the hippocampal circuitry.

This circular pathway of neurons from the entorhinal cortex to the dentate gyrus, the CA3 and CA1 pyramidal neurons of the hippocampus to the subiculum via the amygdala and back to the entorhinal cortex, is heavily damaged in AD.

The Amygdala.

The amygdaloid complex is made up of lateral, central and basal nuclei which lie in the dorsomedial temporal pole, anterior to the hippocampus and close to the caudate nucleus. Collectively the nuclei form the ventral, superior and medial walls of the anterior horn of the lateral ventricle. The lateral nucleus has dorsomedial and ventrolateral subnuclei. The central nucleus has medial and lateral subdivisions. The basal nucleus is commonly divided into a dorsal magnocellular basal nucleus, intermediate parvicellular basal nucleus, and a ventral band of darkly staining cells usually referred to as the paralaminar basal nucleus. The accessory basal nucleus lies medial to the basal nuclear divisions and it is usually divided into dorsal, magnocellular, and ventral parvicellular parts. The lateral, the basal nuclei and the accessory basal nucleus are often referred to as the basolateral area (nuclear group) of the amygdaloid complex. The basolateral area shares characteristics with the cerebral cortex and although it lacks a laminar construction, it has direct, reciprocal connections to the temporal lobe and it projects to the motor or premotor cortex.

A particular area of the amygdala, the parvicellular basal nucleus is the area involved in the circular pathway of neurons damaged in AD (mentioned above).

The organization of the extensive subcortical and cortical interconnections and connections of the amygdala are consistent with a role in emotional behavior. The amygdala is important in evaluating the significance of environmental events, most particularly the association between stimuli and reinforcement.

The onset of AD is insidious and subtle, with changes most noticeable first in memory of recent happenings and in other aspects of mental activity. Emotional disturbances such as depression, anxiety, or odd behavior are prominent in early stages. Progression is usually slow and gradual, which unless other medical conditions supervene, may smolder on for 10 or more years. In the milder cases the manifestations can be those of simple senile dementia. In the advance stages of the disease more severe and unusual disorders of thought and intellect including difficulties of the speech, disorders of the voluntary movement, and abnormal space perception may occur. Terminally ill patients may loose all ability to perceive, think, speak or move.

For years, the only reliable way to confirm the disease was post-mortem by direct study of the brain during autopsy. Current advances in diagnosis are in brain imaging. Sophisticated CT (computerized tomography) scans, MRI (magnetic resonance imaging), BOLD MRI (combination of MRI plus measurements of cerebral blood flow) and PET (positron emission tomography) scans combined with improved neurobehavioral testing make it possible to detect the disease with 90% accuracy even at the early stages.

To date all the treatments for AD are palliative and not preventive or curative of the disease. Acetylcholinesterase inhibitors (e.g. tacrine, donepezil and rivastigmine) and reduction of the oxidative stress with antioxidants are used. Routine use of non-steroidal anti-inflammatory drugs (NSAIDs) appear to reduce the risk of developing AD by curbing the chronic inflammatory response characteristic of the disease. Cholesterol lowering drugs (i.e., statins) may lower the risk for AD by countering the inflammatory response, diminishing atherosclerosis in the vessels of the brain or reduce the A\tilde{β}. formation.

Astrocytes secrete proteases. These proteases can lyse protein aggregates, β-amyloid deposits, neurofibrillary tangles or other aggregates present in AD. Inflammation causes fibrosis in AD. Immune cells recognize the cardinal proteins of AD. Astrocytes and other brain cell types help build the architecture of the brain. Cell types, preferably brain astrocytes, can be expanded in culture and implanted in the affected brain area of AD patients. Astrocytes can dissolve the plaque formation and remove brain tissue scarring which takes place near the Aβ aggregates (e.g., sclerosis, fibrosis). Immune cells, such as microglia or brain macrophages or other body type macrophages (e.g. from skin), can be used to remove the AD plaques. Implantation can be diffuse or in specific areas of destruction, depending on the stage of AD. In particular, the circular pathway of neurons in the hippocampus and amygdala that become heavily damaged are prime locations for the implantation of cells. Neuroglial cells may be implanted to rebuild devastated areas of lost functionality and structure. For central nervous tissue and the brain, injections or perfusions into the brain through the local bloodstream or CSF can be used to introduce cells.

Thus, embodiments of the invention include the introduction of cells, e.g., astrocytes, immune cells, or precursors, into a patient to treat AD using techniques described herein for obtaining, culturing, and introducing cells into a patient. The cells may be introduced with or without the proteins, factors, and supplementing materials described herein. Autologous cells, allogenic cells, or xenogenic cells may be used. Cells include stem cells, various differentiated cells, and their precursors. The site of introduction may be at or near the defect or at a site distant from the defect, as described herein.

Parkinson's Disease

PD is the most common disease presenting bradykinesia, muscular rigidity and tremor with sensorial and intellectual compromise. PD affects approximately 1% of the U.S. population over the age of 50 and over 5% by the age of 85. After Alzheimer's Disease, PD is the second most common age-related neurodegenerative disorder. Typically PD is a chronic, progressive and disabling disorder of middle or later life, affecting men slightly more frequently than women. The cause of the disease remains unknown but it is defmed as a multifactorial, sporadic disease in occurrence, although a low familial incidence is recognized and some genetic susceptibility can be involved. PD appears to be more prevalent in industrialized countries. This suggests that environmental exposure, such as to industrial toxins and contaminated water might play a role in PD. Besides exposure to environmental toxins, head trauma and viral diseases have been associated with PD.

PD is the most common pathological condition affecting the basal ganglia. The histopathological hallmarks of the disease are dopaminergic striatal insufficiency secondary to a loss of dopaminergic neurons in the substantia nigra pars compacta. Another histopathological marker of the disease is the presence of Lewy bodies which are clumps of degenerated pigmented neurons in the substantia nigra composed of fibrils of α synuclein protein. The fundamental mechanisms involved in neuronal cell death is unknown. The biochemical consequences of the neuronal loss are a steady decrease in the levels of the neurotransmitter dopa circulating in the stratium. Dopamine is responsible for allowing the brain to generate signals for smooth, well-regulated motor or muscle function. It is thought that by the time the patient develops symptoms 80% of the dopamine producing neurons have been lost. PET studies reveal a deficit in dopamine storage and reuptake, due to the loss of nigrostriatal terminals, but intact dopamine receptors remain throughout the medium spiny neurons which are the target of the nigrostriatal pathway.

Dopamine appears to have a dual action on medium spiny stratial neurons. It inhibits those in the indirect pathway and excites those in the direct pathway. Consequently, when dopamine is lost from the striatum, the indirect pathway becomes overactive and the direct pathway becomes underactive. Overactivity of the striatal projection to the lateral pallidum results in inhibition of the pallidosubthalamic neurons and, consequently, overactivity of the subthalamic nucleus. Subthalamic efferents mediate excessive excitatory drive to the medial globus pallidus and substantia nigra pars reticulate. This is exacerbated by underactivity of the GABAnergic, inhibitory direct pathway. Overactivity of basal ganglia output then inhibits the motor thalamus and its excitatory thalamocortical connections.

As with other diseases of the CNS, there is a presence of a broad spectrum of inflammatory mediators, which include complement proteins, inflammatory cytokines, prostaglandins and acute phase reactants such as C reactive protein in PD. Chronic inflammation has been widely documented in AD as well as in PD. Neuronal loss stimulates a chronic inflammation reaction with increased amount of astrocytes and microglia that is aimed to clean the debris. Inflammation causes damage to host tissue. There is strong evidence that inflammation exacerbates the neuronal loss in PD as well as in AD.

Basal Ganglia.

The basal ganglia refers to a number of sub-cortical nuclear masses that lie in the inferior part of the cerebral hemisphere, lateral to the thalamus. The basal ganglia includes the corpus striatum and its associated structures in the diencephalons and midbrain, forming a functional complex involved in the control of movement and motivational aspects of behavior. The corpus striatum consists of the caudate nucleus, putamen, and globus pallidus. The putamen and the caudate nucleus together are referred to as the striatum, which is highly cellular and well vascularized.

The Striatum.

Neurons of both dorsal and ventral striatum are mainly medium-sized multipolar cells mixed with a smaller number of large multipolar cells in a ratio of at least 20:1. The most common neuron (usually 75% of the total) is a medium-sized cell with spiny dendrites. These cells utilize γ-aminobutyric acid (GABA) as their neurotransmitter and also express the gene coding for either enkephalin or substance P/dynorphin. Enkephalinergic neurons appear to express D2 dopamine receptors. Substance P/dynorphin neurons have D1 receptors. These neurons are the major, and perhaps exclusive source of striatal efferents to the pallidum and substantia nigra pars reticulate. The remaining medium-sized striatal neurons are aspiny and are intrinsic cells that contain acetylcholinesterase (AChE), choline, acetyltransferase (CAT) and somatostatin. Large neurons with spiny dendrites contain Ache and CAT. Intrinsic synapses are probably largely asymmetric (Type II), while those derived from external sources are symmetric (Type I). The aminergic afferents from the substantia nigra, raphe and locus coeruleus all end as vesicles (the presumed storage site of amine transmitters)

Connections of the stratium are dorsal and ventral and they overlap. In general, the dorsal stratium is predominantly connected with motor and associative areas of the cerebral cortex, while the ventral striatum is connected with the limbic system and orbito-frontal and temporal cortices. For both dorsal and ventral stratium, the pallidum and substantia nigra pars reticulate are key efferent structures. The fundamental arrangement is the same for both divisions. The cerebral cortex projects to the striatum, which in turn projects to the pallidum and substantia nigra pars reticulate. From these efferents leave to influence the cerebral cortex in supplementary motor areas. The greater part of the motor input from the frontal and parietal cerebral cortices to the dorsal striatum arise from small pyramidal cells in layers V and VI of the cortex.

The aminergic inputs to the caudate and putamen are derived from the substantia nigra pars compacta (dopaminergic cell group A9), the retrorubral nucleus (dopaminergic cell group A8), the dorsal raphe nucleus (serotoninergic cell group B7) and the locus coeruleus (noradrenergic cell group A6). This input is known as the “mesostriatal” dopamine pathway. Efferents from the stratium pass to both segments of the globus pallidus and to the substantia nigra pars reticularis where they end in an ordered fashion. Fibers ending in the lateral pallidal segment are grouped in the so-called “indirect pathway”, while fibers ending in the medial pallidal segment are called the “direct pathway”.

A second aminergic outflow is established from the striatum to the pars reticulata of the substantia nigra. The continuity of the ventral and dorsal striata is reinforced by consideration of the aminergic inputs to the ventral stratium. They are derived from the dorsal raphe (serotoninergic cell group B7), the locus coeruleus (noradrenergic cell group A6) and from the paranigral nucleus (dopamine cell group A10) as well as the most medial part of the substantia nigra pars compacta (dopaminergic cell group A9). This pathway is referred as to the “mesolimbic” dopamine pathway.

Globus Pallidus

The globus pallidus lies medial to the putamen and lateral to the internal capsule. It consists of two segments, lateral (external) and medial (internal), which have different connections. The lateral segment projects reciprocally to the subthalamic nucleus via striatopallidal axons as part of the “indirect pathway”. The medial segment is considered to be a homologue of the pars reticulate of the substantia nigra as part of the “direct pathway”. The cell density of the globus pallidus is less than one-twentieth of that of the stratium. The morphology of the majority of cells is identical in the two segments. They are large multipolar GABAnergic neurons that closely resemble the ones in the substantia nigra pars reticulate.

The substantia nigra contains about 400,000 dopaminergic neurons in a normal individual. The substantia nigra is a lamina of nuclear complexes and many multipolar neurons located deep into the crus cerebri in each cerebral peduncle of the midbrain. It consists of a dorsal pars compacta and a ventral pars reticulata. The pars compacta, together with the smaller pars lateralis, corresponds to a group of darkly pigmented neurons, which contain neuromelanin granules, the dopaminergic cell group A9. With the retrorubral nucleus (dopaminergic cell group A8), it makes most of the dopaminergic neuron population of the midbrain and is the source of the mesostriatal dopamine system that projects to the stratium. The pars compacta of each side is continuous with its opposite counterpart through the ventral segmental dopamine cell group A10, which is also known as the paranigral nucleus. This is the source of the mesolimbic dopamine system supplying the ventral stratium and neighboring parts of the dorsal stratium. The dopaminergic cell groups A9 and 10 also contain cholecystokinin (CCK) or somatostatin. The pars compacta projects heavily into the caudate nucleus and putamen. Lesser projections end in the globus pallidus and subthalamic nucleus.

The pars reticulate contains large multipolar cells similar to those in the pallidum. Together they constitute the output neurons of the basal ganglia system. The striatonigral axons utilize GABA and substance P (SP) or enkephalin. The efferent neuronal pathway from the striatum to the superior colliculus, via the substatia nigra pars reticulate is thought to function in the control of gaze. The uncontrolled or fixed-gaze disturbances of advanced Parkinson's tend to support this. Pigmentation of the substantia nigra increases with age, is most abundant in primates, maximal in man, and present even in albinos.

Subthalamic Nucleus.

The subthalamic nucleus is a biconvex, lens-shaped nucleus in the subthalamus of the diencephalons. Within the tissue, small interneurones intermingle with large multipolar cells with very long dendrites. The subthalamic nucleus is encapsulated dorsally by axons, many of which are derived from the subthalamic fasciculus, and which carry a major GABAnergic projection from the lateral segment of the globus pallidus as part of the indirect pathway. The subthalamic nucleus is unique in the basal ganglia in that its cells are glutamatenergic and project excitatory axons to both the globus pallidus and the substantia nigra pars reticulate. The subthalamic nucleus plays a central role in the normal function of the basal ganglia and therefore is crucially involved in the pathophysiology of Parkinson's and other motor disorders. It is the target for Parkinson's neurosurgical treatments. If destroyed, for example, by stroke the result is the development of violent uncontrolled movements known as ballism (ballismus).

Clinical Manifestations.

The disorder typically begins asymmetrically, such as a slight tremor of the fingers of one hand or in one leg that is easily alleviated by relaxation or movement. Although more pronounced in the hands, legs or trunk it may involve the lips, tongue and neck muscles and is seen in the eyelids when lightly closed. As the disease progresses the tremors are accompanied by a stooped posture, stiffness and slowness of movements, the propensity to bend the trunk forward, a fixity of facial expression, a monotonous voice, a typical festinating gait and a characteristic lack of the little spontaneous movements of postural adjustment normal to a healthy individual. Along with the tremors, progressing muscle rigidity and increasing postural “freezing” while moving may make it more difficult for the patients to take care of themselves. Motor symptoms of PD are known to be considerably influenced by emotional factors that are aggravated by anxiety, tension and depression, but minimal when the patient is in a content frame of mind. The autonomic nervous system (ANS) is the part of the nervous system that regulates automatic functions of the body. It is affected by PD. These functions include blood pressure regulation, breathing, swallowing, gastrointestinal function, urination, sweating and sleeping. Diverse symptoms related to impaired ANS function occur as dizziness, saliva drooling, constipation, insomnia, shortness of breath, frequent urination, etc. Intellectual deterioration is not a consistent feature of early PD, yet dementia has been increasingly recognized to be a feature of advanced PD in one-third of the cases.

Diagnosis.

The diagnosis of PD is based on patient symptoms, clinical history and findings on neurological examination. There are no specific CT/MRI brain scan abnormalities or blood tests that confirm the diagnosis of PD. A medical term known as Parkinsonism (emulation of some of the features of PD) is used to describe other neurological disorders that may mimic the disease. A correct diagnosis is made over time when some features disappear or other medical testing reveals the true diagnosis.

Treatment.

Adjustments in the diet of PD patients are usually required to accommodate for a regulated protein intake that will aid the medication regimen. Physical activity is extremely important since inactivity is known to expedite the development of symptoms and their severity. The use of anti-anxiety and anti-depressants agents is common in Parkinson patients.

The pharmacological approach has improved the symptoms of PD. The replacement of deficient dopamine is the gold standard treatment for the disease. Dopamine taken orally does not cross the blood brain barrier (BBB) but its chemical precursor levodopa does. It is converted in the brain to dopamine. Carbidopa is a compound that inhibits the conversion of levodopa to dopamine in other tissues, such as the liver and kidneys. This makes larger amounts of levodopa available to cross the BBB and treat the symptoms. It is usually given to the patient in a drug called Sinemet (carbidopa-L-dopa). Unfortunately, the effects of levodopa therapy eventually wear off after four to ten years of use. Then the dosages needed are so high that significant motor side effects known as dyskinesias (uncontrolled involuntary movements) occur. This secondary effect is explained by involvement of the subthalamic nucleus due to physiological inhibition by overactive pallidosubthalamic neurons secondary to an underactive indirect pathway. DA agonists (chemical agents that mimic the action of DA at the receptor level such as pramipexole or ropinirole) constitute another group of drugs used to treat PD. For some physicians, they constitute the first line of treatment for some patients to be used even before levodopa. A class of drugs known as anti-cholinergics is frequently used in combination with levodopa therapy. Acetylcholine is a major neurotransmitter in the brain, and DA helps to suppress the effects of acetylcholine that are more pronounced in Parkinson patients. Anti-cholinergics agents (trihexyphenidyl and benztropine mesylate) are commonly used. DA is metabolized in the brain by the enzymes MAO-B (monoamino oxidase-B) and COMT (cathechol-o-methyl transferase). By inhibiting these two enzymes steadier brain levels of DA can be maintained. Therefore MAO-B inhibitors (Selegiline) and a COMT inhibitor (Entocapone or Tolcapone) are now available for the treatment of the disease.

Surgical treatments can be employed. The three different surgical approaches to the treatment of PD are pallidotomy/thalamotomy, which is the surgical creation of a small injury in the globus pallidus and/or the thalamus aimed for neuronal ablation of the pathways that send inhibitory signals to the stratium. Deep brain stimulation implants of devices similar to pacemakers in the brain can be used. Experimentally, neural tissue transplantations have been tried (e.g. containing pig, or fetal DA producing cells).

Astrocytes, oligodendrocytes and microglia are the 3 main types of neuroglia or glia in the brain and nervous system. Neuroglia do not conduct electrical impulses but have many other varied functions. Glial cells regulate nerve impulses by interacting with neurotransmitters such as epinephrine or gluatamate (neurotransmitters also thought to aggravate PD), secrete neurotrophic factors to maintain and enhance neuron survival, seal blood vessels in the blood-brain barrier, migrate neurons in brain development, physically support the brain structure by ECM production, deliver nutrients to and remove toxins produced by neurons. Oligodendrocytes myelinate axons in the central nervous system and microglia represent phagocytes. Astrocytes contact neurons, blood vessels and other astrocytes and surround the neuronal synapses.

Cell types that are preferred are those that establish proper connection within the PD disease pathway. This includes cells that produce dopamine. It is a preferred embodiment to implant dopamine neurons to recover the deficiencies of PD, including off-medication dyskinesias for example. Various cell types that can be used such as dopaminergic cells, progenitor cells to dopaminergic cells, stem cells (e.g., fetal, neonatal, adult, germ cells, umbilical cord, embryonic), that are expanded in vitro and then differentiated into dopaminergic neurons or are implanted into the striatum and substantia nigra to establish dopaminergic reinnervation and other PD areas for conversion into dopaminergic neurons. Some adult stem cells are those of the central nervous system (e.g., neural stem cells ) or of the brain or of other tissues such as bone marrow or spleen that can ultimately produce a dopaminergic phenotype. One type of progenitor cell that can be used is the astrocyte isolated from the lining of the brain lateral ventricle.

Cells can be isolated from midbrain or fetal ventral mesenphalic region or other areas of the brain that can provide dopaminergic cells

Other cell types that can produce dopamine or progenitor cells to dopamine producing cells can be used. These cell types include retinal pigment epithelial cells, carotid cell bodies, sympathoadrenal cells containing neural crest derived cells such as sympathoblast, sympathetic neurons, small intensely fluorescent cells of the adrenal medulla and sympathetic ganglia, sympathetic neurons, chromaffm cells of the adrenal medulla and extra-adrenal paragnaglia cells. Such cells express dopaminotrophic factors such as GNDF and TGFβs important for cell survival, proliferation and differentiation of dopaminergic cells and release dopamine and noradrenaline.

Cells such as fibroblasts that are transfected with glial growth factors such as GDNF, GDF-5, neurturin, TGFβs, VEGF or enzyme activities active in increasing levels of dopamine, such as tyrosine hydroxylase or GTP cyclohydolase 1, can be used to enhance the take of implanted cells and the proliferation, differentiation and survival of new and in situ dopamine neurons and other neurons in the brain tissue.

Incubation with and implantation with metabolic accelerators and nutrients such as creatine can help survival the cells implant more efficiently. Astrocytes can be implanted to provide trophic factors for implanted or in situ dopaminergic cells and other cells that improve PD symptoms or causes.

Dopaminergic cells can be implanted into any brain region, preferably in a natural in situ location. Thus beside, the substantia nigra, other sites in the brain such as the hypothalamus, amongst others can be used for implantation.

Long term survival and function can be obtained by populating sufficient numbers of dopamine neurons and/or by co-implanting growth factors, adhesion molecules, survival factors, amongst others. In addition the use of autologous cells or histocompatible cells remove any immune reactions towards the cells that compromise their long term survival.

Astrocytes in any part of the brain can be used and the preferred region is from the lining of lateral ventricle. These cells can migrate to the olfactory bulb. These cells can form mature brain cells, the astrocytes, the microglia and the oligodendrocytes, and the neurons.

Thus, various embodiments of the invention include the introduction of cells, e.g., astrocytes, oligodendrocytes, microglia cells that produce dopamine, and cells such as fibroblasts that are transfected with growth factors, into a patient to treat a PD using techniques described herein for obtaining, culturing, and introducing cells into a patient. The cells may be introduced with or without the proteins, factors, and supplementing materials described herein. Autologous cells, allogenic cells, or xenogenic cells may be used. Cells include stem cells, various differentiated cells, and their precursors. The site of introduction may be at or near the defect or at a site distant from the defect, as described herein. The various techniques for cell culture and introduction of cells may be applied to any defect described herein, as appropriate for the particular defect.

Spinal Cord Injury (SCI)

Spinal cord injury involves damage to the nerves within the spinal canal. Most SCIs are caused by trauma to the vertebral column, thereby affecting the spinal cord's ability to send and receive messages from the brain to the body's systems that control sensory, motor and autonomic function below the level of injury.

The spinal cord and the brain together make up the central nervous system (CNS). The spinal cord coordinates the body's movement and sensation. In transverse section the spinal cord is divided into symmetrical halves by a dorsal (posterior) median septum and a ventral (anterior) median sulcus. The spinal cord consists of an inner core that includes neurons and long nerve fibers called axons, forming the grey matter. Axons in the spinal cord carry signals downward from the brain (along descending pathways) and upward toward the brain (along ascending pathways). Many axons in these pathways are covered by sheaths of an insulating substance called myelin, which gives them a whitish appearance; therefore, the region in which they lie is called “white matter”. In the center of the spinal grey matter, the central canal extends the whole length of the spinal cord. Rostrally it opens into the 4^{th} ventricle and caudally into the conus medullaris. It is lined by a columnar, ciliated epithelium (ependyma) and filled with CSF.

In transverse section the grey matter has a “butterfly shape” or resembles the letter “H”. It consists of four cellular masses, the dorsal and the ventral horns (or columns). The grey matter which immediately surrounds the central canal and unites the two sites is termed the dorsal and ventral grey commisure. The tip of the dorsal horn is separated from the surface of the cord by a thin dorsolateral tract (tract of Lissauer) formed by primary afferents that ascend and descend before terminating in the subjacent grey matter. The dorsal horn is a major receptive zone (zone of termination) of primary afferent fibers, which enter via the dorsal roots of spinal nerves. These afferents carry exteroceptive, propioceptive, and interoceptive information. The ventral horns contain efferent neurons whose axons leave the spinal cord in ventral nerve roots. In general, the spinal grey matter is a complex mixture of neuronal cell bodies (somata), their processes (neurites) and synaptic connections, neuroglia and blood vessels. The neurons are multipolar, vary in size, and in other particular features such as the length of the axon and the arrangement of their dendrites. They are mainly Golgi type I or Golgi type II neurons. Axons and dendrites of Golgi I neurons pass out of the grey matter into ventral spinal roots or spinal tracts. Axons and dendrites of Golgi II neurons are confined to the nearby grey matter. The lateral horn is a small lateral projection of the grey matter located between the dorsal and the ventral horns present from the 8^{th} cervical or 1^{st} thoracic to the 2^{nd} or 3^{rd} lumbar segment. The lateral horn contains the cell bodies of pre-ganglionic sympathetic neurons that are the source of sacral outflow of parasympathetic pre-ganglionic nerve fibers.

At any particular spinal level (as seen in transverse section) the spinal grey matter is considered to consist of ten layers, Rexed's laminae, which are defined on the basis of neuronal size, shape, cytological features and density. The laminae are numbered sequentially in a dorsoventral sequence. Laminae I-IV correspond to the head of the dorsal horn, and are the main receiving areas for cutaneous primary reception. Lamina I (lamina marginalis, at the very tip of the horn) has a reticular appearance and contains small, medium and large neuronal somata. Lamina II occupies most of the head of the dorsal horn and contains densely packed small Golgi type II neurons that characteristically lack myelin and form the substantia gelatinosa. Lamina III consists of Golgi type II somata which are mostly larger and less dense that lamina II, containing also some substantia gelatinosa. Lamina IV is a thick, loosely packed heterogeneous zone with somata varying in shape and shape from small and round, through medium and triangular, to very large and stellate. Lamina V and VI receive most of the terminals of propioceptive primary afferents from skin, muscle and viscera and profuse corticospinal projections from the motor and sensory cortex and subcortical level, that suggest large involvement in the regulation of movement. Lamina V is thick and corresponds to the neck of the dorsal horn, it has a mixed population of small and medium-sized somata entangled in multiple bundles of fibers. Lamina VI is located in the base of the dorsal horn and contains both small and densely packed somata as well as large and loosely packed triangular and stellate ones. Lamina VII includes much of the intermediate (lateral) horn and contains neurons of Clarke's column (large neurons and interneurons). This lamina has extensive ascending and descending connections with the midbrain and cerebellum (via numerous spinal tracts) and is thus involved in regulation of posture and movement as well as autonomic functions. Lamina VIII is a mass of propiospinal interneurons. The axons from these interneurons influence motor neurons bilaterally, directly and/or by excitation of small neurons supplying γ efferent fibers to muscle spindles. Lamina IX is a complex array of cells consisting of α and γ motor neurons and many intemeurons. The large a motor neurons supply motor-end-plates of extrafusal muscle fibres in striated muscle. The smaller γ motor neurons give rise to small-diameter efferent axons which innervate intrafusal muscle fibers in muscle spindles. Lamina X surrounds the central canal and consists of the dorsal and ventral grey commisures.

The ventral horn has neurons that vary in size from very large a motor neurons whose axons emerge in ventral roots to innervate striated skeletal muscles, to intermediate size and small neurons, from which most are γ motor neurons and many interneurons. All these motor neurons utilize acetylcholine as their neurotransmitter. At longitudinal inspection, ventral horn neurons are arranged in elongated groups that form a number of separated columns of essentially a medial, a central and a lateral cell column with some subdivision. They may or may not extend throughout the cord. In general, the medial cell column innervates the axial musculature, and lateral columns innervates the limbs, while the central group innervates the diaphragm and other thoracic and abdominal muscles.

The spinal white matter surrounds the central core of grey matter. It contains nerve fibres, neuroglia and blood vessels. Most of the nerve fibres run longitudinally and are arranged in three large masses, the dorsal, lateral and ventral funiculi, on either side of the cord. Fibers of related functions and those with common origins or destinations are grouped to form ascending, descending or propiospinal tracts within the funiculi. Ascending tracts contain primary afferent fibres, which enter by dorsal roots, and fibers derived from intrinsic spinal neurons. Descending tracts contain long fibers, which descend from various supraspinal sources to synapse with spinal neurons. Propiospinal tracts, both ascending and descending, contain the axons of neurons which are localized entirely to the spinal cord.

The dorsal funiculus on each side of the cord consists of two large ascending tracts, the fasciculus gracilis and fasciculus cuneatus, also known as dorsal columns. The dorsal columns carry propioception (position sense) and exteroceptive (touch-pressure) information. Other main ascending tracts include 1) The two spinocerebellar tracts that carry proprioreceptive and cutaneous information to the cerebellum for the coordination of movement. 2) The spinothalamic tract consisting of second-order neurons which convey pain, temperature, coarse (non-discriminative) touch and pressure information to the somatosensory region of the thalamus.

Descending pathways to the spinal cord originate primarily in the cerebral cortex and in numerous sites within the brain stem. They are concerned with the control of movement, muscle tone and posture, the modulation of spinal reflex mechanisms, and the transmission of afferent information to higher levels. The descending corticospinal tract fibers arise mainly from cells situated in the upper two-thirds of the pre-central motor cortex and mainly from giant pyramidal neurons or Betz cells. They project to neurons that are mostly located in the contralateral side of the spinal cord.

Like the brain, the spinal cord is enclosed in three membranes (meninges) consisting of the pia matter, the innermost layer, the arachnoid, a delicate middle layer, and the dura matter, which is a tougher outer layer. The spinal cord is organized into segments along its length. Nerves from each segment connect to specific regions of the body. The segments in the neck, or cervical region, referred to as C1 through C8, control signals to the neck, arms, and hands. Those in the thoracic or upper back region (T1 through T12) relay signals to the torso and some parts of the arms. Those in the lumbar or mid-back region just below the ribs (L1 through L5) control signals to the hips and legs. The sacral segments (S1 through S5) lie just below the lumbar segments in the mid-back and control signals to the groin, toes, and some parts of the legs. The effects of spinal cord injury at different segments along the spine reflect this organization.

Several types of cells carry out spinal cord functions. Large motor neurons have long axons that control skeletal muscles in the neck, torso, and limbs. Sensory neurons called dorsal root ganglion cells, whose axons form the nerves that carry information from the body into the spinal cord, are found immediately outside the spinal cord. Spinal interneurons, which lie completely within the spinal cord, help integrate sensory information and generate coordinated signals that control muscles. Glia, or supporting cells, far outnumber neurons in the brain and spinal cord and perform many essential functions. One type of glial cell, the oligodendrocyte, creates the myelin sheaths that insulate axons and improve the speed and reliability of nerve signal transmission. Other glia enclose the spinal cord like the rim and spokes of a wheel, providing compartments for the ascending and descending nerve fiber tracts. Astrocytes, large star-shaped glial cells, regulate the composition of the fluids that surround nerve cells. Some of these cells also form scar tissue after injury. Smaller cells called microglia also become activated in response to injury and help clean up waste products. All of these glial cells produce substances that support neuron survival and influence axon growth. However, these cells, if overstimulating, may also impede recovery following injury.

Nerve cells of the brain and spinal cord respond to trauma and damage differently than most other cells of the body, including those in the PNS. After injury, nerve cells, or neurons, of the peripheral nervous system (PNS), which carry signals to the limbs, torso, and other parts of the body, are able to repair themselves. Injured nerves in the CNS, however, are not able to regenerate. The brain and spinal cord are confined within bony cavities that protect them, but this also renders them vulnerable to compression damage caused by swelling or forceful injury. Cells of the CNS have a very high rate of metabolism and rely upon blood glucose for energy—these cells require a full blood supply for healthy functioning. CNS cells are particularly vulnerable to reductions in blood flow (ischemia). Other unique features of the CNS are the “blood-brain-barrier” and the “blood-spinal-cord barrier.” These barriers, formed by cells lining blood vessels in the CNS, protect nerve cells by restricting entry of potentially harmful substances and cells of the immune system. Trauma may compromise these barriers also prevent entry of some potential therapeutic drugs. Also, in the brain and spinal cord, the glia and the extracellular matrix differ from those in peripheral nerves. Each of these differences between the PNS and CNS contributes to their different responses to injury.

The site and the level of damage to the spinal cord determines the particular clinical syndrome (e.g. whether the lesion involves the upper or lower cervical, thoracic or lumbosacral spinal cord). The specific symptoms and signs of the lesion are determined by destruction of segmental tissue (transversal damage) and disconnection of supra and infrasegmental ascending or descending tracts (longitudinal damage). Damage can be also classified as complete and incomplete. Patients with an incomplete injury have some spared sensory or motor function below the level of injury—the spinal cord was not totally damaged or disrupted. In a complete injury, nerve damage obstructs every signal coming from the brain to the body parts below the injury. For example, a complete upper cervical lesion causes spastic tetraplegia with hyperreflexia, extensor plantar responses (secondary to upper motor neuron lesion). It damages the segmental sensory and motor contributions to the nerve roots and brachial plexus causing sensory loss, weakness and wasting of muscles. Disruption of the ascending sensory pathways in the lateral and dorsal columns of the cervical spinal cord leads to complete loss of sensation to pain and temperature (lateral spinothalamic tracts) and touch and propioception (dorsal fasciculi). Damage to the descending corticospinal tracts in the lateral columns of the spinal cord produces the spastic paralysis. Descending pathways to the bladder are interrupted, and this produces incontinence.

Currently, there is no cure for spinal cord injuries. Injury progression, prevention, drug treatments, decompression surgery, and complex drug therapies are all being examined as a means to overcome the effects of spinal cord injury.

The respective injured neurons (e.g. interneurons, motor neurons) near the site of the lesion can be expanded or their progenitor cells can be expanded and implanted at or near or into the specific spinal cord tract. Ancillary cells of the support tissue (e.g., ECM) containing trophic factors and nutrients, the glial cells in particular (e.g., astrocytes), can be implanted in tandem or separately from the neural implantation. Oligodendrocytes can be used to remyelinate the injured axons. Glial cells can be implanted near the axon defect to promote connections between the brain and the sensory and motor neurons below the spinal cord lesion. Astrocytes, in particular those isolated from the lateral ventricle of the brain, can be used as multipotent stem cells that differentiate into appropriate cell types to restore the neuronal and axon fiber functions. Progenitor cells can be expanded and implanted at or near or into the specific spinal cord tract. Multineurotropin-expressing glial-restricted precursor cells can be implanted to promote functional recovery after traumatic spinal cord injury. Mesenchymal stem cells (MSCs) isolated in culture from the mononuclear layer of bone marrow may promote axonal regeneration inside the spinal lesion. Neural precursor cells can be delivered into the injured spinal cord by intrathecal injection at the lumbar cord. Any of these cells, or precursors thereof, may be accompanied by helpful proteins or other useful factors as described herein, e.g., to enhance cellular “take”.

Huntington Disease

Huntington Disease (HD) is an autosomal dominant mutation of the HD gene located on the short arm of the chromosome 4 (4p) which encodes for a protein called huntingtin. The characteristic dysfunction is cell death of cholinergic and GABAnergic neurons within the caudate nucleus which is part of the striatum. In addition, there is a a relative increase in dopaminergic neuron activity due to the mechanisms listed in the above text. This results clinically in choreric (dancelike) movements, severe mood disturbances and progressive dementia. The mechanism for neuronal cell death may involve a hyperactive glutamate receptor (NMDA receptor), resulting in glutamate toxicity. Glutamate toxicity is the result of excessive influx of calcium into the neuron.

Implantation of cholinergic and/or GABAnergic neurons or their progenitor cells into the caudate nucleus can be used to correct this disease. Such cells, or precursors thereof, may be accompanied by helpful proteins or other useful factors as described herein, e.g., to enhance cellular “take”.

Multiple Sclerosis

Multiple sclerosis is a type of autoimmune disease in which the myelin surrounding the nerves of the central nervous system (CNS) is destroyed. This destruction results clinically in paralysis, loss of sensation, and loss of coordination. The exact nature of the defect depends on the specific area of the CNS involved. Oligodendrocytes produce myelin in the CNS. The injection of autologous oligodendrocytes proximal to the nerve damage can be used for repair the myelin damage.

Progenitor Cells

One source of a progenitor cell for specific neurons and other cell types in the brain and nervous system is the use of astrocytes from the lining of lateral ventricle in the brain. These cells can migrate to the olfactory bulb. These cells can form mature brain cells, the astrocytes, the microglia and the oligodendrocytes, and the neurons. Implantation in vivo into the desired location can differentiate these cells into the proper cell type. Alternately, co-culture or ECM from the specific tissue region of interest can be used to differentiate these cells in vitro prior to implantation. Accordingly, these methods may be used to obtain these cells for treatments indicated herein.

Liver Disease Leading to Liver Failure-Liver Defects

The liver plays a central role in the maintenance of metabolic equilibrium. The biochemical functions in which the liver plays a major role include the intermediate metabolism of proteins, glycoproteins and carbohydrates. The absorption of blood glucose is stored as glycogen. Proteins are synthesized and degraded into ammonia and excreted. The liver regulates lipid and cholesterol metabolism, including the production of bilirubin and bile salts from cholesterol and the delivery to the gut, facilitating fat and fat-soluble vitamin absorption. Bile pigments are formed as breakdown products of worn-out red blood cells. Lipid soluble drugs, steroid hormones and alcohol are metabolized and degraded. The liver stores iron, vitamin B12 and folic acid, metabolizes porphyrin and produces clotting factors (e.g. I, II, V, VII, IX, X), amongst other functions.

The liver has a unique dual blood supply in which the portal venous system supplies 75% of its circulation and the hepatic artery the remaining 25%.

Structure and Histology

The liver is a essentially an epithelial-mesenchymal outgrowth of the caudal part of the foregut. It is a fairly homogeneous sponge-like structure organized in units called liver lobules consisting of three components. These are the central vein to which all the venous blood from branches of the portal veins drain; the peripheral portal triad (or portal tracts) set at the angles of the polygons and showing a branch of the portal venous system, a hepatic artery and a branch of the hepatic biliary system (draining bile from the liver); and hepatocytes (parenchymal liver cells) radiating from the central vein as rows of cells separated by vascular sinusoids. About 80% of the liver volume and 60% of its cell number are formed by hepatocytes. They are polyhedral in shape with 5-12 sides and are from 20 to 30 μm across.

The columns of hepatocytes and blood sinusoids are the link between the portal triads and the central veins. The flow of blood is directed from the peripheral margin of the lobule to the central vein (centripetal flow). The bile is secreted into minute canals traveling between the hepatocytes, it flows in the opposite direction toward the portal triads (centrifugal flow).

The hepatocytes form sheets or trabeculae that are usually only one cell thick. At least one of its surfaces faces a blood sinusoid which morphologically is a large capillary. The surface of the hepatocyte that faces the sinusoid exhibits numerous microvilli creating a large area of membrane (70% of the hepatocyte surface- exposed to blood plasma). The nuclei of the hepatocyte is round ad often tetraploid, polyploid or multiple. The hepatocyte exhibits a variety and abundance of cytoplasmic components (mitochondria, endoplasmic reticulum, Golgi apparatus, peroxisomes and all types of lysosomes, among others) reflecting its active metabolism. In histology, the hepatocyte is usually employed as a model of the “typical cell”.

Hepatic venous sinusoids are wider that blood capillaries and lined by a thin fenestrated endothelium lacking a basal lamina. The endothelial cells are flattened with a central nucleus and numerous typical transcytotic vesicles in the cytoplasm. The endothelial discontinuities or fenestrations facilitate delivery and export of substances between the hepatocyte and the blood supply. The narrow space between the cell surface and the sinusoid is called the space of Disse which contains hepatocyte microvilli, type III collagen fibers, and hepatic stellate cells (also called lipocytes or Ito cells) that store vitamin A and produces the collagen fibers and other ECM. The hepatic stellate cells are much less numerous than the hepatocytes and along with fibroblasts, are present in the liver parenchyma. Stellate cells are thought to be mesenchymal in origin and are characterized by multiple cytoplasmic lipid droplets. In response to liver damage, these cells become activated and predominantly myofibroblast-like. They are responsible for the replacement of toxically damaged hepatocytes with collagenous scar tissue—hepatic fibrosis—that can progress to liver cirrhosis.

Macrophages known as Kupffer cells, are long term liver residents derived from circular monocytes. They are located on the inner walls of the vascular sinusoids. They function by phagocytosis to destroy micro-organisms and damaged red blood cells. The Kupffer cells originate in the bone marrow, and form a major part of the mononuclear phagocyte system responsible for removing cellular and microbial debris from the circulation and secreting cytokines involved in defense.

The bile ducts start as bile cannaliculi formed between apposed hepatocyte surface membranes. They are tiny intercellular spaces and form small conduits around the hepatocytes. Through linkages they drain toward the portal triads and ultimately converge, leaving the liver in the system of ducts that carries bile to the gall bladder.

Parenchymal liver disease (disease of the hepatocyte itself) can be classified as acute or chronic hepatitis (e.g, viral, drug-induced, toxic); as cirrhosis (e.g., alcoholic, post-necrotic, biliary, hemochromatosis, other rare types); as infiltration (e.g., glycogen, fat, amyloid, granuloma, lymphoma, leukemia); as storage (e.g., inborn errors of metabolism, iron metabolism, copper homeostasis); as space occupying lesions (e.g., hepatoma, metastatic tumor, abcess, cysts); and as functional disorders associated with jaundice (e.g., Gilbert's, Crigler-Najjar, Dubin- Jhonson-Rotor syndromes, cholestatis of pregnancy).

Clinical Manifestations and Diagnosis

Understanding liver disease and its clinical manifestations can be derived from the knowledge of the fundamental hepatic structure and function outlined above.

There are features of hepatocyte cell biology that contributes to the expression of liver disease. One feature is the absolute tropism of the hepatocyte for infectious agents. This is the case for the hepatitis viruses, which account for a large proportion of both acute and chronic liver disease. Another feature is the potential for proliferation and regeneration, such as the complete recovery which usually occurs following fulminant hepatitis. However, an architectural disordered regeneration in concert with fibrosis is an essential factor in the development of cirrhosis, another predominant hepatic disease. The cardinal pathologic features of cirrhosis reflect irreversible chronic injury of the hepatic parenchyma and include extensive fibrosis in association with the formation of regenerative nodules.

Some of the most important diagnostic possibilities and assessments of the severity of the illness involves if the problem is primary hepatocellular or cholestatic, if the illness onset is abrupt or gradual, if the problem has lead to clinically significant impairment of the function of the liver or portal hypertension (due to fibrosis, scar tissue compressing the vessels, and sclerosis of the portal veins (as occurs with cirrhosis). Severe pain in the right upper quadrant of the abdomen associated with digestive ailments suggest biliary inflammation or obstruction, whereas vague discomfort and hepatomegaly along with anorexia, weight loss, jaundice or pruitus suggest hepatocellular or infiltrative disease. Complaints of easy bruising suggest coagulation problems. Mental confusion should be regarded as ominous signs of either fulminant acute or advance chronic liver disease.

Numerous imaging tests can be conducted to diagnose liver disease. These range from plain abdominal radiographs, ultrasound, computed tomography, magnetic resonance imaging to sophisticated radioisotope scanning. Multiple blood tests reflecting the diversity of the normal liver function are usually necessary to diagnose hepatic disease. Several serum enzyme assays (transaminases, alkaline phosphatase, glutamyltranspeptidases, lactic dehydrogenase, etc.) need to be run to assess liver function. Extensive liver injury may lead to decreased blood levels of albumin, prothrombin and fibrinogen as well as alteration of clotting factors. Elevated blood ammonia levels are reflective of extensive hepatocellular necrosis. A liver biopsy is often required when there is difficulty defining the etiology of the disease in order to better classify it morphologically.

The management of several chronic hepatic diseases that has lead to liver fibrosis and functional liver failure is limited to the medical treatment of the complications, avoidance of drugs, avoidance of excessive protein intake that may induce further inflammation leading to a hepatic coma, and prompt treatment of any kind of infection. In patients with asymptomatic cirrhosis, expectant management alone can be appropriate. In those patients in which post-necrotic cirrhosis has developed as a result of a treatable condition, therapy directed at the primary disorder may limit further progression of the disease.

Orthotopic liver transplantation (replacement of a diseased liver by a healthy organ recovered from a brain-dead individual) is a treatment approach for selected patients whose liver disease is progressive, life-threatening, and beyond the reach of traditional therapy. Liver transplant is a very costly and sophisticate surgical procedure and it is not indicated for a vast number of patients with severe hepatic disease but with other life-threatening systemic diseases, infections, pre-existing cardiovascular or pulmonary disease and metastatic malignancies.

Expanded hepatocytes implanted into the liver parenchyma can be used to repair liver damage that result in liver defects and/or systemic defects. Fibrosis or cirrhosis of the liver can be corrected by removing liver tissue scars with hepatic stellate cells, fibroblasts or myofibroblasts. If needed, further correction of the damage can proceed by resynthesis of the liver parenchyma with hepatic stellate cells or fibroblasts and hepatocytes. Gene altered hepatocytes and fibroblasts, due to the liver's central location to bloodstream (e.g., bloodrich) and its active metabolism, can be used to provide systemic proteins such as coagulation factors.

Pancreatic Insufficiency Leading to Digestive Problems and Diabetes Mellitus

The pancreas has three anatomical components comprising the head, the body and the tail. It is situated transversely across the posterior wall of the abdomen, underneath the peritoneum and closely surrounded by important anatomical structures (e.g., vascular, nervous and organs) accounting for a very difficult surgical access. The pancreas is one of the largest glands in the body with a compound tubulo-alveolar or compound acinar glands having two types of secretory functions performed by two types of glandular tissue: a) endocrine counting for the release into the bloodstream of the two most important pancreatic hormones insulin and glucagons and b) exocrine counting for the release into the digestive system (e.g., duodenum) of over 20 digestive enzymes (pancreatic juice) in which almost a liter is released daily.

The main tissue mass of the pancrease is exocrine in which are embedded islets of endocrine cells. The exocrine pancreas is a branched acinar gland that is surrounded and incompletely lobulated by loose connective tissue. The acinar cells are pyramidal, secretoy cells arranged in spherical clusters (i.e., acini). A narrow intralobular duct originates within each secretory acinus and is lined with flattened or cuboidal centro-acinar cells to form a ductile. The ductules from banches which link between adjacent acini. More distally the branching ductules form the larger interloblular ducts comprised of taller cuboidal and eventually columnar epithelium that have neuroendocrine cells present. These larger ducts are surrounded by loose septal connective tissue that contains stellate cells, fibroblasts, myofibroblasts, smooth muscle, numerous mast cells and autonomic nerve fibers. Fibroblasts and stellate cells produce the majority of the ECM and protease activity in the connective tissue.

The endocrine pancreas consists of the islets of Langerhans comprising 1-2% of the volume of the organ (i.e., body and tail) and contain at least four major cell groups. The human pancreas contains more than a million islets, mostly located in the tail. The islets control glucose homeostasis and are embedded in the exocrine tissue and each is close in proximity to autonomic innervation and fenestrated capillaries. The islet is a mass of polyhedral cells that compose spherical or ellipsoid clusters. The β cells (⅔ of each islet cell population) secrete insulin, the a cells secrete glucagons, the δ cells secrete somatostatin and gastrin, and PP or F cells secrete the pancreatic polypeptide hormone. The autonomic transmitter acetylcholine augments insulin and glucagons release, while noradrenalin inhibit glucose-induced insulin release. Self-duplication of differentiated β cells is the major route of islet cell replacement. The pancreatic juice contains trypsinogens, proteases, elastase, lipase, numerous serine proteases, water and electrolytes. The juice is important for digestion of lipids, proteins and carbohydrates. It is produced by acinar cells with an enormous amount of rough endoplasmic reticulum in their cytoplasm.

The most common pancreatic disease leading to organ failure is inflammatory disease in the form of acute, relapsing or chronic pancreatitis. Acute pancreatitis can be caused by infections (e.g., mumps, viral infections), alchohol ingestion, biliary tract disease (e.g., gallstones), trauma, metabolic, post-operative or post-endoscopic, drug associated or induced, hereditary, connective tissue disease or be idiopathic. Chronic pancreatitis can be caused by alcoholism, Cystic Fibrosis, malnutrition, pancreatic neoplasia, pancreatic resection, gastric surgery with stomach resection and anastomosis, gastrinoma (Zollinger-Ellison syndrome), hereditary, trauma, metabolic or be idiopathic. Pancreatitis can be accompanied by tissue fibrosis.

The relative inaccessibility of the pancreas to direct examination and the nonspecificity of the abdominal pain associated with pancreatitis make the diagnosis of the disease difficult. Greater than 90% of the exocrine pancreas must be damaged before maldigestion of fat and protein is manifested. Other symptoms of pancreatic insufficiency are hyperlipidemia, vitamin B12 malabsorption, hypercalcemia, hypocalcemia, hyperglycemia, ascites, and chronic abdominal pain. Diagnosis of the disease can be made with imaging tests such as ultrasound, simple abdominal X-rays, CT Scan, radionuclide scanning (PIPIDA, HIDA) and MRI. Basic abnormal biochemical tests are serum amylase, bilirubins, alkaline phosphatase and aspartate aminotransferase (AST) measurements.

In most patients (i.e., 85-90%) with acute pancreatitis the disease is self-limited and subsides spontaneously with medical therapy aimed to “put the pancreas at rest”. In the other group of patients either severe medical complications arise from the attack, a pancreatic abscess, phlegmon or pseudocyst appears requiring surgical intervention, or a chronic pancreatitis with exocrine insufficiency of the organ occurs.

Therapy for patients with chronic pancreatitis is directed to manage the three major problems of abdominal pain, malabsorption and maldigestion along with the dietary management of an impaired glucose tolerance. Alcohol, large meals and a high fat diet must be avoided. The pain may call for surgical procedures. Vitamins and mineral supplementation along with potent enzyme preparation with every meal should be administered.

Pancreatic fibrosis occurs from pancreatitis. Additionally, in diabetes type I, fibrosis occurs. Diabetes Mellitus (DM) type 1 is one of the most common endocrine diseases. It is characterized by blood sugar metabolic abnormalities with long-term complications involving the eyes, kidneys, heart and blood vessels. DM is the consequence of the almost certain autoimmune destruction of most of the β cells of the pancreas leading to the production of insufficient amounts of insulin. Clinically DM displays persistently elevated blood sugar levels. Islet cells are the direct target of an autoimmune attack.

Diabetes Mellitus (DM) type II occurs in greatest incidence in people over the age of 60 years and is induced by weight gain. Cells no longer respond to insulin. In the more common form, the islet cells can be eventually lost.

Diabetes results in a shortened lifespan and negatively affects the major organs of the cardiovascular system, the kidneys, liver and eyes, amongst others resulting in diseases such as atherosclerosis, blindness, cataract formation, tissue fibrosis, and hypertension, to name a few.

One aspect of the invention is to improve pancreatic function due to fibrosis that occurs during pancreatitis and diabetes mellitus. Pancreatic stellate cells or fibroblasts can be implanted into the fibrotic areas to remove the tissue scars. In another aspect of the invention, epithelial cells can be used to repair the ductule or tubular duct system. β cells isolated from the islets or ductile system of the pancreas can be expanded in vitro and implanted into islets or embedded into the exocrine region of the pancreas. β cells can also be implanted into the liver parenchyma or other suitable organs that is blood rich and metabolically active. The preferable implantation into the liver is by perfusion of the cells through the portal vein delivered through a catheter. Alternately, HSC stem cells from the bone marrow, peripheral blood or the spleen can be expanded and implanted into the islets or pancreas. These cells can result in new islet functions that can be gained by neovascularization and growth factor release to increase endogenous β cell proliferation of stem cell differentiation to β cells. Islet stem cells can be implanted or infused, in which the cells can home into the pancreas and become differentiated into functional β cells. In a preferred embodiment, spleenic stem cells are the choice of stem cell for β cell formation in the pancreas. EPCS, endothelial cells or other cells and/or proteins that induce neovascularization can be implanted into the pancreas to increase β cell formation.

The Endocrine System

Histology and Function

The endocrine system is composed of distinct glands or tissues that secrete hormones into the circulatory system to stimulate actions e.g., metabolic activity) in designated target tissues or organs. A hormone is defmed as a biologically active substance released into and transported in blood or lymph. In responding to the hormonal stimulus, the target cells/tissues may secrete one or more substances into the circulation which in turn, may regulate the synthesis and or secretion of hormones by the endocrine gland. This system is termed feedback control. In other cases hormones may act directly on target tissues without producing a feedback response.

The principal endocrine glands are the hypothalamus, pituitary (anterior and posterior), pineal, pancreas, adrenals, thyroid and parathyroid tissues. Several organs such as the stomach, intestine, the lungs, the thymus or kidneys have specialized cell types that secrete hormones that may act locally or remotely.

There are four main types of hormones: peptides and protein hormones, steroid hormones, tyrosine or amine-derived hormones and fatty acid derivatives. Peptide hormones are synthesized like other proteins, stored in cytoplasmic granules and exocytosed when secretion is required. Peptide and amine hormones are water soluble, circulating freely for a very limited amount of time and then degraded. Steroids are synthesized in mitochondria and the endoplasmic reticulum and released by diffusion. Thyroid hormones are stored extracellularly in the thyroid gland, then enter the thyroid cells releasing active thyroid hormones into the blood. Steroid and thyroid hormones are lipid soluble and carried by plasma bound proteins in the blood for longer plasma half-lives. Many of the other hormone types are also carried in the blood by transport proteins.

Hormones act on target cells by initiating biologic responses via specific receptors. Receptors for peptides and protein hormones are generally located in cell plasma membranes while receptors for steroid and thyroid hormones are found intracellularly and act on the cell nucleus. When bound to membrane receptors the hormones activate second messengers molecules and/or signalling pathways, which in turn, initiate reactions in the cytoplasm or nucleus. Through nuclear receptors the hormone alters gene transcription and translation.

Feedback regulation, neural control and factors maintaining cyclic, rhythmic or pulsatile patterns of hormone secretion determine how and when hormones are released. Feedback control is usually negative, inhibiting further hormone secretion. Positive feedback loops increase the secretion of the primary endocrine cells. Neural input (i.e. stress) can inhibit or stimulate hormone secretion. Cyclic or pulsatile hormone secretion is modified by circadian rhythms. Sensory pathways connect the central nervous system and some endocrine glands. This and other CNS inputs are mostly regulated by the hypothalamus which in turn regulates the pituitary through neural and vascular connections in a complex neuro-endocrine circuit.

Hypothalamus and Pituitary

The hypothalamus, 4 cm³, consists of groups of neurosecretory neurons which synthesize hormones (mostly peptides) that are transported to the pituitary gland. These hormones are blood-borne releasing hormones acting on the anterior pituitary. Other peptides reach the posterior pituitary by transport down connecting axons. It contains the integrative systems that control fluid and electrolyte balance, food ingestion, energy balance, metabolism, thermoregulation, immune system, reproduction, emotional responses, homeostasis, aging, amongst other physiological actions.

The hypothalamus structure contains areas anteroposteriorly of chiasamatic (supraoptic), tuberal (infundibulo-tuberal) and posterior (mammillary) and mediolaterally of periventricular, intermediate (medial) and lateral regions. The neurons that produce growth hormone-releasing hormone (GHRH) are located mainly in the arcuate nucleus region and while some are in the periventricular nucleus or periventricular peroptic area. GHRH acts on anterior pituitary to release growth hormone, luteinizing hormone and follicle-stimulating hormone in pulses. Neurons located in the periventricular nucleus produce somatostatin (growth hormone release-inhibiting hormone). Somatostatin inhibits thyroid-stimulating hormone and GHRH. Both GHRH and somatostatin are secreted in intermittent reciprocal pulses of 3 to 5 hours. Corticotrophin-releasing hormone (CRH) neurons are located mainly in the parvocellular paraventricular region. These neurons stimulate corticotrophs to release ACTH. Thyrotrophin-releasing hormone (TRH) neurons are distributed in the periventricular, ventromedial and dorsomedial nuclei. TRH stimulates pituitary release of TSH and excites cold-sensitive and inhibits warm-sensitive neurons in the preoptic area. TRH release is influenced by core temperature, is monitored by the anterior hypothalamus and is controlled by the negative feedback of thyroid hormones. Dopamine neurons are located in the arcuate nucleus (A12 group) and have terminals in the infundibulum and median eminence. Dopamine is the main prolactin release inhibiting hormone. Dopamine also inhibits TSH secretion. TSH acts also as a prolactin-releasing hormone.

Five types of cells in the anterior pituitary secrete six main types of hormones. The cell types are described according to the target tissue stimulated by the hormones they secrete. These cell types are epithelial of varying size and shape arranged in cords or irregular follicles between which are thin-walled vascular sinusoids in a foundation of reticular connective tissue. The cells are: 1) Somatotrophs, that secrete Growth Hormone (GH), targeting bone, viscera and soft tissues, promoting tissue growth and metabolism. These cells are acidophils (staining with acidic dyes). 2) Thyrotrophs, that secrete Thyroid Stimulating Hormone (TSH), targeting the thyroid and promoting secretion of thyroid hormones. These cells are basophils. 3) Corticotrophs, that secrete Adrenocorticotrophin (ACTH), targeting the adrenals and promoting secretion of cortisol and other corticosteroids. These cells are basophils. 4) Lactotrophs, that secrete Prolactin (PRL), targeting mammary glands and others tissues and promoting secretion of milk and growth of breast tissue. These cells are acidophils. 5) Gonadotrophs, that secrete Follicle-Stimulating Hormone (FSH) and Luteinizing hormone (LH), targeting the gonads and promoting the production of gametes and sex steroids. These cells are basophils (staining with basic dyes). LH and FSH are influenced by GABA and monoamines, estrogen and progesterone action through other neurons, corticotrophin-releasing factor and endogenous opioids.

Proopiomelanocortin precursor is cleaved into ACTH. β-lipotropin (has lipolytic function) and β-endorphin are some other cleavage products released from the pituitary.

The posterior pituitary consists of nerve fibers from the hypothalamus, their terminals being in close association with capillaries. Posterior pituitary hormones (peptides), synthesized in the hypothalamus and then bound to carrier proteins, are stored in granules in the axon terminals until discharged by exocytosis.

There are two posterior pituitary hormones originating from the hypothalamus: 1) Vasopressin (anti-diuretic hormone ADH), targets the kidneys and vascular smooth muscle, controls the blood pressure and volume and the osmotic pressure by means of promoting re-absorption of water and vasoconstriction. 2) Oxytocin, targets the mammary glands and uterus, controls the suckling stimulus and stretch receptors in milk ejection and parturition.

Suprachiasmatic Nucleus (SCN)

This tissues contains only a few thousand neurons that control day-night cycles in motor activity, plasma concentration of hormones, body temperature, sleeping, waking, renal secretion, physiological and circadian rhythms, amongst other functions. SCN contains many neurotransmitters such as vasopressin, VIP, neuropeptide Y and neurotensin.

Thyroid

Microscopically the two lobes of the thyroid gland are divided into two lobules, containing several dozen follicles each. These follicles are full of colloid and are lined by a single epithelial layer of flattened, cuboidal, or low columnar cells. The thyroid synthesizes and secretes tri-iodothyronine (T3) and tetra-iodothyronine (thyroxine, T4) as components of the colloid which contains almost all thryoglobulin. The follicle concentrates iodine from the blood, made available through the diet, to iodinate the thyroglobulin to T3 and T4. The thyroid secretes greater amounts of T4 than T3, but most of the T4 is converted into T3 in peripheral tissues. The production and secretion of T3 and T4 is stimulated by thyroid-stimulating hormone (TSH) from the anterior pituitary, which in turn is regulated by hypothalamic TRH (thyrotropin releasing hormone). Thyroid hormones suppress TSH secretion by negative feedback. In the interfollicular stroma of the thyroid gland, there are small groups of calcitonin-secreting cells. Calcitonin counteracts the effects of parathyroid hormone, inhibiting bone resorption.

Parathyroid gland

The small parathyroid glands are located on the posterior surface of the thyroid and are normally found in a group of four. The glands secrete parathyroid hormone (PTH), a peptide that controls calcium and phosphate concentrations in the blood. The PTH is synthesized by chief cells, small cuboidal cells with pale cytoplasm, and later in life oxyphil cells appear, no longer producing PTH. The net effect of PTH on bone and renal metabolism is to maintain calcium and phosphate homeostasis. PTH also stimulates the enzyme 1 α-hydroxylase resulting in the formation of the active form of vitamin D. PTH secretion is controlled by plasma calcium concentration acting in a negative feedback mechanism.

Adrenals

Each adrenal gland, located atop the kidney, is composed of two endocrine components, the cortex and the medulla. The cortex is arranged into three zones: 1) The thin zona glomerulosa (cells appear in clumps), that secretes the mineralocorticosteroid aldosterone, which acts in the kidney to regulate electrolyte and fluid balance by promoting sodium reabsorption. 2) The Zona Fasiculata (cells appear in columns) occupy close to 70% of the volume of the cortex. The cells are large with lipid inclusions reflecting the steroidogenic activity, primarily glucocorticoid production in which cortisol is the dominant hormone. Cortisol is essential for life, affecting carbohydrate, protein and fat metabolism, has anti-inflammatory properties and modifies the body's reaction to stress. 3) The Zona Reticularis, the inner and deepest layer (cells appear in an irregular network), is characterized by small eosinophilic cells that secrete DHEA (dehydroepiandrosterone) and androstenedione, which are converted in other tissues into androgens and estrogens.

The adrenal medulla contains cells of neuroectoderm origin designated as chromaffm cells. These cells are neurons with no axons that secrete and store catecholamines (mainly epinephrine and norepinephrine). Chromaffin cells can be used for implantation in other neuron deficiencies, such as in Parkinson's disease.

Endocrine Pancreas is described earlier in this document. The pancreas is one of the largest glands in the body with a compound tubulo-alveolar or compound acinar glands with two types of secretory functions: a) endocrine release into the blood stream of the two most important pancreatic hormones, insulin and glucagons and b) exocrine. The islets of Langerhans comprising 1-2% of the volume of the organ (body and tail) have at least four major cell groups. The β cells (⅔ of each cell population) secrete insulin, the a cells secrete glucagons, the δ cells secrete somatostatin and PP cells secrete the pancreatic polypeptide hormone.

The Pineal Gland

This gland is a very small organ (6 by 4 mm) located in the roof of the diencephalons. The gland contains modified photoreceptors, cords and pinealocytes arranged into clusters that are associated with astrocyte-like neuroglia. These neuroglia are the main cellular part of the pineal stalk. Pinealocytes are highly modified neurons that produce melatonin (synthesized from tryptophan). Pinealocytes contain multiple synaptic ribbons randomly distributed between adjacent cells and coupled by gap junctions. Circulating levels of melatonin show a circadian rhythm as do the enzymes that make it (e.g., serotonin N-acetyltransferase) in which the activities rise during darkness and fall during the day. The cyclical behavior of the pineal gland is controlled by the circadian oscillator in the suprachiasmatic nucleus. The pineal gland modifies the activities (largely inhibitory) of other endocrine glands such as the pancreas, parathyroids, adrenal cortex and medulla, gonads, adenohypophysis, and neurohypophysis. The hormones made are polypeptides or indoleamines (e.g., melatonin). These hormones can inhibit pars anterior synthesis and the release of hormones and hypothalalmic production of releasing factors. Pineal secretions reach target cells via the blood or cerebrospinal fluid.

Dispersed Neuroendocrine System Several organs contain single cells or small groups of neuroendocrine cells secreting hormones. As a group they are called the APUD cells because of their ability to decarboxylate amine precursors into amines. The gastrointestinal tract contains 16 or more neuroendocrine cell types producing more than 30 hormones. The lungs contain neuroendocrine cells known as the epithelial bodies. The skin contains Merkel cells. The kidneys contain juxtaglomerular cells that release renin. Renin is a participant in the renin-angiotensin system (RAS) that regulates the glomerular filtration rate (GFR) and ultimately controls the body fluid homeostasis in response to falls in the blood pressure. The kidneys synthesize 1,25-dihydroxyvitamin D, the active form of Vitamin D as well as erythropoietin (EPO) in the peritubular endothelial cells. The placenta produces chorionic gonadotropin (hCG), placental lactogen (hPL), among other hormones to sustain the human pregnancy.

Disorders and Clinical Conditions.

In the anterior pituitary undersecretion of growth hormone (GH) in children results in short stature or dwarfism, excess fat and reduced muscle strength. The latter symptoms may occur in aging adults with declining growth hormone secretion. Reduced ACTH secretion lowers cortisol production, resulting in hypoglycemia. Undersecretion of gonadotropin (GnRH deficiency) may lead to declining fertility and reproductive function. In the posterior pituitary, reduction or absence of the production of ADH (diabetes insipidus) is characterized by the inability to concentrate urine and conserve water.

In the thyroid secondary hypothyroidism is a condition in which the body lacks sufficient thyroid hormone due to thyroid gland disease. Autoimmune thyroiditis (i.e., inflammation of the thyroid gland) leaves a large percentage of the cells of the thyroid damaged (or dead) and incapable of producing sufficient hormone. The most common cause of thyroid gland failure is called (Hashimoto's thyroiditis), a form of thyroid inflammation caused by the patient's own immune system. The surgical removal of a portion or all of the thyroid gland, such as treatment for cancer, leads to the development of hypothyroidism

In the parathyroid hypoparathyroidism (i.e., depressed plasma calcium levels) or the low secretion of parathyroid hormone is uncommon and occurs usually because of a previous surgical procedure.

Addison's Disease (chronic adrenal insufficiency, or hypocortisolism caused by autoimmune destruction of the adrenal cortex) is characterized by adrenal glands that do not produce enough of the hormone cortisol and in some cases, the hormone aldosterone. Diabetes Mellitus Type I is due tothe autoimmune destruction of the β cells leading to hypoinsulinemia.

The primary dysregulation of the endocrine system occurs as a consequence of aging. The sleep-wake cycle is disturbed in the elderly. This is controlled by the SCN and the pineal gland. Thus implantation of the appropriate cell types either separately or together in the glands can correct the sleep dysregulation in the elderly. Circadian and physiological rhythms are controlled by the SCN. Thus implanted cells to populate the SCN can maintain or re-install a normal physiological homeostasis of the subject.

Different cell types in various endocrine organs and tissues produce diverse hormones, e.g., as described in PCT Application ______ filed Sep. 14, 2006 entitled “Compositions And Methods for the Augmentation and Repair of Defects in Tissue”. Such hormones may be incorporated into cellular compositions for implantation into a patient. Cells that produce the above hormones can be expanded and implanted in vivo to effect production of the needed hormones or inhibitor of hormones and their activities that are reduced as a function of aging or disease. The embodiment of this invention describes a form of treatment for functional endocrine disorders in which there is a reduced production of hormones or inhibitor of hormones and their activities by a particular organ with the injection or direct placement of the particular lineage of autologous cells.

Cells producing the hormone of interest or precursor cells to that particular cell type can be used. Cell types producing different hormones can be used singly or in combination. In general cell types are implanted back into their natural in situ location. However, other tissues may be used (e.g., skin) as an alternate implantation site as long as the desired hormone cell phenotype is maintained and the cells are controllable by normal feedback mechanisms. Some cell types may require the endocrine gland or part of the gland to be regenerated to a more functional or youthful state. This can be accomplished by implanting the appropriate cell types back into the stroma of the tissue. For example, connective tissues cells such as fibroblasts and other cell types that normally inhabit the tissue can be used. Similarly, epithelial cells can be placed into its original location that generally line the stromal tissue and overlie the basement membrane. Implantation of cell types for specific hormones can be used in conjunction with connective tissue and epithelium correction of the gland.

During aging endocrine profiles change. To counteract or improve the profile the hormone producing cell types can be expanded and implanted in vivo.

The Immune System and Defects

The immune system is comprised of lymphocytes that are the body's main defense force against infection and cancer. It heals physical damage (wounds), but can also give rise to auto-immunity and inflammation. An immune response is against all material that is recognized as foreign or “non-self”. The immune system exhibits tolerance to self tissues and does not attack the organism it protects except in the case of auto-immune disease. The immune system operates throughout the body, however it is compartmentalized in certain organs and tissues where the cells of the immune system are organized into specific structures. These are classified as central or primary lymphoid tissue (bone marrow, thymus) and peripheral or secondary lymphoid tissue (lymph nodes, spleen, mucosa-associated lymphoid tissue). The lymphoid structures are functionally unified via blood and lymph vascular systems allowing trafficking, positioning and recirculation of immune cells. Immune cells traverse all tissues such as macrophage surveillance in connective tissue environments.

Central or primary lymphoid tissues comprise bone marrow or the thymus. As the major hematopoietic organ in the human the bone marrow is primarly found in spongy bone. It is a highly cellular tissue that produces all blood cell types (except mature T cells). It contains numerous arterial, venous, and sinusoidal blood vessels, and a reticular stroma. The thymus is divided into two lobes, the cortex and the medulla, and multiple lobules. Both lobes “educate” multipotent T cell precursors that arrive from the bone marrow into mature competent T cells. The thymus removes T cells that recognize and would attack the host.

Peripheral or secondary lymphoid tissues include the spleen, which is formed by reticular and lymphatic tissue and is the largest lymph organ. The cellular material, consisting mainly of lymphocytes and macrophages, is called splenic pulp, and it lies between trabeculae. One of the main functions of the spleen is to bring blood into contact with lymphocytes. As blood flows slowly through the spleen any disease organisms within it are likely to come into contact with lymphocytes in the spleen tissue. This contact activates the lymphocytes, which can then attack the foreign invaders. As blood flows through the spleen, macrophages remove worn-out red (i.e. senescent) and white blood cells and platelets. Also included are lymph nodes, in which the lymph that is drained from the body passes through these structures. Lymph nodes are specialized dilations of lymphatic tissue which are supported within by a meshwork of connective tissue called reticulin fibers and are populated by dense aggregates of B and T lymphocytes and macrophages. Lymph nodes occur along the entire length of the lymphatic system and tend to increase in size as they become closer to the thoracic duct. They are also organized in chains or clusters which drain exclusively a particular organ or region of the body. Lymph nodes are found in larger clusters in the axillary, inguinal and cervical regions of the body. Lymph nodes supply lymphocytes to the blood. Mucosa-associated lymphoid tissue (MALT) consists of a population of immune cells (lymphocytes, plasma cells and macrophages) in the mucosa of many epithelial tissues and is organized into discrete lymphoid follicles (such as the tonsils or Peyer's patches in the ileum). MALT is specialized for sampling and collection of antigens across mucosal epithelia.

The Immune Response.

Two basic functionally distinct immune reaction types are: 1) The innate response. This is the initial and immediately available response that is largely made up of cells with phagocytic functions and includes physical barriers and soluble factors as well. 2) The adaptive response. This slower but highly specific and effective response is made up of specialized lymphocytes producing antibodies

Innate immunity is phylogenetically old, fast to respond and non-specific. Therefore it does not lead to immunologic memory. Cells of the innate system recognize patterns characteristic of all foreign agents instead of antigens specific to a particular agent.

### EXAMPLES OF INNATE DEFENSES ARE

The body physical and chemical barriers (skin, mucus layers of stomach, etc) and body fluids (saliva, tears, stomach fluids)
Intracellular killing of microbes carried out by macrophages and neutrophils (i.e., short-lived products of the myeloid lineage of the bone marrow, PMNs). These are the two major families of immune cells in innate defenses. Macrophages are derived from circulating monocytes, which become distributed in tissues such as macrophages in the dermis, Kupffer cells in the lungs and liver, osteoclasts in bone, mesangial cells in the kidney, or microglial cells in the brain. Macrophages also traverse tissues surviving only for a few days. The bone marrow produces macrophages in vast numbers, which accounts for their large proportion (60%) among circulating white blood cells (leukocytes). Ingestion following binding of receptors on the immune cells induces cytokine and chemokine secretion causing chemoattraction of blood leukocytes and inflammation. Dendritic cells, NK cells and complement assists the neutrophils and macrophages.
Extracellular killing provides additional protection, served by natural killer (NK) cells and eosinophils. NK cells are derived from hematopoietic stem cells and circulate in the blood. NK cells bind to foreign antigens on infected cells or foreign cells. NK cells kill these cells by release of cytotoxic granules that cause apoptosis. NK cells kill tumor cells and virus-infected cells. NK cells can act without preactivation or immunization and can be activated by interferon or macrophage-derived cytokines.
The antigen present cells (APCs) are dendritic cells primarily, although macrophages and B cells are amongst other cells that can be APCs. DCs are long-lived phagocytes that migrate from bone marrow to peripheral tissues and when present in the lymph nodes display antigens to naïve T lymphocytes.
Complement (plasma proteins produced by the liver that form a triggered enzyme system) are activated locally after the innate immune system recognizes foreign organisms. Complement promotes inflammation.

The first reaction of the innate immune system is conducted by neutrophils that produce superoxide anions to kill the pathogens they have ingested. IL-2, IFN-□, certain growth factors (i.e. GM-CSF), and bacterial products (LPS) prevent apoptosis of neutrophils. As part of inflammation neutrophils are guided to the sites of infection by binding to cell adhesion molecules produced by endothelial cells that line the blood vessels of the tissues.

The macrophages phagocytose foreign organisms, infected cells, kill tumor cells and activate other macrophages to release cytokines and chemokines such as IL-1, IL-6, IL-8, IL-10, IL-12, IFN-□, TNF□, prostaglandin E₂ and other products such as reactive oxygen and nitrogen molecules. The cytokines stimulate the activation and interaction of yet other immune cells to initiate the adaptive response as well as turning off the immune pathways when the pathogen is removed.

The innate immune cells and other cells at the site of infection secrete cytokines and factors that further activates the immune system and proinflammation resulting in increased blood delivery to the infected tissue that enhances the defense. If the innate response does not eliminate the infection then the adaptive immune system is activated.

The innate and adaptive pathways are linked. The innate pathway initiates the adaptive pathway by APC action. APCs, in particular DCs, initiate the adaptive pathway upon presentation of antigens of the foreign body to T cells. Macrophages use their toll-like receptor (TLRS) membrane proteins to bind antigens. Antigen binding causes cytokine release and chemoattraction of other immune cells, including B and T cells. Macrophages phagocytize protein, DNA, membranes and deliver the degraded macromolecules to B and T cells which initiates the adaptive immune response. APCs such as the DCs also secrete cytokines IL-12 that enhance NK, B and T cell-mediated immunity. Stromal cells, especially fibroblasts, play a key role in the transition from innate to adaptive immunity. Thus, infusion in the bloodstream or implantation to an infected or diseased organ with stromal cells such as those obtained from a healthy tissue can be used to boost the immune response to infection (e.g., sepsis) and disease.

The adaptive immunity, which is phylogenetically new, is slow-reacting but highly flexible, specific and able to respond to an almost infinite range of different organisms and antigens. This is due to a sophisticated membrane receptor-antigen recognition system that ultimately leads to immune memory. The key cells for this system are the lymphocytes (T and B cells), originating from the bone marrow in the adult or (from the liver in the fetus), and account for 20-30% of the circulating leukocytes. T lymphocytes mature in the thymus, having previously entered this organ, via the blood, as non-functional precursors from the bone marrow. B lymphocytes are made in the bone marrow. The surface receptor on B cells is an immunoglobulin (Ig), or antibody, occurring as a secretory product of antigen-activated B cells. The receptor (TCR) on T cells for antigen occurs only on the surface membrane. B cells produce antibodies that circulate in the blood and lymph and attach to foreign antigens that mark them for destruction by other immune cells. The receptors on these cells interact with antigen on the surface of infected or abnormal host cells. Binding of antigen on the TCR allows the clonal selection and expansion of T cells. Each clone of T cells have different arranged TCRs. Ancillary co-receptor molecules stabilize the APC interaction and co-stimulatory molecules on the T cells enhance T cell activation. The memory T cells produced respond with greater intensity and faster kinetics upon re-exposure to the same antigen and is a basis for vaccinations. To optimize T cell activation in vivo an APC is required that quickly synthesizes, processes and presents antigen at the same time. This timing is due to spatial and temporal factors for the supply of peptides to the MHC molecules. The half-life of the peptide and MHC is critical (˜4 hr for class I and up to 1 day for class II). APCs such as dendritic cells (DCs) react with T cells in lymph nodes within one day and the DCs' peptide display at the cell surface in conjunction with co-stimulatory molecules activates T cells. Activated B cells as well as resting B cells can activate CD4 and CD8 T cells, depending on sufficient co-stimulation by B7 and CD40 surface proteins. Secondary lymphoid organs, in which antigen is present in sufficient amounts and length of time, are important for the activation to take place. These structural and spatial factors in secondary lymphoid organs containing co-stimulatory signals determine the timing of clonal expansion and kinetics of the immune response.

Humoral immunity is part of the adaptive immune response. B cells constitute antibody-mediated or humoral immunity. This is because the antibodies circulate in blood and lymph. Antibodies recognize foreign antigens and mark them for destruction. These antibodies are basic templates with a special region that is highly specific to target a given antigen. The antibody's frame remains constant, but through chemical and cellular messages, the immune system selects the special variable region to combat the particular invader. Infections (bacterial, viral, etc.) prompt humoral immunity.

Cell-mediated immunity is the other part of the adaptive immune response. T lymphocytes are responsible for cell-mediated immunity (or cellular immunity). Certain T cells, which also patrol the blood and lymph for foreign invaders, can do more than mark the antigens. These T cells attack and destroy diseased cells that they recognize as foreign. T cells orchestrate, regulate and coordinate the overall immune response. T cells can be classified into suppressor, helper, and cytotoxic subtypes.

T cells depend on unique cell surface molecules, the major histocompatibility complex (MHC), to help them recognize antigen fragments. Helper T cells, for example, also known as CD4 positive T cells (CD4+ T cells), activate B cells to start making antibodies. Cytotoxic T cells, by binding to antigen and releasing cytokines (i.e. IL-2), chemoattract and increase the proliferation of immune cells. Helper T cells also can activate other T cells, macrophages and influence which type of antibody is produced. Certain T cells, called CD8 positive T cells (CD8+ T cells), can become killer cells that attack and destroy infected cells, host cells that display on their surface antigens of the infective agent. The killer T cells are also called cytotoxic T cells or CTLs (cytotoxic lymphocytes). T cells are activated or differentiated into effector T cells when precursor resting T cells recognize antigen on specific antigen-presenting cells. Thus antigen stimulates growth and proliferation of the T cells and B cells that are specific to the antigen. These cells can change into effector cells, the activated T and B cells or change into memory cells which remain dormant but ready to act upon re-exposure to the antigen. Naïve T cells and memory cells produce cytokines to activate and increase proliferation of T and other immune cells. IL-2 is a predominant cytokine produced.

Dendritic cells are the main antigen presenting cells (APCs) that stimulate T cells, although macrophages and B cells can also serve as an APCs. Antigenic peptides of 8-9 amino acids, the degradation products of cytosolic proteins, bind MHC class I molecules and induce cytotoxic T lymphocyte (CTL). Antigenic peptides of 13-17 amino acids, the degradation of internalized exogenous antigens, bind MHC class II molecules that induce CD4+ T helper cells. Co-stimulatory molecules, for example, are CD28 or CD45RA surface proteins on memory T cells, that help stimulate the cells to divide in the presence of antigen.

T cell development starts in the bone marrow of adults where stem cells differentiate into lymphatic cells. A proportion of the T cell precursors migrate to the thymus medulla, where under thymic hormone exposure the pre-T cells begin to express membrane antigens. In the medulla the pre-T cells come into contact with foreign and endogenous antigens, which is the basis for the cells to distinguish between self and nonself. It is in the epithelial cells of the cortical stroma of the thymus where most of the T cell maturation occurs. Maturation involves expression of different versions of the antigen recognition molecule, the T cell receptor (TCR). The endothelial cells express MHC (major histocompatibility complex) class I and II molecules and maturation occurs when in contact with the surface receptor of the developing T cells. Lymphocytes are released as mature naïve T cells. Maintenance of the thymus gland (e.g., the medullary region) can be obtained by introduction of thymic lymphocytes.

All thymic epithelium is derived from a single stem cell type and later co-expresses molecules that distinguish between the mature cortical and medullary epithelial subpopulations. The major change in the thymus with age is quantitative, thus the major lymphoid and microenvironmental cell populations are present through out the lifespan but the thymic volume and thus thymic cell numbers decrease with age. Thymus involution corresponds to many of the specific immune functions decline. Thymus atrophy begins early in life. Involution and diminishment of thymic epithelial cell function occurs in which fat cells replace the thymocytes and T cell output declines. By the end of the sixth decade of life, a functional decline of the immune system is due primarily to quantitative changes of the thymus-dependent part of the immune system that brings about increase in infections, autoimmune diseases and cancer initiation and promotion. By augmenting the thymus with thymocytes, immune functions can be restored that include augmentation of recruited lymphocytes, T-cell differentiation (i.e. receptor rearrangement), induction of activation markers and cytokine production. Thymic fibroblasts can be implanted to promote thymus rejuvenation and T-cell development.

T cells can also develop by a thymus independent pathway in the lymph nodes. The process can by enhanced in the presence of oncostatin M.

B cells originate from precursor cells in bone marrow assisted by nonlymphoid stromal cells. The connective tissue stromal cells adhere to the precursors and secrete growth factors to enhance their proliferation and differentiation. B cells remain immature and migrate to peripheral lymphoid organs. Maturation occurs then by the rearrangement and expression of immunoglobulin genes that result in many different types of antigen receptors on the B cell surface. B cell activation occurs upon binding of foreign antigens expressed on activated T cell surfaces to the antigen receptors on the B cell surface. CD40 expression on the T cell surface is required for activation and differentiation of B cells. B cell activation differentiates B cells into antibody-secreting cells. Secreted antibodies then permeate tissue extracellular space and matrix to control infection from invading cells. In the invention stromal cells (e.g., fibroblasts) can be added to bone marrow to maintain effective production of B cells during aging and disease.

Dysregulation of the immune system causes autoimmune disease, allergy, inflammation and affects negatively tissue integrity and lifespan. Both innate and adaptive pathways are affected in failing immune systems due to age, chronic infection or cancer. The elderly's health is typified by chronic infection, infections hard to get rid of, inflammation, malignancies, abnormal organ function, medication, unhealthy lifestyle, tissue aging, all of which can be effect poorer immune responses. Dysregulation is predominant in the elderly.

In aging the immune response to foreign antigens decreases while an increased prevalence of autoantibodies occurs. Elderly are more susceptible to bacterial, viral, protozoan and neoplasias than the young. Additionally, chronic inflammatory responses appear, which can be related to tissue damage, Alzheimer's disease and atherosclerosis, amongst others. In old age only small numbers of new T cells are produced in the thymus. Growth hormone and insulin can stimulate the elderly thymus to produce more T cells. Also, in old age a decrease in bone marrow stem cells result in less naïve T cells and thus more memory T cells exist. Implantation of expanded bone marrow stem cells can be used to increase T cell production in the aged and diseased.

Much less T cells are produced, differentiated and activated in the elderly. The most dramatic difference in the elderly versus the young is the low T cell numbers present. T cells are less responsive to mitogens and antigens. T cell cytotoxicity is less. A shift occurs from mainly naïve T cell populatons in the young to mostly memory T cells in the elderly. Furthermore, the memory cells carry a single clone of TCR with age so that relatively small number of different clones of T cells are available. This can result from a lifetime exposure to antigens and the production of much fewer naïve T cells by the thymus or the peripheral microenvironment of aged systems may cause the transfer of naïve to memory T cells.

The higher ratio of naïve to memory cells can dictate longer lifespans of organisms. With less naïve T cells less IL-2 is produced, a cytokine which promotes proliferation and activation of T cells and other immune cells. T cells can be less active in forming germinal centers in lymph nodes and less active in inducing B cells to rearrange their antibody genes. In aging fewer CD8+T cells overproduce whereas in young immune systems, thousands of unique CD8+T cells recognize different antigens. Thus in the young, more different CD8+T cells attack a pathogen. There are many T cell clones to many different antigens in the young whereas in the old T cell clones may be limited to a small amount and predominate thus an antigen represented by a T cell clone may not quench the infection in the elderly. Thus increasing number of T cells, in particular naïve T cells, can compensate for these decreased T cell activities.

Clonal replicative senescence of T cells can compromise immue response and it is important to put back in naïve T cells to allow higher numbers of clonal T cells. In contrast to T cells, B cells show little decrease in number or antibody numbers with age. B cells show a decrease in activation, proliferation and antibody production. The avidity of the antibody may decrease. In the invention, T cell (e.g., naïve T cells) introduction can compensate for any loss of B cell activity with age.

Antigen presentation, IL-12 production by immune cells, and T cell stimulatory molecules produced by DCs are reduced. IL-12 spurs T cell proliferation and secretion of IFN□. Thus more immune cells (T cells) are to be used in the invention to compensate for the reduction of immune cell produced factors (cytokines, etc).

In aging the innate immune cells can change. NK cells lose some killing capacity but an increase in numbers can compensate.

Macrophages can lose some of their TLRs and produce less cytokines. Thus more macrophages can assist the immune response including the adaptive immune response through the release of more cytokines. In the invention macrophages are implanted or infused to increase the number of macrophages to increase both innate and adaptive immune response. Thus, more T cells can increase these functions.

Innate immune cells produce IL-2, IFN-δ, certain growth factors (i.e. GM-CSF), and bacterial products (LPS) that prevent apoptosis of neutrophils, but in the elderly, the apoptosis occurs more readily. Thus this may prevent neutrophils from accumulating in tissues and can be why the elderly are more susceptible to infections. Thus, more neutrophils can be added to combat the infections.

Vaccines depend on foreign antigens multiplying specific T and B cell clones with long-lives. Vaccination results in the production of specific antibodies to antigens, but in the elderly this is largely impaired. Vaccinations are compromised or not effective in the aged. Infections leading to a high mortality rate are influenza, pneumococcal pneumonia, bacteremia, cholecystitis and tetanus. Adjuvants are helpful to increase the immune response. In the invention naïve T cells can be used to increase the immune response to vaccinations.

In aging there is an increase in IL-10, IL-6 and TNF□ secretion by immune cells. Also there is increased risk with poor T cell proliferative responses, low B cell numbers, CD8+, CD28−, CD57+ cells are increased, CD4+ to CD8+ ratio is less than 1. T cells don't make cytokines as well as the young. CD28 is the best marker yet for aging cytokine production and these cells are not as prevalent in the old. TNFα regulates CD28 expression. CD28 levels are critical for T cell activation. CD28 is a co-stimulatory molecule. CD28− CD4+ cells make high amount of IL-2 and IFNα after stimulation with immobilized anti-CD3. Other co-stimulatory T cell molecules are CD 134 and 154.

Thus a number of diseases can be addressed by introduction of appropriate immune cells to the subject.

In the aged there is a loss of new bone formation due to immune cell changes. For example, in post-menopausal females the loss of estrogen increases IL-1 production by monocytes and macrophages. IL-1 then increases production of IL-6 by osteoblasts which induces bone resportion can cause osteoporosis. Cells that decrease IL-1 or IL-6 production or a balanced T cell system can prevent bone resorption by this mechanism. Estrogen alone or in conjunction with cells can be used.

Cells that control other detrimental cytokines such as IL-6, IL-10, TNFα can be used to counter the effects of an aging immune system.

Innate immune components can contribute to atherosclerosis. Macrophages in particular can produce pro-inflammatory cytokines (due to interaction with proteins produced by vascular cells as a consequence of oxidized cholesterol accumulation and injury). Also, activated T cells are among the first cells found in the arterial intima sites that are disposed to become atherosclerotic.

Chronic inflammation damages tissue, promotes aging and related diseases such as Alzheimer's disease (AD) and atherosclerosis and is common in the elderly as the adaptive immune response wanes. For example in AD, β-amyloid aggregates occurring in brain parenchyma and its vasculature, cause complement and microglia to become involved triggering inflammation from prostaglandins, acute phase reactants and proinflammatory cytokines. In atherosclerosis the antibodies to oxidized lipoproteins can promote inflammation that damages the vessel tissue. This embodiment of the invention may use immune cells implanted in the brain parenchyma and associated vasculature to degrade amyloid plaque and neurofibrillary tangles. Macrophages and microglial cells are the preferred cells.

Tumor cells display foreign antigens on their surface and thus spur on immune reactions involving T cells, NK cells and macrophages. These immune cells can be expanded in vitro to combat tumors.

Autoimmunity has both humoral and cellular components. Rheumatoid arthritis is another example of an autoimmune disease. Autoimmunity can be provoked by abnormal modifications of macromolecules such as oxidation or glycosylation (AGEs) which the macromoleucles are recognized as nonself. CD5+B cells produce most autoantibodies and CD8+T cells can inhibit these B cells from proliferating. Thus more T cells can decrease autoimmunity and disease associated with autoimmunity. Suppression of cell-mediated immunity and DC maturation can be controlled by T cells, monocytes, and macrophages that secrete IL-10. IL-10 is elevated in the elderly. Use of these immune cells can control autoimmune reactions. Decreasing inflammation is an important goal with the immune system. This can be primarily accomplished with the addition of T cells. In tandem or separate stromal cells implanted into specific tissues or infused into the bloodstream can decrease inflammation.

Healthy elderly are free of tissue autoantibodies, cancer, dementia, diabetes, cataracts, and cardiac disease. Their T cells have full proliferative capability only showing a delay in time to reach highest T cell proliferation. In a preferred embodiment T cells are used to correct dysregulation of the immune system, in particular for the aged. In the invention it is important to maintain good numbers of naïve T cells for healthy lifespan and to combat some of the disorders of autoimmunity, cancer, dementia, diabetes, cataracts, and cardiac disease, amongst other dysfunctions.

Since the ability of T cells to proliferate in the elderly diminishes it is important to put high number of T cells grown in vitro. Furthermore, young serum instead of older serum in vitro can be used to more effectively increase the proliferation and activation of T cells in vitro.

To increase the power of the adaptive pathway, a preferred approach is to culture T cells. Non-selected populations of T cells or monoclonal T cells can be expanded in culture before large numbers are introduced to the subject. Specific or monoclonal populations of T cells can be selected by affinity binding of specific antigens of interest and then expansion in vitro. Alternately non-selected populations can be selected in vitro with presentation of the antigens of interest. Antigens can be presented in acellular or cellular form. The preferred embodiment is the form that stimulates proliferation of the desired clone of T cells. Thus ancillary cells, antigen and cytokines such as IL-2 and IL-4 can be used to select and expand T cells. Cellular forms can be B cells or other cells presenting the antigen or antibody to the antigen to the T cells to stimulate selective T cell proliferation.

To enhance the adaptive pathway, naïve (not encountered antigen) T cells can be matured and increased in numbers by improvement or regeneration of the critical areas of the thymus gland. In vitro cultured thymocytes can be grown and implanted into areas of the thymus including the cortical and medullary regions. Endothelial cells, EPCs, or pericytes can be cultured and reimplanted into the thymus gland to enhance angiogenesis in the tissue.

APC cells can be cultured in vitro and presented in vivo. These cells can be dendritic cells or macrophages containing the antigen of interest. In the invention the addition of APCs that are activated in vitro are preferred, although addition of APCs alone in high numbers in vivo can be used.

In an alternate enablement, immune cells are genetically altered so as to increase proliferation capacity and avidity to pathogens and altered cells.

Immune cells can be obtained from their endogenous locations described above in addition to peripheral locations such as the blood or lymph. For example, T cells can be obtained from the donor from peripheral or from T cell progenitors in the bone marrow or spleen.

Antibodies can be used to select certain subsets of T cells. For example, antibodies to specific surface receptors on T cells can be used to discern and isolate CD4 from CD8 and other subtypes of these T cells into naïve and memory cells.

Clonal B cells can be grown in vitro by co-culture with T cells. Other co-culture of immune cells can be used.

T cells and B cells proliferate with IL-2. NK cells respond to IL-12. These cytokines can be used to enhance the in vitro proliferation of the immune cells.

Local infections can be treated by implantation or infusion of immune cells into the infected area. Alternately, systemic infusions (i.e. intravenous) can be used. For pervasive infections or systemic infections (e.g., sepsis), systemic infisions are preferred. A similar strategy can be employed to repair or regenerate tissues of the body with inunune cells.

Expansion of immune cells can also be done by the presence of younger serum in culture. Alternately, the appropriate quantity and quality of specific growth factors, hormones can be used. Clonal senescence can be addressed in this manner. T cells, for example, senesce in culture as do other somatic cells (e.g., connective tissue cells). In vitro T cell replicative senescence can be delayed or eliminated when medium contains IL-2 and IL-4 without antigen and accessory cells. Thus, isolated polyclonal or monoclonal T cells can be grown in long-term culture by intermittent reactivation via the antigen receptor and exogenous interleukins. Alternately, revival of clonal expansion through the introduction of telomere addition (via telomerase activation, e.g., hTERT) can be performed to obtain appropriate numbers of specific lymphocytes to combat the antigen.

Infections

Chronic infections can be treated with immune cell placement into the infected area. Amongst many functions, fibroblasts from a healthy tissue can be implanted into an infected tissue to enhance the infection fighting ability of the immune cells. Fibroblasts can build a healthy architecture for the tissue to assist in quenching the infectious state. Fibroblasts can be used to fight off and quench infections. Fibroblasts can also be used against systemic infections, such as sepsis.

Chronic Inflammation

Chronic inflammation damages tissue, promotes aging and related diseases such as Alzheimer's disease (AD) and atherosclerosis. Disease, injury, cancer, invasion of pathogens or foreign antigens can result in inflammatory processes, primarily due to the immune response and released cytokines and chemokines. Inflammation results in increased blood flow, lymphocyte entry, chemoattraction of immune cells and other cell types such as those involved in tissue repair, and self-containment of the infection. Inflammation can cause swelling, heat and pain. Decreasing inflammation occurs in which immune cells and other cell types that were recruited and expanded, are removed.

Chronic inflammation is a dysregulated inflammatory process. Local fibroblasts in the inflamed area do not turn off chemoattractants and other inflammatory signals. This failure leads to retention and inappropriate survival of immune cells. Stromal fibroblasts can produce survival signals during inflammation and at the end of the inflammation response the cells can turn off survival signals that lead to apoptosis and subsequent phagocytosis of unneeded effector cells. Although immune cells such as macrophages, dendritic cells and lymphocytes interact with each other and other immune cells, fibroblast activation plays a key role in the modulation and interaction with immune cells. Fibroblasts modify the local cellular, ECM and cytokine microenvironment that controls the nature and kinetics of the inflammatory infiltrate reflective of the damage. Chronic inflammation can result when an acute inflammation resolving transition to an acquired immune response is derailed into a chronic persistent tissue damaging inflammation by dysregulation of stromal fibroblasts at the site of the damage. Appropriate fibroblast action such as providing proper ECM, cytokine and chemokine environments can prevent chronic inflammation. Fibroblasts can control cytokine production through NF-kB pathway regulation.

A typical transition from innate immunity to an acquired immune response initially involves the acute inflammation response in which antigens or dead cells, for example, activate tissue macrophages and fibroblasts to produce cytokines and chemokines that recruit more immune inflammatory cells. Immature dendritic cells also become activated and migrate with antigen to lymph nodes where the acquired immune response is predominantly made. Tissue repair and immune memory follows under normal circumstances. In chronic inflammation fibroblasts continue to secrete chemokines and cytokines, such as pro-survival factors (i.e. IFN-β) and pro-retentive factors (i.e. SDF-1), that increase the accumulation of immune cells within the tissue, appearing as lymphoid aggregates and preventing tissue repair.

In a preferred embodiment chronic inflammation can be treated with stromal fibroblasts obtained from non-inflammed tissue. The treatment can be done by infusion of fibroblasts into the bloodstream. Alternately, if the chronic inflammation is localized, such as to a tissue, stromal fibroblasts can be implanted at or near the tissue.

Additionally, such as in rheumatoid arthritis, non-rheumatoid fibroblasts can be used to quench the inflammatory reactions of the joint. Other autoimmune diseases can be countered in a similar fashion.

Tissue Fibrosis

Fibrosis can be described as the dysregulation of normal tissue repair and maintenance process, resulting in tissue scarring. Fibrosis often results in hardening of the tissues. Tissue fibrosis is the fmal common pathogenic pathway for most forms of chronic tissue injury. The cause can be due to inflammation, infection, aging, sclerosis, vascular dysfunction, metabolic dysfunction, autoimmune disease, lymphedema (fibrosis due to swelling of non-draining lymph nodes), chemotherapy, radiation therapy, host vs. graft reaction, burns, wounds, hypertension, diabetic conditions, prolonged swelling or edema, environmental insults, genetic disease, amongst others. Fibrosis ends in organ compromise and failure in which there has been progressive replacement of the normal tissue environment with fibrotic lesions. Fibrosis results in distortion of the tissue architecture or microenvironment resulting in tissue dysfunction. Fibrosis can be caused by excess cell production, such as fibroblasts in connective tissue, excess release of growth factors, cytokines and chemokines such as TGFβ, excess production and deposition of excess ECM including collagen and transdifferentiation of fibroblasts to myofibroblasts.

Tissue fibrosis contains excess collagen deposition in the tissues. Much of the tissue location of fibrosis is classified as interstitital, or between cells. Tissue fibrosis can occur in most tissues. Major organs include the skin, heart, lung, kidney, liver, and bone marrow. Other tissues include, muscle, lens, pancreas, bone, blood vessels, nerve fibers, tendon, ligaments, esophagus, GI tract, intestine, bowels, esophagus, reproductive structures, endocrine organs such as the thyroid, pituitary gland, and hypothalamus, tubule structure such as ureters and urethras, amongst other tissue or organ types. Fibrosis mainly affects tissues locally, but can be systemic, such as in systemic scleroderma.

Fibrosis occurs in many sclerotic conditions including systemic sclerosis, mixed connective tissue diseases, bone sclerosis, multiple sclerosis, vasculitis, amongst others.

In systemic sclerosis, diffuse fibrosis is present in the skin, articular tissues, and internal organs such as the heart, kidney, lung, GI tract and the esophagus. In vasculitis any layer of the vessel wall can become fibrotic (primarily due to inflammation) at the affected sites, along with intimal hypertrophy and destruction of the elastic lamina. The main vessel affected is the artery, although arterioles, vein, venules and capillaries can be involved.

Fibrosis notable contains excess collagen fibers and ECM. Sclerosis can describe the excess non-fibrillar deposition of ECM, sometimes of a hyaline nature. Fibrosis that is described herein includes both the fibrosis and sclerosis molecular characteristics and the invention applies to both.

Scar tissue containing new fibroblasts and excess collagen are often in proximity to epithelial cells. Thus there is an accumulation of fibroblasts in epithelial organs such as in kidney fibrosis. The epithelial to mesenchymal transition or transdifferentiation of epithelial cells into a specific set of fibroblasts can take place in fibrosis. Other processes can induce fibrosis. Coagulation factors such as thrombin and factor Xa are profibrotic due to PAR-1 proteolytic activation and the subseqeunt release of PDGF sand CTGF ECM growth factors. AGEs can induce tubular epithelial to myofibroblast transition through the RAGE-ERK1/2MAP kinase signaling pathway.

The interstitial fibroblasts are the main effector cells in organ fibrosis such as the kidneys, lungs and liver. These fibroblasts come from the tissue itself, from the epithelial to fibroblast conversion and some can come from bone marrow. The fibroblast can represent a subset of fibroblasts in the tissue representing heterogeneity in the fibrogenic phenotype of fibroblasts in fibrotic tissue. Fibroblasts to myofibroblast conversion can be induced by TG\tilde{F}β. Specific myofibroblast phenotypes can produce fibrosis in contrast to TGFβ independent nonfibrogenic myofibroblast phenotypes.Ang II, a 8 aa peptide, is profibrogenic by upregulating TGFμ. Other cell types that can contribute to fibrosis include immune cells such as macrophages, monocytes, eosinophils and T cells, bone marrow progenitor cells, platelets and inflammatory cells that release growth-modulating mediators (e.g., spurred on by endothelial cell damage), hepatic stellate cells in liver and stellate cells in the pancreas.

HGF can prevent fibrosis and acts by suppressing expression of TGFβ, increasing collagenase activity, stimulating hepatocyte proliferation, suppressing hepatocyte apoptosis and modulating myofibroblasts (which in liver is a main cell type responsible for fibrotic change). Fibroblasts and hepatocytes make HGF.

In the skin tissue, scarring is usually due to a wound healing response, and can be hypotrophic or hypertrophic including keloid formation. Additionally dermal and subcutaneous fibrosis, lipodermatosclerosis, the progressive hardening of the skin and subcutaneous layers occurs due to other causes, such as venous disease or autoimmune disease (e.g., scleroderma).

Liver cirrhosis is due to chronic hepatic injury by alcohol or virus infection, for example, and is characterized by extensive fibrous scarring of the liver and dysfunction. Examples of other liver diseases including biliary type liver fibrosis due to bile duct injury in chronic cholestatic liver diseases, cystic fibrosis associated liver disease or chemical toxins. Fibroblasts and hepatocytes make HGF and these cell types can be used to remove liver fibrosis.

Bone fibrosis can disintegrate bone due to impairment of osteoblast acitivity. This is caused by excess ECM and loss of MMP activity. The fibrosis attracts osteoclasts. Impaired osteoblast function leads to osteopenia and craniofacial dysmorphism. Increase osteoclast activity occurs in arthritis, osteoloysis and osteoporosis due to increase tissue destruction. Implantation of osteoblasts or fibroblasts can remove the bone fibrosis.

Bone marrow fibrosis can inhibit production of stem cells affecting the replenishment of cells in many organ and tissues. Bone marrow fibrosis can be removed by implantation of stromal fibroblasts, in particular, from bone marrow stroma.

Renal fibrosis can be caused by many different kidney diseases. Glomerulosclerosis (focal-segmental) occurs during aging. Others include diabetic nephropathy, lupus nephritis, hypertensive glomerular injury, renal scleroderma, IgA nephropathy, sickle cell nephropathy, glomerulonephritis, nephritic syndrome, chronic graft dysfunction after renal transplantation with tubular loss, amongst others. Renal fibrosis can be classed as interstitial or tubulo-interstitial fibrosis. Epithelial transdifferentiation to fibroblasts, RAGE action, renin-angiotensin and endothelin system loss are some of the mechanisms causing renal fibrosis. ACE inhibitors may prevent renal fibrosis (e.g., age-related). Mesangial cells can degrade ECM and can be used as can renal fibroblasts to remove renal fibrosis.

Cardiac fibrosis can occur from inflammation, heart failure with age, heart trauma, cardiac hypertrophy, amongst other causes.

Fibrosis occurs in many tissues such as granulomatous autoimmue thyroiditis, in nasal polyps (due to inflammatory cells), in inflammatory bowel disease (intestinal myofibroblasts involved), in muscle tissue (e.g., denervated skeletal muscle), in chronic pancreatitis (pancreatic stellate cells involved), in venous disease resulting in soft tissue lipodermatosclerosis, in lens opacification (cataracts) due to continued growth of lens via lens epithelial cell mitosis and differentiation into elongated fiber cells.

Lung fibrosis is initiated with a lung injury, followed by inflammation, fibrous proliferation (e.g., specific interstitial fibroblast and myofibroblast profibrogenic phenotypes), and ending with fibrosis (ECM deposition, adverse remodeling of the parenchyma, lung dysfunction and failure). Pulmonary fibrosis is a progressive and chronic inflammatory lung disease characterized by epithelial cell injury (i.e. type II alveolar cells), mesenchymal cell (fibroblast, myofibrolbast) proliferation, and remodeling of the lung parenchyma. A variety of cytokines, chemokines, and growth factors can be released from epithelial cells to influence fibroblasts and myofibroblast proliferation and differentiaton, and regulation of apoptosis implicated in its development and progression. Epithelial injury can recruit the coagulation mechanisms also. Bone marrow progenitor cells and fibroblasts can be recruited in pulmonary fibrosis. Alveolar epithelial cell activation can result in formation of fibroblast and myofibroblast phenotype conversion. Pulmonary fibroblasts recruited to the lung injury become dysregulated to promote fibrosis.

Pulmonary fibrosis is the abnormal formation of fiberlike scar tissue in the lungs in which the scar formation is preceded by inflammation due to disease or environmental insults. Pulmonary fibrosis causes stiffening of the lungs making it difficult to breathe and is a terminal lung disease. The alveoli (air sacs that exchange oxygen and carbon dioxide), lung capillaries and the interstitium space between alveoli are distorted and scarred due to fibrosis. Pulmonary fibrosis is also known as interstitial pulmonary fibrosis, fibrosing alveolitis, interstitial pneumonitis, and Hamman-Rich syndrome. Most common types of pulmonary fibrosis are idiopathic from unknown causes, occupation disease and sarcoidosis. These include COPD, IIPs (idiopathic interstitial pneumonias), IPF (idiopathic pulmonary fibrosis or interstitial pulmonary fibrosis, DIP, and UID, in which DIP and UID defme IPF in its different stages), graft-versus-host disease after bone marrow and organ transplantation, occupational inhalation of dust particles, post-radiation and chemotherapy, amongst others.

Pleural fibrosis occurs in emphysema. Fibrosis occurs in asthma, chronic bronchitis and chronic lung disease of prematurity (CLD), as well as from infections and disease such as tuberculosis, allergens, autoimmune disease (rheumatoid arthritis, systemic lupus erythematosis, systemic sclerosis, scleroderma), silica, asbestos (in mesothelial cells), and other occupational inhaled particles. Drugs such as methotrexate, bleomycin, cyclophopshamide, amiodarone, and nitiofurantoin can also cause fibrosis.

There is no current treatment for fibrosis. Dexamethasone does not reduce pulmonary fibroproliferation but can reduce inflammation.

COPD progression accumulates inflammatory mucous exudates in the lumen and infiltration of the wall by innate and adapative inflammatory immune cells that form lymphoid follicles. These changes are coupled to a repair or remodeling process that increase the thickness of the wall of these airways. The IIPs (idiopathic interstitial pneumonias) comprise 5 subgroups: usual interstitial pneumonia (UIP), bronchiolitis interstitial pneumonia (BIP), desquamative interstitial pneumonia (DIP), giant cell interstitial pneumonina and lymphoid interstitial pneumonia.

Fibrosis can be diffuse or patchy in the lung. Patchy fibrosis display alternating zones of normal and inflammatory/fibrosing lung parenchyma. Diffuse fibrosis envelops the entire pulmonary parenchyma that is affected by the inflammatory process and has no normal lung parenchyma associated with the disease. Anatomic locations affected by the common chronic inflammatory lung disease are subpleural or paraseptal distributed. In injury, the distal portion of the lobule and acinus is defmed by inflammation and fibrosis from the subpleural region centripetally into the pulmonary parenchyma. Bronchiolocentric distribution in inflammatory processes is localized to the bronchovascular bundle with extension into the contiguous peribronchiolar alveolar septa. Alveolar septal distribution is thickened alveolar septa due to inflammation or fibrosis throughout the lobule. The process is lymphangitic if the inflammation tracks along the visceral pleura, interlobular septa and bronchovascular bundles with little sparing of the septa. UIP is a patchy subpleural and paraseptal distribution of parenchymal injury. The lung injury from nonspecific interstitial pneumonia (NIP) is diffuse with alveolar septal patterning. DIP is diffuse, and is a smoker's type of injury accompanied by alveolar septal inflammation and fibrosis with airspace filling by smoker's macrophages. The alveolar septa is lined by reactive pneumocytes and thickened by mononuclear infiltrate and a mild increase in septal collagen. Respiratory bronchioloitis associated intersitital lung disease is patchy and bronchiolocentric in distribution as is mild peribronchiolar birosis. Cryptogenic organizing pneumonia is a patchy bronchiolocentric and temporally homogenous process with fibromyxoid connective tissue plugs in airway and airspaces. Lymphoid interstitial pneumonia is a dense, diffuse lymphoid infiltration that is mainly alveolar septa in distribution and comprised of T cells, plasma cells and macrophages. Typical features of pulmonary fibrosis in sarcoidosis is different than in IPF or UIP. It begins in the mid and upper lung zones and results in upper lobe volume loss with hilar retraction, traction emphysema and fibrocystic changes and is mainly due to the granulomatous inflammation in pulmonary sarcoidosis. Granular formation begins with the tissue deposition of poorly soluble antigenic material. This is phagocytosed by mononulcear phagocytes and presented as peptides within the class II MHC displayed on the surface of antigen presenting cells for reaction with CD4+ T cells. Cytokines and chemokines produced by these T cells and mononuclear phagocytes develop granulomas. In sarcodiosis, granulomas may resolve by leaving behind residual scar tissue. In the patient with persistent inflammation, the granulomas develop fibrotic changes starting at the periphery of the granuloma and progressing towards the center with hyalizination and collagen deposition.

IPF is classified as a collection of fibrotic lung disorders of unknown etiology. In early IPF there is alveolitis dominated by macrophages and fewer numbers of neutrophils, lymphocytes, and eosinophils and an increase in type II alveolar cells in the epithelium. In the middle phase of IPF, thickening of alveolar walls occur with fibrosis. In the late phase, there is marked change in normal architecture with inflammation and widening of alveolar walls with fibrosis. In the brain, astrocytes or glial cells can be used to removed scarring of neural tissue.

The use of ECM degrading cells or cells with protease secreting activity (MMPs) can remove tissue scarring. Granulomas, cysts and polyps can be treated in a like fashion. In a preferred embodiment, fibroblasts are used. The fibroblasts that typically inhabit the tissue, but removed in location from the fibrosis, can be isolated, expanded in vitro and implanted. Alternately, other types of fibroblasts, such as bone marrow fibroblasts isolated from the bone or from the peripheral circulation or spleen can be used. Alternately, other fibroblasts (e.g., dermal fibroblasts) can be used. Other cell types such as immune cells (e.g., macrophages) can be used.

Tissue functionality can be regained by scar removal. Tissue fibrosis impairs the function of a patient's cells, such as normal fibroblast phenotype in many tissues. The tissue functionality can be augmented by implanting the functional cells of the tissue with fibrosis removing cells or after the fibrosis has been removed.

Fibroids

Uterine fibroids (“myomas,” “fibromyomas,” or “leimyomas are usually benign (non-cancerous) growths that appear within the muscle and connective tissue of the uterus. They usually develop from a single smooth muscle cell that continues to grow. Fibroids can vary considerably in size. Most of the time fibroids grow slowly but others develop more quickly. They typically grow larger over time. Depending on their location in the uterus, how many there are and their size, fibroids can cause discomfort ranging from mild pelvic pressure to quite severe pain, heavy menstrual bleeding, pain during sex, miscarriages and problems conceiving. According to their location in the uterus they can be submucosal, intramural or pedunculated subserosal. Implanted fibroblasts can be used to decrease the size or eliminate fibroid tissue.

Adhesions

Adhesions are a common and occasionally serious outcome of surgery of all kinds, including common gynecologic procedures such as dilation and curettage, cesarean section, hysterectomy, surgical treatment of endometriosis myomectomy (fibroid removal), ovarian surgery and reconstructive tubal surgery. Adhesions that form after surgery in the pelvic area are among the leading causes of post-operative pelvic pain, infertility, and small bowel obstruction.

All of the abdominal and pelvic organs, except the ovaries, are at least partially wrapped in the peritoneum. When the peritoneum is traumatized during surgery or in some other way, the site of the trauma becomes inflamed. Inflammation also contributes to adhesion formation by encouraging the development of fibrous bands of scar tissue (e.g., fibrin matrix). Normally, these fibrin bands eventually dissolve through fibrinolysis and the traumatized site continues to heal. Sometimes the nature of the surgery results in decreased blood flow to these areas (ischemia) which can suppress fibrinolysis. If the fibrin bands do not dissolve, they may develop into adhesions that grow to connect or bind together pelvic organs or tissues that normally are separate. Implanted fibroblasts in or near the site of adhesions can be used to remove or decrease the adhesion. Cells that increase blood flow, such as endothelial cells, can be used to release fibrinolytic proteins and factors to degrade the fibrin matrix and remove the adhesion.

Blood and its Disorders—Anemia

Anemia is a condition of lower than normal number of red blood cells (erythrocytes) in the blood, usually measured by a decrease in the amount of hemoglobin. Hemoglobin is the red pigment in red blood cells that transports oxygen. Erythropoiesis (red blood cell development) starts with the pluripotent hematopoietic stem cell (HSC) differentiating into a myeloid line and forming a colony forming unit erythroid (CFU-E). The CFU-E differentiates into pronormoblasts (proerythroblasts) that mature into normoblasts and synthesize hemoglobin. These cells then extrude their nucleus to become marrow reticulocytes that circulate in the blood for two days before becoming mature erythrocytes. Erythropoietin, (EPO), a glycoprotein hormone produced primarily by cells of the peritubular capillary endothelium of the kidney, is responsible for the regulation of red blood cell production in the bone marrow. Secondary amounts of the hormone are synthesized in liver hepatocytes of healthy adults. In premature as well as full term infants, the liver is the primary site of EPO production. The kidney becomes the primary site of EPO synthesis shortly after birth. EPO production is stimulated by reduced oxygen content in the renal arterial circulation. Circulating EPO binds to EPO receptors on the surface of erythroid progenitors resulting in replication and maturation to functional erythrocytes.

There are many types and potential causes of anemia that can be treated by the invention. One type of anemia, due to vitamin B12 deficiency, is pernicious anemia. This anemia is caused by a lack of intrinsic factor, a substance produced by the parietal cells of the stomach gland needed to absorb vitamin B12. Vitamin B12, in turn, is necessary for the formation of red blood cells. Such deficiencies can be caused by surgical removal of the stomach, inherited conditions, other diseases or aging. This invention describes a form of treatment by injection or placement of autologous gastric parietal cells. Another type of anemia is secondary to a chronic disease. Chronic renal failure or dysfunction occurs over a number of years as the internal structures of the kidney are slowly damaged (e.g., due to aging) causing dysfunctional cell changes in the production of erythropoietin. The resulting anemia is due to a lack of proper stimulation from EPO to the bone marrow to produce red blood cells. This embodiment of the invention includes a form of treatment by injection or placement of autologous renal peritubular endothelial cells into the kidney for EPO production resulting in increased red blood cell numbers. This method can be used in lieu of blood transfusions. The method can also be used for other conditons of the body that compromises red blood cell production, such as chemotherapy or radiation treatment of the bone marrow.

Blood transfusions are increasing in demand due to an aging society that requires transfusions for medical treatments and surgeries. In addition, anemias such as aplastic, pernicious, sickle-cell, due to infections (e.g., malaria) and those due to aging require more red blood cells in the bloodstream. In addition to the above methods to increase red blood cell production in vivo by implantation of ancillary cells, another embodiment of the invention is to obtain red blood cells by the in vitro expansion of progenitor cells, which can then be infused into the subject after expansion as progenitor cells or after differentiation into mature red blood cells in vitro.

Oxygen therapeutics such as non-toxic forms of hemoglobin do not work well due to a short half-life of only a few days. Mature red blood cells however have a lifespan of 120 days. Stromal cells (secreted regulatory and growth factors and ECM) and stem cells in the presence of IL-3, GM-CSF and EPO progress through the erythroid lineage. CD34+ hematopoietic progenitor cells derived from bone marrow, peripheral blood, umbilical cord blood or other sources can be used as the stem cell source. The preferred embodiment is an autologous source. Progenitor cells can be proliferated in vitro and differentiated in vitro with cytokines (e.g., EPO, IL-3, stem cell factor) and co-culture with stromal cells, for example. Erythrocytes can be used or mature red blood cells can be used. Mature red blood cells can be produced in vitro by withdrawing exogenous factors, but maintaining stromal co-culture. Other cell types present in the bone marrow environment may be used in vitro for the proliferation of erythroid cells and differentiation of these cells (e.g., macrophages to induce enucleation). Stages of in vitro production of erythroid cells can be the proliferation of early lineage progenitor cells, followed by differentiation of these cells into later erythroid lineage cells and the maturation of these cells into functional enucleated cells. At any stage of red blood cell development, cells can be used, but the preferred embodiment is the mature red blood cell that is enucleated.

Cancer

Cancer is a disease of altered genes. Over time, DNA accumulates changes that activate proto-oncogenes and inactivate tumor-suppressor genes creating an imbalance of DNA errors that cannot be corrected by DNA-repair machinery. Cancers are diseases in which unremitting clonal expansion of somatic cells kills by invading, subverting and eroding normal tissues. The development of cancer, neoplasia or malignancy usually takes several steps: 1) Initiation in which damage occurs to the cell, changing proteins, DNA or signaling pathways. In most cases cancer originates from a single stem cell which proliferates to form a clone of malignant cells. 2) Promotion in which damage that would normally be removed is instead allowed to persist and further damage the cell. 3) Carcinogenesis in which the cell has now left the normal program of differentiation (anaplasia) and proliferation. Growth is not properly regulated by the normal biochemical pathways, and abnormal growth, angiogenesis (new vessel formation) invasion and metastasis occurs. 4) Clinical Disease show nass effects and tissue dysfunction creating a highly variable clinical presentation. 5) Metastasis is characterized by microscopic groups of cancer cells that develop the capacity for discontinuous growth and dissemination to other parts of the body. Initiation and promotion can be endogenous (e.g., genetic predisposition, genetic mutation, uncontrolled gene expression or abnormal activity by the oncogenes) or exogenous (e.g., exposure to carcinogens, environmental influences and aging). The cancer cell phenotype has six “hallmark features“: loss of signals to stop proliferating and of signals to differentiate, enhanced capacity for sustained proliferation, evasion of apoptosis, invasion of tissue and angiogenesis.

Individual tumor cells do not growth faster than normal cells, even though the total tumor mass often expands rapidly. Several factors limit the optimal potential for tumoral growth and determine the kinetics of tumor growth. These include the need for a blood supply, hence the importance of angiogenesis. Physical barriers allow some tumors to retain growth feedback mechanisms like contact inhibition. Functional tumor suppressors as p53 slow down tumor growth, poor proliferation and immune responses to genetic derangements in cancer create highly antigenic tumors.

Once a tumor “take” has occurred, every increase in tumor cell population must be preceded by an increase in new capillaries that converge upon the tumor. Thus, angiogenesis is important for cancer because in most cases tumor growth, invasion and metastasis will depend on the ability to form new vessels that assure blood supply to the tumor. In cancer two types of angiogenesis occur. The tumor itself elaborates pro-angiogenic factors in direct angiogenesis. During indirect angiogenesis the stroma tissue, responding to either the hypoxia or inflammation caused by the tumor, elaborates growth factors.

All malignant tumors invade locally, and most will metastasize over time. Tumors spread in four different patterns. 1) In direct invasion the tumor leaves the capsule invading and destroying adjacent tissue. Tumors invade basement membranes through the binding of cell adhesion proteins such as laminins, fibronectin and proteoglycans and by proteolytic activity. 2) Seeding of body cavities occur with loose clusters of cells. 3) Lymphatic spread occurs when cancer cells enter the lymphatic vessels. 4) Hematogenous spread of cancer cells usually follow the pattern of organ drainage.

The site of metastasis is determined by anatomy in which cancer cells extravasate to the first capillary bed they enter. Through tropism, certain tissues express specific receptors that attract specific cancer cells. The severity of the metastasis will be determined by the tumor cells survival and colonization at the new site.

Cancer types can be classified according to the type of tissue involved. Adenocarcinoma is cancer that begins in cells lining certain internal organs and that have glandular (secretory) properties. Sarcoma represents cancer of the bone, cartilage, fat, muscle, blood vessels, or other connective or supportive tissue. Squamous cell (epithelial, epidermoid) cancer involves the epithelium of the organ.

The most common cancers are lung cancers that involve small cell carcinoma (squamous carcinoma) and non-small cell carcinoma (epidermoid type of squamous carcinoma, adenocarcinoma, and large cell carcinoma). Breast cancer is characterized by up to 80% invasive or infiltrative cancers that are ductal (i.e., duct cells). Most colon cancers are adenocarcinomas. More than 95% of primary prostate cancers are adenocarcinomas.

Childhood cancers include leukemias, cancer of the blood originating from lymphocytes or other blood cell types. Lymphomas are from any lymphatic tissue or lymphatic node. Bone cancers include osteosarcoma arising from osteoblasts or osteoclasts, Ewing's sarcoma, and chondrosarcoma arising from cartilage cells. Liver cancers are primarily hepatomas. Soft tissue sarcomas include rhabdomyosarcoma arising from muscle cells. Other cancers include brain tumors such as glioblastomas arising from glial cells, nephroblastoma in the kidney, retinoblastoma in the retina, and neuroblastoma arising from nerve cells.

Cancers of blood and lymphatic systems include Hodgkin's Disease of the lymphatic nodes deeper in the body, the leukemias, the lymphomas of the lymphatic nodes in the upper body and multiple myeloma arising from plasma cells.

Skin cancers include malignant melanoma arising from melanocytes, squamous cell carcinoma arising from squamous epithelial cells, cutaneous T-cell lymphoma, Kaposi's sarcoma which is a cancer arising from the endothelial cells of blood vessels inthe skin (Most commonly related to AIDS).

Cancers of the digestive tract include the head and neck cancers that are laryngeal, oral cavity, lip and oropharyngeal and of the oral cavity or lip. These cancers arise from epithelial squamous cells. Esophageal cancer can be about 50% adenocarcinomas and about 50% squamous cell carcinomas. Stomach cancer is primarily due to adenocarcinomas. Pancreatic cancer is greater than 90% from duct, acinar and papillary cells. Liver cancer are adenocarcinomas, with 2 major cell types: hepatocellular (hepatocytes) and cholangiocarcinoma (arising from bile ducts). Colon and rectal cancers are adenocarcinomas. Anal cancer are squamous cell carcinomas.

Cancers of male genitalia and urinary systems include kidney, bladder, testis and prostate. Approximately 85% of renal cell cancers are adenocarcinomas from the distal tubule and may be clear cell or granular cell carcinomas. Bladder cancer is about 90% transitional cell carcinomas derived from the uroepithelium. 6% to 8% are squamous cell carcinomas and 2% are adenocarcinomas. Testis cancer with tumors showing a single cell type are 27% seminomas, 3% embryonic carcinomas, 3% teratomas, 2% yolk sac tumors, and 0.03% choriocarcinomas. The remainder of the cancers involve more than one cell type.

Cancers specific to women and urinary systems include kidney and bladder cancers, breast cancer, ovarian cancer arising from epithelial cells (adenocarcinomas) or from germ cells. Gynecological cancers of the uterus corpus are endometrial adenocarcinomas from the endometrial glands and sarcomas arising from the muscle cells. Cancer of the cervix arise from epithelial cells (i.e., squamous- columnar junction). Vaginal cancer arises from epithelial squamous cells, vulva cancer arises from epithelial squamous cells, epithelial basal cells and or are sarcomas. Choriocarcinomas arise from trophoblastic epithelium during pregnancy.

Endocrine cancers include adrenocortical carcinoma arising from cells of the three layers of the adrenal cortex (i.e., Zona glomerularis, fasiculata and reticularis), carcinoid tumors, gastrointestinal cancers arising from APUD cells, islet cell carcinomas from the endocrine pancreas, parathyroidcancer, pheochromocytoma of the adrenal chromaffin cells, pituitary tumor cancer involving somatotrophs secreting growth hormone, thyrotrophs secreting thyroid stimulating hormone, corticotrophs secreting adrenocorticotrophin, lactotrophs secreting prolactin, and gonadotrophs secreting follicle-stimulating hormone and luteinizing hormone. Thyroid cancers include papillary cell carcinomas, follicular cell carcinomas, Hurthle cell carcinomas and medullary carcinomas.

Many other cancers exist. For example, brain tumors include glial tumors arising from astrocytes, ependymal cells, and oligodendrocytes. Non-glial tumors include pineal tumors from pineocytes or pineoblasts, germ cell tumors, meningiomas, and choroids plexus tumors. Bone tumors, carcinoid tumors, retroperitoneal sarcomas, soft tissue tumors and cancers of unknown primary site are more examples. Several cancer therapeutic modalities exist. Surgery is the best selection for operable localized tumors but not for metastatic disease. Radiation is used to destroy cancer cell DNA. Chemotherapy works best with hematologic malignancies and targets highly proliferative cells. Several types of chemotherapeutics are alkylating agents that bind and crosslink DNA, anti-metabolites that inhibit DNA synthesis by “poisoning” several key enzymes, and natural products. Biological therapies can be angiogenesis inhibitors, immune therapy using antibodies, vaccines or cytokines agains the cancer cells, gene therapy, and bone marrow and peripheral blood stem cell transplantation.

Culture of Immune Cells for Cancer Therapies and Immunization

Certain embodiments of this invention directed to treatment of cancer. As already described, immune cells can be obtained and cultured in vitro and may thus be expanded from a small sample to large number of cells. Similarly, cancer cells can be obtained from a patient and expanded in culture. Cultured immune cells or cancer cells may be introduced into the patient to treat cancer in the patient. The following embodiments are described in terms of autologous cells but allogeneic cells, cells from matched donors, cells from genetically related donors, and cells from younger donors may all be used, as well as suitable stem cells and precursor cells fated or manipulated to achieve an immunophenotype. Further, cells may be reintroduced at one time or in a series over time, or repeated as needed to achieve a clinically observable effect. Moreover, various helpful proteins, as described herein, may also be introduced, e.g., to enhance the “take” of the immune cells. The cells may be introduced remotely, at or near the tumor, or into a region near the tumor, particularly into blood vessels that feed the tumor, e.g., at a distance of 1-50 cm from the tumor.

In one embodiment, cancer cells are obtained from a cancer patient. The cells are disrupted and their contents are optionally denatured, e.g., by mild heat or chemical denaturants. The disrupted cancer cells or portions thereof are reintroduced into the patient. The re-presentation of the antigens of the cancer cells triggers the immune system to effect an improvement in the cancer condition of the patient. The cells may be infused into the blood stream or introduced into portions of the body that serve as reservoirs of immune cells, e.g., bone marrow spaces.

In another embodiment, immune cells are obtained from a patient and expanded in culture. The immune cells may be those cells that are particularly sensitive to identification of cancer antigens, e.g., macrophages, cytotoxic T-cells, natural killer cells, B-cells, or mixtures thereof. The cultured immune cells may be used in a variety of techniques. The immune cells may be in a purified form, enriched with respect to other cells types, or present with a mixture of other cell types.

In a first technique, the cultured immune cells are re-introduced into the patient to boost the patient's immune system. Without being limited to a particular theory, the increased number of cells serves to bolster the immune system's response. In some embodiments, the immune cells are introduced into the blood stream, tissue, or bone marrow. In other embodiments, the immune cells are introduced into the site of a tumor. A single tumor or a plurality of tumors are injected with the immune cells so as to activate the patient's immune system. Alternatively, all or substantially all of the patient's tumors may be injected, with the introduced immune cells directly attacking the tumor and/or activating the immune system of the patient.

In a second technique, the cultured immune cells are cultured with, or mixed with, cancer cells from the patient. The cancer cells may be primary cells or cells cultured from cancer cells taken from the patient. The immune cells and cancer cells may be expanded together or, alternatively, expanded separately and then introduced to each other. The immune cells are introduced into the patient with or without the cancer cells. Without being limited to a particular theory, the immune cells are activated to respond to the cancer cells or to trigger further responses in the immune system of the patient. Biological techniques for activating immune cells to respond to cancer cells may be employed in combination with the co-culture or mixing steps.

In some aspects, the immune cells are introduced as markers of cancer. The immune cells are sensitized to the cancer and imbued with suitable markers that allow the cancer to be visualized. The cancer may then be accurately diagnosed and treated.

Furthermore, a variety of tumor cells alone or with extracellular matrix can be injected to treat the cancer. Cells can be expanded in vitro, denatured, and then infused back into the bloodstream or put at or near the tumor site. Cells plus ECM can be used to optimally stimulate the patient's immune response to the cancer cells. ECM may act as an adjuvant to the cancer cell antigens. ECM from the cancer cells expanded in vitro alone can be used to stimulate the immune response to the specific cancer. In another aspect of the invention, the patient's T cells or B cells (e.g., isolated peripheral bloodstream) can be activated in vitro in the presence of the cancer cells and then re-infused into the subject. In another aspect of the invention, autologous cells, cancer or normal (e.g., fibroblasts) can be genetically modified to deliver anti-cancer proteins such as tumor suppressors.

Cartilage Defects

Cartilage usually develops from the mesenchyme. Mesenchymal cells proliferate and become tightly packed. The cells become rounded, with prominent round or oval nuclei. Gap junctions are present between the cells. Differentiation into chondroblasts is characterized by the cells secreting a surrounding basophilic halo of matrix, composed of a delicate network of fine type II collagen filaments, type IX collagen and cartilage proteoglycan core protein. In some sites, continued secretion of matrix further separates the cells, and produces typical hyaline cartilage. Elsewhere, many cells become fibroblasts, and collagen synthesis predominates. Chondroblastic activity appears only in isolated groups or rows of cells which become surrounded by dense bundles of collagen fibers to form white fibrocartilage. In other sites, the matrix of early cellular cartilage is permeated first by anastomosing oxytalan fibers, and later by elastin fibers. In all cases, developing cartilage is surrounded by condensed mesenchyme which differentiates into a bilaminar perichondrium. The cells of the outer layer become fibroblasts and secrete a dense collagenous matrix lined externally by vascular mesenchyme. The cells of the inner layer contain differentiated, but mainly resting, chondroblasts or prechondroblasts.

Cartilage is a type of load-bearing connective tissue and thus its location covering all the skeletal joints and as a component of several other human body structures. It has a capacity for continued and often rapid interstitial and appositional growth. Appositional growth is the result of continued proliferation of cells of the internal, chondrogenic layer of the perichondrium. Cartilage has a high resistance to tension, compression and shearing, with some resilience and elasticity. Cartilage is covered by a fibrous perichondrium except at its junctions with bone and at synovial surfaces, which are lubricated by a secreted nutrient rich synovial fluid.

The cartilage is formed by extracellular matrix (ECM) and two types of cells, chondroblasts and chondrocytes. Similar to other connective tissues, the ECM is a dominant component and gives the tissue its distinguishing characteristics. According to the type of cartilage (e.g., hyaline, elastic or fibrocartilage) the ECM varies in appearance, composition and in the nature of its fibers.

Cartilage cells occupy small lacunae in the matrix they secrete. Early cells in cartilage development (i.e., chondroblasts) are small, flat and irregular in contour. Newly generated chondroblasts often retain intercellular contacts, including gap junctions. These are lost when daughter cells are separated by the synthesis of new matrix. Mature chondrocytes are mature cartilage cells that lose the ability to divide, become metabolically less active, larger and rounder. The ultrastructure of chondrocytes is typical of cells which are active in making and secreting proteins.

Most cartilage cells are located distant from blood vessels, which are mostly perichondrial. Nutrient substances and metabolites diffuse along concentration gradients across the matrix between the perichondrial capillary network and chondrocytes. This arrangement makes cartilage practically avascular, limiting the thickness of the tissue. Cartilage cells situated further than this from a nutrient vessel do not survive, and their surrounding matrix typically becomes calcified. In the larger cartilages and during the rapid growth of some fetal cartilages, vascular cartilage canals penetrate the tissue at intervals, providing an additional source of nutrients.

The ECM is composed of collagen and, in some cases, elastic fibers, embedded in a highly hydrated ground subsbtance. The components are unique to cartilage giving it its unusual mechanical properties. The ground substance has a complex chemistry. It consists mainly of water and dissolved salts, held in a meshwork of long interwoven proteoglycan molecules together with various other minor constituents, mainly proteins or glycoproteins. Collagen type II forms up to 50% of the dry weight of cartilage. It is chemically distinct from that of most other tissues to the extent that is mainly found elsewhere in the notochord, the nucleus pulposus of the intervertebral disc, the vitreous body of the eye, and in the primary corneal stroma. Collagen in the outer layers of the perichondrium and much of the collagen in white fibrocartilage is collagen type I. The collagen fibers of cartilage are relatively short and thin with a characteristic cross-banding, creating a three-dimensional meshwork linked by lateral projections of the proteoglycans associated with their surfaces. Proteoglycans and other organic molecules link collagen fibers with the interfibrillar ground substance and with cartilage cells. In articular cartilage, collagen fibers close to the surfaces of cells are particularly narrow and resemble fibers of type II collagen in non-cartilaginous tissue, such as the vitreous body of the eye. Cartilage contains minor quantities of other classes unique to cartilage, including types IX, X and XI. In general, proteoglycans are similar to those of general connective tissue, although some features as how chondroitin sulphate and keratan sulphate help in water retention are peculiar to cartilage. Chondrocytes synthesize and secrete all of the major components of the matrix. Collagen is synthesized within the rough endoplasmic reticulum in the same way as in fibroblasts, except that type II rather than type I procollagen chains are made.

Cartilage Types are Comprised of Hyaline, Articular, Fibro and Elastic Cartilage.

Hyaline cartilage has a glassy, bluish opalescent appearance. It is firm and somehow elastic and can be found in the ribs, nose, parts of the larynx, trachea, and bronchea. All temporary and most articular cartilages are hyaline. Shape and arrangement of cells, fibers and proteoglycan composition vary at different sites and with age. The chondrocytes are flat near the perichondrium and rounded or angular deeper in the tissue. They are often grouped in pairs or more, forming cell nests which are the offspring of a common parent chondroblast. The matrix is typically basophilic and metachromatic, particularly in the lacunar capsule, where recently formed, territorial matrix borders the lacuna of a chondrocyte. Fine collagen fibers are arranged in a basket-like network, but are often absent from a narrow zone immediately surrounding the lacuna. A cell nest, together with the enclosing pericellular matrix, is sometimes referred to as a chondron. Hyaline cartilages are prone to calcification after adolescence especially in costal and laryngeal sites and its regenerative capacity is poor.

Articular hyaline cartilage covers articular surfaces in synovial joints providing a smooth, resistant surface bathed by synovial fluid, which allows almost frictionless movement. The principal function of articular cartilage is variable load-bearing through a range of motion and in functional activity. Its elasticity, together with that of other articular structures, dissipates stress, and gives the whole articulation some flexibility, particularly in extreme movements. Articular cartilage is particularly effective as a shock-absorber that reduces the stress on subchondral bone and minimizes the friction. Articular cartilage does not ossify and is moulded to the shape of the underlying bone. It is thickest centrally on convex osseous surfaces, and the reverse is true of concave surfaces. Its thickness decreases from maturity to old age. The surface of articular cartilage lacks a perichondrium. Synovial membrane overlaps and then merges into its structure circumferentially.

Adult articular cartilage exhibits a structural morphologic zonation into four layers from the surface to the center of the articular surface. Zone 1, the Superficial or Tangential layer, is a free articular surface which is a thin and cell-free layer of 3 μm. It contains fine collagen type II fibrils covered superficially by a protein coating. Deeper into the zone are cells that are small, oval or elongated. They are flat and parallel to the surface, relatively inactive, and surrounded by fme tangential fibers. The collagen fibers deeper within this zone are regularly tangential, their diameters and density increase with depth. Zone 2, the Transitional or Intermediate layer, contain cells that are larger, rounder and are either single or in cell nests. Most cells are typical active chondrocytes, surrounded by oblique collagen fibers. Zone 3, the Radiate layer, is a deeper layer containing large, round cells often disposed in vertical colurnns, with intervening radial collagen fibers. As elsewhere, the cells, either singly or in groups, are encapsulated in pericellular matrix which has fine fibrils and contains fibronectin and types II, IX and XI collagen. Zone 4, the Deeper or Calcified layer, lies adjacent to the subchondral bone (i.e., hypochondral osseous lamina) of the epiphysis. The junction between zones 3 and 4 is called the tidemark. With age, articular cartilage thins and degenerates by advancement of the tidemark zone, and the replacement of calcified cartilage by bone. Concentrations of GAGs vary according to site and, in particular, with age. The proportion of keratan sulphate increases linearly with depth, mainly in the older matrix between cell nests, whereas chondroitin sulphates are concentrated around lacunae. The turnover rates of GAGs in cartilage are faster than those of collagen, but decreasing with age and distance from the cells.

The above structural organization exists in cartilaginous growth plates. It follows radial epiphyseal growth by the extension of endochondral ossification into overlying calcified cartilage. This ceases in maturity, but the zones persist throughout life.

Athough cells of articular cartilage can divide, the proliferation rate is low except in young bones. With aging superficial cells are lost progressively from normal joint surfaces, to be replaced by cells from deeper layers. Degenerating cells may occur in any of the four zones. This accounts for the progressive reduction in cellularity of cartilage with advancing age, particularly in superficial layers. Articular cartilages derive nutrients by diffusion from vessels of the synovial membrane, synovial fluid and hypochondral vessels of an adjacent medullary cavity.

After a full-thickness articular cartilage injury, healing produces type I collagen and resultant fibrous cartilage rather than the preferred hyaline cartilage. This “repair” cartilage” has little resilience and poor wear characteristics making it perfect prey for the development of osteoarthritis. The clinical consequence of full-thickness articular cartilage defects of the knee are pain, swelling, mechanical symptoms, functional and athletic disability and ultimately, osteoarthritis.

Fibrocartilage is a dense, fasciculated, opaque white fibrous tissue. It contains fibroblasts and small interfascicular groups of chondrocytes. Structures such as the intervertebral discs contain large amounts of fibrocartilage and have great tensile strength and elasticity. Structures with lesser amounts of fibrocartilage, include articular discs, glenoid and acetabular labra, the cartilaginous lining of bony grooves for tendons and some articular cartilages. These are less, elastic but more resistant to repeated pressure and friction. Fibrocartilage differs from other types of cartilages by the enormous amount of type I collagen and proteoglycans synthesized by the fibroblasts in its matrix that form dense parallel bundles of thick collagen fibers mostly in Zone 1. Fibrocartilage in joints often lack type II collagen altogether, possibly representing a distinct class of connective tissue. Fibrocartilage degenerates very little with age.

Elastic cartilage occurs in the external ear, corniculate cartilages, epiglottis and apices of the arytenoids. It contains typical chondrocytes, but its matrix is pervaded by yellow elastic fibers. Most sites in which elastic cartilage occur have vibrational functions, such as laryngeal sound wave production, or the collection and transmission of sound waves in the ear. Elastic cartilage is resistant to degeneration and it can regenerate to a limited degree following traumatic injury.

Expanded chondrocytes may be implanted with growth factors, apoptosis inhibiting factors, protease inhibiting factors or proteins that stimulate blood flow (vasodilators, angiogenesis proteins) or possible immunogenic proteins or pro-inflammatory proteins, nutrients, transport proteins, into of sites of degeneration. Cartilage cells, precursors thereof, or ex vivo cultured cartilage may be implanted with helpful proteins or other factors as described herein, e.g., to enhance “take” of the cells or tissue.

Articular or hyaline chondrocytes can be implanted preferably into the tidemark line that changes with age. Chondrocytes or chondroblasts from earlier zones such as zone 1 or 2 can be used to implant into the tidemark to reduce the hardening or calcification of the aging cartilage region.

Some embodiments are a method for treatment of full-thickness articular hyaline cartilage lesions of major joints principally involving the knee or shoulder by arthroscopic injection of chondrocytes, e.g., autologous chondrocytes, expanded in vitro. The autologous chondrocytes for implantation may be obtained from a biopsy through the arthroscope from a healthy and minor load-bearing area of the joint to be repair. The implanted cells may originate from cells taken from other healthy locations of cartilage. Progenitor cells to chondrocytes can be used. Perichondrium stem cells can be used. Chondroblasts can be used. Cells located from zones 1-3 are preferred for the isolation of the cells. Chondrocytes or progenitor cells from different types of cartilage (e.g. fibrocartilage, hyaline, articular, and elastic) are preferred to be used for the natural locations of the cells in situ. In an alternate method, cells from one cartilage type can be used for another cartilage type.

Autologous chondrocytes may be expanded in vitro using chondrogenic potentiating growth factors, basic fibroblast growth factors (bFGF), insulin growth factor (IGF) and transforming growth factor β (TGF-β). Methods include treating a is of the hyaline cartilage of the ribs or nose caused by, e.g., a fracture. Methods include treating a lesion of the larynx that is producing alterations in the voice to be repaired by injection of autologous chondrocytes to produce elastic cartilage.

Meniscus

The meniscus is a half moon shaped piece of cartilage that lies underneath the patella. There are two menisci in a normal knee and their role is to absorb about a third of the impact load to the patella. The meniscus is avascular for the most part and this counts for very poor healing conditions after traumatic tears or breaks. It is an embodiment of this invention the repair of lesions of the meniscus include using the injection, seeding or application of precursors of the chondrocytes, chondrocytes or stem cells derived from the bone marrow.

Intervertebral Discs

Intervertebral discs are the chief bonds between the adjacent surfaces of the vertebral bodies from the second cervical vertebra to the sacrum. Their thickness varies in different regions and within individual discs. Discs are the thinnest in the upper thoracic region and thickest in the lumbar region. Each disc consists of an outer lamellated annulus fibrosus and an inner nucleus pulposus. The annulus fibrosus contains a great amount of fibrocartilage and a trace amounts of hyaline cartilage surrounded by an outer collagenous zone (rich in type I and II collagen). These three structures are organized into lamellae.

The inner core of the intervertebral disc, the nucleus pulposus, is composed of a soft gelatinous material rich in notochordal cells at birth. These cells disappear after the first decade of life and the mucoid material is gradually replaced by fibroblast and cartilage cells. The nucleus is very soft at birth due to the high content in water-absorbing aggregated proteoglycans and hardens with time as it is progressively invaded by fibroblasts and cartilage cells that produce collagen fibers and fibrocartilage. The overall proportion of fibrocartilage in the disc increases with age.

Certain embodiments are related to cases wherein the lesion is degeneration, rupture, herniation or atrophy of the intervertebral disc to be repaired, remodeled or bulked by injection of a composition of (e.g., autologous) chondrocytes to produce hyaline cartilage and fibrocartilage. Alternately, cells that produce a similar ECM to the disc can be used, especially those cells producing proteoglycans, such as fibroblasts. An alternate method wherein said lesion is degeneration, rupture, herniation or atrophy of the intervertebral disc to be repaired, remodeled or bulked by injection of a composition of autologous chondrocytes producing aggregated proteoglycans to reverse the hardening of the nucleus pulposus. In an alternate aspect, genetically altered cells (e.g., chondrocytes, fibroblasts) can be used to produce the proteoglycans. Adult mesenchymal stem cells or other cell types such as listed above with hyaluronan gel or with proteoglycans as a carrier can be used.

Fistulas

A fistula is a chronic wound resulting from an abnormal passage from one epithelialized surface to another epithelialized surface commonly compromising and exposing a hollow internal organ (e.g., the intestine or the anus). Fistulas may occur in many parts of the body. The rate of spontaneous closure of a fistula is around 70%.

A fistula fails to heal for a variety of medical reasons. The most common is concurrent infection and degeneration of the adjacent tissues. An internal fistula is the communication between adjacent internal organs or tissues that is between the same organ or tissue (e.g, two portions of the gastrointestinal tract such as an enterocolonic fistula) or different organs or tissues (e.g., rectovaginal fistula). An external fistula involves the skin or another external surface epithelium with an internal organ or tissue, such as in an enterocutaneous fistula.

Enterocutaneous fistula, one of the most common type of fistulas, is the result of complications from surgical procedures in 85% of the cases. Medical treatments, traumatic or instrumented delivery, chronic wounds, trauma, infection or chronic unresolved tissue inflammation are also common causes. Enterocutaneous fistulae drain fluid externally and can be classified as “high input fistulas” when the drainage is more than 500 ml per day, or “low input fistulas” if drainage is less than 200 ml per day. The drained fluid contains water, electrolytes, proteins and other nutrients therefore causing significant morbidity do to malnutrition, dehydration and electrolyte unbalance with a high risk of infection and sepsis from the external exposure of a normally enclosed organ.

Inflammatory bowel disease, such as ulcerative colitis or Crohn's disease, is an example of a disease which leads to fistulae, from one portion of the intestine into another (entero-enteral fistula) or the intestine and skin (enterocutaneous fistula). Up to 30% of the patients with Crohn's disease will develop a fistula at some point. Some other fistulas represent congenital defects such as a tracheo-esophagic fistula. A communication between the fetal trachea and the esophagus can cause severe pregnancy or neonatal complications that can be fatal.

Anal Fistula (Fistula in Ano)

Suppurative anorectal infection can be divided into two categories—anorectal abscess and anorectal fistula. Drainage of an anorectal abscess results in a cure for about 50% of the patients. The remaining 50% develop a persistent fistula in ano. While the majority of fistulas are infectious in origin, trauma, Crohn's disease, cancer, radiation or unusual infections may also produce fistulas. A fistula in ano is usually diagnosed by the presence of a red, granular papula from which pus or fluid is expressed.

All anorectal fistulae are anatomically divided into one of four groups. The classification is important to determine tissue involvement and predict complications after treatment. When other tissues, particularly muscular structures important for continence are involved, the risk of fecal incontinence after treatment increases. The most common type of fistula in ano is the intersphincteric fistula, in which the fistula ramifies in the tissue between the internal and the external sphincters. Transsphincteric fistulas pass from the tissue between the two sphincters into the ischiorectal fossa. Suprasphincteric fistulae pass upward over the puborectalis muscle and extrashincteric fistulae pass from the perianal skin through the ischiorectal fat and elevator muscles into the rectum.

A rectovaginal fistula is a connection between the vagina and the rectum or anal canal. Patients describe symptoms varying from the sensation of passing flatus from the vagina to the passage of solid stool from the vagina. It is frequently associated with vaginal infections and fecal incontinence. Rectovaginal fistulas are classified as low when the vaginal opening is close to the vulva, middle when the vaginal opening is higher but lower than the cervix and high when the vaginal opening is higher than the cervix. Low rectovaginal fistulas are commonly caused by obstetric injuries. Middle fistulas may result from more severe obstetric injury, but also occur after surgical resection of rectal neoplasm, radiation injury, or drainage of a posterior rectal abscess. High fistulas result from operative or radiation injury. Crohn's disease can cause rectovaginal fistulas at all levels as well as enterovaginal fistulas between higher portions of the bowel and the vagina.

Fistulas may occur in many other parts of the body. Some of these are arteriovenous (between an artery and vein), biliary (created during gallbladder surgery connecting bile ducts to the surface of the skin), bladder (communication between the bladder and the bowel, or bladder and the vagina are the most common), bronchopleural (between the bronchi and the pleural space), cervical (such as an abnormal opening in the uterine cervix or in the neck), craniosinus (between the intracranial space and the paranasal sinus), gastric (from the stomach to the surface of the skin), metroperitoneal (between the uterus and the peritoneal cavity), periodontal (communication between a tooth root canal and the gum), pulmonary arteriovenous (in the lung, between an artery and a vein), and umbilical (connection between the umbilicus and the gut).

Current approaches to promote fistula healing usually involve surgical procedures that are time consuming and costly. Sealants have had limited success in the closure of a fistulas. These reports show limited success. It is desirable to provide a safe, minimally invasive and efficacious method to treat and close fistulas.

Embodiments thus include a method to achieve healing and closure of a fistula as a type of wound by implanting (e.g., autologous) fibroblasts into a patient, e.g., along the entire fistulous tract. The autologous fibroblasts may be derived from a tissue with the same characteristics as the tissue(s) of which the fistula is comprised. The autologous fibroblasts may be derived from a tissue that it is the same to the tissue of which the fistula is comprised. The autologous fibroblasts may be derived from a tissue different to the tissue of which the fistula is comprised. Other mesenchymal cells and stem cells and wound healing cell types can be employed.

The autologous fibroblasts may be administered more than once and in different amounts as repetitive treatments preferably by not exclusively in the form of injections, endoscopic injections or topical application as to attempt complete closure of the defect. The treated defects may include: an a iatrogenic fistula, a spontaneous fistula, a fistula due to radiation treatment for cancer, a fistula due to ischemia, a fistula due to inflammation secondary mainly but not exclusively to infection, an enterocutaneous fistula of the gastric, duodenal, pancreatic, jejunal, colonic or anal tissues. And the fistula may be a bladder, vaginal, uterovesical or vesicovaginal fistula. The fistula may be a tracheo-esophageal, tracheocutaneous, esophagocutaneous or bronchopleural fistula.

Gut

The average adult human intestine is a 10 meter-long tube. It constitutes a two-dimensional structure folded into valleys and hills, the proliferative crypts and the differentiated villi. The villi has an unprecedented cell self-renewal rate (replaced at a rate of ˜70 billion per day). The inner layer of the gut, the intestinal epithelium, constitutes a barrier between the body and the outside world, absorbing nutrients and defending against would-be pathogens.

The epithelium of the adult small intestine forms a contiguous two-dimensional sheet. New cells are added into the crypts and removed by apoptosis upon reaching the villus tips a few days later. Stem cells and Paneth cells at the crypt bottom escape this flow. Paneth cells occupy positions 1 to 3 from crypt bottom to up and the stem cells are found at position 4 going up. The cell harboring crypt niche lays apposed to a sheath of specialized fibroblasts (i.e., myo-epithelial fibroblasts) separated only by the basal lamina. The intestinal epithelium consists of a single layer of fragile epithelial cells. These cells digest food and absorb the resulting mix of biological building blocks while keeping indigestible bulk and associated microflora inside the lumen. All these tasks are distributed and performed by four types of differentiated cells. All these cells are located in an adult intestinal crypt and derive from only one stem cell. Two main lineages of differentiated cell types exist within the intestinal epithelium, the enterocyte or absorbtive lineage and the secretory lineage. The secretory lineage encompasses goblet cells, the enteroendocrine lineage and the Paneth cells. Enterocytes are abundant in the small intestine, secreting hydrolases and absorbing nutrients. The goblet cells secret protecting mucins. Enteroendocrine lineage cells can be further subdivided on the basis of the hormones they secrete e.g., serotonin, substance P, or secretine. Paneth cells residing in the very bottom of the crypt secrete anti-microbial agents and lysozyme to control the microbial content of the intestine.

Glycocalyx enterocytes are surface absorptive cells that are joined together by tight junctions and contain microvilli coated with filamentous glycoproteins. The glyocalyx contains the enzymes lactase, maltase, sucrase, α-dextrinase, trehalase, aminopeptidases and enterokinase. Lactose intolerance is due to a deficiency in lactase. This deficiency is widespread in a majority of populations and increases with age infections.

Absorption changes with age or is disease. Absorption can be improved with the use of stem cells. Implantation of the cells into the position 4 of the crypt is preferred. The stem cell can be genetically altered, for example, to include lactase so that the cells can be used to correct lactose intolerance of the subject. Precursors to parietal cells that absorb vitamin B12 along the gut can be implanted to improve pernicious anemia. The implantation of parietal cells can improve the gut absorption of vitamin D. Such cells may be implanted to address defects or conditions associated with the gut.

Olfactory Sense

The peripheral receptors for olfactory sensation are located bilaterally in areas of sensory epithelium lining the posterodorsal parts of the nasal cavities. The sensory epithelium occupies an area of c0.5 cm², covering the posterior upper parts of the lateral nasal walls as a pigmented yellowish brown color in contrast to the pinkish color of the rest of the respiratory mucosa of the nasal cavities. The complete structure is known as the olfactory mucosa. The mucosa consists of an epithelium thicker than the respiratory epithelium, and measuring up to 100 μm. This epithelium is columnar, ciliated and pseudostratified. It contains the olfactory receptor neurons situated among columnar sustentacular or supportive cells that contain microvilli and two classes of basal cells. Horizontal basal cells are the closest and flattened against the basal lamina. The globose basal cells are rounded and elliptical in shape. The olfactory epithelium sits on top of an underlying lamina propia that contains the axons of the olfactory receptor neurons and subepithelial olfactory glands (of Bowman) that secrete a thin fluid layer in which sensory cilia and the microvilli of the sustentacular cells are embedded.

The olfactory receptor neurons are slender ciliated bipolar neurons with a nucleus located in the middle zone of the epithelium, a single unbranched apical dendrite and a basal unmyelinated axon. Several axons form small intraepithelial fascicles that penetrate the basal lamina and are immediately ensheathed by olfactory ensheathing glial cells. Groups of up to 50 fascicles join to form larger olfactory nerve roots that penetrate the bone structure at the roof of the nasal cavity known as the cribiform plate to enter the olfactory bulb, which is situated at the anterior end of the olfactory sulcus on the orbital surface of the frontal lobe. There is a clear laminar structure in the olfactory bulb. From the surface inwards are the olfactory nerve layer, glomerular layer, external plexiform layer (constituted by the principal and secondary dendrites of mitral and tufted cells), mitral cell layer, internal plexiform layer and granule cell layer. The principal neurons of the olfactory bulb are the mitral and tufted cells which axons make synapsis with secondary sensory neurons to form the olfactory tract and later the 1^{st} cranial nerve, the olfactory nerve.

Hence the olfactory epithelium is a neuroepithelium and its neurons are the only nerve cells that continually regenerate from the basal cells after neuron damage or loss. Individual receptor neurons have a lifespan averaging 1-3 months, when they degenerate dead cells are either shed or phagocytosed by sustentacular cells. Stem cells situated near the base of of the epithelium undergo periodic mitotic divisions giving rise to new olfactory receptor neurons that differentiate growing a dendrite and an axon. The rate of receptor cell loss and replacement increases after exposure to damaging stimuli. Their capacity to turnover declines slowly but steadily with age contributing to the diminished olfactory sensory function so typical of the elderly.

Membrane receptors in the cilia detect odorants and among the millions of sensory cells (the neurons) each receptor detects a subset of the 10.000 or so different detectable odors. When odorant molecules bind to receptors, nerve cell depolarization and action potentials are triggered. The number of primary odors ranges from six to several dozens depending on the method of classification. The repertoire of distinct receptor populations for odorants in humans is possibly about 30, since there are about this number of specific anosmias (inability to detect a particular odorant). The odorant response is terminated by two mechanisms. First, there is an increase in the airflow created by sniffing aided by the watery dilution of the odorant molecule by secretions delivered by the Bowman glands. Second, the odorant molecule is inactivated by the sustentacular cells and their enzymes via hydroxylation and glucoronidation.

Some embodiments, accordingly, are to implant basal stem cells into the epithelium base, e.g., to provide new olfactory receptor neurons for improving smell that is a common loss in the aged or due to disease or is a desired augmentation. In another aspect of the invention, isolation of astrocytes in any part of the brain can be used but the preferred region is from the lining of lateral ventricle. These cells can migrate to the olfactory bulb. These cells be used to replenish the basal stem cells. Such cells, or their precursors, may be isolated, expanded, and implanted as described herein, with or without associated helpful proteins, factors, or ECM.

Taste

The sense of taste is dependent on scattered groups of several thousands of sensory cells called the taste buds. The taste buds are small barrel shaped intraepithelial specializations of the oral cavity mucosa and occur chiefly in the tongue with a few located in the epiglottis, soft palate, and pharynx. The taste buds reside mainly in the fungiform papillae formations of the dorsal mucosa of the posterior part of the tongue with fewer numbers scattered over the anterior two-thirds of the tongue. About 1000 taste buds are distributed over the sides of the tongue. Each taste bud is approximately 50 μm in diameter and consists of a barrel shaped cluster of 50-150 fisiform epithelial-like cells of three types, the: tall, slender taste sensory cells, supporting cells and small basal cells. Each cluster lies within an oval cavity in the epithelium of the mucosa and converges apically on a gustatory pore, a 2 μm opening on the mucosa surface through which the saliva carrying the tasting object enters causing nerve depolarization of the sensory cells. The sensory cells are characterized by a cell membrane full of microvilli holding multiple receptors and the absence of dendrite or axon formations.

The taste buds have a life span of about 14 days. New taste buds are formed in response to innervation of the lingual epithelium, which is thought to stimulate development of the basal cells into taste and supporting cells. The supporting cells are can be a stage in the cell cycle of taste-cell differentiation.

Serous secretions delivered to the surface epithelium from exocrine glands intrinsic to the tongue assist with washing the taste buds, allowing detection and solubilization of molecules that excite the taste receptors inside the microvilli of the sensory cells. The receptor taste capabilities are grouped into four main categories, sweet, sour, salty and bitter. These taste stimuli are detected by entry into the gustatory pole to contact the sensory cell receptors depolarizing the cell with resulting action potentials releasing neurotransmitters, which stimulate afferent nerve terminals in the taste bud, passing signals to several cranial nerves and then into the cerebral cortex.

A single afferent nerve can carry more than one type of signal depending on the type of chemical stimulus. Therefore one taste bud can be excited by several or all four primary taste stimuli. Sweet and salty tastes are mainly detected on the tip of the tongue, sour taste on the lateral margins of the tongue, and bitter taste mainly on the posterior surface of the tongue. Although the areas stated above may mainly detect a particular taste, all areas can be responsive to all tastes. Taste wanes with aging and particular diseases.

Thus some embodiments of the invention are directed to implantation of stem cells of the lingual epithelium e.g., as can develop into basal taste cells and supporting cells to improve taste loss during aging or disease or for a desired augmentation.

Aging Tissue and Organs

Aging can be defined as a physiologic dysfunction that represents a shift from optimal tissue and organ function in one's lifetime. Aging predisposes the subject to disease, deleterious conditions and cellular activities, amongst others described throughout the text and those known in the art.

A major change in the phenotype of aging tissue is an alteration of the connective tissue component. In general a decrease in the quantity of connective tissue is observed. Some of the connective tissue proteins and molecules involved are the different forms of collagen (types I-IX), the different forms of fibronectin, the proteoglycans biglycan, decorin, versican, aggrecan, heparin binding proteoglycans, vitronectin, thrombospondin, osteonectin, elastin, fibrillins, lamellins, hyaluronic acid, elastin, amongst others.

Tissues become dystrophic with age, altering or compromising its function. Often, there is a hypertrophy of the tissue due to higher production of structural proteins versus protease degradation of certain cell types of the tissue. Sometimes there is atrophy, in which less structural proteins or ECM is produced than in younger tissue. Dystrophy can be a combination of specific areas of the tissue undergoing hypertrophy and others atrophy. For example, MMP acitivities are higher in aged or photodamaged skin and structural proteins are lower in abundance than in younger skin. Cell implantation of connective tissue forming fibroblasts can change any or all of these activities improving the function and structure of aged or photodamaged skin.

Additionally, there is a loss of elasticity of the tissue due to the connective tissue component alteration (e.g., elastin, proteoglycans). For example, this is reflected in a marked decrease in functionality of lung tissue and a 40% decrease in functionality of kidney tissue in the elderly compared to the young adult. Additionally there is a loss of moisture or hydration (e.g. less proteoglycans) in aged tissue. Furthermore, there is a loss of turgor in aged tissue. Additionally there is loss of volume of the tissue due primarily to the decreased connective tissue component alteration.

Aging and diseased tissue become dysfunctional in large part due to loss of appropriate numbers of cell types. This in turn results in lower cell populations and changing gene expression that alter ECM matrix, protein and enzymatic activities (proteases), cell adhesion, cell migration, cell proliferation, cell differentiation, hormone and growth factor production, signaling pathways, feedback mechanisms, tissue homeostasis and dystrophic tissue morphology, amongst other actions. Increased numbers of cells implanted or in tandem with specific proteins that diminish with aging can improve the aged tissue. For example, the addition of fibronectin to increase ECM interactions with the implanted cells can improve the implantation or 37 take” of the cells and improve the aged tissue.

In many aging tissues, cells that are added may be more effective when specific growth factors and hormones are implanted in tandem, to provide assistance to any cellular intrinsic deficiencies.

In one aspect of the invention, bone marrow progenitor cells are implanted or infused into the bone marrow (e.g., stroma) to replenish the numbers of progenitor cells that can be used to rejuvenate all tissue and organs that have become dysfunctional or less functional due to the process of aging. This invention can be used to rejuvenate the body as a whole. In a preferred embodiment younger cells are used in older patients. In another preferred embodiment younger whole blood/fractionated blood/plama/serum is infused into older patients at regular, repeating intervals to improve tissue and/or physiological functions(s). Alternately, if a certain tissue needs replenishment of progenitor cells autologous progenitor cells, younger cells (autologous or non-autologous) and/or younger whole blood/fractionated blood/plasma/serum can be infused or implanted into the tissue of interest.

Alternately, the progenitor cells can be used by direct implantation into the organ or tissue of choice.

The loss of cell number during the aging of tissue can be restored in the invention. Replenishment of the cells and/or extracellular matrix present in the tissue can restore or improve tissue and organ functionality. Cells and/or extracellular matrix can also be used from other types of connective tissue to restore or improve the tissue. Another example is the use of cells from the tissue or connective tissue component of an organ that is physiologically younger from the same individual into another tissue of the same individual. An example is the use of fibroblasts from a connective tissue source that is not subjected to an environmental insult such as radiation, sunlight, temperature or chemicals. Alternately, cells and/or extracellular matrix from the tissue from a younger donor can be used in the same or different tissue of another or older host. Other youthful and functional properties can be used by the use of younger cells and younger blood/plasma/serum, as described elsewhere herein.

Organ Tissue Engineering and Organ Tissue Regeneration

Organ Replacement and Synthesis

There are approaches to the problem of a missing, completely failing or aged degenerated organ such as autograft, transplant, implant, in vivo synthesis (tissue regeneration) or in vitro synthesis (tissue engineering). Autografts are surgical solutions often limited by lack of donor tissue. Transplantation from another individual involves a major, complicated and costly surgical intervention and also suffers often from lack of availability as well as problems of immunological rejection. Synthetic implants are quite useful in some medical conditions but have such problems as longevity. Tissue engineering and tissue regeneration can be used to develop organs to replace the function of failing ones or correct the aging related decline of the organs by implanting with increased numbers of cells or by supplementing the old cells in the organ with younger or multiplied cells to return the organ to normal functioning.

Biological tissues and organs consist of specialized cells that are situated within a complex molecular framework known as the extracellular matrix (ECM). In addition to providing tissues with appropriate 3D architecture, ECM has been to promote signaling pathways that influence key cell function as migration, proliferation and differentiation.

The tissue engineering discipline with three-dimensional biomaterials basically involves the selection of the optimal material for the scaffold to promote and sustain tissue growth followed by the retrieval, isolation, and in-vitro culture and seeding of the proper cell type according to the needed of the tissue.

Nearly every scaffold is formed by either a synthetic or natural polymer. Synthetic polymers commonly utilized for tissue engineering applications include poly α hydroxy acids, polyorthoesters, polyurethanes and hydrogels. Collagen-based materials are widely used natural polymers. Among several challenges, the issue of the optimal scaffold to create a cellular environments to optimally develop a determined tissue is a crucial one. A variety of 3D bioengineering, biodegradable scaffolds provide can provide adhesive substrates and serve as a 3D physical support matrix for in vitro cell culture as well as in vivo tissue regeneration.

The cells needed can be harvested from the individual by a biopsy procedure to tissue engineer or regenerate the organ in an autologous way. When this is not possible because of total organ damage and failure a different source can be tapped as undifferentiated mesenchymal cells coaxed to differentiate into the desired cell, embryonic or adult stem cells or preferably a cell from a donor. Cells may be expanded, loaded and seeded into the chosen scaffold until an optimal cell density is achieved; thus good organ function is reestablished.

Younger Cell Types, Tissue Sources Protected from Light and Chemical Exposure, ECM, and Serum

Autologous cells with or without human or autologous may be used for implantation into a patient. Younger, rather than older, autologous cells and/or serum can be used, and can be obtained and stored (e.g., by cryopreservation) from previous chronological biopsies of the subject. In another preferred embodiment, genetically similar cells or serum can be substituted for autologous cells. In some embodiments, autologous cells are derived from cells taken from the patient a number of years prior to the date of cellular reintroduction, e.g., between 1-80 years, e.g., 5, 10, or 15 years, with all ranges and values between the explicitly stated values being contemplated.

Additionally, non-sun, chemical or radiation exposed cells may be used for introduction into a patient. For instance, some tissue sources are naturally protected from sun and chemical exposure, e.g., tissue from behind the ear or buttocks region. The cell phenotype can be chosen to be similar to the host's tissue site after is implanted. The types of cells from specific tissues described in the text can be implanted at a site used for the construction of organs most resembling the natural destination tissue in the patient.

ECM synthesized in three or two dimensions can be used. The ECM can be included in the implantate. Xenogenic, allogenic or autologous ECM or its constituents can be used with autologous or non-autologous cells. Matrices that can be used include natural and synthetic, are preferably biodegradable and can contain immunogenic determinants that with time are removed by degradation or other mechanisms. Matrices can contain matrikines, motifs or domains of ECM proteins, MMPs or inhibitors of, ECM receptors such as integrins, growth factors, cytokines, chemokines, pro-coagulation sequences, plasmin degradation sites, proinflammation sequences, amongst many other possibilities, that can promote wanted cell proliferation, differentiation and other functional outcomes. Cells in culture can produce dense 3-D matrices (e.g. via proper serum supplementation that overcome contact inhibition) and cells within these 3-D matrices form a distinct class of adhesion. ECM may be included in culture or with cells implanted into a patient.

Co-culture of stem cells or other cells that normally reside in vivo with underlying stromal fibroblasts can be used to promote proliferation, differentiation and survival of these cells, such as endothelial, epithelial or stem cells. Such co-culture can be augmented using autologous serum and/or younger serum.

Other Aspects

In general, repair of structures can be done with somatic cells or progenitor cells in the area. For example, immature fibroblasts (mesenchymal fibroblasts) lie within the same tissue spaces alongside mature fibroblasts and fibroblasts of distinct fibroblast lineages. Fibroblasts from different anatomical sites display characteristic phenotypes. Fibroblasts in the head and neck region can be from the neural crest tissue (ectodermal in origin) not mesodermal. And fibroblasts are heterogenous with respect to number of phenotypic and functional features that is due to different cellular origins.

In general, it is noted that stem cells are often not restricted in their potential to differentiate and regenerate tissue in which they reside. Bone marrow stem cells can differentiate into hematopoietic or nonhematopoietic mesenchymal stem cells, muscle, heart, liver, vascular cells and other mesenchymal cell types and are recruited as progenitors for tissue fibroblasts via the circulation to populate peripheral organs.

The brain can be regenerated by addition of astrocytes that behave as stem cells. Although astrocytes in any part of the brain can be used, the preferred source region is from the lining of lateral ventricle. These cells can migrate to the olfactory bulb. These cells can form mature brain cells, the astrocytes, the microglia and the oligodendrocytes, and the neurons. Useful for PD, motor and sensory systems of the brain, AD, perhaps not the higher regions because of memory, etc. could change.

Thus some embodiments include obtaining cells and/or extracellular matrix from tissue. And autologous cells and/or extracellular matrix may be obtained from tissue. Cell culture may use autologous serum and other serums for cell culture. Cells and/or extracellular matrix derived from a tissue may be introduced into the same tissue from which the cells or ECM was originally derived. Alternatively, cells or ECM may be reimplanted into a different tissue. Further, cells can be obtained from other human donors or younger human donors such as neonatal, fetal or physiologically younger.

Gastroesophageal-Reflux Disease

The esophagus is a muscular canal, about 8 inches in length extending from the pharynx to the stomach. The esophagus has three coats: an external or muscular composed by two groups of thick muscular fibers running longitudinally and circular; a middle or areolar coat of connective tissue which is thick and shows a distinctive layer of smooth muscle forming the muscularis mucosae in contact with the third coat an internal or mucous one consistent of a highly dynamic squamous epithelium. The upper and lower ends of the esophagus have sphincters; the upper one at the level of the cricoid cartilage that remains close by the elastic properties of its walls and the action of pharyngeal muscles; in contrast the lower esophageal sphincter (LES) remains close because of its intrinsic myogenic tone and a neural pathway of pre and postganglionic neurons, therefore it is affected by multiple substances contained in food, hormones and neurotransmitters as well as subtle changes in the abdominal pressure that lowers or eliminates the gradient of pressure between the LES and the stomach. The lower sphincter is not histologically distinct.

The preferred route to deliver embodiments of the invention for treating gastroesophageal reflux (GER) or also stated as gastroesophageal reflux disease (GERD) is through the endoscope which is introduced in to the esophagus lumen and its tip is located at a proper visual distance of abnormally distended LES lumen and a needle is introduced through the working channel of the endoscope and advanced into the LES surrounding tissue injecting the preparation preferably but not exclusively into the muscular layer of the LES until the remodeling/bulking and ideally narrowing of the LES lumen is achieved. Injection may be aliquoted in two at the 3 and 9 o'clock positions. Care must be exercised in performing a single precise injection because if multiple ones are needed the material will be lost to extravasation. The needle is kept in position for 2-3 minutes before withdrawal for the same reason. Preferred cell types to be used are fibroblasts and/or preadipocytes/adipoctyes into the connective tissue area of the sphincter and myoblasts, smooth muscle cells, striated muscle cells, into the muscle tissue area of the sphincter. Additionally, mesenchymal stem cells and epithelial cells may be used. Alternately, connective tissue cells can be implanted into the muscle area and muscle cell types or stem cells into the connective tissue area of the sphincter. Preferably, one cell type is used and injected into the area of the sphincter either in the connective tissue area or muscle area or both. In a preferred embodiment, fibroblasts and/or preadipocytes are implanted into the connective tissue area of the sphincter or into the sphincter area.The cell types can be obtained from the sphincter area or from other tissues. Preferably autologous cells are used.

In addition, there is an alternative use of the invention during open surgery or laparoscopic to treat diaphragmatic hernia as it is the injection of the viable cell compounds directly in to the surgical repaired tissues during surgery to reinforce the frequent poor results of the surgical treatments.

Significant details applicable to GERD are provided in the applications incorporated herein by reference, i.e., U.S. patent application Ser. Nos. 09/632,581 (filed Aug. 3, 2000) that claims priority to 60/037,961; 10/129,180 (filed May 3, 2002) that claims priority to 60/163,734; and PCT Application ______ filed Sep. 14, 2006 entitled “Compositions And Methods for the Augmentation and Repair of Defects in Tissue”. These applications provide additional detailed information that is applicable to GERD and form part of this disclosure.

Cell Types and Culture

Certain embodiments herein are described with respect to autologous cells. Non-autologous cells can be used, however, as appropriate for the application, for example in the case where autologous cells could be detrimental, as in genetic diseases that confer dysfunctional characteristics. In some instances, immune suppression may be needed to sustain non-autologous cells with significantly distinct immunotype characteristics.

Different cell types or modified cell types (e.g., genetically altered) than those that exist in the subject's tissue can be used to treat a tissue defect providing that these other cell types appropriately emulate or simulate the functionality of the subject's tissue to thereby treat the tissue defect. Cell types native to the tissue that has the defect may be used in the treatment. Native cell refers to a cell type that is the same, or functionally equivalent, to the cell type that is being replaced in a tissue or the type of cell that is in the site that is receiving the cell. Native cells can be obtained from the site of injury, from the same tissue type but one that is uninjured, or from a corresponding tissue from a donor other than the patient. Amongst the cell types that can be used according to the methods set forth herein include those described elsewhere herein and in the following classification which provides examples of cells that may be used: keratinizing epithelial cells, wet stratified barrier epithelial cells, exocrine secretory epithelial cells, hormone secreting cells, epithelial absorptive cells (gut, exocrine glands and urogenital tract), metabolism and storage cells, barrier function cells (lung, gut, exocrine glands and urogenital tract), epithelial cells lining closed internal body cavities, ciliated cells with propulsive function, extracellular matrix secretion cells, contractile cells, blood and immune system cells, sensory transducer cells, autonomic neuron cells, sense organ and peripheral neuron supporting cells, central nervous system neurons and glial cells, lens cells, pigment cells, germ cells, and nurse cells.

Keratinizing epithelial cells: Keratinizing epithelial cells are present in various tissues in the body, as indicated, and include, e.g.: epidermal keratinocyte (differentiating epidermal cell), epidermal basal cell (stem cell), keratinocyte of fingernails and toenails, nail bed basal cell (stem cell), medullary hair shaft cell, cortical hair shaft cell, cuticular hair shaft cell, cuticular hair root sheath cell, hair root sheath cell of huxley's layer, hair root sheath cell of henle's layer, external hair root sheath cell, and hair matrix cell (stem cell).

Wet stratified barrier epithelial cells: Wet stratified barrier epithelial cells are present in various tissues in the body, as indicated, and include, e.g.: surface epithelial cell of stratified squamous epithelium of cornea, tongue, oral cavity, esophagus, anal canal, distal urethra and vagina; basal cell (stem cell) of epithelia of cornea, tongue, oral cavity, esophagus, anal canal, distal urethra and vagina; and urinary epithelium cell (lining urinary bladder and urinary ducts).

Exocrine secretory epithelial cells: Exocrine secretory epithelial cells are present in various tissues in the body, as indicated. Exocrine secretory epithelial cells include, e.g.: salivary gland mucous cell (polysaccharide-rich secretion), salivary gland serous cell (glycoprotein enzyme-rich secretion), von ebner's gland cell in tongue (washes taste buds), mammary gland cell (milk secretion), lacrimal gland cell (tear secretion), ceruminous gland cell in ear (wax secretion), eccrine sweat gland dark cell (glycoprotein secretion), eccrine sweat gland clear cell (small molecule secretion), apocrine sweat gland cell (odoriferous secretion, sex-hormone sensitive), gland of moll cell in eyelid (specialized sweat gland), sebaceous gland cell (lipid-rich sebum secretion), bowman's gland cell in nose (washes olfactory epithelium), brunner's gland cell in duodenum (enzymes and alkaline mucus), seminal vesicle cell (secretes seminal fluid components, including fructose for swimming sperm), prostate gland cell (secretes seminal fluid components), bulbourethral gland cell (mucus secretion), gland cell (vaginal lubricant secretion), gland of littre cell (mucus secretion), uterus endometrium cell (carbohydrate secretion), isolated goblet cell of respiratory and digestive tracts (mucus secretion), stomach lining mucous cell (mucus secretion), gastric gland zymogenic cell (pepsinogen secretion), gastric gland oxyntic cell (hydrochloric acid secretion), pancreatic acinar cell (bicarbonate and digestive enzyme secretion), paneth cell of small intestine (lysozyme secretion), type ii pneumocyte of lung (surfactant secretion), and clara cell of the lung.

Hormone secreting cells: Hormone secreting cells are present in various tissues in the body, as indicated, and include, e.g.: anterior pituitary cells such as anterior pituitary cells, somatotropes, lactotropes, thyrotropes, gonadotropes, corticotropes; intermediate pituitary cell, secreting melanocyte-stimulating hormone; magnocellular neurosecretory cells secreting, e.g., oxytocin or vasopressin; gut and respiratory tract cells secreting, e.g, serotonin, endorphin, somatostatin, gastrin, secretin, cholecystokinin, insulin, glucagon, or bombesin; thyroid gland cells such as thyroid epithelial cell or parafollicular cell; parathyroid gland cells such as parathyroid chief cell and oxyphil cell; adrenal gland cells such as chromaffm cells secreting, e.g., steroid hormones (mineralcorticoids and gluco corticoids); leydig cell of testes secreting testosterone; theca interna cell of ovarian follicle secreting estrogen; corpus luteum cell of ruptured ovarian follicle secreting progesterone; kidney juxtaglomerular apparatus cell (renin secretion); macula densa cell of kidney; peripolar cell of kidney; and mesangial cell of kidney.

Epithelial absorptive cells: Epithelial absorptive cells are present in various tissues in the body, as indicated. Epithelial absorptive cells (as in the gut, exocrine glands and urogenital tract) include, e.g,: intestinal brush border cells (with microvilli), exocrine gland striated duct cells, gall bladder epithelial cells, kidney proximal tubule brush border cells, kidney distal tubule cells, ductulus efferens nonciliated cells, epididymal principal cells and epididymal basal cells.

Metabolism and storage cells: Metabolism and storage cells are present in various tissues in the body, as indicated, and include, e.g.: hepatocytes (liver cell), white fat cells, brown fat cells, and liver lipocytes.

Barrier function cells: Barrier function cells are present in various tissues in the body, as indicated. Barrier function cells (as in the lung, exocrine glands and urogenital tract) include, e.g,: Type I pneumocytes (lining air space of lung), Pancreatic duct cells (centroacinar cell), Nonstriated duct cells (of sweat gland, salivary gland, mammary gland, etc.), Kidney glomerulus parietal cells, Kidney glomerulus podocytes, Loop of Henle thin segment cells (in kidney), Kidney collecting duct cells, and Duct cells (of seminal vesicle, prostate gland, etc.).

Epithelial cells lining closed internal body cavities: Epithelial cells lining closed internal body cavities are present in various tissues in the body, as indicated, and include, e.g.: blood vessel and lymphatic vascular endothelial fenestrated cells, blood vessel and lymphatic vascular endothelial continuous cells, blood vessel and lymphatic vascular endothelial splenic cells, synovial cells (lining joint cavities, hyaluronic acid secretion), serosal cells (lining peritoneal, pleural, and pericardial cavities), squamous cells (lining perilymphatic space of ear), squamous cells (lining endolymphatic space of ear), columnar cells of endolymphatic sac with microvilli (lining endolymphatic space of ear), columnar cells of endolymphatic sac without microvilli (lining endolymphatic space of ear), dark cells (lining endolymphatic space of ear), vestibular membrane cells (lining endolymphatic space of ear), stria vascularis basal cells (lining endolymphatic space of ear), stria vascularis marginal cells (lining endolymphatic space of ear), cells of claudius (lining endolymphatic space of ear), cells of boettcher (lining endolymphatic space of ear), choroid plexus cells (cerebrospinal fluid secretion), pia-arachnoid squamous cells, pigmented ciliary epithelium cells of eye, nonpigmented ciliary epithelium cells of eye, and corneal endothelial cells.

Ciliated cells with propulsive function: Ciliated cells with propulsive function are present in various tissues in the body, as indicated, and include, e.g.: respiratory tract ciliated cells, oviduct ciliated cells (in female), uterine endometrial ciliated cells (in female), rete testis cilated cells (in male), ductulus efferens ciliated cells (in male), and ciliated ependymal cells of central nervous system (lining brain cavities).

Extracellular matrix secretion cells: Extracellular matrix secretion cells are present in various tissues in the body, as indicated, and include, e.g.: ameloblast epithelial cells (tooth enamel secretion), planum semilunatum epithelial cells of vestibular apparatus of ear (proteoglycan secretion), organ of corti interdental epithelial cells (secreting tectorial membrane covering hair cells), loose connective tissue fibroblasts, fibroblasts, tendon fibroblasts, bone marrow reticular tissue fibroblasts, other nonepithelial fibroblasts, blood capillary pericyte, nucleus pulposus cell of intervertebral disc, cementoblast /cementocyte (tooth root bonelike cementum secretion), odontoblast /odontocyte (tooth dentin secretion), hyaline cartilage chondrocyte, fibrocartilage chondrocyte, elastic cartilage chondrocyte, osteoblast/osteocyte, osteoprogenitor cell (stem cell of osteoblasts), hyalocyte of vitreous body of eye, stellate cell of perilymphatic space of ear, contractile cells, red skeletal muscle cell (slow), white skeletal muscle cell (fast), intermediate skeletal muscle cell, nuclear bag cell of muscle spindle, nuclear chain cell of muscle spindle, satellite cell (stem cell), ordinary heart muscle cell, nodal heart muscle cell, purkinje fiber cell, smooth muscle cell (various types), myoepithelial cell of iris, myoepithelial cell of exocrine glands, and red blood cells.

Blood and immune system cells: Blood and immune system cells are present in various tissues in the body, as indicated, and include, e.g.: erythrocytes (red blood cell), megakaryocytes (platelet precursor), monocytes, connective tissue macrophages (various types), epidermal langerhans cells, osteoclasts (in bone), dendritic cells (in lymphoid tissues), microglial cells (in central nervous system), neutrophil granulocytes, eosinophil granulocytes, basophil granulocytes, mast cells, helper t cells, suppressor t cells, cytotoxic t cells, b cells, natural killer cells, reticulocytes, stem cells and committed progenitors for the blood and immune system (various types). Sensory transducer cells: Sensory transducer cells are present in various tissues in the body, as indicated, and include, e.g.: photoreceptor rod cells of eye, photoreceptor blue-sensitive cone cells of eye, photoreceptor green-sensitive cone cells of eye, photoreceptor red-sensitive cone celsl of eye, auditory inner hair cells of organ of corti, auditory outer hair cells of organ of corti, i hair cells of vestibular apparatus of ear (acceleration and gravity), type ii hair cells of vestibular apparatus of ear (acceleration and gravity), type i taste bud cells, olfactory receptor neurons, basal cells of olfactory epithelium (stem cell for olfactory neurons), type i carotid body cells (blood ph sensor), type ii carotid body cells (blood ph sensor), merkel cells of epidermis (touch sensor), touch-sensitive primary sensory neurons (various types), cold-sensitive primary sensory neurons, -sensitive primary sensory neurons, pain-sensitive primary sensory neurons (various types), proprioceptive primary sensory neurons (various types); autonomic neuron cells such as Cholinergic neural cells (various types) Adrenergic neural cells (various types), Peptidergic neural cells (various types); Sense organ and peripheral neuron supporting cells such as Inner pillar cells of organ of Corti, Outer pillar cells of organ of Corti, Inner phalangeal cells of organ of Corti, phalangeal cells of organ of Corti, Border cells of organ of Corti, cells of organ of Corti, Vestibular apparatus supporting cells, Type I taste bud supporting cells, Olfactory epithelium supporting cells, Schwann cells, Satellite cells (encapsulating peripheral nerve cell bodies), Enteric glial cells; Central nervous system neurons and glial cells such as Neuron cells (variety of types), Astrocytes (various types), and Oligodendrocytes; Lens cells such as Anterior lens epithelial cells and Crystallin-containing lens fiber cells; Pigment cells such as Melanocytes and Retinal pigmented epithelial cells; Germ cells such as Oogonium/Oocytes, Spermatids, Spermatocytes, Spermatogonium cells (stem cells for spermatocytes), and Spermatozoons; and Nurse cells such as Ovarian follicle cells, Sertoli cells (in testis) and Thymus epithelial cells.

These cells are thus available for implantation and/or culture with proteins and various factors described herein, by using the methods set forth herein. In some embodiments, a method for correction of a defect in a human subject of a defect may comprise the steps of using mammalian cells by culturing a plurality of viable cells in vitro to expand the number of viable cells and to make in vitro cultured cells and/or ECM; and placing an effective volume of the in vitro cultured cells and/or protein into a tissue of the subject to treat the defect. As explained, such cells may include stem cells, embryonic stem cells, cells are cloned by somatic cell nuclear transfer, cell types transdifferentiated or otherwise converted into other cell types. Cells may be cultured as decribed herein, e.g., in medium containing autologous serum or in serum free medium.

In general, cell types, descriptions of cell types in tissues, and suitable cell and tissue culture techniques are available for the isolation and expansion of the cells, including these various cell types, primary cells, stem cells, and pluripotent cells e.g., in Atlas of Functional Histology, Kerr, J. B., Mosby, 1999, Gray's Anatomy: The Anatomical Basis of Clinical Practice, 39^{th} Edition, Standring, S., Ed., Elsevier, 2005, Culture of Animal Cells: A Manual of Basic Techniques, Freshney, R. I., ed., (Alan R. Liss & Co., New York 1987); Animal Cell Culture: A Practical Approach, Freshney, R. I. ed., (IRL Press, Oxford, England (1986); Culture of Animal Cells: A Manual of Basic Techniques, Freshney, R. I., Wiley-Liss, Inc., New York, 2000, and Methods in Molecular Biology Volume 290 Basic Cell Culture Protocols 3^{rd} Edition Cheryl D. Helgason and Cindy L. Miller Human Press Inc., Totowa, N.J., 2005, each of which are hereby incorporated herein by reference. Certain techniques for isolating and culturing some cell types, including fibroblasts, papillary and reticular fibroblasts are set forth in U.S. patent application Ser. Nos. 09/632,581 (filed Aug. 3, 2000) and 10/129,180 (filed May 3, 2002), which are hereby incorporated by reference herein. Isolation refers to obtaining a purified group of cells from a tissue sample. Expansion refers to increasing the number of cells. In general, expansion and differentiation are inversely related to each other, so that culture conditions that tend to differentiate the cells tend to suppress expansion. Enzymatic digestion of tissue or methods to start out with high numbers of cells extracted from tissue are preferred since these cells will be harvested for introduction into the subject with less cell doublings, thus avoiding the use of near senescent or senescent cells that may be harmful or not active in treating the defect.

The embodiments already described herein may be used in combination with materials and methods described in priority document PCT Application ______ filed Sep. 14, 2006 entitled “Compositions and Methods for the Augmentation and Repair of Defects in Tissue”.

All patents, patent applications, publications, journal articles, and publications mentioned herein are hereby incorporated by reference herein to the extent that the incorporated subject matter is not contradictory with the explicit disclosure herein. The elements for the various embodiments set forth herein may be combined with each other and mixed-and-matched as appropriate to obtain a functional embodiment.

1. A method of treating a tissue defect in a subject, comprising choosing the defect and
(a) introducing an effective amount of protein and/or (b), obtaining cells, expanding the cells in vitro, and placing the cells into the subject a composition comprising an effective quantity of the cells,
wherein the defect is a member of the group consisting of urological sphincter defects resulting in urinary incontinence, fecal incontinence, vesicoureteral reflux, gastroesophageal sphincter defects, gastroesophageal reflux, wrinkles, rhytids, depressed scar or other cutaneous depression, stretch marks, hypoplasia of the lip, prominent nasolabial fold, prominent melolabial fold, acne vulgaris scar, post-rhinoplasty irregularity, hypotrophic scar, hypertrophic scar, wounds, cellulite, skin laxness, aging skin, need for skin augmentation, and skin thinning, breast tissue deficiency, wounds, burns, hernias, periodontal disease, tendon tears, ligament tears, baldness, tissue mass adjustment, tissue or organ fibrosis or sclerosis, tissue scarring, tissue wounds, anal fissures, fistulas, hearing loss, bone defects, osteoporosis, osteomalacia, osteopenia, bone fractures, osteodystophy, bone metabolism defects, alveolar bone defects, cancer, cardiovascular disease, heart disease, arterial disease, venous disease, joint defects, cartilage defects, intervertebral disc defects, Alzheimer's disease, Parkinson's disease, neurological disease, spinal cord injury, spinal disc defects, hair graying, skin tanning, skin pigmentation, psoriasis, eczema, eye disease, cataracts, myopia, presbyopia, hyperopia, macular degeneration, eye muscle dysfunction, night vision, colorblindness, lacrimal gland dysfunction, interstitial lung disease, lung diseases, kidney dysfunction, renal osteodystrophy, liver dysfunction, dysfunctional pancreas, pancreatitis, diabetes mellitus, endocrine organ dysfunction, disease of a thyroid, parathyroid, hypothalamus, pituitary, adrenal, pineal, suprachiasmatic nucleus, or endocrine pancreas, immune system disorder, chronic inflammation, adhesions, fibroids, infections, taste or smell defects, gut defects, blood disorders, blood pressure, tooth growth, tissue cushioning, body thermoregulation, mechanical strength of tissues, foot enhancement, organ or tissue replacement, organ or tissue synthesis, and whole body rejuvenation.

2. The method of claim 1 wherein the cells are placed within or proximal to the tissue defect site.

3. The method of claim 1 wherein the cells are stem cells, cells taken from the subject at least five years before the placing of the cells into the subject, cells derived from tissue sources protected from light and chemical exposure, or fetal-derived cells.

4. The method of claim 1 wherein the cells are autologous cells.

5. The method of claim 4 wherein the autologous cells are expanded in culture medium comprising autologous serum.

6. The method of claim 5 wherein the autologus cells are free of contact with serum that is non-autologous.

7. The method of claim 4 further comprising introducing extracellular matrix into the subject with the autologous cells.

8. The method of claim 4 wherein the autologous cells are native to the tissue that is treated.

9. The method of claim 4 wherein (a) the defect is a bone defect caused by a bone resorption disease, and wherein the autologous cells comprise osteoblasts or osteoblast progenitor cells or (b) wherein the defect is a bone defect in a bone that is osteoporitic, broken, or fractured, and the autologous cells comprise bone cells or bone precursor cells.

10. The method of claim 9 wherein the autologous cells are placed into the defect by injecting the cells into a vein of the subject that flows through a body area having the defect.

11. The method of claim 4 wherein the defect is a bone defect caused by osteoporosis, osteopenia or osteomalacia, wherein the autologus cells comprise fibroblasts and the composition is placed into skin of the patient.

12. The method of claim 4 wherein the defect is an ear defect, and the autologous cells comprise hair cells of the cochlea or hair progenitor cells of the cochlea.

13. The method of claim 4 wherein the defect is an ear defect that is an abnormality of the patency and functionality of the Eustachian tube, further comprising introduction of the composition into a cartilaginous portion of the Eustachian tube.

14. The method of claim 4 wherein the defect is an eye disease defect and the autologous cells are (a) muscle cells and the composition is introduced into the eye to enhance a muscle of the eye, (b) lens cells introduced into the eye to restore a refraction error, (c) corneal fibroblasts, or (d) taken from an eye of the subject.

15. The method of claim 4 wherein the defect is an eye disease defect that (a) is macular degeneration and the composition is introduced into a retina of the eye. (b) includes a cataract and the autologous cells comprise ciliary muscle cells, (c) is strabismus and the autologous cells comprise muscle cells, (d) is glaucoma and the composition is placed into a sclera of the eye, or (e) is colorblindness or nightblindness and the autologous cells comprise rod-cells.

16. The method of claim 4 wherein the defect is an eye disease defect that is a vision defect affected by accommodation, wherein the autologous cells comprise fibroblasts, rod cells, progenitor cells to the rod cells, wound healing fibroblasts, myofibroblasts, Pericytes, retinal pigmented epithelial cells, or corneal epithelial cells.

17. The method of claim 4 wherein the defect is an eye disease defect that comprises eye trauma and the autologous cells comprise cells native to the injured area.

18. The method of claim 4 wherein the defect is an eye disease defect and is dry eye, and the autologous cells comprise tear gland cells, connective tissue cells, or keratocytes.

19. The method of claim 4 wherein the defect is a sphincter defect and the composition is introduced into the regions surrounding the external anal sphincter or the internal anal sphincter or directly into a pocket created in the region to be repaired or augmented and the composition comprises fibroblasts, smooth muscle cells, striated muscle cells, preadipocytes/adipoctes, or mesenchymal stem cells.

20. The method of claim 4 wherein the defect is an anal fissure and the autologous cells comprise fibroblasts.

21. The method of claim 4 wherein the defect is skin tanning and the autologous cells comprise melanocytes, melanoblasts, or progenitor cells or stem cells that produce melanocytes.

22. The method of claim 4 wherein the defect is hair graying and the autologous cells comprise melanocytes can be obtained from non-greying hair follicles, melanoblasts, melanocyte stem cells, or progenitor cells to melanocytes.

23. The method of claim 4 wherein the defect is psoriasis or eczema and the autologous cells comprise (a) papillary fibroblasts from skin tissue taken from an unaffected skin site and the composition is implanted into the upper dermis, (b) fibroblasts or progenitor cells to fibroblasts and the composition is placed into the dermis or a subcutaneous layer, (c) immune cells or progenitor immune cells.

24. The method of claim 4 wherein the defect is a tooth defect or alveolar bone defect.

25. The method of claim 4 wherein the defect is a foot enhancement wherein the composition is introduced to a natural fat pad that overlays the calcaneal bone in a heel of the subject.

26. The method of claim 4 wherein the defect is a heart defect and the autologous cells are obtained from the group consisting of pericardium, outer fibrous layers, inner parietal layers, pericardial cavity, epicardium, myocardium, heart muscle fibers, endocardium, papillary muscles, and muscles that assist opening and shutting of heart valves.

27. The method of claim 4 wherein the defect is a blood vessel defect and the autologous cells comprise endothelial cells, endothelial precursor cells, or pericytes and the composition is used to a blood vessel to produce new vasculature or to repair vasculature.

28. The method of claim 4 wherein the defect is a blood vessel defect and the autologous cells comprise fibroblasts or smooth muscle cells and the composition is introduced to a damaged blood vessel valve.

29. The method of claim 4 wherein the defect is an atherosclerotic plaque, the autologous cells comprise fibroblasts, macrophages, or smooth muscle cells, and the composition is introduced into the vascular media and/or vascular intima proximal to the plaque.

30. The method of claim 4 wherein the defect is a blood vessel defect previously treated at a site with a coronary stent, angioplasty, clot removal, or plaque removal, the autologous cells comprise connective tissue cells, smooth muscle cells, or fibroblasts and the composition is introduced at the site.

31. The method of claim 4 wherein the defect is a lung defect and (a) the autologous cells are fibroblasts, with the composition being introduced into the lung to reduce fibrosis or scar tissue, or (b) the autologous cells are alveolar cells that produce a lung surfactant, with the composition being introduced into lung tissue.

32. The method of claim 4 wherein the defect is a kidney defect and (a) the autologous cells comprise mesangial cells and/or macula densa cells and/or juxtaglomerular cells, with the composition being placed into a renal corpuscle to increase nephron functioning or to increase nephron number, or (b) the autologous cells comprise podocytes or epithelial cells of the parietal layer and the composition is introduced to the Bowman's capsule, wherein (a) or (b) treats a glomerular filtration rate, regulates blood pressure, regulates electrolyte balance abnormalities, or treats deficiencies in urine concentration.

33. The method of claim 4 wherein the defect is a kidney defect and the autologous cells comprise fibroblasts or mesangial cells to remove fibrosis or sclerosis of the glomerulus to improve glomerular function.

34. The method of claim 4 wherein the defect is a kidney defect and the autologous cells comprise epithelial cells of a distal convoluted tube, and the composition is introduced into a distal convoluted tube to improve resorption function.

35. The method of claim 4 wherein the defect is a kidney defect and the autologous cells comprise renal cells and the composition is introduced into a cortex or medulla to produce erythropoietin to increase red blood cell production from bone marrow.

36. The method of claim 4 wherein the defect is Alzheimer's disease and the autologous cells are neuroglial cells, astrocytes, or immune cells.

37. The method of claim 4 wherein the defect is Parkinson's disease and the autologous cells comprise cells that secrete dopamine, retinal pigment epithelial cells, carotid cell bodies, sympathoadrenal cells, sympathetic neurons, sympathetic neurons, chromaffm cells of the adrenal medulla extra-adrenal paraganglia cells, glial cells, or astrocytes.

38. The method of claim 4 wherein the defect is a spinal cord injury, and the autologous cells comprise mesenchymal stem cells, mesenchymal cells and/or glial cells that promote neuronal guidance and repair with the composition being introduced proximal to the lesion.

39. The method of claim 4 wherein the defect is multiple sclerosis, the autologous cells comprise oligodendrocytes, with the composition being introduced proximal to demyelinated nerves.

40. The method of claim 4 wherein the defect is a liver defect and (a) the autologous cells comprise hepatocytes, hepatic stellate cells, or fibroblasts with the composition being implanted into a liver parenchyma, (b) the autologous cells comprise with hepatic stellate cells, fibroblasts or myofibroblasts and the composition is implanted into a liver tissue scar, or (c) the autologous cells comprise cells transfected with coagulation proteins and the composition is introduced to a liver.

41. The method of claim 4 wherein the defect is a pancreatic defect and (a) the autologous cells comprise pancreatic stellate cells or fibroblasts, with the composition being introduced into fibrotic areas to remove tissue scars, (b) the autologous cells comprise epithelial cells and the composition is introduced into the ductule or tubular duct system, (c) the autologous cells comprise β cells isolated from islets or ductile system of the pancreas, with the composition being introduced into islets, an exocrine region of the pancreas, or a liver parenchyma.

42. The method of claim 4 wherein the defect is an immune defect and the autologous cells comprise immune cells, with the composition being introduced in a brain parenchyma or associated vasculature to degrade amyloid plaque or neurofibrillary tangles.

43. The method of claim 4 wherein the defect is an immune defect and (a) the autologous cells comprise thymocytes, with the composition being introduced into a thymus, or (b) the autologous cells comprise endothelial cells, EPCs, or pericytes to enhance angiogenesis in the tissue.

44. The method of claim 4 wherein the defect is an infection and the autologous cells comprise immune cells or fibroblasts, with the composition being introduced into the infection.

45. The method of claim 4 wherein the defect is chronic inflammation and (a) the autologous cells comprise fibroblasts, with the composition being introduced into inflamed tissue, or (b) the autologous cells comprise fibroblasts, with the composition being introduced into a rheumatoid arthritis joint.

46. The method of claim 4 wherein the defect is tissue fibrosis or a fibroid and the autologous cells comprise fibroblasts, with the composition being introduced into a fibrotic tissue or fibroid.

47. The method of claim 4 wherein the defect is the endocrine system and the autologous cells comprise hormone secreting cells and/or fibroblasts, with the composition being introduced into a hormonal tissue.

48. The method of claim 4 wherein the defect is cancer and the autologous cells comprise cancer cells and/or the extracellular matrix of the cancer cells.

49. The method of claim 4, wherein the defect is a deficiency caused by aging chosen from the group consisting of tissue dysfunction, tissue dystrophy, laxness, thinning, loss of elasticity, altered protein profile, diminished tissue mass, decreased amounts of extracellular matrix, decreased proteoglycan, decreased tissue turgor, increased amounts of protease activity, loss of cell numbers, decreased tissue moisture, decreased thermoregulation, decreased cushioning, or decreased mechanical strength.

50. The method of claim 49 comprising the protein, wherein the protein is an extcellular matrix molecule.

51. The method of claim 4 wherein the defect is an adhesion and the autologous cells comprise fibroblasts or endothelial cells, with the composition being introduced at or near a site of an adhesion.

52. The method of claim 4 wherein the defect is anemia and the autologous cells comprise renal peritubular endothelial cells with the composition being introduced to a kidney.

53. The method of claim 4 wherein the defect is degeneration, rupture, herniation or atrophy of an intervertebral disc wherein the autologous cells comprise chondrocytes, chondrocyte precursors, perichondrium chondrocytes, or fibroblasts.

54. The method of claim 4 wherein the defect is a fistula and the autologous cells comprise fibroblasts.

55. The method of claim 4 wherein the defect is in a gut and the autologous cells comprise stem cells that produce lactase or precursors to parietal cells that absorb vitamin B12.

56. The method of claim 4 wherein the defect is related to aging and the autologous cells comprise bone marrow progenitor cells introduced into bone marrow to increase a number of native bone marrow progenitor cells.

57. The method of claim 4 wherein the defect is gastroesophogeal reflux disease and the autologous cells comprise fibroblasts, smooth muscle cells, striated muscle cells, preadipocytes/adipoctes, or mesenchymal stem cells with the composition being introduced to an esophageal sphincter.

58. The method of claim 4, with the composition comprising an in vitro preparation of the autologous cells and an immunogenic cell-absorbable protein.

59. The method of claim 58, wherein the protein is a recombinant protein, soluble protein, insoluble protein, in a gellable solution, an extracellular matrix molecule, a serum protein, albumin, a growth factor, a hormone, a cytokine, a chemokine, a cell adhesion protein, or a non-autologous protein.

60. The method of claim 58, wherein the protein is used during the culture of the cells or is added to the cells after culturing of the cells is completed.

61. The method of claim 58, wherein the protein is an apoptosis inhibiting protein, an anoikis inhibiting protein, an angiogenesis protein, a vasodilator protein, a pro-inflammatory protein, a filler or augmenting protein, a differentiation protein, a cell mitogen, a promoter of extracellular matrix production, a chemoattractant, a cell culture medium serum-derived protein, a procoagulation protein, a transport protein, or a protease inhibiting factor.

62. The method of claim 4, with the composition comprising an apoptosis inhibiting protein, an anoikis inhibiting protein, a protease inhibiting factor, a transport protein, a procoagulation protein, a cell mitogen, a differentiation protein, a filler or augmenting protein, a pro-inflammatory protein, a vasodilator protein, an angiogenesis protein, a chemoattractant, a vasodilator, a promoter of ECM production, a cell proliferation protein, a differentiation protein, or a cell culture medium serum-derived protein.
